(19) United States
(12) Patent Application Publication
(10) Pub. No.: US 2023/0158084 A1
(43) Pub. Date: May 25, 2023

(54) PROBIOTICS, METABOLITES, AND USES THEREOF

(71) Applicant: California Institute of Technology, Pasadena, CA (US)

(72) Inventors: Gil SHARON, Los Angeles, CA (US); Sarkis K MAZMANIAN, Glendale, CA (US)

(21) Appl. No.: 16/959,998

(22) PCT Filed: Jan. 4, 2019
(86) PCT No.: PCT/US2019/012297

§ 371 Date: Jul. 2, 2020

Related U.S. Application Data
(60) Provisional application No. 62/614,187, filed on Jan. 5, 2018.

(51) Int. Cl. A61K 35/741; A61K 31/198; C12N 1/20

(52) U.S. Cl. CPC A61K 35/741; A61K 31/198; C12N 1/20

(57) ABSTRACT

Some embodiments herein relate generally to compositions comprising microbial organisms and/or metabolites for improving the behavior of a subject, such as a subject having autism spectrum disorder (ASD), and methods of using the same. Some embodiments herein relate generally to profiles of gut bacteria and/or metabolites useful for determining a risk, presence, and/or severity of ASD.

### INCORPORATION BY REFERENCE TO ANY PRIORITY APPLICATIONS

This application claims the benefit of U.S. Provisional App. No. 62/614,187, filed Jan. 5, 2018, which is hereby incorporated by reference in its entirety. Any and all applications for which a foreign or domestic priority claim is identified in the Application Data Sheet as filed with the present application are hereby expressly incorporated by reference under 37 CFR 1.57.

### REFERENCE TO SEQUENCE LISTING IN ELECTRONIC FORMAT

This application is filed with an electronic sequence listing entitled CALTE127WOSEQLIST.txt, created on Jan. 3, 2019 which is 10,672 bytes in size. The information in the electronic sequence listing is hereby incorporated by reference in its entirety.

### FIELD

Some embodiments of the present invention described herein relate to compositions comprising microbial organisms and/or metabolites useful in methods for improving the behavior of a subject, including a subject that is identified as having autism spectrum disorder (ASD) or a subject selected as a member of a population of subjects that present one or more symptoms or conditions associated with ASD. Additional embodiments described herein concern profiles of gut bacteria and/or metabolites useful for determining a risk, presence, and/or severity of ASD.

### BACKGROUND

Autism spectrum disorders (ASD) are a group of neurodevelopmental conditions with a broad range of manifestations involving altered social communication and interaction, as well as repetitive, stereotyped behaviors. The prevalence of ASD in the US, as of 2012, is 14.6 per 1,000 children (Christensen et al., 2016), with 1 million cases currently and evidence that diagnoses are rising (Fombonne, 2009; King and Bearman, 2009). ASD heritability has been estimated as high as 83%, and the non-shared environmental influence at 17% (Sandin et al., 2017). The need for therapies for these disorders is manifest.

### SUMMARY

Some embodiments include a composition or product combination comprising (a) a bacteria selected from the group consisting of: Bacteriodetes, Anaerofilum, Anaerotruncus, Christensenella, Pseudoramibacter eubacterium, Holdemania, Clostridiales, and a mixture of two or more of the listed bacteria, and (b) a taurine precursor and/or a 5-Aminovaleric acid (5AV) precursor. Components (a) and (b) can be provided in the same formulation or can be provided in separate formulations in a product combination. In some embodiments, the bacteria is selected from the group consisting of Bacteroides, Butyricimonas, Paraprevotallacae, and a mixture of two or more of the listed bacteria. In some embodiments, the bacteria is selected from the group consisting of Bacteriodetes, Anaerqfilum, Anaerotruncus, Christensenella, Pseudoramibacter eubacterium, Holdemania, and a mixture of two or more of the listed bacteria. In some embodiments, the bacteria is selected from the group consisting of Bacteroidetes, Holdemania, and a mixture of two or more of the listed bacteria. In some embodiments, the bacteria comprises, consists essentially of, or consists of a Bacteroidetes selected from the group consisting of Bacteroidaceae, Paraprevotellacae, Banesiellaceae, Rikenellaceae, Odoribacteraceae, Bacteroides, Butyricimonas, and a mixture of two or more of the listed bacteria. In some embodiments, the bacteria comprises, consists essentially of, or consists of a bacteria selected from the group consisting of Bacteroides ovatus, Parabacteroides merdae, and Bacteroides thetaiotaomicron, and a mixture of two or more of the listed bacteria. In some embodiments, the bacteria comprises, consists essentially of, or consists of a Clostridiales selected from the group consisting of Lachnospiraceae and Clostridium. In some embodiments, the composition or product combination does not comprise any bacteria other than Bacteriodetes, Anaerofilum, Anaerotruncus, Christensenella, Pseudoramibacter eubacterium, Holdemania, and/or Clostridiales. In some embodiments, the composition or product combination does not comprise any bacteria of Eggerthella, Alstipes, Burkolderiales, Enterococcaceae, Clostridium, or Ruminococcus. In some embodiments, the composition or product combination does not comprise any bacteria of Ruminococcaceace, Oscillospira, and Suterella. In some embodiments, the composition or product combination does not comprise Eisenbergiela tayi. In some embodiments, the composition or product combination comprises the taurine precursor and the 5AV precursor. In some embodiments, the taurine precursor is selected from the group consisting of: cysteine, cysteine sulfinic acid, homocysteine, cystathionine, hypotaurine, and a mixture of two or more of the listed items. In some embodiments, the 5AV precursor is selected from the group consisting of: lysine, cadaverine, 1-piperideine, and a mixture of two or more of the listed items. In some embodiments, the composition or product combination comprises Bacteroides ovatus and Parabacteroides merdae. In some embodiments, the composition or product combination does not comprises any bacteria other than Bacteroides ovatus and/or Parabacteroides merdae. In some embodiments, the composition or product combination comprises Bacteroides ovarus and Bacteroides thetaiotaomicron. In some embodiments, the composition or product combination does not comprises any bacteria other than Bacteroides ovatus and/or Bacteroides thetaiotaomicron. In some embodiments, the composition or product combination comprises Parabacteroides merdae and Bacteroides thetaiotaomicron. In some embodiments, the composition or product combination does not comprises any bacteria other than Parabacteroides merdae and/or Bacteroides thetaiotaomicron. In some embodiments, the composition or product combination comprises Bacteroides ovatus, Parabacteroides merdae, and Bacteroides thetaiotaomicron. In some embodiments, the composition or product combination does not comprises any bacteria other than Bacteroides ovatus, Parabacteroides merdae, and/or Bacteroides thetaiotaomicron. In some embodiments, the composition or product combination further comprises Lactobacillus reuteri. In some embodiments, the composition or product combination consists essentially of the bacteria; and the taurine precursor and/or the 5AV precursor. In some embodiments, the composition or product combination as described herein is for use in reducing one or more symptoms of Autism Spectrum Disorder (ASD) in a subject after birth, in which said composition or product combination is administered to said subject prenatally. In some embodiments, the said composition or product combination is administered directly to said subject prenatally. In some embodiments, said composition or product combination is administered to the mother of said subject prenatally, thereby administering said composition or product combination prenatally. In some embodiments, the composition or product combination is administered to the mother in a single administration. In some embodiments, the composition or product combination is administered to the mother via multiple administrations over a period of time. In some embodiments, the composition or product combination is for use as described herein, and the one or more symptoms of ASD is selected from the group consisting of: repetitive behavior, hyperactivity, anxiety, and a communication disorder. In some embodiments, the composition or product composition comprises the bacteria in amount sufficient to establish a colony in the gut of a human subject when administered for microbiome transplant or probiotic treatment. In some embodiments, the colony persists in the gut for at least 1, 2, 3, 4 or more weeks post-inoculation. In some embodiments, the composition or product combination does not comprise an antibiotic.

Some embodiments include a method of reducing or preventing a symptom of ASD in a selected prenatal subject after birth. The method can comprise administering a composition or product combination comprising an amount of taurine and/or 5AV to a subject prenatally. The amount can be effective to reduce or prevent the symptom of ASD in the subject after birth. In some embodiments, the composition or product combination comprises the taurine and the 5AV. In some embodiments, the symptom of ASD comprises a sociability disorder, anxiety, and/or a repetitive behavior. In some embodiments, the prenatal subject is selected as one being at risk of developing ASD or a symptom of ASD. In some embodiments, at the time of administration, the blood-brain barrier of the prenatal subject is permeable to the taurine and/or 5AV. In some embodiments, the composition or product combination is administered to the mother of the subject. In some embodiments, the prenatal subject is selected as being at risk of developing ASD or a symptom of ASD due to the mother of the prenatal subject having a sample, preferably a fecal sample, comprising: a reduced level of taurine and/or 5AV compared to a sample from control mother of a non-ASD offspring; and/or an elevated level of 3-aminoisobutyric acid (3AIBA) compared to a sample from the control mother of a non-ASD offspring. In some embodiments, the prenatal subject or the mother of the prenatal subject is selected as having: reduced levels of taurine and/or 5AV compared to a non-ASD control or a mother of a non-ASD control; and/or elevated levels of 3-aminoisobutyric acid (3AIBA) compared to a non-ASD control or a mother of a non-ASD control. In some embodiments, the composition or product combination comprising an amount of taurine and/or 5AV is administered to the prenatal subject or the mother of the prenatal subject in multiple administrations over a period of time, for example, daily, weekly, biweekly, or monthly.

Some embodiments include a method of reducing a symptom of ASD in a selected subject, the method comprising administering a composition or product combination (e.g., more than one composition) comprising bacteria selected from the group consisting of: Bacteriodetes, Anaerofilum, Anaerotruncus, Christensenella, Pseudoramibacter eubacterium, Holdemania, Clostridiales, and a mixture of two or more of the listed bacteria to the subject, so that the symptom of ASD is reduced in the subject after birth. In the method of some embodiments, the composition or product combination does not comprise any bacteria other than Bacteriodetes, Anaerofilum, Anaerotruncus, Christensenella, Pseudoramibacter eubacterium, Holdemania, and/or Clostridiales. In the method of some embodiments, the composition or product combination comprises a bacteria selected from the group consisting of Bacteroides ovatus, Parabacteroides merdae, and Bacteroides thetaiotaomicron, and a mixture of two or more of the listed bacteria. In the method of some embodiments, the composition or product combination is substantially free of Eisenbergiela tayi. In the method of some embodiments, the composition or product combination further comprises a taurine precursor and/or a 5-Aminovaleric acid (5AV) precursor. In the method of some embodiments, a taurine precursor and/or a 5-Aminovaleric acid (5AV) precursor is administered to the subject prenatally (e.g., to the subject's mother, or directly to the subject prenatally), and the bacteria is administered to the subject after birth. In the method of some embodiments, a taurine precursor and/or a 5-Aminovaleric acid (5AV) precursor and the bacteria are administered to the subject prenatally (e.g., to the subject's mother, or directly to the subject prenatally), either in separate compositions, or in a single composition. In the method of some embodiments, the composition or product combination comprises a bacteria selected from the group consisting of Bacteroides, Butyricimonas, Paraprevotallacae, and a mixture of two or more of the listed bacteria. In the method of some embodiments, the composition or product combination comprises a bacteria selected from the group consisting of Bacteriodetes, Anaerofilum, Anaerotruncus, Christensenella, Pseudoramibacter eubacterium, Holdemania, and a mixture of two or more of the listed bacteria. In the method of some embodiments, the composition or product combination comprises a bacteria selected from the group consisting of Bacteroidetes, Holdemania, and a mixture of two or more of the listed bacteria. In the method of some embodiments, the bacteria of the composition or product combination comprises a Bacteroidetes selected from the group consisting of Bacteroidaceae, Paraprevotellacae, Banesiellaceae, Rikenellaceae, Odoribacteraceae, Bacteroides, Butyricimonas, and a mixture of two or more of the listed bacteria. In the method of some embodiments, the composition or product combination comprises a Bacteroidetes selected from the group consisting of: Bacteroides ovatus, Parabacteroides merdae, and Bacteroides thetaiotaomicron, and a mixture of two or more of the listed bacteria. In the method of some embodiments, the composition or product combination comprises a bacteria selected from the group consisting of: Bacteroides ovatus, Parabacteroides merdae, and Bacteroides thetaiotaomicron. In the method of some embodiments, the composition or product combination comprises a bacteria comprising a Clostridiales selected from the group consisting of Lachnospiraceae and Clostridium. In some embodiments, the method further comprises administering Lactobacillus reuteri to the subject. In some embodiments, the composition or product combination further comprises Lactobacillus reuteri. In the method of some embodiments, the composition or product combination does not comprise any bacteria of Eggerthella, Alstipes, Burkolderiales, Enterococcaceae, Clostridium, or Ruminococcus. In the method of some embodiments, the composition or product combination does not comprise any bacteria of Ruminococcaceace, Oscillospira, and Suterella. In some embodiments, the administering comprises colonizing a region of the subject's gastrointestinal tract. In some embodiments, the administering comprises one or more fecal transplants. In some embodiments, the composition or product combination is stabilized. In some embodiments, the composition or product combination is administered prenatally. In some embodiments, the composition or product combination is administered directly to the selected subject prenatally. In some embodiments, the composition or product combination is administered to the mother of the selected subject prenatally, thereby administering said composition or product combination to the selected subject prenatally. In some embodiments, said subject is selected as one being at risk of developing ASD or a symptom of ASD. In some embodiments, said subject has a colon sample showing at least: reduced levels of taurine and/or 5AV compared to a non-ASD control; and/or elevated levels of 3-aminoisobutyric acid (3AIBA) compared to the non-ASD control, and is thus selected as being at risk of developing ASD or a symptom of ASD. In some embodiments, the mother of the subject has a colon sample showing at least: reduced levels of taurine and/or 5AV compared to a control mother of a non-ASD offspring; and/or elevated levels of 3-aminoisobutyric acid (3AIBA) compared to the control mother of a non-ASD offspring, and the subject is thus selected as being at risk of developing ASD or a symptom of ASD. In some embodiments, the composition or product combination administered to the subject comprises bacteria in amount sufficient to establish a colony in the gut of the subject when administered for microbiome transplant or probiotic treatment. In some embodiments, the colony persists for at least 1, 2, 3, 4 or more weeks post-inoculation.

Some embodiments include a method of determining a profile of a sample of a subject, the method comprising detecting at least one of: (a) a presence and/or level of a gut bacterium selected from the group consisting of: Bacteroides ovatus, Parabacteroides merdae, Bacteroides thetaiotaomicron, or a combination of two or more of the listed bacteria; and a presence and/or level of Eisenbergiela tayi in the gut; (b) a presence or gene product level of a gut microbiota gene that is an ortholog of KEGG ortholog K0681, and/or a presence or gene product level of a gene that is an ortholog of KEGG ortholog K1442, wherein the sample comprises gut, feces, or gut and feces material of the subject; (c) a level of colon taurine, 5-Aminovaleric acid (5AV), lysine, 3-aminoisobutyric acid (3-AIBA), genistein, daidzein, lysine, 5-aminopentanoate, cellobiose, glyceric acid, a level of serum D ribose, ribitol, ribonic acid, L-tyrosine, or Lipocalin-2 (LCN2) or a rate of degradation thereof; (d) a level of a product of a gene of the subject selected from the group consisting of: Gm26944 (or an ortholog thereof), Gm13016 (or an ortholog thereof), Gm17259 (or an ortholog thereof), 4930539E08Rik (or ortholog thereof), Dag1b (or an ortholog thereof), a human ortholog of any of the listed genes, or a combination of two or more of the listed genes; and/or (e) a level of a splice variant of the subject selected from the group consisting of: a mutually exclusive exon in Neurexin 2; a mutually exclusive exon in Ankryin 2; a skipped exon in Fmr1; a skipped exon in Ube3a; a skipped exon in Rims1; a skipped exon of Cacna1c; a retained intron of Ads1; a skipped exon of a pogo transferrable element derived with ZNF domain Pogz; or a skipped exon of Trip12. The profile can comprise the detected presence and/or levels of (a), (b), (c), (d), (e), or a combination of two or more of (a), (b), (c), (d), and (e). In some embodiments, the profile comprises, consists essentially of, or consists of (a) and (b). In some embodiments, the profile comprises, consists essentially of, or consists of (a) and (c). In some embodiments, the profile comprises, consists essentially of, or consists of (a) and (d). In some embodiments, the profile comprises, consists essentially of, or consists of (a) and (e). In some embodiments, the profile comprises, consists essentially of, or consists of (b) and (c). In some embodiments, the profile comprises, consists essentially of, or consists of (b) and (d). In some embodiments, the profile comprises (b) and (e). In some embodiments, the profile comprises, consists essentially of, or consists of (c) and (d). In some embodiments, the profile comprises, consists essentially of, or consists of (c) and (e). In some embodiments, the profile comprises, consists essentially of, or consists of (d) and (e). In some embodiments, the profile comprises, consists essentially of, or consists of (a), (b), and (c). In some embodiments, the profile comprises, consists essentially of, or consists of (a), (b), and (d). In some embodiments, the profile comprises, consists essentially of, or consists of (a), (b), and (e). In some embodiments, the profile comprises, consists essentially of, or consists of (a), (c), and (d). In some embodiments, the profile comprises, consists essentially of, or consists of (a), (c), and (e). In some embodiments, the profile comprises, consists essentially of, or consists of (a), (d), and (e). In some embodiments, the profile comprises, consists essentially of, or consists of (b), (c), and (d). In some embodiments, the profile comprises, consists essentially of, or consists of (b), (c), and (e). In some embodiments, the profile comprises, consists essentially of, or consists of (b), (d), and (e). In some embodiments, the profile comprises, consists essentially of, or consists of (c), (d), and (e). In some embodiments, the profile comprises, consists essentially of, or consists of (a), (b), (c) and (d). In some embodiments, the profile comprises, consists essentially of, or consists of (a), (b), (c) and (e). In some embodiments, the profile comprises, consists essentially of, or consists of (b), (c), (d) and (e). In some embodiments, the profile comprises, consists essentially of, or consists of (a), (c), (d), and (e). In some embodiments, the profile comprises, consists essentially of, or consists of (a), (b), (d), and (e). In some embodiments, the profile comprises, consists essentially of, or consists of (a), (b), (c), (d), and (e). In some embodiments, the subject is human. In some embodiments, determining the profile comprises determining (a), wherein the sample comprises gut and/or feces material of the subject, and wherein elevated risk of ASD is indicated by reduced levels of Bacteroides ovatus, Parabacteroides merdae, Bacteroides thetaiotaomicron; or increased levels of Eisenbergiela tayi, relative to levels present in a non-ASD control subject. In some embodiments, the profile comprises detecting (b), in which the sample comprises gut and/or feces material of the mother of the subject, and wherein elevated risk of ASD is indicated by increased levels of the gut microbiota gene that is the ortholog of KEGG ortholog K0681 and/or decreased levels of the gene that is the ortholog of KEGG ortholog K01442 relative to levels present in a non-ASD control subject. In some embodiments, (c) comprises a level of colon taurine, 5-Aminovaleric acid (5AV), lysine, 3-aminoisobutyric acid (3-AIBA), genistein, daidzein, lysine, 5-aminopentanoate, cellobiose, glyceric acid, a level of serum D ribose, ribitol, ribonic acid, L-tyrosine, or a rate of degradation thereof. In some embodiments, determining the profile comprises detecting (c), in which the sample comprises colon contents of the subject and/or serum of the subject, and in which colon levels of taurine, 5AV, 5-aminopentanoate, or cellobiose, below a non-ASD control, serum levels of ribitol or L-tyrosine below a non-ASD control, colon levels of 3AIBA, lysine, glyceric acid, genistein, and/or daidzein above a non-ASD control; and/or serum levels of D ribose and/or ribonic acid and/or LCN2 above a non-ASD control indicate an increased risk of developing and/or severity of ASD in the subject. In some embodiments, determining the profile comprises detecting (c), in which the sample comprises colon contents of the subject and/or serum of the subject, and in which colon levels of taurine, 5AV, 5-aminopentanoate, or cellobiose, below a non-ASD control, serum levels of ribitol or L-tyrosine below a non-ASD control, colon levels of 3AIBA, lysine, glyceric acid, genistein, and/or daidzein above a non-ASD control, and/or serum levels of D ribose or ribonic acid above a non-ASD control indicate an increased risk of developing and/or severity of ASD in the subject. In some embodiments, determining the profile comprises detecting (c), in which the sample comprises colon contents of the subject, and in which colon levels of taurine, 5AV, 5-aminopentanoate, or cellobiose below a non-ASD control, and/or colon levels of 3AIBA, lysine, glyceric acid, genistein, and/or daidzein above a non-ASD control indicate an increased risk of developing and/or severity of ASD in the subject. In some embodiments, determining the profile comprises detecting (c), in which the sample comprises serum of the subject, and in which serum levels of ribitol or L-tyrosine below a non-ASD control, and/or serum levels of D ribose or ribonic acid above a non-ASD control indicate an increased risk of developing and/or severity of ASD in the subject. In some embodiments, determining the profile comprises determining (d), and wherein levels of Gm26944, Gm13016, and/or Gm17259 gene product greater than a non-ASD control, and/or levels of 4930539E08Rik and Dag1b, or a human ortholog of any of the listed genes below a non-ASD control indicate a presence or elevated risk of ASD. In some embodiments, determining the profile comprises determining (e), and wherein levels of Gm26944, Gm13016, Gm17259, 4930539E08Rik, and Dag1b gene products or human orthologs thereof are determined. In some embodiments, determining the profile comprises determining (e), and levels of the mutually exclusive exon in Neurexin 2 above a non-ASD control; levels of the mutually exclusive exon in Ankryin 2 below the non-ASD control; levels of the skipped exon of Cacna1c above the non-ASD control; levels of the retained intron of Ads1 above a non-ASD control; and/or levels of the skipped exon of the pogo transferrable element above a non-ASD control indicate an increased risk of ASD. In some embodiments, determining the profile comprises determining (e), and levels of the mutually exclusive exon in Neurexin 2 above a non-ASD control; levels of the mutually exclusive exon in Ankryin 2 below the non-ASD control; levels of the skipped exon of Cacna1c above the non-ASD control; levels of the retained intron of Ads1 above a non-ASD control; and/or levels of the skipped exon of the pogo transferrable element above a non-ASD control indicate an increased severity of ASD. In some embodiments, the method detects ASD or a symptom of ASD, predicts a risk of ASD and/or a symptom of ASD, and/or predicts the severity of ASD in the subject. In some embodiments, the sample for (a), (b), and/or (c) comprises a gut or fecal sample of the subject. In some embodiments, the sample for (d) and/or (e) comprises a cerebrospinal fluid (CSF; it is noted that CSF may also be referred to as “cerebral spinal fluid,” and the term “cerebral spinal fluid” shall also be understood herein to refer to CSF) or central nervous system (CNS) tissue sample, such as prefrontal cortex (PFC) and/or striatum (STR). In some embodiments, the sample comprises colon contents of the subject. In some embodiments, the method further comprises determining the subject as having or being at risk of developing ASD based on (a), (b), (c), (d), and/or (e). In some embodiments, the method further comprises prenatally increasing a level of taurine and/or 5AV in the subject. In some embodiments, the level of taurine and/or 5AV is prenatally increased by administering taurine and/or 5AV to the mother of the subject. In some embodiments, the level of taurine and/or 5AV is prenatally increased by administering a composition or product combination comprising Bacteroides ovatus, Parabacteroides merdae, and/or Bacteroides thetaiotaomicron to the mother of the subject. In some embodiments, the level of taurine and/or 5AV is prenatally increased by administering a composition or product combination comprising Bacteroides ovatus, Parabacteroides merdae, and/or Bacteroides thetaiotaomicron to the subject. In some embodiments, the method comprising prenatally administering a composition or product combination comprising a precursor of taurine and/or 5AV to the subject, thus increasing the level of taurine and/or 5AV in the subject. In some embodiments, the profile comprises a level of a product of a gene of the subject selected from the group consisting of: Gm26944 (or an ortholog thereof), Gm13016 (or an ortholog thereof), Gm17259 (or an ortholog thereof), 4930539E08Rik (or ortholog thereof), Dag1b (or an ortholog thereof), a human ortholog of any of the listed genes, or a combination of two or more of the listed genes; and/or level of a splice variant of the subject selected from the group consisting of: a mutually exclusive exon in Neurexin 2; a mutually exclusive exon in Ankryin 2; a skipped exon in Fmr1; a skipped exon in Ube3a; a skipped exon in Rims1; a skipped exon of Cacna1c; a retained intron of Ads1; a skipped exon of a pogo transferrable element derived with ZNF domain Pogz; or a skipped exon of Trip12. In some embodiments, the method is an in vitro method. In some embodiments, the level of the gene product or splice variant is determined in central nervous system tissue of the subject in vivo, for example by fMRI or PET.

In some embodiments, the level of taurine and/or 5AV is prenatally increased by administering a composition or product combination as described herein to the mother of the subject. In some embodiments, the level of taurine and/or 5AV is prenatally increased by administering a composition or product combination as described herein to the prenatal subject. In some embodiments, the method comprises administering to the subject a composition or product combination comprising, consisting essentially of, or consisting of bacteria selected from the group consisting of: Bacteriodetes, Anaerofilum, Anaerotnrncus, Christensenella, Pseudormmibacter eubacterium, Holdemania, Clostridiales, and a mixture of two or more of the listed bacteria, when determining the profile indicates an increased risk or severity of ASD. In some embodiments, the composition or product combination comprises, consists essentially of, or consists of bacteria selected from the group consisting of Bacteroides, Butyricimonas, Paraprevotallacae, and a mixture of two or more of the listed bacteria. In some embodiments, the composition or product combination comprises, consists essentially of, or consists of a bacteria comprising a Bacteroidetes selected from the group consisting of: Bacteroides ovatus, Parabacteroides merdae, and Bacteroides thetaiotaomicron, and a mixture of two or more of the listed bacteria. In some embodiments, the composition or product combination comprises, consists essentially of, or consists of Bacteroides bacteria. In some embodiments, the Bacteroides bacteria comprises, consists essentially of, or consists of B. fragilis. In some embodiments, the composition does not comprise E. tayi. In some embodiments, the composition does not comprise any of Eggerthella, Alstipes, Burkolderiales, Enterococcaceae, Clostridium, or Ruminococcus. In some embodiments, the method further comprises administering Lactobacillus reuteri to the subject, for example as part of the composition or product combination, or separately.

Some embodiments include a composition or product combination comprising a) a bacteria that maps to an sOTU selected from the group consisting of: b20cd_Bacteroides, and 4ae7e_Parabacteroides; and b) a taurine precursor and/or a 5-Aminovaleric acid (5AV) precursor. Components (a) and (b) can be provided in the same formulation or can be provided in separate formulations in a product combination. In some embodiments, the composition or product combination comprises the taurine precursor and the 5AV precursor. In some embodiments, the composition or product combination does not comprise Eisenbergiela tayi. In some embodiments, the taurine precursor is selected from the group consisting of: cysteine, cysteine sulfinic acid, homocysteine, cystathionine, hypotaurine, and a mixture of two or more of the listed items. In some embodiments, the 5AV precursor is selected from the group consisting of: lysine, cadaverine, 1-piperideine, and a mixture of two or more of the listed items. In some embodiments, the composition or product combination comprises a bacteria that maps to the sOTU b20cd_Bacteroides. In some embodiments, the composition or product combination comprises a bacteria that maps to the sOTU 4ae7e_Parabacteroides. In some embodiments, the composition or product combination comprises a bacteria that maps to the sOTU b20cd_Bacteroides and a bacteria that maps to the sOTU 4ae7e_Parabacteroides. In some embodiments, a bacteria maps to an sOTU when the bacteria comprises a 16S rRNA sequence of at least 100 nucleotides that is least 97% identical to a reference 16S rRNA sequence of the sOTU. In some embodiments, a bacteria maps to an sOTU when the bacteria comprises a 16S rRNA sequence of at least 100 nucleotides that is least 99% identical to a reference 16S rRNA sequence of the sOTU. In some embodiments, the composition or product combination further comprises Lactobacillus reuteri. In some embodiments, the composition or product combination does not comprise any bacteria that maps to the sOTU 02b40_Lachnospiraceae and/or 29857_Lachnospiraceae. In some embodiments, the composition or product combination does not comprise any bacteria of Eggerthella, Alstipes, Burkolderiales, Enterococcaceae, Clostridium, or Rurminococcus. In some embodiments, the composition or product combination does not comprise any bacteria of Ruminococcaceace, Oscillospira, and Suterella. In some embodiments, the composition or product combination as described herein is for use in reducing one or more symptoms of ASD in a subject after birth, in which said composition or product combination is administered to said subject prenatally. In some embodiments, the said composition or product combination is administered directly to said subject prenatally. In some embodiments, said composition or product combination is administered to the mother of said subject prenatally, thereby administering said composition or product combination prenatally. In some embodiments, the composition or product combination is for use as described herein, and the one or more symptoms of ASD is selected from the group consisting of; repetitive behavior, hyperactivity, anxiety, and a communication disorder. In some embodiments, the composition or product combination comprises the bacteria in amount sufficient to establish a colony in the gut of a human subject when administered for microbiome transplant or probiotic treatment. In some embodiments, the colony persists for at least 1, 2, 3, 4 or more weeks post-inoculation.

Some embodiments include a composition or product combination comprising a) bacteria selected from the group consisting of: Bacteroides ovatus, Parabacteroides merdae, and Bacteroides thetaiotaomicron, and a mixture of two or more of the listed bacteria; and b) a metabolite that is expressed differently in ASD and non-ASD (which may also be referred to as “TD”) subjects as shown in any of Tables 3-1, 3-2, and/or 3-3, or a precursor thereof. Components (a) and (b) can provided in the same formulation, or can be provided in separate formulations in a product combination. In some embodiments the bacteria is in amount sufficient to establish a colony in the gut of a human subject when administered for microbiome transplant or probiotic treatment. In some embodiments, the colony persists for at least 1, 2, 3, 4 or more weeks post-inoculation. In some embodiments, the bacteria is selected from the group consisting of: Bacteroides ovatus, Parabacteroides merdae, and Bacteroides thetaiotaomicron, and a mixture of two or more of the listed bacteria. In some embodiments, the bacteria is selected from the group consisting of Bacteroides, Butyricimonas, Paraprevotallacae, and a mixture of two or more of the listed bacteria. In some embodiments, the bacteria is selected from the group consisting of: Bacteriodetes, Anaerofilum, Anaerotruncus, Christensenella, Pseudoramibacter eubacterium, Holdemania, and a mixture of two or more of the listed bacteria. In some embodiments, the bacteria is selected from the group consisting of Bacteroidetes, Holdemania, and a mixture of two or more of the listed bacteria. In some embodiments, the bacteria comprises, consists essentially of, or consists of a Bacteroidetes selected from the group consisting of Bacteroidaceae, Paraprevotellacae, Banesiellaceae, Rikenellaceae, Odoribacteraceae, Bacteroides, Butyricimonas, and a mixture of two or more of the listed bacteria. In some embodiments, the bacteria comprises a Bacteroidetes selected from the group consisting of: Bacteroides ovatus, Parabacteroides merdae, and Bacteroides thetaiotaomicron, and a mixture of two or more of the listed bacteria. In some embodiments, the bacteria comprises a Clostridiales selected from the group consisting of Lachnospiraceae and Clostridium. In some embodiments, the composition or product combination does not comprise any bacteria of Eggerthella, Alstipes, Burkolderiales, Enterococcaceae, Clostridiur, or Ruminococcus. In some embodiments, the composition or product combination does not comprise any bacteria of Ruminococcaceace, Oscillospira, and Sulerella. In some embodiments, the composition or product combination does not comprise Eisenbergiela tayi. In some embodiments, the metabolite comprises, consists essentially of, or consists of taurine and/or 5AV. In some embodiments, the composition or product combination comprises the taurine precursor and the 5AV precursor. In some embodiments, the taurine precursor is selected from the group consisting of: cysteine, cysteine sulfinic acid, homocysteine, cystathionine, hypotaurine, and a mixture of two or more of the listed items. In some embodiments, the 5AV precursor is selected from the group consisting of; lysine, cadaverine, 1-piperideine, and a mixture of two or more of the listed items.

Some embodiments include method of reducing or preventing a symptom of ASD in a selected prenatal subject after birth. The method can comprise administering a composition or product combination comprising an amount a metabolite to a subject prenatally. The amount can be effective to reduce or prevent the symptom of ASD in the subject after birth. The metabolite can be a metabolite that is expressed differently in ASD and non-ASD subjects as shown in any of Tables 3-1, 3-2, and/or 3-3.

### BRIEF DESCRIPTION OF THE DRAWINGS

FIGS. 1A-O are a series of drawings reflecting the observation that colonization with ASD microbiomes recapitulates behavioral deficits in C57BL/6J mice in accordance with some embodiments herein. FIG. 1A: Experimental design: Mice were colonized with fecal samples from autism spectrum disorder (ASD) or typically developing (TD) donors at weaning and bred at 7-8 weeks of age. Offspring of colonized mice were behaviorally tested starting 6 weeks of age and various tissues and samples were collected at P45. FIG. 1B: First three axes of a PCoA of unweighted unifrac distances from TD (circles) and ASD (squares) male offspring mice (colored by donor). TD and ASD mice were significantly different, as tested by pairwise PERMANOVA. N=4-7 male offspring per donor. FIG. 1C: The bacterial population of TD and ASD male offspring mice, by donor at the phylum level (by relative abundance). N=4-7 male offspring per donor. FIG. 1D: ASD-relevant behavioral assays: marble burying, direct social interaction, and open field testing in colonized offspring, colored by donor and stratified by mouse gender. Overlaid bar plots note the mean and S.E.M. Hypothesis testing for differences of the means were tested by a random effects analysis and p-values from a chi-square test. NASD=121, NTD=85 (8-23 mice per donor, per gender). Data presented is the aggregate of all experiments. FIG. 1E: Spearman correlation between mouse behavior and donor metadata (Tables 1.1-1.4). Benjamini-Hochberg adjusted p-values for significant (α≤0.05) correlations are noted. Color scale denotes Spearman ρ. FIG. 1F: ASD-relevant behavioral assays: marble burying, 3-chamber sociability, open field testing, and communication by USV in colonized offspring, colored by donor and stratified by mouse gender, in complete the dataset (16 donors). Overlaid bar plots note the mean and S.E.M. Hypothesis testing for differences of the means were tested by a random effects analysis and p-values from a chi-square test. NASD=179, NASD-Mild=44, NTD=111 (4-23 mice per donor, per gender). Data presented is the aggregate of all experiments. FIG. 1G: First two axes of a PCoA of unweighted Unifrac distances from TD (circles) and ASD (squares) donors, male and female recipients, and male offspring mice. FIG. 1H: Box plots of pairwise distances of donor, recipients, and offspring mice to donor samples by unweighted Unifrac distances. p-values from pairwise PERMANOVA test. FIG. 1I: Box plots of alpha diversity, as measured by the number of observed species. p-values from Kruskal-Wallis test. FIG. 1J: GraPhLan plot of LefSe linear discriminant analysis of microbiome profiles up to the genus level. Highlights denote significant taxonomic differences between TD and ASD mice (n=4-7 male offspring per donor). Significantly increased in TD were Bacteriodetes, Bacteroidia, Bacteriodales, Bacteriodaceae, Rikenellaceae, Paraprevotellaceae, Odoribacteraceae, Barneciellaceae, Bacteroides, Paraprevotella, Butyricimonas, Holdemania, Pseudoramibacter eubacterium, Christensenella, Anaerotruncus, and Anaerofilum. Significantly increased in ASD were Betaproteobacteria, Burkholderiales, Enterococcaceae, Lactobacillales, Clostridiaceae, Eggerthella, Alistipes. Enterococcus, Clostridium, and Ruminococcus. FIG. 1K Graph of alpha diversity in humanized TD and ASD mice (which may also be referred to as “oTD” and “oASD” mice, respectively) as measured by observed amplicon sequence variants (ASVs). Hypothesis testing was done by mixed effects analysis. N=4-7 male offspring per donor. FIGS. 1L-M: Graphs showing the first three axes of a principal coordinate analysis (PCoA) of unweighted UniFrac distances from TD (circles) and ASD (squares) male offspring mice. Group differences were tested pairwise by PERMANOVA. N=4-7 male offspring per donor. Shown are Axis 1 (24.29%) (FIG. 1L), Axis 2 (15.1%), and Axis 3 (12.35%) (FIG. 1M). FIG. 1N: Graph of alpha diversity in human TD (circles) and ASD (squares) as measured by observed amplicon sequence variants (ASVs). Hypothesis testing was done by two-tailed t-test. Eight samples used downstream are in dark grey. FIG. 1O: Graph of the first two axes of a PcOA of unweighted UniFrac distances from 16S rRNA gene sequencing of human TD and ASD donors. Group differences were tested by pairwise PERMANOVA. N_{TD}=5, N_{mild ASD}=3, N_{ADS}=8. Dark symbols denote samples followed up on.

FIGS. 2A-G are a series of drawings that relate to Lachnospiraceae, Bacteroides and Parabacteroides being observed to be differentially abundant in the TD- and ASD-offspring microbiomes in accordance with some embodiments herein. FIG. 2A: Volcano plot of differential abundance analysis by DESeq2. Significantly different taxa (α≤0.001) are colored according to their phylum, and annotated by the genus (or next available taxonomic level by GreenGenes identification). FIG. 2B: Heatmap of differentially abundant taxa by DESeq2 (α≤0.001). Features are named according to best available taxonomy by GreenGenes with a unique feature identifier. Samples are clustered by Bray-Curtis distances. FIG. 2C: Microbiome features contributing <1% to classification between TD and ASD samples by RandomForest. FIG. 2D: Relative abundance of select features in the microbiome of male offspring, colored by donor. Hypothesis testing for differences of the means were tested by a random effects analysis and p-values from a chi-square test. NASD=20, NTD=15 (4-7 mice per donor). FIG. 2E: The abundance of select feature in the offspring microbiome is correlated with behavior of males. Spearman correlation between the microbiome and mouse behavior, by donor (See FIG. 1). Benjamini-Hochberg adjusted p-values (α≤0.05) for significant correlations are noted. Scale denotes Spearman p. FIGS. 2F-G: Relative abundance of P. merdae (FIG. 2F) and E. tayi (FIG. 2G) in the original human cohorts. Hypothesis testing is by one-tailed Mann-Whitney U test NTD=32, NASD=42.

FIGS. 3A-J are a series of drawings relating to the observation that the ASD microbiome has modest impacts on gene expression in the brain, while exerting significant, ASD-relevant effects on alternative splicing, in accordance with some embodiments herein. FIG. 3A: Relative expression of differentially expressed genes in TD- and ASD-colonized mice (FDR≤0.1). Protein coding genes (4930539E08Rik, Dag1b) in aggregated data from both STR and PFC, and long noncoding RNAs (Gm26944, Gm13016, Gm17259) in PFC. Data points colored by donor. PFC: NASD=19, NTD=14, STR: NASD=20, NTD=14 (3-6 mouse samples per donor/tissue). FIG. 3B: Venn diagram of differentially spliced genes in either the STA and PFC between TD- and ASD-offspring mice (ASD microbiome Spliced; FDR≤0.05), and their relevance to known ASD genes as curated by SPARK and SFARI. FIG. 3C: Examples of differential splicing events (FDR≤0.05) in genes present in both SPARK and SFARI Gene. Spliced in rates of differentially spliced isoforms of Nrxn2 (MXE event, STR), Ank2 (MXE event, STR), Fmr1 (SE event, STR), Ube3a (SE event, STR), Rims1 (SE event, STR), Cacna1a (SE event, STR), Pogz (SE event, PFC), Trip12 (SE event, PFC), and Ads1 (RI event, STR). PFC: N_{ASD}=19, N_{TD}=14, STR: N_{ASD}=20, N_{TD}=14 (3-6 mouse samples per donor/tissue). Benjamini-Hochberg p-values were calculated by rMATS differential splicing analysis. FIG. 3D: Examples of differential splicing events (FDR≤50.05) in genes present in both SPARK and SFARI Gene based on both STR and PFC samples. Spliced in rates of differentially spliced isoforms of Gigyf2 (A3SS event) and Nrxn1 (A5SS event). Data points colored by donor. PFC: NASD=19, NTD=14, STR: N_{ASD}=20, N_{TD}=14 (3-6 mouse samples per donor/tissue). FIGS. 3E-F: Volcano plots of genes expressed in the STR, PFC, and aggregated data from both brain regions of TD- and ASD-colonized offspring (FDR≤0.1). FIGS. 3G-H: Graphs of KEGG pathways upregulated (FIG. 3G) and downregulated (FIG. 3H) in the brains of ASD mice by Gene Set Enrichment Analysis (GSEA). FIG. 3I: Graph of cell-type enrichment analysis of differentially-splicing events in brains of ASD mice. Odds-ratio and 95% confidence intervals are presented. FIG. 3J: Graph of enrichment of differentially-splicing events amongst previously reported targets of specific RNA-binding proteins (RBPs) and activity-dependent events in the brain.

FIGS. 4A-H are a series of drawings that relate to the observation that the ASD microbiome affects the metabolome in the colon and serum of colonized mice in accordance with some embodiments herein. FIGS. 4A-C are a series of volcano plots of differentially abundant metabolites identified by an untargeted metabolomics of (FIG. 4A) colon contents by GC-MS, (FIG. 4B) colon contents by 1H NMR, and (FIG. 4C) serum by GC-MS. Significantly different metabolites with more than 50% difference marked in red, and those with modest effects marked in yellow. NASD=20, NTD=15 (4-7 mice per donor). It is noted that several identified differently-abundant substances had mass spectrometry fragmentation patterns that did not fit any of the databases searched, and as such are unlabeled on FIG. 4A. FIGS. 4D-F are a series of heat maps of differentially abundant metabolites identified by an untargeted metabolomics of (FIG. 4D) colon contents by GC-MS, (FIG. 4E) colon contents by 1H NMR, and (FIG. 4F) serum by GC-MS. N_{ASD}=20, N_{TD}=15 (4-7 mice per donor). FIGS. 4G-H illustrate normalized concentrations of select metabolites in (FIG. 4G) colon contents or (FIG. 4H) serum. Data point color denotes Donor. N_{ASD}=20, N_{TD}=15 (4-7 mice per donor). Bar graphs denote mean and S.E.M. The raw NMR and mass spectrometry data are provided as large Tables 3-1, 3-2, and 3-3.

FIGS. 4I-L are a series of drawings illustrating potential taxonomic contributors to variation in metabolite concentrations in accordance with some embodiments herein. FIG. 4I: Relative abundance of amino acids identified by NMR analysis from colon contents, relative to their correspondence to Mimosa prediction. FIG. 4J: Relative abundance of amino acids identified by GC-MS analysis from colon contents, relative to their correspondence to Mimosa prediction. FIG. 4K: Mimosa-model prediction of sOTUs involved in production or degradation of specific metabolites. Columns correspond to 88 Greengenes OTUs with an average rarefied abundance of at least one read out of 11,000 in the control and/or autism donor samples. Rows correspond to the metabolites across all three metabolomics assays that were significantly consistent with metabolic potential at a q-value threshold of 0.1. Blue squares indicate that the estimated metabolic potential of the OTU in question is consistent with contributing to variation in that metabolite. The area of the colored points along the bottom shows the relative abundances of each taxon in control and ASD donor samples. The segments along the bottom indicate the relative ratio of each taxon in control versus ASD samples. The segments along the left side show the average difference in metabolite concentration Z-scores between the control and ASD donor samples. The column of colored tiles indicates the Mimosa correlation between metabolic potential scores and metabolite concentrations for each metabolite. FIG. 4L: KEGG (Kyoto Encyclopedia of Genes and Genomes; accessible on the world wide web) orthologues that contribute to taurine and their abundance in TD and ASD microbiomes (colored by donor), as predicted by PICRUSt.

FIGS. 5A-D are a series of drawings illustrating that differential metabolites impact ASD-relevant behaviors in SPF mice in accordance with some embodiments herein. FIG. 5A: The abundance of select metabolites in the offspring microbiome is correlated with behavior of male offspring. Spearman correlation between the microbiome and mouse behavior, by donor (See FIGS. 1A-0). Benjamini-Hochberg adjusted p-values (α≤0.05) for significant correlations are noted. Color scale denotes Spearman ρ. FIGS. 5B-D: Taurine and 5AV ameliorate ASD-related behavioral deficits in the BTBR mouse model for ASD. Groups of mice were orally administered 10 mM Taurine or 5AV in drinking water before mating, and throughout their lifetime. Offspring were tested by marble burying, direct social interaction, and open field tests, and compared to untreated vehicle controls. Results are aggregated from 2 independent experiments. NControl=42, N5AV=52, NTaurine=36. Bar plots denote mean and S.E.M. Hypothesis on differences in means were tested by ANOVA on trimmed means (10%) and subsequent posthoc tests. FIGS. 5E-L: qPCR of BTBR brains after 5AV and taurine administration. Results are aggregated from 2 independent experiments. Hypothesis on differences in means were tested by ANOVA on trimmed means (10%) and subsequent posthoc tests. FIGS. 5M-V: series of graphs illustrating E18 B6 feeding 5AV and Taurine targeted metabolomics.

FIGS. 6A-F are a series of graphs illustrating engraftment fidelity of colonization with human ASD microbiomes in mice in accordance with some embodiments herein. FIG. 6A: alpha diversity as measured by observed amplicon sequence variants (ASVs) in TD and ASD individuals from which donor samples in this study were used. Hypothesis testing was done by two-tailed t-test. Sixteen samples used downstream are in dark grey. Black bar represents the mean. FIG. 6B: First three axes of a PcOA of unweighted UniFrac distances from TD (circles) and ASD (squares) donors, male and female recipients, and male offspring mice. Group differences were tested by pairwise PERMANOVA. N_{Donors}=8, N_{Recipients}=71, N_{Offspring}=35. FIG. 6C: box plots of alpha diversity, as measured by the number of observed species. Group differences tested by Kruskal-Wallis test. FIGS. 6D-E: First two axes of a PCoA of unweighted UniFrac distances from 16S rRNA gene sequencing of human TD and ASD donor populations. Group differences were tested by pairwise PERMANOVA. N_{TD}=32, N_{ASD}=40. Dark symbols denote samples followed up on. FIG. 6F: Graph of taxa engraftment in mice at the species level. The fraction of taxa present in mice and respective donor, as well as the cumulative relative abundance of shared taxa in the donor are plotted.

FIGS. 7A-J are a series of graphs illustrating gastrointestinal physiology and gene expression in offspring mice in accordance with some embodiments herein. Measured parameters include mouse weight at 12 weeks of age (FIG. 7A), intestinal permeability as measured by FITC-dextran (4 kDa) in serum following gavage (FIG. 7B), intestinal transit time as measured by carmine-red gavage and detection in feces (FIG. 7C), serum Lipcalin-2 (LCN2) concentration as measured by ELISA (FIG. 7D), expression of the tight-junction genes Occludin (Ocldn) in the distal ileum (FIG. 7E), Zonula Occludens 1 (ZO1) in the distal ileum (FIG. 7F), Zonula Occludens 2 (ZO2) in the distal ileum (FIG. 7G), Ocldn in the proximal colon (FIG. 7H), ZO1 in the proximal colon (FIG. 7I), and ZO2 in the proximal colon (FIG. 7J).

FIGS. 8A-E are a series of graphs illustrating effects of microbial metabolites on brain activity of mice in accordance with some embodiments herein. Mice were orally administered 10 mM Taurine or 5AV in drinking water starting 3-4 weeks of age mating, and throughout their lifetime. FIG. 8A illustrates effects on amplitude and frequency of mEPSCs in pyramidal neurons in the L5 of the mPFC in acute slices from BTBR mice treated with 5AV, Taurine, or control. FIG. 8B illustrates effects on amplitude and frequency of mIPSCs in pyramidal neurons in the L5 of the mPFC in acute slices from BTBR mice treated with 5AV, Taurine, or control. Hypotheses for FIGS. 8A-B were tested by ANOVA on trimmed means (10%) and subsequent post-hoc tests. FIG. 8C illustrates effects on excitability of pyramidal neurons in the L5 of the mPFC in acute slices from BTBR mice treated with 5AV, Taurine, or control in response to step-wise injection current, as measured by the number of action potential spikes. 2-way ANOVA.

FIGS. 9A-R are a series of graphs illustrating that metabolite administration (5AV and Taurine) post weaning has no effects on ASD-related behaviors in BTBR mice in accordance with some embodiments herein. In FIGS. 9A-K, mice were orally administered 10 mM Taurine or 5AV in drinking water starting 3-4 weeks of age mating, and throughout their lifetime. Offspring were tested by marble burying, direct social interaction, and open field tests, and compared to untreated vehicle controls. Results are aggregated from 2 independent experiments. NControl=20, N5AV=18, NTaurine=20. Bar plots denote mean and S.E.M. hypothesis on differences means were tested by ANOVA on trimmed means (10%) and subsequent posthoc tests. FIGS. 9L-Q illustrate basal properties of L5 pyramidal neurons in the mPFC. FIG. 9R illustrates a proportion of GABA-excitable cortical rat neurons as a function of days in culture (DIV), treated with either 5AV, Taurine, or control.

FIGS. 10A-10T are sequences of 16S RNA sequences of bacteria identified as differing between the gut of ASD-colonized and NT-colonized wild type germ free mice according to some embodiments.

### DETAILED DESCRIPTION

Without being limited by theory, it has been observed that gut bacterial communities differ between individuals diagnosed with ASD and typically-developing (TD) individuals (De Angelis et al., 2013; Gondalia et al., 2012; Kang et al., 2013; Kushak et al., 2016; Son et al., 2015; Strati et al., 2017; Williams et al., 2011), as well as in mouse models of ASD (Buffington et al., 2016; Coretti et al., 2017; Hsiao et al., 2013; de Theije et al., 2014). Fecal microbiome profiles are most divergent in ASD subjects presenting with GI dysfunction (Gondalia et al., 2012; Son et al., 2015), a common comorbidity of autism (Chaidez et al., 2014; Gorrindo et al., 2012). In addition, bacterial-based interventions, including fecal transplants, antibiotics and probiotics, have shown promise in some open-label human trials (Kang et al., 2017; Sandler et al., 2000). Some gut microbes have also demonstrated therapeutic potential in animal models of ASD (Buffington et al., 2016; Hsiao et al., 2013).

The microbiome harbors a considerable genetic capacity, and changes in the microbiome result in altered metabolic profiles, impacting the availability and diversity of nutrients and microbial secondary metabolites. Metabolomic analyses of serum and urine from ASD subjects have uncovered differences in various molecules compared to typically-developing controls, with many compounds that differ between the two groups being of microbial origin (De Angelis et al., 2013; Ming et al., 2012; Mussap et al., 2016). Notably, amino-acid transport and degradation capabilities have been implicated to differ between TD and ASD individuals (Aldred et al., 2003; Evans et al., 2008; Fernell et al., 2007; Naushad et al., 2013), with amino acids serving as substrates for many potent neuroactive molecules.

Disclosed herein are experimental evidence that elucidate differences in the microbiomes and metabolism of ASD subjects and their prenatal environment in comparison to non-ASD (“typically developing” (TD)) controls, that contribute to specific aspects of ASD symptoms. It has been discovered that gut microbiomes of individuals with ASD contribute to behavioral and metabolic differences from that of the TD population. It is reported herein that colonization of germ-free (GF) C57BL/6J wild-type mice with fecal microbiomes from certain ASD patients is sufficient to induce core behavioral phenotypes in their offspring (See Example 1). Additionally, data presented herein show that individuals exhibiting ASD symptoms and TD controls can be differentiated based on different colonizing gut bacteria (See Example 2). Accordingly, in some embodiments, a profile of a sample of a subject (e.g. a sample of colon contents such as feces) comprises one or more detected gut bacteria. It is further shown that colon and serum metabolite levels can differ in subjects colonized with these bacteria and exhibiting ASD symptoms, compared to TD controls colonized with bacteria that did not induce ASD symptoms (See Example 4). Accordingly, some embodiments relate to methods of determining a profile of a sample of a subject comprising determining or establishing a profile of one or more metabolites and/or metabolite precursors from a tested subject and comparing that profile to one or more profiles generated from subjects that have ASD (ASD controls) and/or healthy subjects (non-ASD controls). For example, in some embodiments, levels or amounts of taurine, 5AV, 5-aminopentanoate, cellobiose, D ribose, ribonic acid, 3AIBA, lysine, glyceric acid, geristein, daidzein, and/or lipocalin-2 (LCN2) are determined in a tested sample and these detected levels or amounts from the tested sample are compared to the levels or amounts of these compounds found in one or more profiles generated from subjects that have ASD and/or healthy subjects (controls). For example, in some embodiments, levels or amounts of taurine, 5AV, 5-aminopentanoate, cellobiose, D ribose, ribonic acid, 3AIBA, lysine, glyceric acid, geristein, and/or daidzein are determined in a tested sample and these detected levels or amounts from the tested sample are compared to the levels or amounts of these compounds found in one or more profiles generated from subjects that have ASD and/or healthy subjects (controls). Preferably, the levels or amounts of taurine, 5AV, 5-aminopentanoate, cellobiose, 3AIBA, lysine, glyceric acid, geristein, and/or daidzein are determined by analyzing a fecal sample and levels of D ribose, ribonic acid, and/or LCN2 are determined by analyzing a serum sample. Colon levels or amounts of taurine, 5AV, 5-aminopentanoate, or cellobiose, below a non-ASD control (e.g., a healthy subject), serum levels of ribitol or L-tyrosine below a non-ASD control (e.g., a healthy subject), colon levels of 3AIBA, lysine, glyceric acid, geristein, and/or daidzein above a non-ASD control (e.g., a healthy subject), and/or serum levels of D ribose or ribonic acid or LCN2 above a non-ASD control (e.g., a healthy subject) indicate an increased risk of developing and/or severity of ASD in the subject after birth. Similarly, levels or amounts of taurine, 5AV, 5-aminopentanoate, or cellobiose, equal to or below that of an ASD control (e.g., a subject having ASD or an ASD symptom), serum levels of ribitol or L-tyrosine equal to or below that of an ASD control (e.g., a subject having ASD or an ASD symptom), colon levels of 3AIBA, lysine, glyceric acid, geristein, and/or daidzein equal to or greater than that of an ASD control (e.g., a subject having ASD or an ASD symptom), and/or serum levels of D ribose or ribonic acid equal to or greater than that of an ASD control (e.g., a subject having ASD or an ASD symptom) indicate an increased risk of developing and/or severity of ASD in the subject after birth. In some embodiments, and ASD control is identified as having ASD based on a behavioral assessment, for example the Autism Behavior Checklist (ABC), Autism diagnostic Interview-Revised (ADI-R), childhood autism Rating Scale (CARS), and/or Pre-Linguistic Autism Diagnostic Observation Schedule (PL-ADOS).

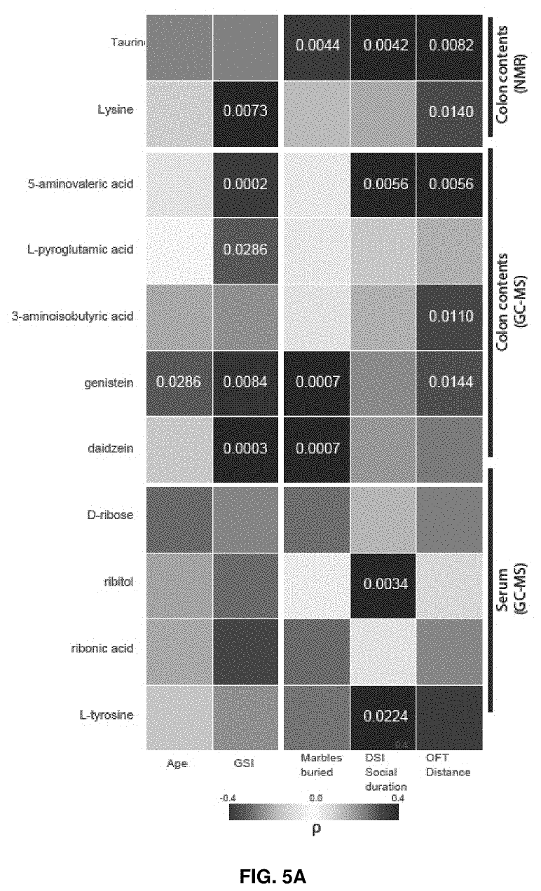

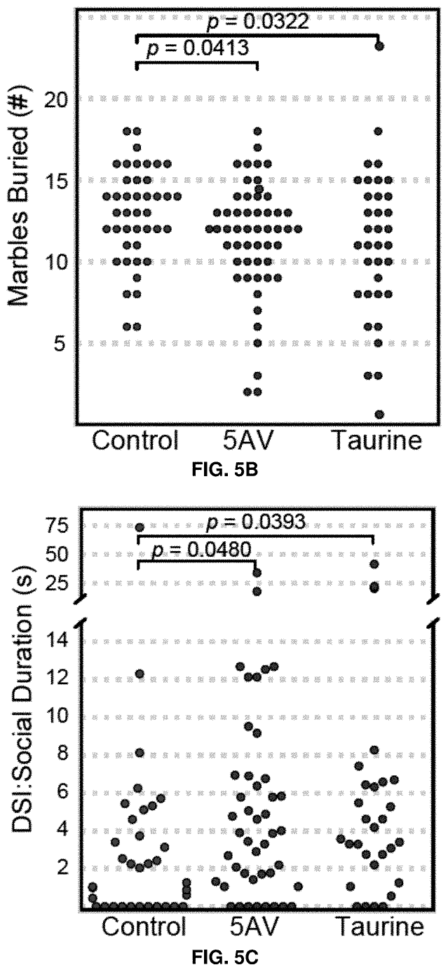

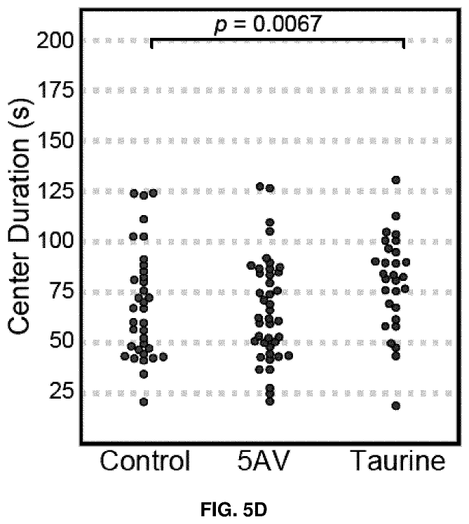

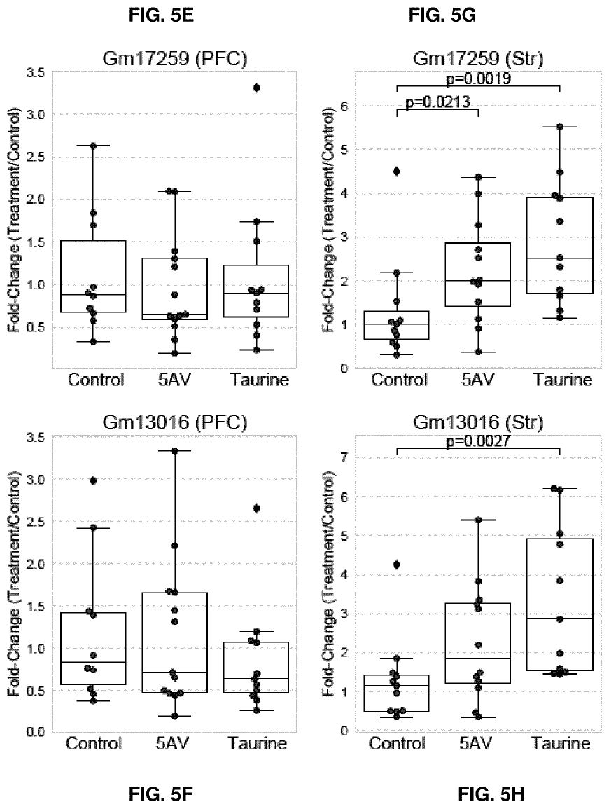

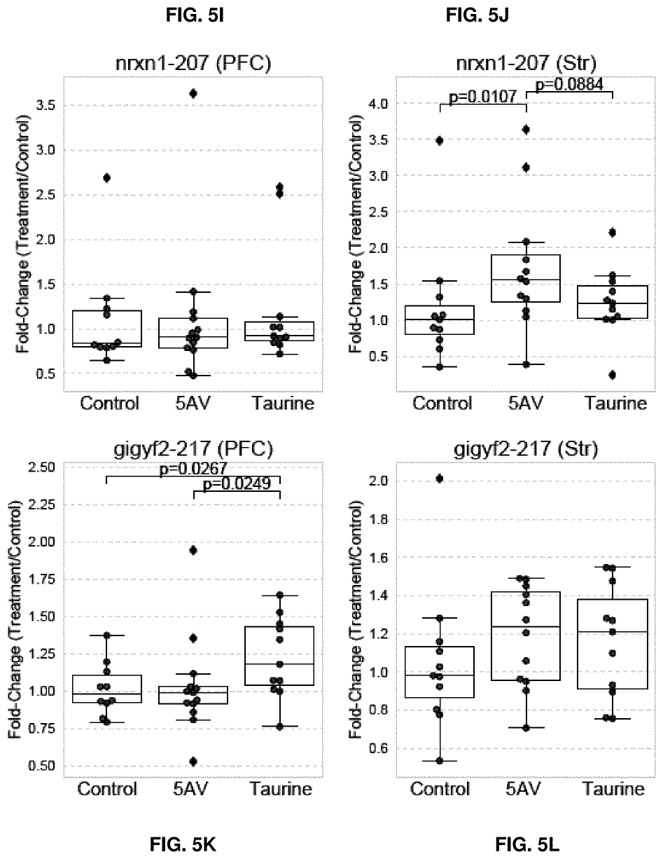

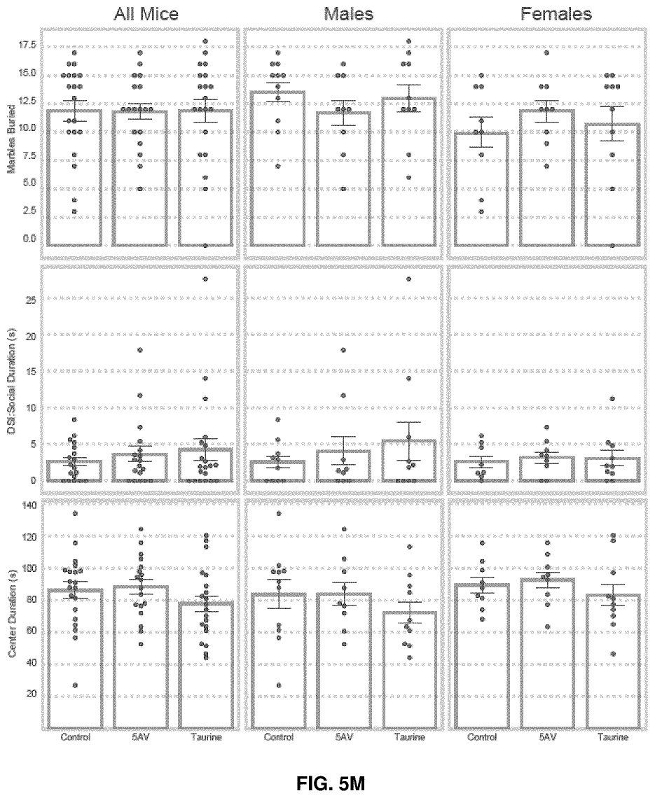

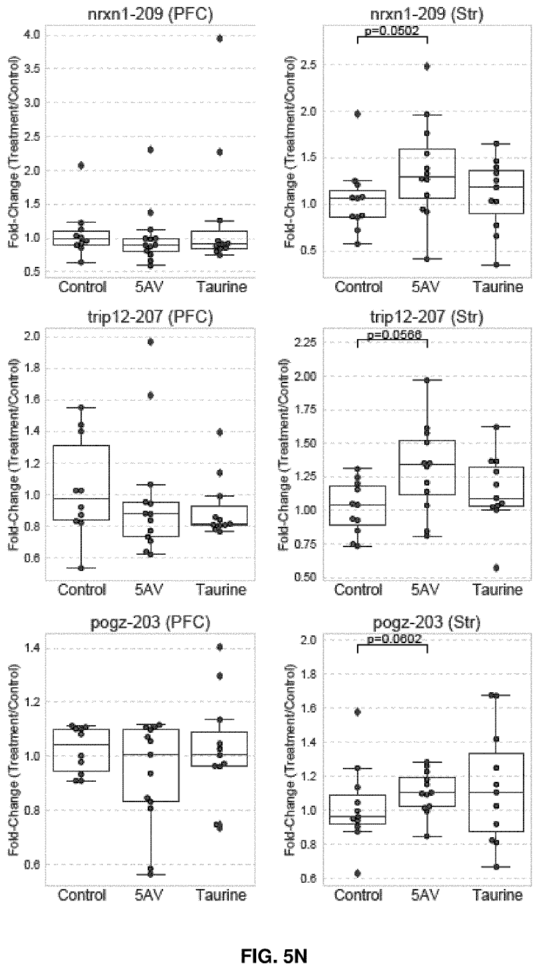

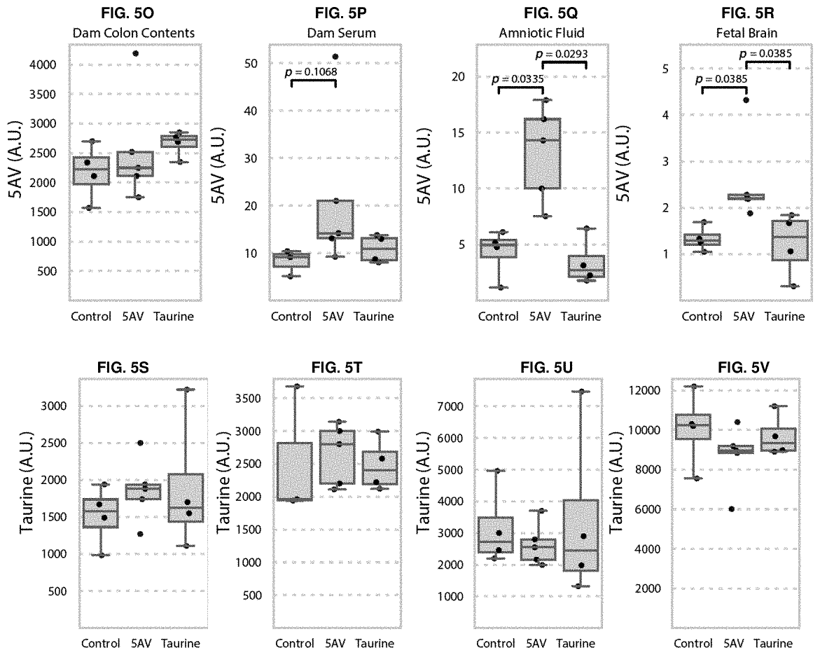

Moreover, it is described herein that human ASD microbiomes alter the metabolomic profile of the colon and serum of mice, and regulate processing of genes shown to be affected in the brains of subjects with ASD (&e Examples 3). Accordingly, some embodiments concern methods of determining a profile of a sample of a subject comprising determining a profile (e.g., presence, transcription levels, and/or splicing patterns) of genes that exhibit different profiles in subjects with ASD relative to non-ASD subjects (controls). For example, in some embodiments, an elevated risk and/or severity of ASD is indicated by presenting increased levels or amounts (e.g., in a gut sample of the subject, such as feces and/or colon contents) of an ortholog of KEGG ortholog K0681 and/or decreased levels of an ortholog of KEGG ortholog K01442, relative to levels present in a control typically developing subject (also referred to throughout as a healthy subject, such as a non-ASD individual, for example an individual that does not exhibit ASD and/or does not exhibit the noted ASD symptoms). For example, in some embodiments, levels or amounts of Gm26944, Gm13016, Gm17259, 4930539E08Rik, and/or Dag1b gene products (e.g., transcripts and/or polypeptides thereby produced); levels or amounts of the mutually exclusive exon in Neurexin 2, levels or amounts of the mutually exclusive exon in Ankryin 2; levels or amounts of the skipped exon of Cacna1c; and/or levels or amounts of the retained intron of Ads1 are detected or determined in a sample from a tested subject, such as a biological sample comprising central nervous system (CNS) tissue and/or cerebrospinal fluid (CSF). For example, in some embodiments, levels or amounts of Gm26944, Gm13016, Gm17259, 4930539E08Rik, and/or Dag1b gene products (e.g., transcripts and/or polypeptides thereby produced); levels or amounts of the mutually exclusive exon in Neurexin 2, levels or amounts of the mutually exclusive exon in Ankryin 2; levels or amounts of the skipped exon in Fmr1; levels or amounts of the skipped exon in Ube3a; levels or amounts of the skipped exon in Rims1; levels or amounts of the skipped exon of Cacna1c; levels or amounts of the retained intron of Ads1; a skipped exon of a pogo transferrable element derived with ZNF domain Pogz; and/or a skipped exon of Trip12 are detected or determined in a sample from a tested subject, such as a biological sample comprising CNS tissue and/or CSF. Levels of Gm26944, Gm13016, and/or Gm17259 gene product greater than a non-ASD control (e.g., a healthy subject) or below a non-ASD control (e.g., a healthy subject) indicate a presence, elevated risk of, and/or elevated severity of ASD after birth. Similarly, levels of Gm26944, Gm13016, and/or Gm17259 gene product equivalent to that of a non-ASD control (e.g., a healthy subject) indicates that the tested subject does not have ASD or does not present an elevated risk of and/or elevated severity of ASD after birth. Levels of 4930539E08Rik and/or Dag1b below a non-ASD control (e.g., a healthy subject) also indicates a presence of ASD or an elevated risk of, and/or elevated severity of ASD in the tested subject after birth. Levels of the mutually exclusive exon in Neurexin 2 above a non-ASD control (e.g., a healthy subject), levels of the mutually exclusive exon in Ankryin 2 below the non-ASD control (e.g., a healthy subject), levels of the skipped exon of Fmr1 below the non-ASD control, levels of the skipped exon of Ube3a below the non-ASD control, levels of the skipped exon of Rims) above the non-ASD control; levels of the skipped exon of Cacna1c above the non-ASD control (e.g., a healthy subject), levels of the retained intron of Ads1 above a non-ASD control (e.g., a healthy subject), levels of the skipped exon of the pogo transferrable element above a non-ASD control (e.g., a healthy subject) and/or levels of the skipped exon of Trip12 above the non-ASD control indicate an increased risk of ASD also indicate an increased risk of ASD after birth and/or elevated severity of ASD after birth. Some embodiments comprise determining the levels or amounts of any of the gene products described herein in central nervouse system tissue (e.g., brain tissue) of a subject in vivo, for example via functional Magnetic Resonance Imaging (fMRI) or Positron Emission Tomography (PET). Accordingly, wherever determining a profile of a sample of a subject comprising determining a profile (e.g., presence, transcription levels, and/or splicing patterns) of genes is described herein, determining the same information by in vivo imaging of the relevant central nervous system tissues where the gene product is differentially expressed in AD and TD subjects (e.g., striatum and/or PFC; See FIGS. 5E-L) is also contemplated.

Additionally, administration of specific metabolites identified herein as depleted in ASD-colonized mice to the BTBR T+ Itpr3tf/J (BTBR) mouse model of ASD protects against the onset of ASD symptoms after birth, while supplementing with a metabolite elevated relative to controls induces ASD symptoms in wild-type animals (Example 5). Example ASD symptoms (after birth) for which protection is observed in accordance with some embodiments herein include impaired sociability, repetitive behaviors, and anxiety, or a combination or two or more of these items. These findings demonstrate mechanisms by which the microbiome contributes to behavioral and physiological features of ASD, as well as methods and compositions for reducing, ameliorating, and/or preventing ASD or symptoms of ASD in a subject after birth.

Evidence is provided herein that prenatal levels or amounts of certain metabolites (e.g., prenatal levels or amounts of taurine and/or 5-Aminovaleric acid (5AV) that are less than the prenatal levels or amounts of taurine and/or 5AV found in healthy subjects (such as non-ASD subjects) and/or prenatal levels or amounts of 3-aminoisobutyric acid (3AIBA) that are higher than the prenatal levels or amounts of 3AIBA found in healthy subjects) are associated with and contribute to an increased risk or severity of ASD symptoms and/or conditions including, but not limited to, repetitive behaviors, sociability difficulties, and/or communication deficits in infants. Accordingly, embodiments described herein concern methods and compositions for increasing levels of certain metabolites, such as taurine and/or 5AV and, optionally lowering levels of certain metabolites, such as 3AIBA, in prenatal subjects to reduce the risk and/or severity of ASD symptoms and/or conditions after birth including, but not limited to, repetitive behaviors, sociability difficulties, communication deficits, and/or anxiety. In some embodiments, the level of a selected metabolite in a prenatal subject is modulated directly by administering the metabolite itself (e.g., taurine and/or 5AV) to the prenatal subject. In some embodiments, the level of a selected metabolite is modulated by administering the metabolite itself (e.g., taurine and/or 5AV) to the mother in a manner that the metabolite is circulated or delivered to the prenatal subject.

In some embodiments, the level of a selected metabolite is modulated indirectly by administering to the prenatal subject or the mother of the prenatal subject one or more bacteria that contribute to the production of the selected metabolite, and/or the degradation of a selected metabolite, which one desires to reduce in amount or presence. For example, in some embodiments, Bacteroides ovatus, Parabacteroides merdae, and/or Bacteroides thetaiotaomicron are administered to the gut of a selected subject, optionally in combination with a precursor of 5AV and/or taurine. By this approach, the administered bacteria facilitate the conversion of the precursor into the metabolite (5AV and/or taurine) in the selected subject. In some embodiments, the bacteria colonize at least a portion of the gut.

In some embodiments, the presence of ASD, the severity of ASD, and/or the risk of developing ASD is determined based on the generation of a profile indicating the presence and/or amount or level of one or more metabolites (e.g., taurine, 5AV, and/or 3AIBA) in a selected subject (e.g., a prenatal subject or the mother of a prenatal subject). In some embodiments, the risk and/or severity of ASD symptoms or conditions or symptoms and conditions including, but not limited to, repetitive behaviors, sociability difficulties, and/or communication deficits is determined by comparing profiles of gut bacteria (e.g., bacteria associated with the production and/or degradation of particular metabolites such as, Bacteroides ovatus, Parabacteroides merdae, Bacteroides thetaiotaomicron, and/or Eisenbergiela tayi) of healthy subjects (such as non-ASD subjects) and/or subjects expressing ASD symptoms and/or conditions including, but not limited to, repetitive behaviors, sociability difficulties, and/or communication deficits to a profile of gut bacteria generated for the selected subject.

Compositions and/or Product Combinations Comprising Bacteria and/or Metabolite Precursors

Compositions and/or product combinations comprising, consisting essentially of, or consisting of certain bacteria and/or metabolite precursors are useful for reducing the risk and/or severity of symptoms of ASD. Without being limited by theory, it is contemplated that certain bacteria contribute to the production and/or inhibit the degradation of certain metabolites that reduce the risk and/or severity of ASD symptoms. As such, some embodiments concern one or more compositions that comprise, consist essentially of, or consist of one or more bacteria and a metabolite precursor that is useful in reducing the risk and/or severity of ASD symptoms in subjects. It noted that in some embodiments, the components of any of the noted compositions can be provided separately as “product combinations” in which the components are provided in two or more precursor compositions, which can either be combined to form the final composition (e.g., mix bacteria with a metabolite precursor to arrive at a final composition comprising bacteria and a metabolite precursor), or used in conjunction to achieve an effect similar to the single composition (e.g., administer bacteria and a metabolite precursor to a subject simultaneously or sequentially). Accordingly, wherever a composition comprising two or more components is described herein, a corresponding “product combination,” which collectively contains the components of the composition is also expressly contemplated.

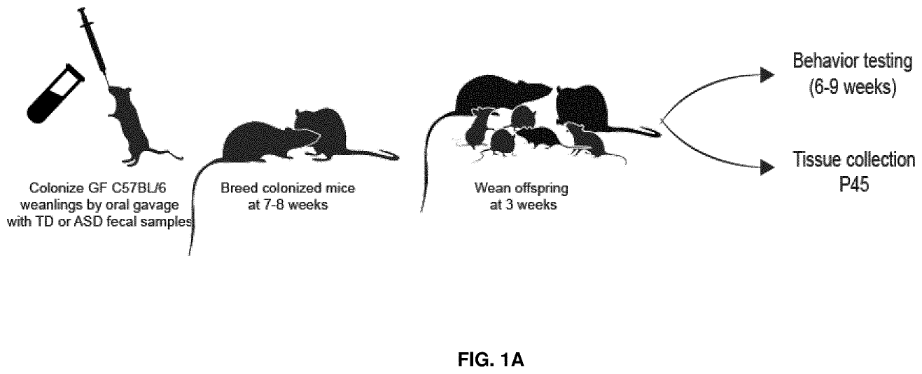

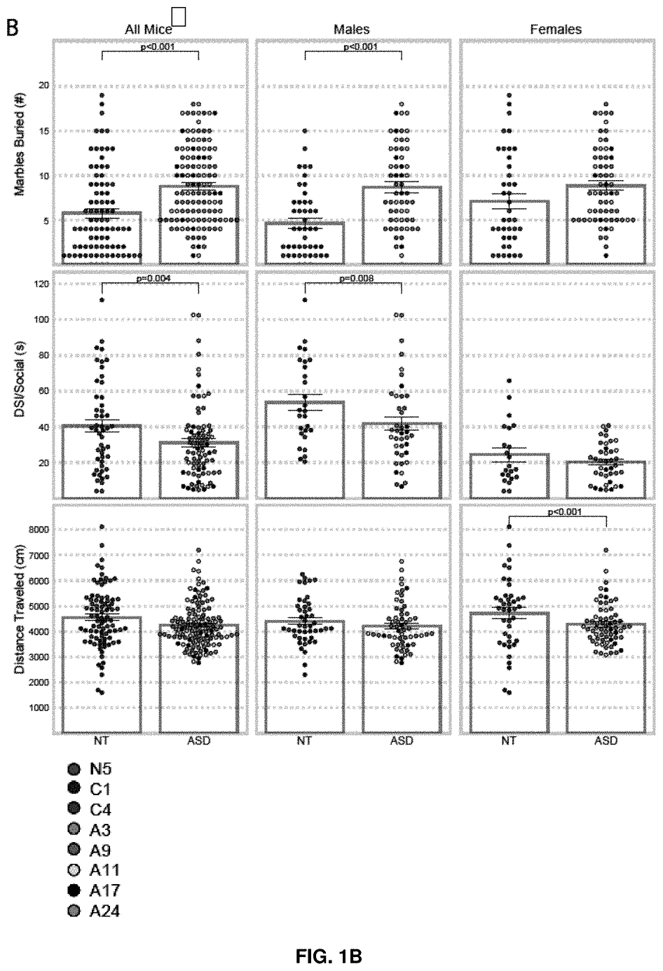

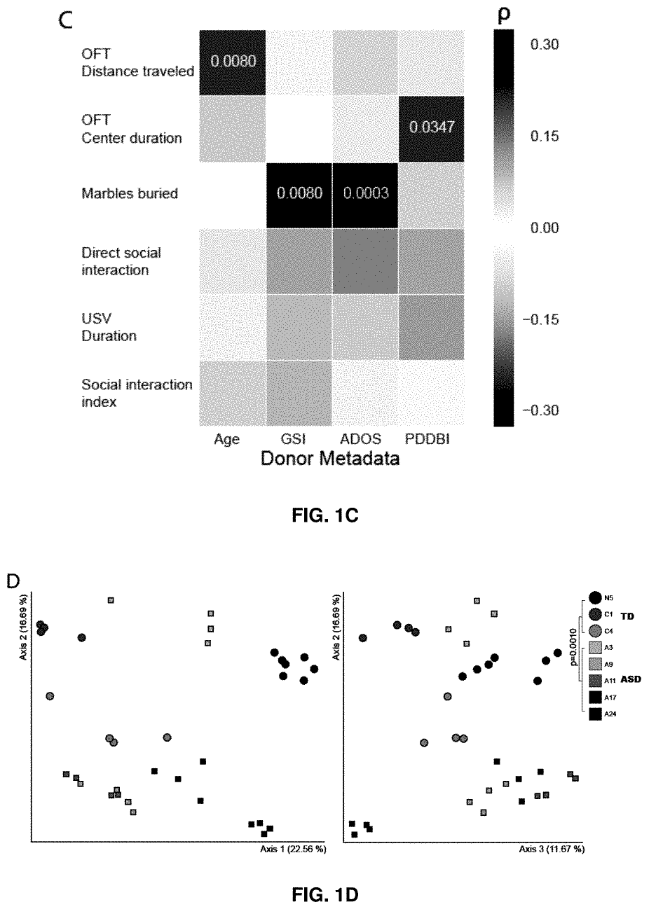

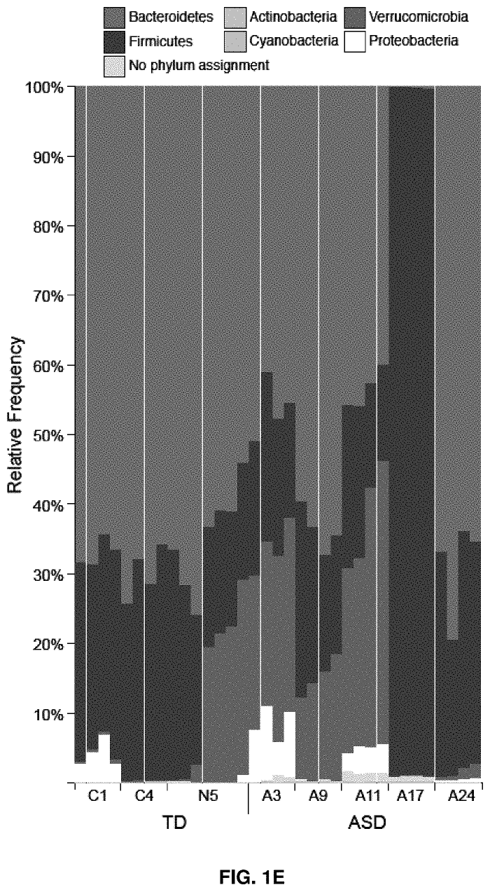

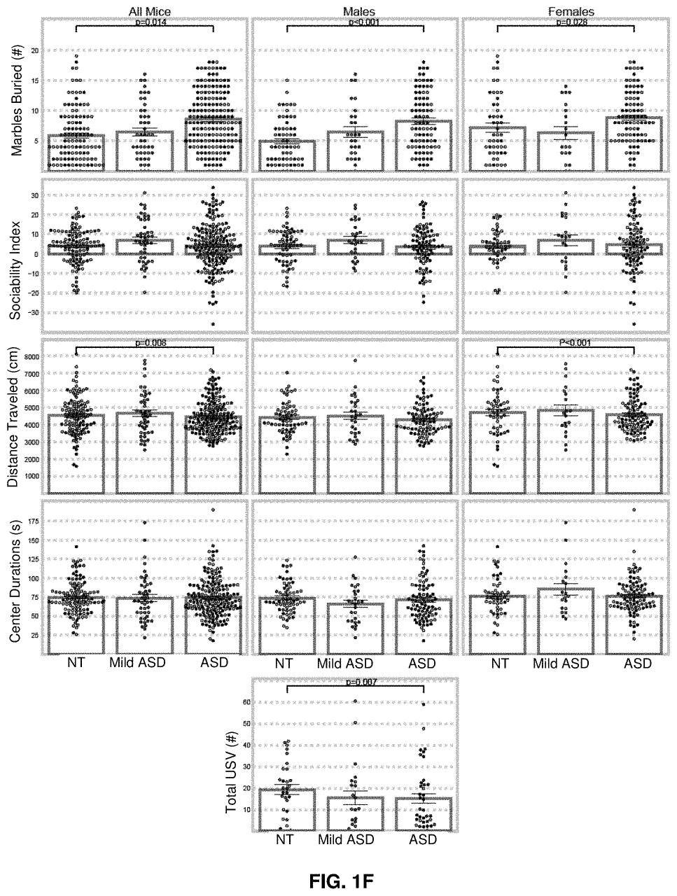

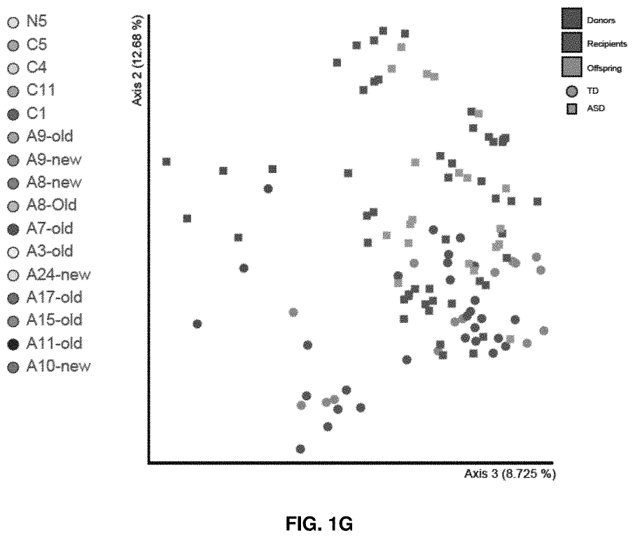

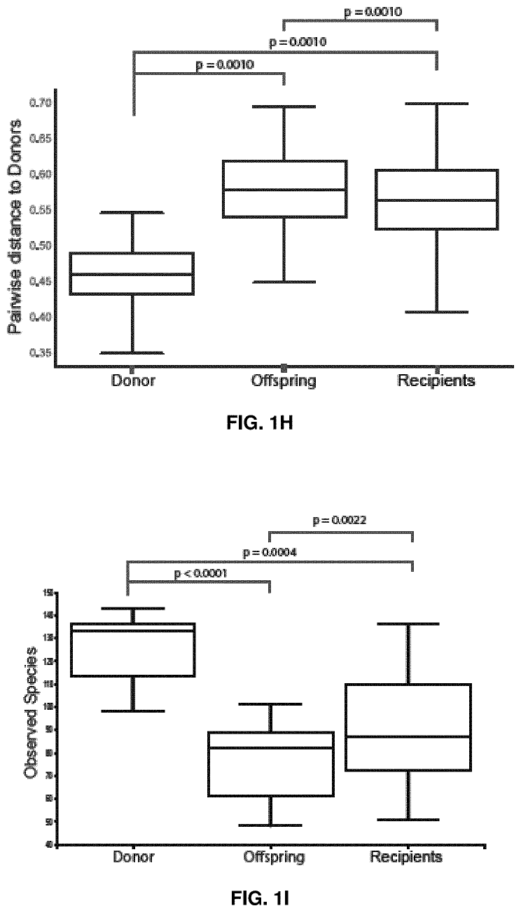

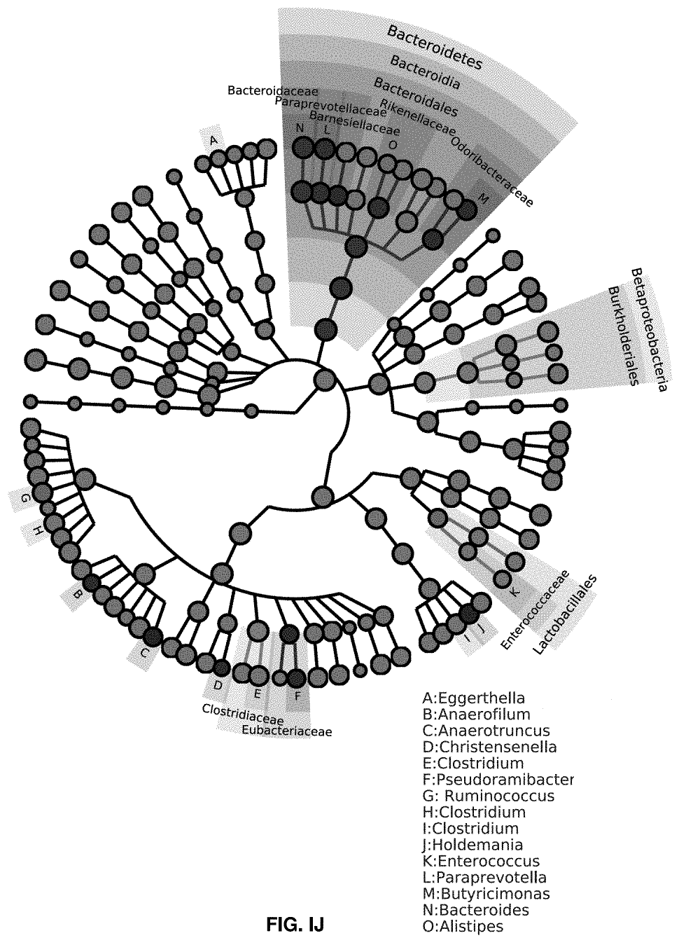

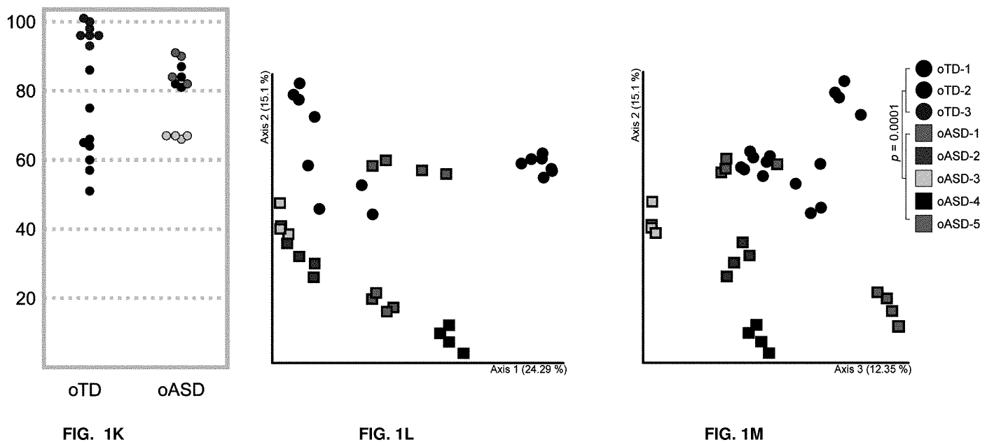

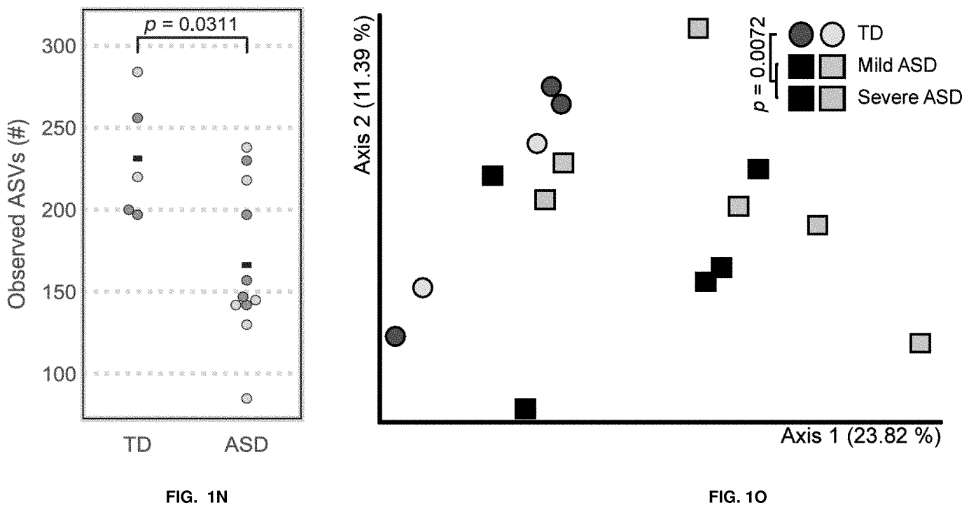

It is contemplated that in some embodiments, a composition and/or product combination as described herein is useful for reducing the risk and/or severity of, and/or ameliorating the effects of one of more symptoms of ASD in a subject after birth. For example, it has been observed that transplantation of gut bacteria can induce ASD symptoms in a subject, and that some bacteria are underrepresented in the guts of subjects suffering from ASD compared to TD (control) subject (See Example 1 and FIG. 1J). Accordingly, it is contemplated that in some embodiments, ASD can be inhibited, reduced, ameliorated, treated, and/or delayed in onset by administering one or more bacteria that are underrepresented in the gut of an ASD subject compared to a TD (control subject), for example as shown in FIG. 1J. In some embodiments, the metabolite or metabolite precursor is administered to the subject prenatally (e.g., to the subject, or to the subject's mother), and the bacteria are administered to the subject after birth. It is further contemplated that there is no need to administer bacteria that are overrepresented in the guts of subjects suffering from ASD compared to TD controls. For example, without being limited by theory, it is contemplated that some bacteria overrepresented in the guts of subjects suffering from ASD compared to TD controls can contribute to ASD or symptoms thereof.

Accordingly, in some embodiments, the composition and/or product combination comprises, consists essentially of, or consists of (a) bacteria selected from the group consisting of Bacteriodetes, Anaeroflum, Anaerotruncus, Christensenella, Pseudoramibacter eubacterium, Holdemania, and Clostridiales, or a mixture of two or more of the listed bacteria. The composition and/or product combination also comprises (b) a taurine precursor and/or a 5-Aminovaleric acid (5AV) precursor. Components (a) and (b) can provided in the same formulation or can be provided in separate formulations in the product combination. In some embodiments, the composition and/or product combination comprises, consists essentially of, or consists of bacteria selected from the group consisting of: Bacteroides ovatus, Parabacteroides merdae, and Bacteroides thetaiotaomicron, or a mixture of two or more of the listed bacteria, and taurine, a taurine precursor, 5-Aminovaleric acid (5AV), and/or a 5-Aminovaleric acid (5AV) precursor. In some embodiments, the composition comprises, consists essentially of, or consists of Bacteroides, Butyricimonas, Paraprevotallacae, or a mixture of two or more of the listed bacteria. In some embodiments, the composition comprises, consists essentially of, or consists of Bacteriodetes, Anaerofilum, Anaerotruncus, Christensenella, Pseudoramibacter eubacterium, Holdemania, or a mixture of two or more of the listed bacteria. In some embodiments, the composition comprises, consists essentially of, or consists of Bacteroidetes, Holdemania, and a mixture of two or more of the listed bacteria. In some embodiments, the composition comprises, consists essentially of, or consists of a Bacteroidetes selected from the group consisting of Bacteroidaceae, Paraprevotellacae, Banesiellaceae, Rikenellaceae, Odoribacteraceae, Bacteroides, Buryricimonas, or a mixture of two or more of the listed bacteria. In some embodiments, the composition comprises, consists essentially of, or consists of Bacteroides ovatus, Parabacteroides merdae, and Bacteroides thetaiotaomicron, or a mixture of two or more of the listed bacteria. In some embodiments, the composition comprises, consists essentially of, or consists of a Clostridiales selected from the group consisting of Lachnospiraceae and Clostridium. In some embodiments, the composition comprises, consists essentially of, or consists of Bacteroides ovatus and Parabacteroides merdae and the taurine, taurine precursor, 5AV and/or 5AV precursor. In some embodiments, the composition and/or product combination comprises, consists essentially of, or consists of Bacteroides ovatus and Bacteroides thetaiotaomicron and the taurine, taurine precursor, 5AV and/or 5AV precursor. In some embodiments, the composition and/or product combination comprises, consists essentially of, or consists of Parabacteroides merdae and Bacteroides thetaiotaomicron and the taurine, taurine precursor, 5AV and/or 5AV precursor. In some embodiments, the composition and/or product combination comprises, consists essentially of, or consists of Bacteroides ovatus, Parabacteroides merdae, and Bacteroides thetaiotaomicron and the taurine, taurine precursor, 5AV and/or 5AV precursor. In some embodiments, the compositions and/or product combinations as described herein do not comprise, or are substantially free of Eisenbergiela tayi. In some embodiments, the compositions and/or product combinations as described herein do not comprise, or are substantially free of Eggerthella, Alstipes, Burkolderiales, Enterococcaceae, Clostridium, and/or Ruminococcus. In some embodiments, the aforementioned compositions and/or product combinations do not comprise, or are substantially free of Ruminococcaceace, Oscillospira, and/or Suterella. In some embodiments, the composition further comprises Lactobacillus reuteri. Without being limited by theory, it is contemplated that colonization of the gut by L. reuteri can further contribute to the inhibition, amelioration, prevention, delay in onset, or reduction in severity of ASD or a symptom thereof, for example impaired social behavior (See, e.g,. Buffington et al. (2016) Cell 165: 1762-1775, which is incorporated herein by reference in its entirety). In some embodiments, the composition and/or product combination comprises, consists essentially of, or consists of bacteria selected from the group consisting of: Bacteroides ovatus, Parabacteroides merdae, and Bacteroides thetaiotaomicron, or a mixture of two or more of the listed bacteria (e.g., Bacteroides ovatus and Parabacteroides merdae; Bacteroides ovatus and Bacteroides thetaiotaomicron; Parabacteoides merdae, and Bacteroides thetaiotaomicron; or Bacteroides ovatus, Parabacteroides merdae, and Bacteroides thetaiotaomicron), and one or more metabolites that are expressed differently in ASD and non-ASD subjects as shown in any of Tables 3-1, 3-2, and/or 3-3. In some embodiments, the taurine, taurine precursor, 5AV and/or 5AV precursor are administered to the subject prenatally (e.g., to the subject, or to the subject's mother), and the bacteria are administered to the subject after birth.

In some embodiments, a first composition comprising, consisting essentially of, or consisting of the bacteria is provided along with a second composition comprising, consisting essentially of, or consisting of taurine, a taurine precursor, 5-Aminovaleric acid (5AV), and/or a 5-Aminovaleric acid (5AV) precursor (e.g., as a product combination). For example, a first composition comprising the Bacteroides ovatus, Parabacteroides merdae, and Bacteroides thetaiotaomicron, or a mixture of two or more of the listed bacteria can be provided along with a second composition comprising taurine, a taurine precursor, 5-Aminovaleric acid (5AV), and/or a 5-Aminovaleric acid (5AV) precursor (e.g., as a product combination). The two compositions can be administered to the subject separately or simultaneously, so as to administer the bacteria and metabolite or metabolite precursor to the subject. In some embodiments, the two compositions are for use in preparing a combined composition that comprises, consists essentially of, or consists of the bacteria and the comprising taurine, taurine precursor, 5-Aminovaleric acid (5AV), and/or 5-Aminovaleric acid (5AV) precursor as described herein. For example, in some embodiments, the two compositions are for use in preparing a combined composition that comprises the Bacteroides ovatus, Parabacteroides merdae, and Bacteroides thetaiotaomicron, or a mixture of two or more of the listed bacteria and the comprising taurine, taurine precursor, 5-Aminovaleric acid (5AV), and/or 5-Aminovaleric acid (5AV) precursor as described herein.

In some embodiments, the composition and/or product combination comprises Bacteroides ovatus and Parabacteroides merdae. In some embodiments, the composition and/or product combination comprises Bacteroides ovatus and Bacteroides thetaiotaomicron. In some embodiments, the composition and/or product combination comprises Parabacteroides merdae, and Bacteroides thetaiotaomicron. In some embodiments, the composition and/or product combination comprises Bacteroides ovatus, Parabacteroides merdae, and Bacteroides thetaiotaomicron. In some embodiments, the aforementioned compositions and/or product combinations do not comprise, or are substantially free of Eisenbergiela tayi. In some embodiments, the aforementioned compositions and/or product combinations do not comprise, or are substantially free of Eggerthella, Alstipes, Burkolderiales, Enterococcaceae, Clostridium, and/or Ruminococcus. In some embodiments, the aforementioned compositions and/or product combinations do not comprise, or are substantially free of Ruminococcaceace, Oscillospira, and/or Suterella.

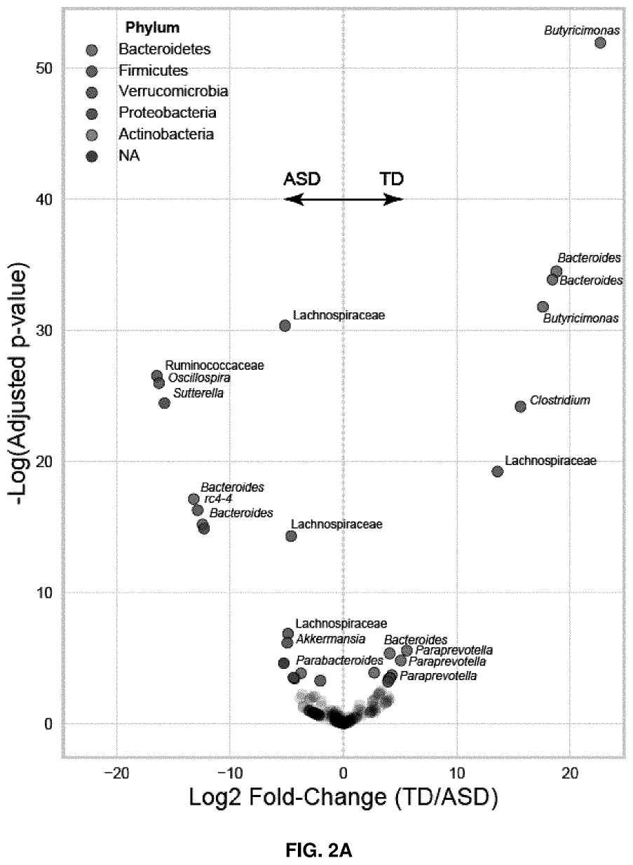

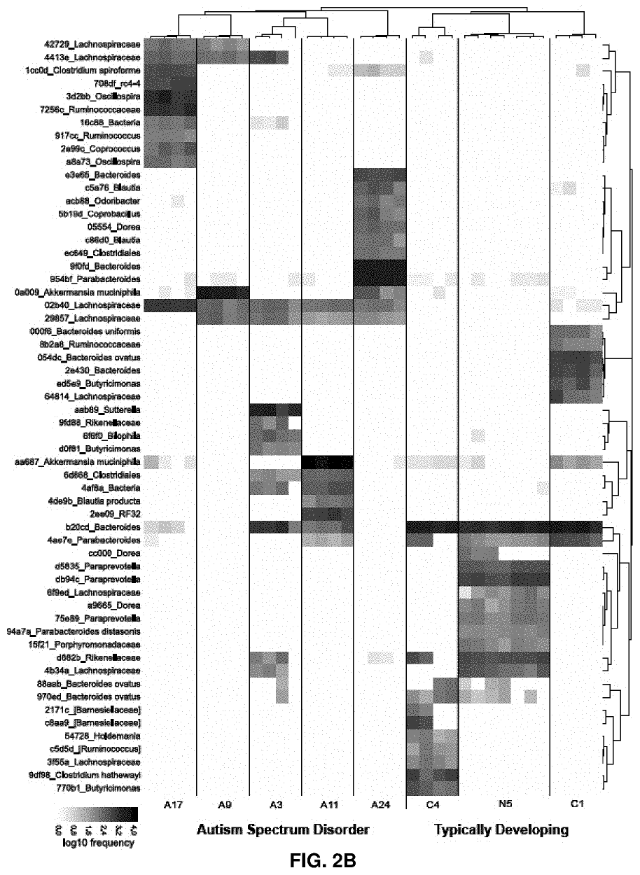

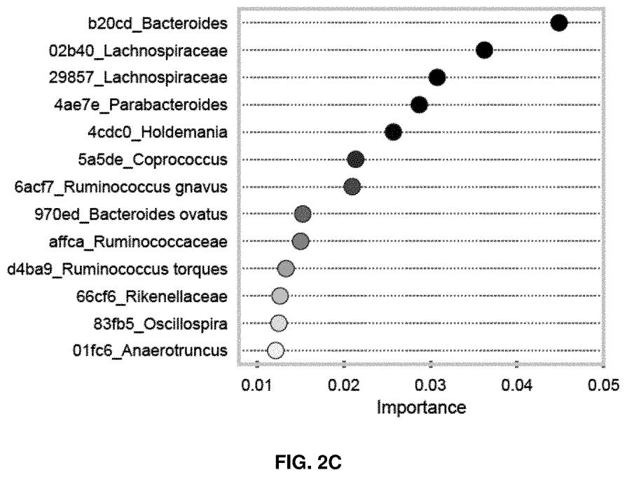

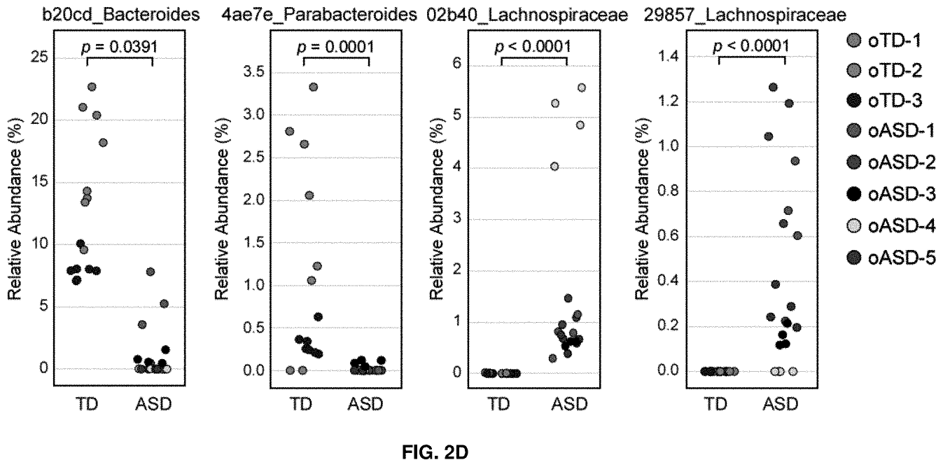

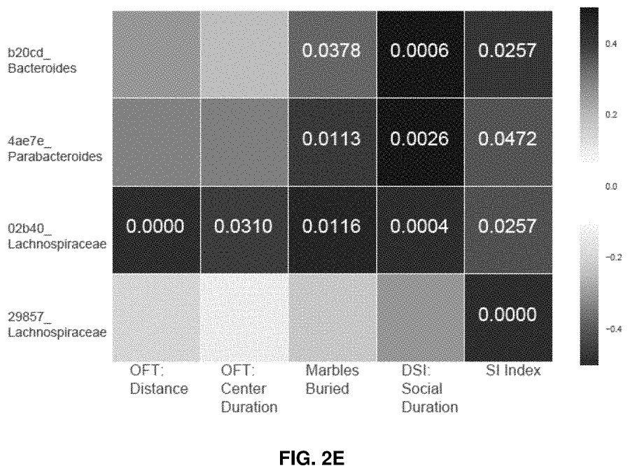

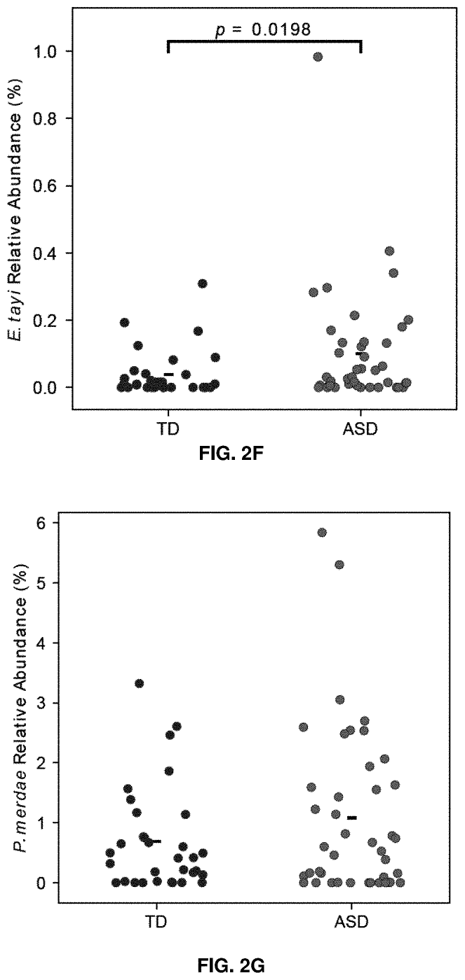

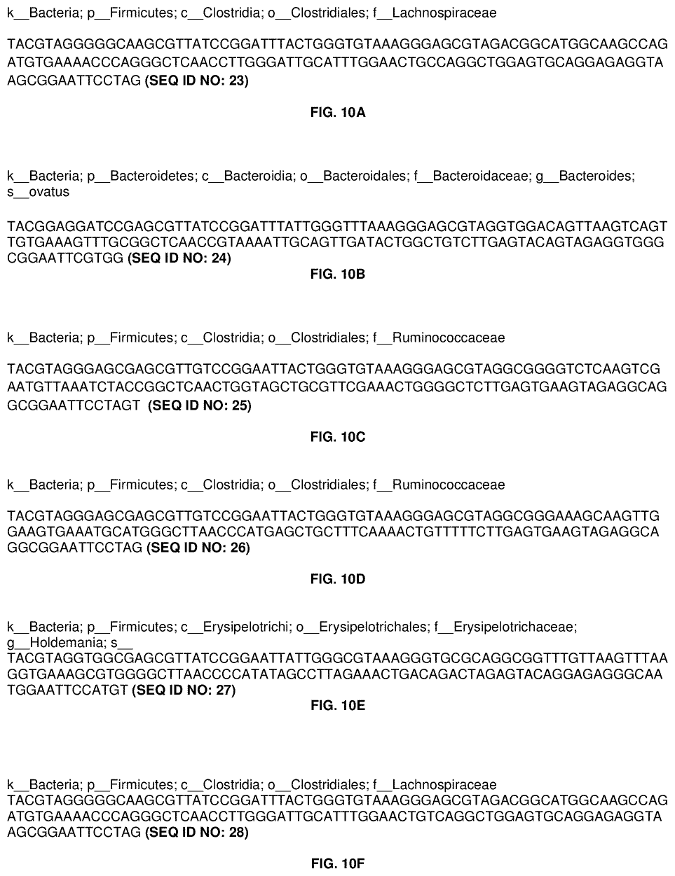

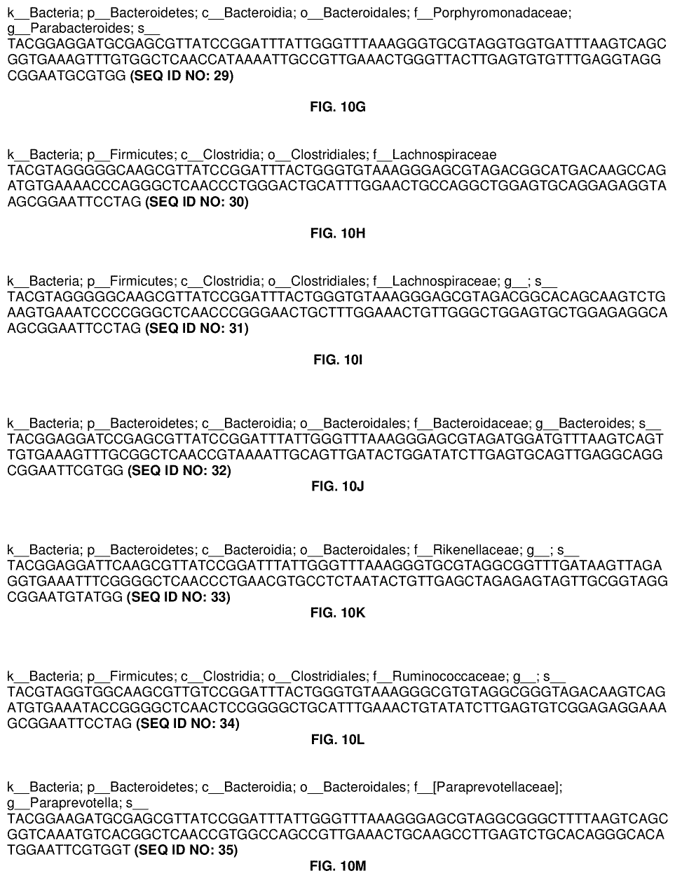

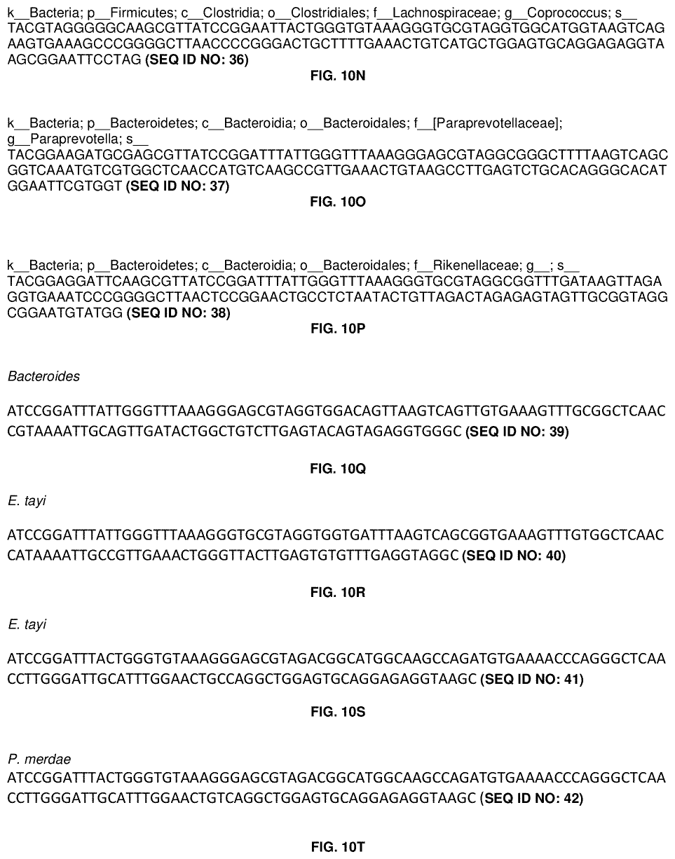

In some embodiments, the composition and/or product combination comprises, consists essentially of, or consists of Bacteroides ovatus and Parabacteroides merdae and the taurine and/or taurine precursor. In some embodiments, the composition and/or product combination comprises, consists essentially of, or consists of Bacteroides ovatus and Bacteroides thetaiotaomicron and the taurine and/or taurine precursor. In some embodiments, the composition and/or product combination comprises, consists essentially of, or consists of Parabacteroides merdae and Bacteroides thetaiotaomicron and the taurine and/or taurine precursor. In some embodiments, the composition and/or product combination comprises, consists essentially of, or consists of Bacteroides ovatus, Parabacteroides merdae, and Bacteroides thetaiotaomicron and the taurine and/or taurine precursor. In some embodiments, the composition and/or product combination comprises, consists essentially of, or consists of Bacteroides ovatus and Parabacteroides merdae and the 5AV and/or 5AV precursor. In some embodiments, the composition and/or product combination comprises, consists essentially of, or consists of Bacteroides ovatus and Bacteroides thetaiotaomicron and the 5AV and/or 5AV precursor. In some embodiments, the composition and/or product combination comprises, consists essentially of, or consists of Parabacteroides merdae and Bacteroides thetaiotaomicron and the 5AV and/or 5AV precursor. In some embodiments, the composition and/or product combination comprises, consists essentially of, or consists of Bacteroides ovatus, Parabacteroides merdae, and Bacteroides thetaiotaomicron and the 5AV and/or 5AV precursor. In some embodiments, the composition and/or product combination comprises, consists essentially of, or consists of Bacteroides ovatus and Parabacteroides merdae and the taurine and/or taurine precursor. In some embodiments, the composition and/or product combination comprises, consists essentially of, or consists of Bacteroides ovatus and Bacteroides thetaiotaomicron and the taurine and/or taurine precursor. In some embodiments, the composition and/or product combination comprises, consists essentially of, or consists of Parabacteroides merdae and Bacteroides thetaiotaomicron and the taurine and/or taurine precursor. In some embodiments, the composition and/or product combination comprises, consists essentially of, or consists of Bacteroides ovatus, Parabacteroides merdae, and Bacteroides thetaiotaomicron and the taurine and/or taurine precursor. In some embodiments, the aforementioned compositions and/or product combinations do not comprise, or are substantially free of Eisenbergiela tayi. It is further contemplated that as analysis of sub-operational-taxonomic units (sOTUs) based on 16S RNA sequencing identified taxa that differ between ASD and non-ASD gut microbiota (see Example 2), suitable bacteria for the compositions and/or product combinations in accordance with some embodiments herein map to the differently-present sOTU (see, e.g., FIG. 2D). In view of the present disclosure, it will be understood whether a particular bacterium maps to a noted sOTU, for example by comparing a 16S RNA sequence of that bacteria to the noted OTU. By way of example, the GreenGenes 13_8 99% OTU table can be used for such a comparison, and is available on QIIME, accessible on the world wide web (See, Caporaso, J. G. et al. QIIME allows analysis of high-throughput community sequencing data. Nature Methods 7, 335-336 (2010), which is hereby incorporated by reference in its entirety). Example 16S sequences of bacteria described herein are shown in FIGS. 10A-T. In some embodiments, the composition and/or product combination comprises, consists essentially of, or consists one or more bacterial species that map to an sOTU selected from the group consisting of: b20cd_Bacteroides, and 4ae7e_Parabacteroides. In some embodiments, the composition and/or product combination comprises, consists essentially of, or consists of one or more bacterial species that map to the sOTU of b20cd_Bacteroides, and one or more bacterial species that map to the sOTU of 4ae7e_Parabacteroides. In some embodiments, the composition and/or product combination comprises, consists essentially of, or consists one or more bacterial species that map to the sOTU of b20cd_Bacteroides. In some embodiments, the composition and/or product combination comprises, consists essentially of two, three, four, five, six, seven, eight, nine, or ten bacterial species that each map to the sOTU of b20cd_Bacteroides. In some embodiments, the composition and/or product combination comprises, consists essentially of, or consists one or more bacterial species that map the sOTU of 4ae7e_Parabacteroides. In some embodiments, the composition and/or product combination comprises, consists essentially of two, three, four, five, six, seven, eight, nine, or ten bacterial species that each map to the sOTU of 4ae7e_Parabacteroides. In some embodiments, a bacteria maps to an sOTU when the bacteria comprises a 16S rRNA sequence of at least 100 nucleotides that has at least 97% identity to a reference 16S rRNA sequence of the sOTU. In some embodiments, a bacteria maps to an sOTU when the bacteria comprises a 16S rRNA sequence of at least 100 nucleotides that has at least 99% identity to a reference 16S rRNA sequence of the sOTU. In some embodiments, the composition or product combination as described herein is for use in reducing one or more symptoms of Autism Spectrum Disorder (ASD) in a subject after birth, in which the composition or product combination is administered to said subject prenatally.

In some embodiments described herein, the taurine precursor is selected from the group consisting of; cysteine, cysteine sulfinic acid, homocysteine, cystathionine, hypotaurine, and a mixture of two or more of the listed items (for example cysteine and cysteine sulfinic acid, cysteine and homocysteine, cysteine and cystathionine, cysteine and hypotaurine, cysteine sulfinic acid and homocysteine, cysteine sulfinic acid and cystathionine, cysteine sulfinic acid and hypotaurine, and/or cystathionine and hypotaurine).

In some of the embodiments described herein, the 5AV precursor is selected from the group consisting of; lysine, cadaverine, 1-piperideine, and a mixture of two or more of the listed items (for example lysine and cadaverine, lysine and 1-piperideine, or cadaverine and 1-piperidine).

In some embodiments, the composition and/or product combination consists essentially of the bacteria and the taurine and/or taurine precursor or the bacteria and the 5AV and/or 5AV precursor.

In some embodiments, the composition and/or product combination is for use in reducing one or more symptoms of Autism Spectrum Disorder (ASD), for example, repetitive behavior, hyperactivity, anxiety, and/or a communication disorder in a subject after birth. In some embodiments, the subject is a human.

In some embodiments, the composition, product combination, use, and/or method comprises an amount of bacteria establish a colony (e.g., a colony that persists for at least 1, 2, 3, 4 or more weeks post-inoculation) in the gut of a human subject when administered in a standard manner for microbiome transplant, probiotic treatment or equivalent procedures. Such an amount of bacteria may be referred to herein as an “inoculum.” In some embodiments, the amount of bacteria in the composition, product combination, use, or method includes at least 10⁴ colony forming units (cfu), for example at least 10⁴, 10⁵, 10⁶, 10⁷, 10⁸, 10⁹, 10¹⁰, 10¹¹, 10¹², or 10¹³ cfu, including ranges between any of the listed values, for example 10⁴-10⁸ cfu, 10⁴-10⁹ cfu, 10⁴-10¹⁰ cfu, 10⁴-10¹¹ cfu, 10⁴-10¹² cfu, 10⁴-10¹² cfu, 10⁵-10⁸ cfu, 10⁵-10⁹ cfu, 10⁵-10¹⁰ cfu, 10⁵-10¹¹ cfu, 10⁵-10¹² cfu, 10⁵-10¹² cfu, 10⁶-10⁸ cfu, 10⁶-10⁹ cfu, 10⁶-10¹⁰ cfu, 10⁶-10¹¹ cfu, 10⁶-10¹² cfu, 10⁶-10¹² cfu, 10⁷-10⁸ cfu, 10⁷-10⁹ cfu, 10⁷-10¹⁰ cfu, 10⁷-10¹¹ cfu, 10⁷-10¹² cfu, 10⁷-10¹² cfu, -10⁹ cfu, 10⁸-10¹⁰ cfu, 10⁸-10¹¹ cfu, 10⁸-10¹² cfu, or 10⁸-10¹² cfu. In some embodiments, the composition, product combination, use, and/or method comprises a log phase (at 37° C.) of bacteria for administration to the subject. In some embodiments, the composition, product combination, use, and/or method comprises a stationary phase (at 37° C.) of bacteria for administration to the subject. In some embodiments, the bacteria of the composition, product combination, use, and/or method are isolated bacteria.

In some embodiments, the composition and/or product combination does not comprise, or is substantially free of Eisenbergiela tayi. Without being limited by theory, it is contemplated that the presence of Eisenbergiela tayi correlates with ASD symptoms (See Example 2; FIGS. 2A and 2D), and as such, a composition that is free or substantially free of Eisenbergiela tayi is useful for colonizing or recolonizing a gut of a subject so as to reduce the risk and/or severity of a symptom of ASD. As used herein, “substantially free” and variations of this root term has its customary and ordinary meaning as understood by one of skill in the art in view of this disclosure. It refers to a composition and/or product combination having no more than trace amounts of a substance (e.g., a bacteria such as Eisenbergiela tayi), and/or the amount or presence of the substance having no appreciable effect (e.g. behavioral effect) on the subject. For example, in some embodiments, a composition and/or product combination substantially free of a bacteria comprises less than about 10⁶ cfu of that bacteria, for example less than 10⁶ cfu, 10⁵ cfu, 10⁴ cfu, 10³ cfu, 10² cfu, or 10 cfu. In some embodiments, a composition and/or product combination substantially free of a bacteria comprises less than about 10⁴ cfu of that bacteria, for example less than 10⁴ cfu, 10³ cfu, 10² cfu, or 10 cfu. Accordingly, a composition and/or product combination substantially free of Eisenbergiela tayi in accordance with compositions, methods, and uses of some embodiments herein, may comprise Eisenbergiela tayi in trace amounts, and/or the amount or presence of Eisenbergiela tayi has no appreciable behavioral effect on the subject. By way of example, in some embodiments, a composition or product combination is substantially free of Eisenbergiela tayi when it comprises less than 10⁴ cfu, 10³ cfu, 10² cfu, or 10 cfu of Eisenbergiela tayi. In some embodiments, the composition or product combination is free or substantially free of a bacteria that is expressed at least 2-fold more in TD compared to ASD patients as shown in FIG. 2A. In some embodiments, the compositions and/or product combinations as described herein do not comprise, or are substantially free of Eggerthella, Alstipes, Burkolderiales, Enterococcaceae, Clostridium, Ruminococcus, Ruminococcaceace, Oscillospira, and/or Suterella. In some embodiments, the compositions and/or product combinations as described herein do not comprise, or are substantially free of Eggerthella, Alstipes, Burkolderiales, Enterococcaceae, Clostridium, and/or Ruminococcus.

In some embodiments, the composition and/or product combination is stabilized. For example, in some embodiments, the composition and/or product combination is stabilized in that it comprises live bacteria that are not in a logarithmic growth phase. For example, in some embodiments, the composition and/or product combination is stabilized in that it comprises a substance that maintains one or more conditions of the composition, for example a buffer, insulation, and/or a sealant. In some embodiments, the substance that maintains one or more conditions of the composition is selected from the group consisting of a buffer, an insulation, a sealant, a cryoprotectant, an anti-oxidant, and a coating, or a combination of two or more of the listed items.

In some embodiments, the composition and/or product combination includes nutrients or media in which the bacteria were cultured or additional nutrients that increase the likelihood of successfully establishing the colony. In some embodiments, the composition and/or product combination further comprises a pharmaceutically acceptable carrier or excipient. “Pharmaceutically acceptable” carriers have their ordinary and customary meaning as would be understood by one of skill in the art in view of this disclosure, and include ones which are nontoxic to the cell or mammal being exposed thereto at the dosages and concentrations employed. Example “Pharmaceutically acceptable” carriers in accordance with methods and uses and compositions and product combinations herein can comprise, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. In some embodiments, a composition or product combination is encapsulated, for example in a pill, capsule, bead, matrix, or gel. In some embodiments, the composition or product combination is encapsulated in a pH-sensitive coating or matrix that dissolves at a pH of the small intestine (typically about pH 5.5-6.8) or the colon (typically about pH 6.5-7.0), but does not dissolve at the pH of the stomach (typically about pH 1.5-3.5). For example, the coating can comprise, consist essentially of, or consist of a hydrogel, acrylic acid, and/or cellulose. For example, without being limited by theory, it is contemplated that encapsulating a bacteria can protect a bacteria as described herein in the conditions of the stomach, so that the bacteria can survive passage through the stomach and colonize the GI tract. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextran, chelating agents such as EDTA, sugar alcohols such as mannitol or sorbitol, salt-forming counterions such as sodium, and nonionic surfactants such as and nonionic surfactants such as TWEEN™ surfactant, polyethylene glycol (PEG), and PLURONICS™ surfactant. Auxiliary, stabilizer, emulsifier, lubricant, binder, pH adjustor controller, isotonic agent and other conventional additives may also be added to the carriers. In some embodiments, the composition is formulated for oral administration, rectal administration, or oral and rectal administration. In some embodiments, the composition and/or product combination comprises, consists essentially of, or consists of a probiotic. In some embodiments, the composition and/or product combination does not comprise an antibiotic. In some embodiments, a method as described herein comprises administering a composition or product combination as described herein, but does not comprise administering an antibiotic. In some embodiments, a method as described herein comprises administering bacteria and/or a metabolite or metabolite precursor as described herein, but does not comprise administering an antibiotic.

As used herein, a “taurine precursor” has its customary and ordinary meaning as understood by one of skill in the art in view of this disclosure. Example taurine precursors suitable for compositions, product combinations, uses, and methods of embodiments described herein include, but are not limited to: cysteine, cysteine sulfinic acid, homocysteine, cystathionine, hypotaurine, and a mixture of two or more of the listed items, for example cysteine and cysteine sulfinic acid, cysteine and homocysteine, cysteine and cystathionine, cysteine and hypotaurine, cysteine sulfinic acid and homocysteine, cysteine sulfinic acid and cystathionine, cysteine sulfinic acid and hypotaurine, and/or cystathionine and hypotaurine. In some embodiments, a taurine precursor is a compound which is substantially (e.g., >10%) or primarily (e.g., >50%) converted into taurine by the normal metabolic pathways of enteric bacteria (e.g., Bacteroides and Parabacteroides species) when provided to such bacteria in vivo.

As used herein, a “5AV precursor” has its customary and ordinary meaning as understood by one of skill in the art in view of this disclosure. Example 5AV precursors suitable for compositions, product combinations, uses, and methods of embodiments described herein include, but are not limited to: lysine, cadaverine, 1-piperideine, and a mixture of two or more of the listed items, for example lysine and cadaverine, lysine and 1-piperideine, or cadaverine and 1-piperidine. In some embodiments, a 5AV precursor is a compound which is substantially (e.g., >10%) or primarily (e.g., >50%) converted into 5AV by the normal metabolic pathways of enteric bacteria (e.g., Bacteroides and Parabacteroides species) when provided to such bacteria in vivo.

### Methods and Compositions Comprising Metabolites for Reducing Symptoms of ASD

It has been observed herein that prenatal administration of taurine and/or 5AV (e.g., by way of administration to the pregnant mother) reduces symptoms of ASD in the infant (See Example 5). Accordingly, some embodiments include methods of reducing or preventing one or more symptoms of ASD in a selected subject after birth. The method can comprise administering a composition comprising, consisting essentially of, or consisting of an amount of taurine and/or 5AV to the prenatal subject (e.g., by way of administration of to the pregnant mother), the amount being effective to reduce or prevent one or more symptoms of ASD in the subject after birth. Additionally, in some embodiments, the prenatal subject is identified as being a part of a selected subpopulation, for example a subpopulation of prenatal subjects at risk of developing ASD. In some embodiments, the prenatal subject is identified by detecting a bacteria and/or metabolite profile in the subject or the subject's mother, as described herein. In some embodiments, the symptom of ASD comprises a sociability disorder and/or a repetitive or compulsive behavior and/or anxiety (as would be observed after birth of the prenatal subject). In some embodiments, the prenatal subject is a human.

In some embodiments, a composition comprising, consisting essentially of, or consisting of taurine, 5AV, or a combination of taurine and 5AV is provided for use in reducing or preventing one or more symptoms of ASD in a subject in need thereof after birth. For example, the subject can be a prenatal subject at risk of ASD, at risk of having at least one symptom of ASD after birth. In some embodiments, the one or more symptoms of ASD comprises, consists essentially of, or consists of a sociability disorder, a repetitive and/or a compulsive behavior, and/or anxiety (for example, a sociability disorder and a repetitive and/or a compulsive behavior, a sociability disorder and anxiety, anxiety and a repetitive and/or a compulsive behavior, or a sociability disorder and anxiety and a repetitive and/or a compulsive behavior). In some embodiments, the composition comprises, consists essentially of, or consists of an amount of taurine effective to reduce or prevent the one or more symptoms of ASD in the subject after birth. In some embodiments, the composition comprises, consists essentially of, or consists of an amount of 5AV effective to reduce or prevent the one or more symptoms of ASD in the subject after birth. In some embodiments, the composition comprises, consists essentially of, or consists of an amount of taurine and 5AV effective to reduce or prevent the one or more symptoms of ASD in the subject after birth. In some embodiments, the composition comprises, consists essentially of, or consists of one or more metabolites that are expressed differently in ASD and non-ASD subjects as shown in any of Tables 3-1, 3-2, and/or 3-3.

In some embodiments, the method comprises administering the taurine and the 5AV to the subject (e.g., either directly to the prenatal subject or indirectly to the prenatal subject by administering the taurine and/or 5AV to the prenatal subject's mother in a manner that the taurine and/or 5AV is circulated or delivered to the prenatal subject). In some embodiments, the taurine and 5AV are administered to the subject in a single composition (e.g., either directly to the prenatal subject or indirectly to the prenatal subject by administering the taurine and/or 5AV to the prenatal subject's mother in a manner that the taurine and/or 5AV is circulated or delivered to the prenatal subject). In some embodiments, the taurine and 5AV are administered to the subject in two or more compositions (e.g., either directly to the prenatal subject or indirectly to the prenatal subject by administering the taurine and/or 5AV to the prenatal subject's mother in a manner that the taurine and/or 5AV is circulated or delivered to the prenatal subject). For example, one composition can comprise taurine, and one composition can comprise 5AV. In some embodiments, the taurine and/or 5AV are administered in a unit dose, in which the unit dose is sufficient to reduce or prevent the symptom of ASD in the subject after birth (e.g., either directly to the prenatal subject or indirectly to the prenatal subject by administering the taurine and/or 5AV to the prenatal subject's mother in a manner that the taurine and/or 5AV is circulated or delivered to the prenatal subject). In some embodiments, the method comprises administering a composition comprising, consisting essentially of, or consisting of one or more metabolites that are expressed differently in ASD and non-ASD subjects as shown in any of Tables 3-1, 3-2, and/or 3-3 to the prenatal subject (e.g., either directly to the prenatal subject or indirectly to the prenatal subject by administering the taurine and/or 5AV to the prenatal subject's mother in a manner that the taurine and/or 5AV is circulated or delivered to the prenatal subject).

It has been discovered that prenatal administration of taurine (e.g., by way of administration of taurine to the pregnant mother) reduces anxiety, reduces repetitive behavior, and increases sociability of the infant in an art-recognized mouse model of ASD, the BTBR model (See Example 5). Moreover, prenatal administration of 5AV (e.g., by way of administration of 5AV to the pregnant mother) increases sociability in the BTBR model (See Example 5). Accordingly, in some embodiments, the one or more symptom of ASD that is reduced in the subject after birth by these methods comprises, consists essentially of, or consists of a sociability disorder, a repetitive behavior, anxiety, a sociability disorder and/or a repetitive behavior, a sociability disorder and/or anxiety, or anxiety and/or a repetitive behavior.

In some embodiments, a composition comprising, consisting essentially of, or consisting of taurine is administered to the prenatal subject (e.g., by way of administration of taurine to the pregnant mother) to reduce one or more ASD symptoms in the subject after birth, and the ASD symptoms that are reduced in the subject after birth comprise, consist essentially of, or consist of a sociability disorder, or a repetitive behavior, or a sociability disorder and a repetitive behavior. In some embodiments, a composition comprising, consisting essentially of, or consisting of taurine is administered to the prenatal subject (e.g., by way of administration of taurine to the pregnant mother) to reduce one or more ASD symptoms in the subject after birth, and the ASD symptoms that are reduced in the subject include: a sociability disorder, a repetitive behavior, and/or anxiety or any combination thereof. In some embodiments, a composition comprising, consisting essentially of, or consisting of 5AV is administered to the prenatal subject (e.g., by way of administration of 5AV to the pregnant mother) to reduce one or more ASD symptoms in the prenatal subject after birth, and the ASD symptoms that are reduced in the prenatal subject after birth comprise a sociability disorder. In some embodiments, a composition comprising, consisting essentially of, or consisting of taurine and 5AV is administered to the prenatal subject (e.g., by way of administration of taurine and 5AV to the pregnant mother) to reduce one or more ASD symptoms in the prenatal subject after birth, and the ASD symptoms that are reduced in the prenatal subject after birth include: a sociability disorder, a repetitive behavior, or a sociability disorder or any combination thereof. In some embodiments, a composition comprising, consisting essentially of, or consisting of taurine and 5AV is administered to the prenatal subject (e.g., by way of administration of taurine to the pregnant mother) to reduce one or more ASD symptoms in the prenatal subject after birth, and the ASD symptoms that are reduced in the prenatal subject after birth include: a sociability disorder, a repetitive behavior, or anxiety or any combination thereof.

In some embodiments, the prenatal subject is selected as one being at risk of developing ASD or a symptom of ASD. In some embodiments, the prenatal subject is selected as being at risk of developing ASD or a symptom of ASD after birth, based on a profile comprising one or more of:
(a) a presence and/or level of a gut bacterium selected from the group consisting of; Bacteroides ovatus, Parabacteroides merdae, Bacteroides thetaiotaomicron, or a combination of two or more of the listed bacteria; and a presence and/or level of Eisenbergiela tayi in a sample from the prenatal subject or the prenatal subject's mother;
(b) a presence or gene product level of a gut microbiota gene that is an ortholog of KEGG ortholog K0681, and/or a presence or gene product level of a gene that is an ortholog of KEGG ortholog K1442, in a sample from the prenatal subject or the prenatal subject's mother, preferably by screening a sample from the gut, feces, or gut and feces material of the subject or the subject's mother;
(c) a level of colon taurine, 5-Aminovaleric acid (5AV), lysine, 3-aminoisobutyric acid (3-AIBA), genistein, daidzein, lysine, 5-aminopentanoate, cellobiose, glyceric acid or genistein, a level of serum D ribose, ribitol, ribonic acid, or L-tyrosine, or a rate of degradation thereof in a sample from the prenatal subject or the prenatal subject's mother;
(d) a level of a product of a gene of the subject selected from the group consisting of: Gm26944 (or a human ortholog thereof), Gm13016 (or an ortholog thereof), Gm17259 (or a human ortholog thereof), 4930539E08Rik (or a human ortholog thereof), Dag1b (or a human ortholog thereof), or a combination of two or more of the listed genes in a sample from the prenatal subject or the prenatal subject's mother and/or
(e) a level of a splice variant of the subject selected from the group consisting of: a mutually exclusive exon in Neurexin 2 (or a human ortholog thereof); a mutually exclusive exon in Ankryin 2 (or a human ortholog thereof); a skipped exon in Fmr1 (or a human ortholog thereof); a skipped exon in Ube3a (or a human ortholog thereof); a skipped exon in Rims1 (or a human ortholog thereof); a skipped exon of Cacna1c (or a human ortholog thereof); a retained intron of Ads1 (or a human ortholog thereof); a skipped exon of a pogo transferrable element derived with ZNF domain Pogz (or a human ortholog thereof in a sample from the prenatal subject or the prenatal subject's mother); or a skipped exon of Trip12 (or a human ortholog thereof). In some embodiments, (e) comprises a level of a splice variant of the subject selected from the group consisting of: a mutually exclusive exon in Neurexin 2 (or a human ortholog thereof); a mutually exclusive exon in Ankryin 2 (or a human ortholog thereof); a skipped exon of Cacna1c (or a human ortholog thereof); a retained intron of Ads1 (or a human ortholog thereof); or a skipped exon of a pogo transferrable element derived with ZNF domain Pogz (or a human ortholog thereof in a sample from the prenatal subject or the prenatal subject's mother).
   In some embodiments, the profile comprises the detected presence and/or levels of (a), (b), (c), (d), (e), or a combination of two or more of (a), (b), (c), (d), and (e). In some embodiments, the profile is of a sample of the subject. Profiles that are indicative or predictive of a presence, increased risk, and/or increased severity of ASD or one or more symptoms of ASD are described herein (&ee, e.g., the section entitled “Methods of Determining A Profile Of A Sample Of A Subject”). In some embodiments, the profile is detected and interpreted according to at least one methods of determining a profile of a sample of a subject as described herein, for example a fecal and/or serum sample. In some embodiments, the profile is of a sample of the subject's mother, for example a fecal and/or serum sample of the subject mother. In some embodiments, the sample of the subject comprises CNS tissue and/or CSF of the subject. In some embodiments the profile comprises detecting the presence and/or level one or more metabolites that are expressed differently in ASD and non-ASD subjects as shown in any of Tables 3-1, 3-2, and/or 3-3.

In some embodiments, the prenatal subject is selected as being at risk of developing ASD or a symptom of ASD after birth due to the mother of the prenatal subject having a colon comprising reduced levels of taurine and/or 5AV compared to a control mother of a non-ASD offspring, and/or elevated levels of 3-aminoisobutyric acid (3AIBA) compared to the control mother of a non-ASD offspring. In accordance with methods, compositions, and product combinations of some embodiments herein, a control mother can comprise an individual mother (e.g., an-offspring bearing mammal of the same species as the mother of the subject of interest) and/or a population of mothers. A suitable control mother will be appreciated in view of the present disclosure, and in the context of the particular subject (e.g., prenatal subject), ASD symptom(s), sample type, and profile of interest. By way of example, in some embodiments, a control mother (or population of control mothers) is confirmed to have a non-ASD offspring, and/or offspring that are not at risk for the relevant ASD symptoms. For example, the offspring of the control mother can have an ADOS score of three or less, for example three, two, or one. Optionally, samples from the control mother can be obtained prenatally at an earlier point in time than those of the subject mother, so as to facilitate testing of the control mother's offspring for ASD or one or more symptoms thereof. For example, in some embodiments, if a risk of repetitive behavior is being determined, a sample can be collected prenatally from a control mother (or population of control mothers), saved for later (for example, frozen, fixed, or the like), and the offspring can be confirmed after birth not to exhibit the repetitive behavior, so as to confirm that the saved sample is a suitable “non-ASD” control for repetitive behavior.

In some embodiments, the subject is selected as having or being expected to have reduced levels of taurine and/or 5AV compared to a control non-ASD control; and/or elevated levels of 3-aminoisobutyric acid (3AIBA) compared to the non-ASD control. By way of the example, in some embodiments, the non-ASD control does not have ASD, and/or does not exhibit relevant ASD symptoms. For example, the non-ASD control can have an ADOS score of three or less, for example three, two, or one.

It is observed herein that administering taurine and/or 5AV prenatally reduces and/or prevents symptoms of ASD in an art-recognized mouse model of ASD, the BTBR model after birth of the animals. However, the taurine and 5AV did not have these effects when administered to juveniles (See Example 5). As such, in some embodiments, the composition comprising, consisting essentially of, or consisting of taurine, 5AV, or combination of taurine and 5AV is administered to the prenatal subject (i.e. prior to the birth of the subject). In some embodiments, the composition comprising, consisting essentially of, or consisting of taurine, 5AV, or combination of taurine and 5AV is administered directly to the prenatal subject (i.e. prior to the birth of the subject). In some embodiments, the composition comprising, consisting essentially of, or consisting of taurine, 5AV, or combination of taurine and 5AV is administered to the mother of a prenatal subject (prior to the birth of the prenatal subject). Without being limited by theory, it is contemplated that the blood-brain barrier of prenatal subjects is permeable to taurine and 5AV (so that metabolites such as taurine and 5AV and precursors thereof can travel from the blood to the brain), while the blood-brain barrier of older subjects is not permeable to these substances. Accordingly, in some embodiments, the composition comprising, consisting essentially of, or consisting of taurine, 5AV, or combination of taurine and 5AV is administered a subject that has a blood-brain barrier that is permeable to the taurine, 5AV, or combination thereof. In some embodiments, the composition is not administered to the subject postpartum.

The composition comprising, consisting essentially of, or consisting of taurine and/or 5AV in accordance with some embodiments herein can be administered to the subject by various routes. For example, the composition can be administered to the subject via oral administration, rectum administration, transdermal administration, intranasal administration, intravenous administration, subcutaneous administration, or inhalation. In some embodiments, the composition comprising, consisting essentially of, or consisting of taurine and/or 5AV is administered directly to a prenatal subject, and/or is administered indirectly through administration to the subject's mother. For example, the composition can be administered orally, rectally, transdermally, intranasally, intravenously, subcutaneously, via inhalation, and/or via fecal transplant to the subject's mother. For example, the composition can be administered orally, rectally, and/or via fecal transplant directly to the subject. For example, the composition can be administered orally, rectally, and/or via fecal transplant to the subject's mother, and can also be administered orally, rectally, transdermally, intranasally, intravenously, subcutaneously, via inhalation, and/or via fecal transplant to the prenatal subject.

In some embodiments, the composition is administered to the orally to the prenatal subject's mother and/or administered orally directly to the prenatal subject. For example, the composition can be a probiotic composition, a dietary supplement, a pharmaceutical composition, or a mixture thereof. Each dosage for human and animal subjects preferably contains a predetermined quantity of the bacteria calculated in an amount sufficient to produce the desired effect. The dosage forms of some embodiments can depend on the particular bacteria employed and the effect to be achieved. The composition can be administered alone or in combination with one or more additional probiotic, a dietary supplement, or therapeutic agents. Administration “in combination with” one or more further additional probiotic, a dietary supplement, or therapeutic agents includes both simultaneous (at the same time) and consecutive administration in any order. Administration can be chronic or intermittent, as deemed appropriate by the supervising practitioner, particularly in view of any change in the disease state or any undesirable side effects. “Chronic” administration refers to administration of the composition in a continuous manner while “intermittent” administration refers to treatment that is done with interruption.

Methods and Compositions Comprising Bacteria and/or Metabolite Precursors for Reducing Symptoms of ASD

It has been observed that compositions comprising certain gut bacteria are sufficient to induce symptoms of ASD in a subject (See Examples 1). Moreover, particular species of bacteria correlate with reduced symptoms of ASD. For example, Bacteroides species (b20cd_Bacteroides; maps to several different Bacteroides species) and P. merdae (4ae7e_Parabacteroides) both correlate with reduced repetitive behavior and increased social behavior (See Example 2). On the other hand, the Lacnospiraceae species E. tayi correlates with symptoms of ASD, including increased repetitive behavior and social interaction deficits (Example 2). By way of example, E. tayi can be identified as 02b40_Lacnospiraceae and/or 29857_Lacnospiraceae. Furthermore, following gut colonization with bacteria that correlate with symptoms of ASD, certain colon and serum metabolite levels are altered (Example 4), while prenatal administration of the metabolites taurine and 5AV (which were are lower levels in subjects having symptoms of ASD) reduced the symptoms of ASD (Example 5). Accordingly, it is contemplated herein that certain ASD-symptom-associated gut microbes involved in the production and degradation of ASD-symptom-associated metabolites impact symptoms of ASD through the production and/or degradation of these metabolites. As such, in some embodiments herein, a composition comprising, consisting essentially of, or consisting of bacteria and/or metabolite precursors is administered to a subject having, or at risk of having one or more symptoms of ASD, for example, repetitive behavior, deficient social behavior, and/or anxiety, and can reduce or prevent one or more of these symptoms of ASD after birth. It is further contemplated that in some embodiments, the subject is selected as being from a particular subpopulation of subjects having, or at risk of having symptoms of ASD. The subject can be selected as being part of the subpopulation based on a profile comprising gut bacteria, metabolites (e.g. in the colon and/or serum) of the subject or its mother, and/or on a profile of gene expression and/or splicing patterns in the subject as described herein. In some embodiments, the subject is human.

In some embodiments, a method of reducing a symptom of ASD in a selected subject comprises administering a composition comprising, consisting essentially of, or consisting of bacteria selected from the group consisting of: Bacteroides ovatus, Parabacteroides merdae, Bacteroides thetaiotaomicron, and a mixture of two or more of the listed bacteria to the subject (e.g., Bacteroides ovatus and Bacteroides thetaiotaomicron, Parabacteroides merdae and Bacteroides thetaiotaomicron, Bacteroides ovatus and Parabacteroides merdae, or Bacteroides ovatus and Parabacteroides merdae and Bacteroides thetaiotaomicron), whereby the symptom of ASD is reduced in the subject after birth. In some embodiments, the composition is free or substantially free of Eisenbergiela tayi as described herein. In some embodiments, the composition comprises, consists essentially of or consists of the noted bacteria (e.g., Bacteroides ovatus, Parabacteroides merdae, Bacteroides thetaiotaomicron, Bacteroides ovatus and Bacteroides thetaiotaomicron, Parabacteroides merdae and Bacteroides thetaiotaomicron, Bacteroides ovatus and Parabacteroides merdae, or Bacteroides ovatus Parabacteroides merdae, and Bacteroides thetaiotaomicron) but does not comprise a metabolite precursor.

In some embodiments, the composition further comprises a metabolite precursor, for example a taurine precursor, and/or a 5-Aminovaleric acid (5AV) precursor. As such, in some embodiments the composition comprises, consists essentially of or consists of the bacteria and the taurine precursor. In some embodiments the composition comprises, consists essentially of, or consists of the bacteria and the 5AV precursor. In some embodiments the composition comprises, consists essentially of or consists of the bacteria and the taurine precursor and the 5AV precursor. Examples of suitable compositions comprising bacteria and taurine and/or 5AV precursors, as well as, suitable taurine and/or 5AV precursors are described herein, for example, under the heading “Compositions Comprising Bacteria and/or Metabolite Precursors.” In some embodiments, the composition is stabilized.

As the presence of certain bacteria in the gastrointestinal tract results in symptoms of ASD, and the presence or absence of certain bacteria correlate with the symptoms of ASD (See Examples 1-2), in some embodiments, the administration of the composition comprise colonizing a region of the subject's gastrointestinal tract. In some embodiments, the colon of the subject is colonized. In some embodiments, the composition is administered orally. In some embodiments, the composition is administered rectally. In some embodiments, the composition is administered by fecal transplant, for example in a single fecal transplant, or in two or more fecal transplants.

It is further noted that in some embodiments, the composition comprising bacteria and/or metabolite precursors are administered to the selected subject at particular time frames. As noted herein, modulating the levels of certain metabolites prenatally can reduce or prevent symptoms of ASD (See Example 5). Accordingly, in some embodiments, the composition comprising bacteria and/or metabolite precursors is administered to the subject prenatally. In some embodiments, the composition comprising bacteria and/or metabolite precursors is administered prenatally by administering the composition to the subject's mother, for example, orally, rectally, and/or via a fecal transplant. In some embodiments, the composition comprising bacteria and/or metabolite precursors is administered prenatally by administering the composition directly to the subject, for example, orally, rectally, and/or via a fecal transplant. In some embodiments, the composition is not administered to the subject postpartum.

In some embodiments, the subject is selected as one being at risk of developing ASD or a symptom of ASD. The subject can be selected as being at risk of developing ASD or a symptom of ASD prior to administration of the composition comprising bacteria and/or metabolite precursors, or can be selected at the time of administration.

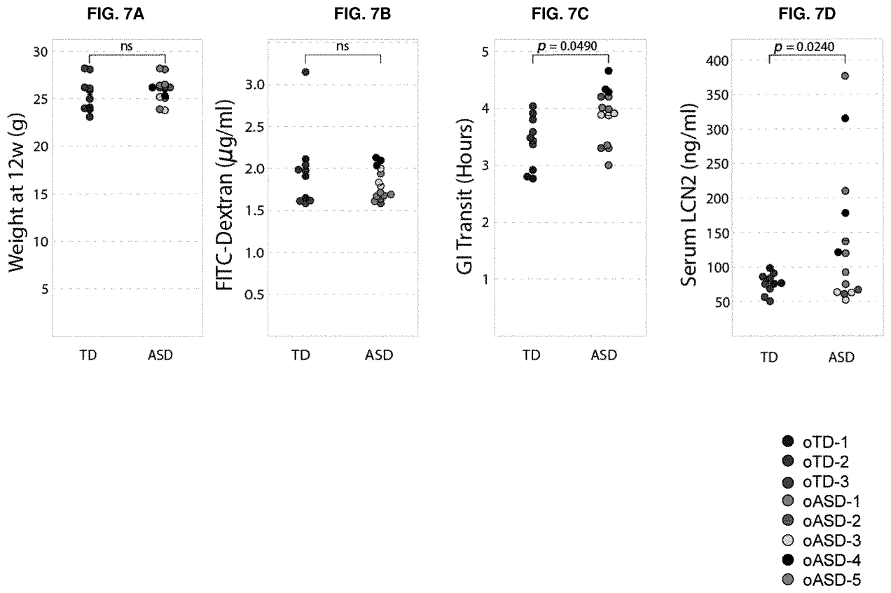

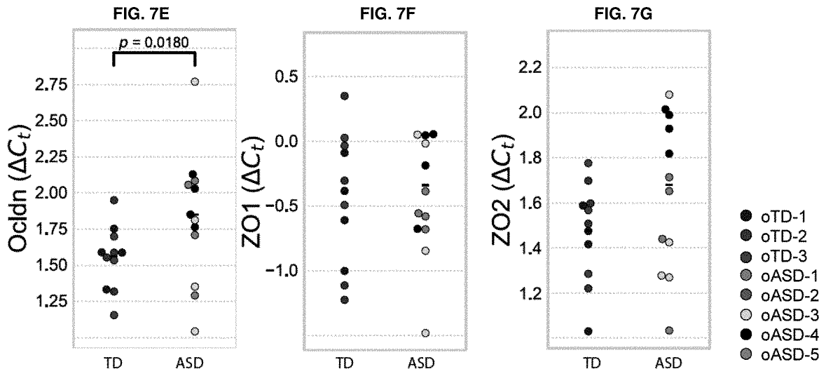

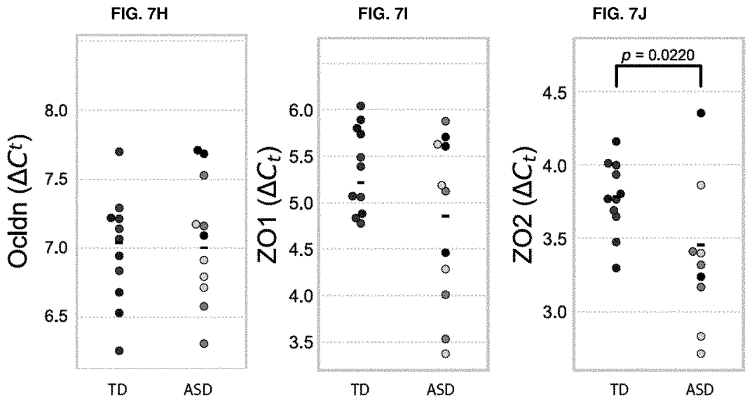

In some embodiments, the subject is selected as being at risk of developing ASD or a symptom of ASD based on a profile comprising one or more of:
(a) a presence and/or level of a gut bacterium selected from the group consisting of: Bacteroides ovatus, Parabacteroides merdae, Bacteroides thetaiotaomicron, or a combination of two or more of the listed bacteria; and a presence and/or level of Eisenbergiela tayi genes in a sample from the prenatal subject or the prenatal subject's mother;
(b) a presence or gene product level of a gut microbiota gene that is an ortholog of KEGG ortholog K0681, and/or a presence or gene product level of a gene that is an ortholog of KEGG ortholog K1442 genes in a sample from the prenatal subject or the prenatal subject's mother, wherein the sample preferably comprises gut, feces, or gut and feces material of the subject;
(c) a level of colon taurine, 5-Aminovaleric acid (5AV), lysine, 3-aminoisobutyric acid (3-AIBA), genistein, daidzein, lysine, 5-aminopentanoate, cellobiose, glyceric acid or genistein, a level of serum D ribose, ribitol, ribonic acid, or L-tyrosine, or a rate of degradation thereof genes in a sample from the prenatal subject or the prenatal subject's mother;
(d) a level of a product of a gene of the subject selected from the group consisting of: Gm26944 (or a human ortholog thereof), Gm13016 (or an ortholog thereof), Gm17259 (or a human ortholog thereof), 4930539E08Rik (or a human ortholog thereof), Dag1b (or a human ortholog thereof), or a combination of two or more of the listed genes in a sample from the prenatal subject or the prenatal subject's mother;
(e) a level of a splice variant of the subject selected from the group consisting of: a mutually exclusive exon in Neurexin 2 (or a human ortholog thereof); a mutually exclusive exon in Ankryin 2 (or a human ortholog thereof); a skipped exon of Cacna1c (or a human ortholog thereof); a retained intron of Ads1 (or a human ortholog thereof);
or a skipped exon of a pogo transferrable element derived with ZNF domain Pogz (or a human ortholog thereof) genes in a sample from the prenatal subject or the prenatal subject's mother; and/or
(f) a presence and/or level of a gut bacterium selected from the group consisting of: Bacteroides ovatus, Parabacteroides merdae, of Lachnospiraceae, or a combination of two or more of the listed bacteria. By way of example, B. ovatus can be identified and/or detected by SEQ ID NO: 24, Parabacteroides merdae can be identified and/or detected by SEQ ID NO: 42, and E. tayi can be identified and/or detected by SEQ ID NO: 40 and/or 41.
   In some embodiments, the profile comprises the detected presence and/or levels of (a), (b), (c), (d), and/or (e), or a combination of two or more of (a), (b), (c), (d), and (e), for example (a), (b), (c), (d), and (e). In some embodiments, the profile comprises the detected presence and/or levels of (a), (b), (c), (d), (e), and/or (f) or a combination of two or more of (a), (b), (c), (d), (e), and/or (f) for example, (a) and (b); (a) and (c); (a) and (d); (a) and (e); (a) and (f); (b) and (c); (b) and (d); (b) and (e); (b) and (f); (c) and (d); (c) and (e); (c) and (f); (d) and (e); (d) and (f); (e) and (f); (a) (b) and (c); (a) (b) and (d); (a) (b) and (e); (a) (b) and (f); (b) (c) and (d); (b) (c) and (e); (b) (c) and (f); (c) (d) and (e); (c) (d) and (f); (d) (e) and (f); or (a) (b), (c), (d), (e), and (f). In some embodiments, the profile further comprises determining a microbiome diversity of the gut of the subject (for example if the sample is a fecal sample). The microbiome diversity can be determined, for example, by alpha diversity of amplicon sequence variants in the sample. In some embodiments, the subject is determined to have ASD, a severity of ASD, or be at risk of ASD if the sample comprises a lower alpha diversity than that of a typically developing control. In some embodiments, the profile further comprises at least one of a level of serum Lipocalin-2 (LCN2) in the subject (it has been observed that LCN2 levels are lower in ASD subject serum than TD controls; See FIG. 7D); expression of Occludin (Ocldn) in the distal ileum of the subject (it has been observed that Ocldn levels in the distal ileum are higher in ASD subject serum than TD controls; See FIG. 7E); or Zonula Occludens 2 (ZO2) expression in the proximal colon of the subject (it has been observed that ZO2 levels in the proximal colon are lower in ASD subject serum than TD controls; See FIG. 7J). In some embodiments, the profile further comprises intestinal transit time of the subject relative to a non-ASD subject (Control) (it has been observed that intestinal transit time is shorter in ASD subjects; See FIG. 7C). In some embodiments, the profile is of a sample of the subject. Profiles that are indicative or predictive of a presence, increased risk, and/or increased severity of ASD or one or more symptoms of ASD are described herein (See, e.g., the section entitled “Methods of Determining A Profile Of A Sample Of A Subject”). In some embodiments, the profile is detected and understood according to at least one methods of determining a profile of a sample of a subject as described herein, for example a fecal and/or serum sample. In some embodiments, the profile is of a sample of the subject's mother, for example a fecal and/or serum sample.

In some embodiments the subject is selected prenatally. In some embodiments, the subject is selected as being at risk of developing ASD or a symptom of ASD due to the mother of the subject having a colon comprising reduced levels of taurine and/or 5AV compared to a control mother of a non-ASD offspring, and/or elevated levels of 3-aminoisobutyric acid (3AIBA) compared to the control mother of a non-ASD offspring. By way of example, in some embodiments, a control mother will be confirmed to have a non-ASD offspring, and/or offspring that are not at risk for the relevant ASD symptoms. For example, the offspring of the control mother can have an ADOS score of three or less, for example three, two, or one. Optionally, samples from the control mother can be obtained prenatally at an earlier point in time than those of the subject mother, so as to facilitate testing of the control mother's offspring for ASD or one or more symptoms thereof. In some embodiments, the level of the metabolite in the control is provided as a stored reference value. For example, the level of the control can be electronically stored.

In some embodiments, the subject is selected as having or being expected to have reduced levels of taurine and/or 5AV compared to a control non-ASD control; and/or elevated levels of 3-aminoisobutyric acid (3AIBA) compared to the non-ASD control. By way of the example, in some embodiments, the non-ASD control does not have ASD, and/or does not exhibit relevant ASD symptoms. For example, the non-ASD control can have an ADOS score of three or less, for example three, two, or one.

### Methods of Determining a Profile of a Sample of a Subject

It has been observed herein that certain profiles of subject samples (or samples of subjects' mothers) are characteristic of a presence, increased risk, or elevated severity of one or more symptoms of ASD after birth, and that modulation of metabolites in subjects exhibiting these profiles can reduce and/or prevent symptoms of ASD after birth See Examples 2-5. Accordingly, it is contemplated that profiles of subject samples can be useful for determining a risk, presence, and or severity of one or more symptoms of ASD in a subject after birth, and further, can be useful in identifying selected subject that respond to compositions comprising metabolites, bacteria, and/or metabolite precursors in conjunction with some embodiments herein.

In some embodiments, a method of determining a profile of a sample of a subject is provided. The method can comprising detecting at least one of:
(a) a presence and/or level of a gut bacterium selected from the group consisting of: Bacteroides ovatus, Parabacteroides merdae, Bacteroides thetaiotaomicron, or a combination of two or more of the listed bacteria; and a presence and/or level of Eisenbergiela tayi genes in a sample from the prenatal subject or the prenatal subject's mother. For example, in some embodiments, (a) comprises a presence and/or level of a Bacteroides ovatus, Parabacteroides merdae, Bacteroides thetaiotaomicron, and E. tayi. For example, in some embodiments, (a) comprises a presence and/or level of a Parabacteroides merdae, Bacteroides thetaiotaomicron, and E. tayi. For example, in some embodiments, (a) comprises a presence and/or level of a Bacteroides ovatus, Bacteroides thetaiotaomicron, and E. tayi. For example, in some embodiments, (a) comprises a presence and/or level of a Bacteroides ovatus, Parabacteroides merdae, and E. tayi. For example, in some embodiments, (a) comprises a presence and/or level of a Bacteroides ovatus, Parabacteroides merdae, and Bacteroides thetaiotaomicron. By way of example, B. ovatus can be identified and/or detected by SEQ ID NO: 24, Parabacteroides merdae can be identified and/or detected by SEQ ID NO: 42, and E. tayi can be identified and/or detected by SEQ ID NO: 40 and/or 41;
(b) a presence or gene product level of a gut microbiota gene that is an ortholog of KEGG ortholog K0681, and/or a presence or gene product level of a gene that is an ortholog of KEGG ortholog K1442 in a sample from the prenatal subject or the prenatal subject's mother, wherein preferably the sample comprises gut, feces, or gut and feces material of the subject;
(c) a level of colon taurine, 5-Aminovaleric acid (5AV), lysine, 3-aminoisobutyric acid (3-AIBA), genistein, daidzein, lysine, 5-aminopentanoate, cellobiose, glyceric acid,
a level of serum D ribose, ribitol, ribonic acid, or L-tyrosine,
or a rate of degradation thereof in a sample from the prenatal subject or the prenatal subject's mother;
(d) a level of a product of a gene of the subject selected from the group consisting of: Gm26944 (or an ortholog thereof), Gm13016 (or an ortholog thereof), Gm17259 (or an ortholog thereof), 4930539E08Rik (or ortholog thereof), Dag1b (or an ortholog thereof), or a combination of two or more of the listed genes in a sample from the prenatal subject or the prenatal subject's mother;
(e) a level of a splice variant of the subject selected from the group consisting of: a mutually exclusive exon in Neurexin 2 (or a human ortholog thereof); a mutually exclusive exon in Ankryin 2 (or a human ortholog thereof); a skipped exon in FMr (or a human ortholog thereof); a skipped exon in Ube3a (or a human ortholog thereof); a skipped exon in Rims1 (or a human ortholog thereof); a skipped exon of Cacna1c (or a human ortholog thereof); a retained intron of Ads1 (or a human ortholog thereof); a skipped exon of a pogo transferrable element derived with ZNF domain Pogz (or a human ortholog thereof in a sample from the prenatal subject or the prenatal subject's mother); or a skipped exon of Trip12 (or a human ortholog thereof). In some embodiments, (e) comprises a level of a splice variant of the subject selected from the group consisting of; a mutually exclusive exon in Neurexin 2 (or a human ortholog thereof); a mutually exclusive exon in Ankryin 2 (or a human ortholog thereof); a skipped exon of Cacna1c (or a human ortholog thereof); a retained intron of Ads1 (or a human ortholog thereof); or a skipped exon of a pogo transferrable element derived with ZNF domain Pogz (or a human ortholog thereof in a sample from the prenatal subject or the prenatal subject's mother); and/or
(f) a relative microbiome diversity in the gut of the subject or the mother of the subject compared to a typically developing control. By way of example, the relative diversity can be determined alpha diversity as measured by observed amplicon sequence variants (ASCs), for example those of 16S rRNA.
   The profile can comprise the detected presence and/or levels of (a), (b), (c), (d), (e), and/or (f) or a combination of two or more of (a), (b), (c), (d), (e), and (f). In some embodiments, the profile comprises the detected presence and/or levels of (a), (b), (c), (d), (e), or a combination of two or more of (a), (b), (c), (d), and (e). In some embodiments, the profile comprises, consists essentially of, or consists of (a) and (b). In some embodiments, the profile comprises, consists essentially of, or consists of (a) and (c). In some embodiments, the profile comprises, consists essentially of, or consists of (a) and (d). In some embodiments, the profile comprises, consists essentially of, or consists of (a) and (e). In some embodiments, the profile comprises, consists essentially of, or consists of (a) and (f). In some embodiments, the profile comprises, consists essentially of, or consists of (b) and (c). In some embodiments, the profile comprises, consists essentially of, or consists of (b) and (d). In some embodiments, the profile comprises (b) and (e). In some embodiments, the profile comprises (b) and (f). In some embodiments, the profile comprises, consists essentially of, or consists of (c) and (d). In some embodiments, the profile comprises, consists essentially of, or consists of (c) and (e). In some embodiments, the profile comprises, consists essentially of, or consists of (c) and (f). In some embodiments, the profile comprises, consists essentially of, or consists of (d) and (e). In some embodiments, the profile comprises, consists essentially of, or consists of (d) and (f). In some embodiments, the profile comprises, consists essentially of, or consists of (a), (b), and (c). In some embodiments, the profile comprises, consists essentially of, or consists of (a), (b), and (d). In some embodiments, the profile comprises, consists essentially of, or consists of (a), (b), and (e). In some embodiments, the profile comprises, consists essentially of, or consists of (a), (b), and (f). In some embodiments, the profile comprises, consists essentially of, or consists of (a), (c), and (d). In some embodiments, the profile comprises, consists essentially of, or consists of (a), (c), and (e). In some embodiments, the profile comprises, consists essentially of, or consists of (a), (c), and (f). In some embodiments, the profile comprises, consists essentially of, or consists of (a), (d), and (e). In some embodiments, the profile comprises, consists essentially of, or consists of (a), (d), and (f). In some embodiments, the profile comprises, consists essentially of, or consists of (b), (c), and (d). In some embodiments, the profile comprises, consists essentially of, or consists of (b), (c), and (e). In some embodiments, the profile comprises, consists essentially of, or consists of (b), (c), and (f). In some embodiments, the profile comprises, consists essentially of, or consists of (b), (d), and (e). In some embodiments, the profile comprises, consists essentially of, or consists of (b), (d), and (f). In some embodiments, the profile comprises, consists essentially of, or consists of (c), (d), and (e). In some embodiments, the profile comprises, consists essentially of, or consists of (c), (d), and (f). In some embodiments, the profile comprises, consists essentially of, or consists of (d), (e), and (f). In some embodiments, the profile comprises, consists essentially of, or consists of (a), (b), (c) and (d). In some embodiments, the profile comprises, consists essentially of, or consists of (a), (b), (c) and (e). In some embodiments, the profile comprises, consists essentially of, or consists of (a), (b), (c) and (f). In some embodiments, the profile comprises, consists essentially of, or consists of (b), (c), (d) and (e). In some embodiments, the profile comprises, consists essentially of, or consists of (b), (c), (d) and (f). In some embodiments, the profile comprises, consists essentially of, or consists of (b), (c), (d), (e), and (f). In some embodiments, the profile comprises, consists essentially of, or consists of (a), (c), (d), and (e). In some embodiments, the profile comprises, consists essentially of, or consists of (a), (c), (d), and (f). In some embodiments, the profile comprises, consists essentially of, or consists of (a), (b), (d), and (e). In some embodiments, the profile comprises, consists essentially of, or consists of (a), (b), (d), and (f). In some embodiments, the profile comprises, consists essentially of, or consists of (a), (b), (c), (d), and (e). In some embodiments, the profile comprises, consists essentially of, or consists of (a), (b), (c), (d), and (f). In some embodiments, the profile comprises, consists essentially of, or consists of (a), (b), (c), (d), (e), and (f). In some embodiments, the subject is human. In some embodiments the profile comprises the detected the presence and/or level one or more metabolites that are expressed differently in ASD and non-ASD subjects as shown in any of Tables 3-1, 3-2, and/or 3-3. In some embodiments, the profile further comprises at least one of a level of serum Lipocalin-2 (LCN2) in the subject (it has been observed that LCN2 levels are lower in ASD subject serum than TD controls; See FIG. 7D); expression of Occludin (Ocldn) in the distal ileum of the subject (it has been observed that Ocldn levels in the distal ileum are higher in ASD subject serum than TD controls; See FIG. 7E); Zonula Occludens 2 (ZO2) expression in the proximal colon of the subject (it has been observed that ZO2 levels in the proximal colon are lower in ASD subject serum than TD controls; See FIG. 7J). In some embodiments, the profile further comprises intestinal transit time of the subject relative to a non-ASD subject (Control) (it has been observed that intestinal transit time is shorter in ASD subjects; See FIG. 7C).

Regarding (a), it is observed that in colon content samples of subjects, reduced levels of Bacteroides ovatus, Parabacteroides merdae, Bacteroides thetaiotaomicron;
or increased levels of Eisenbergiela tayi, relative to levels in a control typically developing subject are indicative of symptoms of ASD (See Example 2). Accordingly, in some embodiments, the method of determining the profile comprises determining (a), and the sample comprises gut and/or feces material of the subject, and elevated risk of ASD (after birth) is indicated by reduced levels of Bacteroides ovatus, Parabacteroides merdae, Bacteroides thetaiotaomicron; or increased levels of Eisenbergiela tayi, relative to levels present in a control typically developing subject. For example, increase levels of Eisenbergiela tayi in the colon of the subject relative to the control can be indicated by a presence of Eisenbergiela tayi in the sample of the subject, and an absence (or lower level) of Eisenbergiela tayi in the sample of the control.

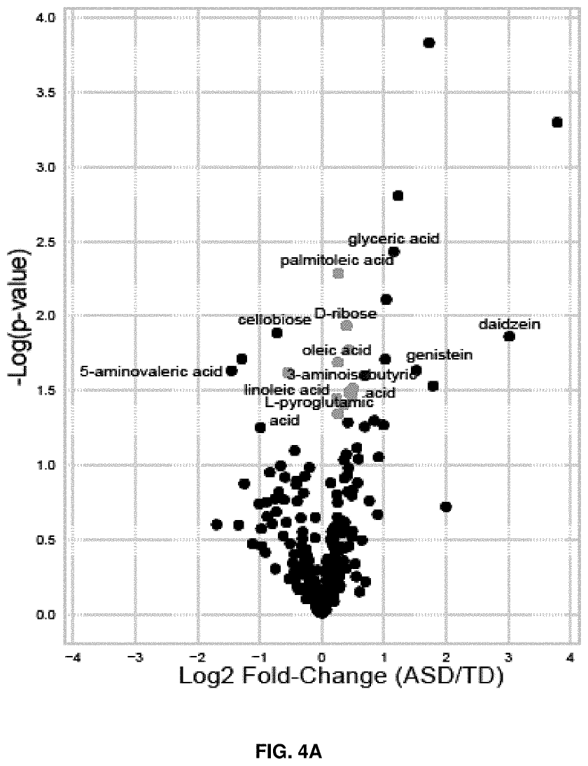

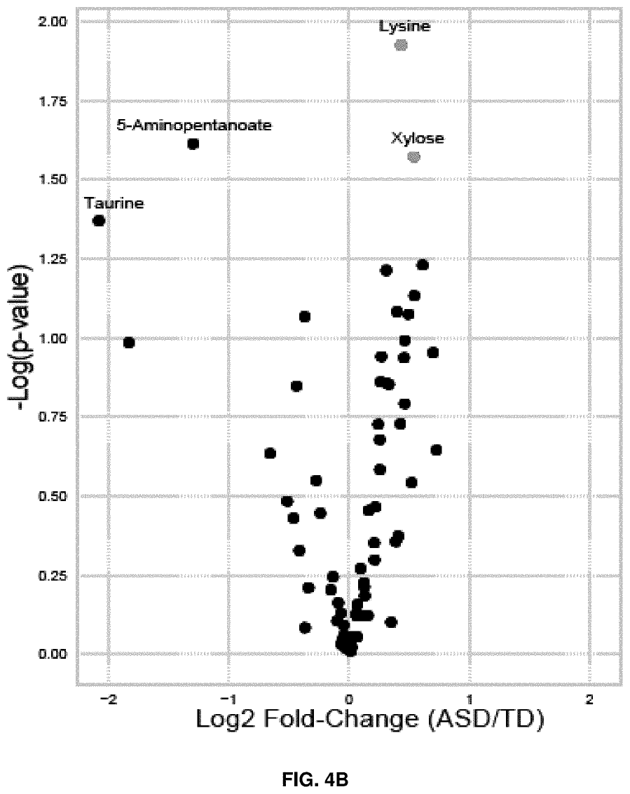

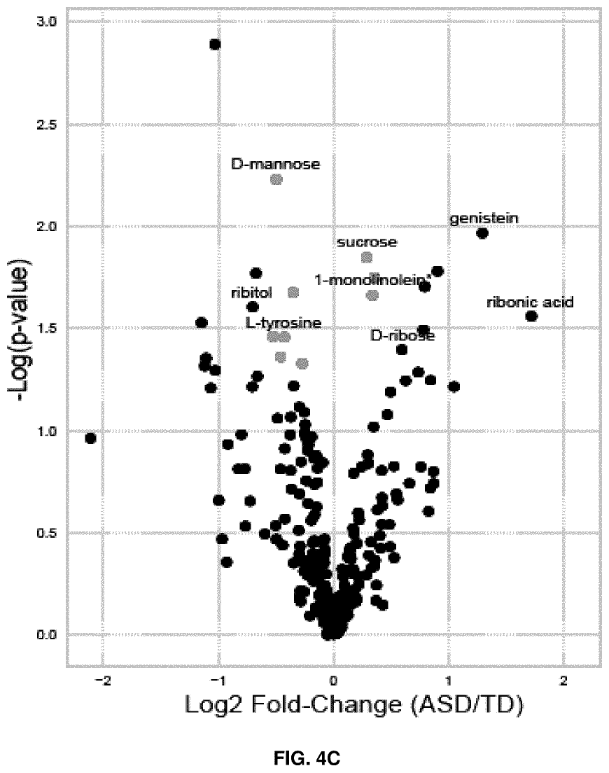

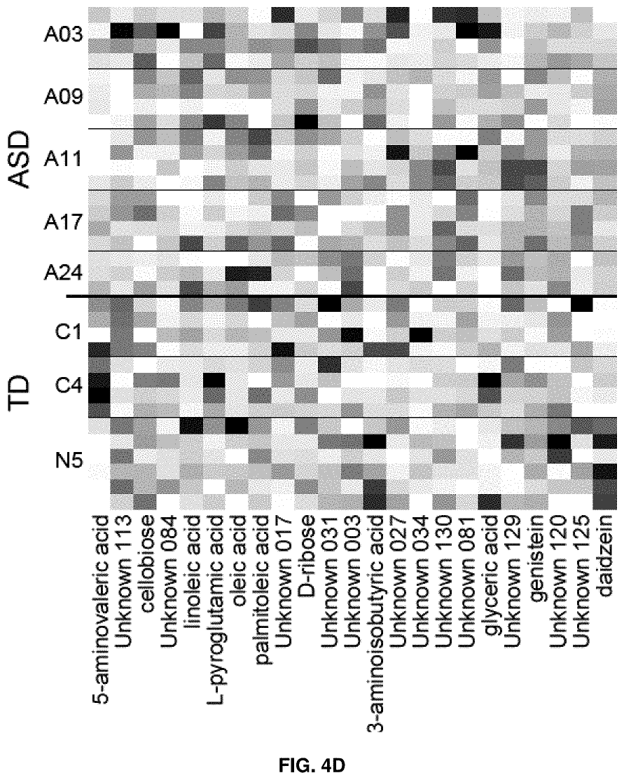

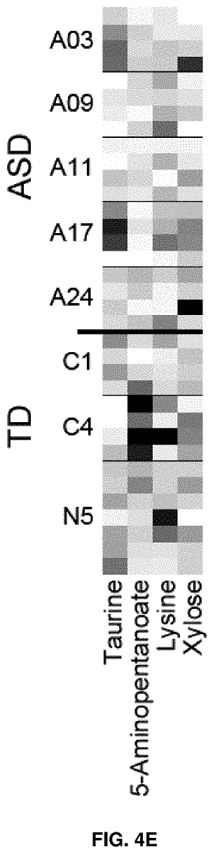

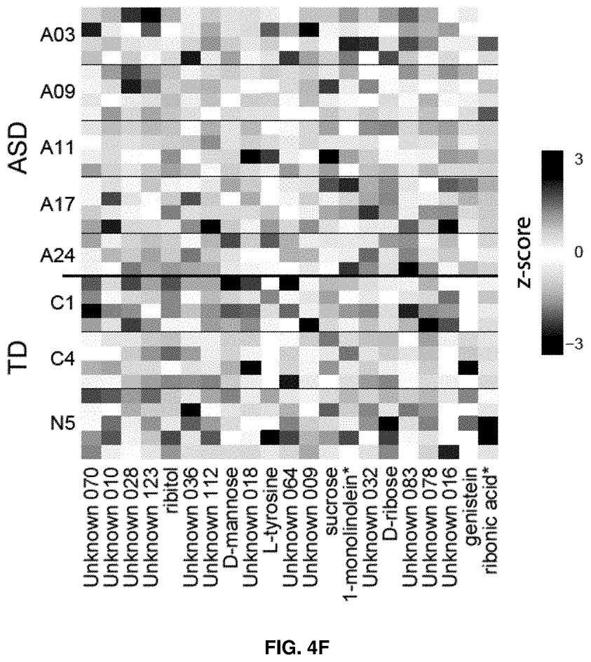

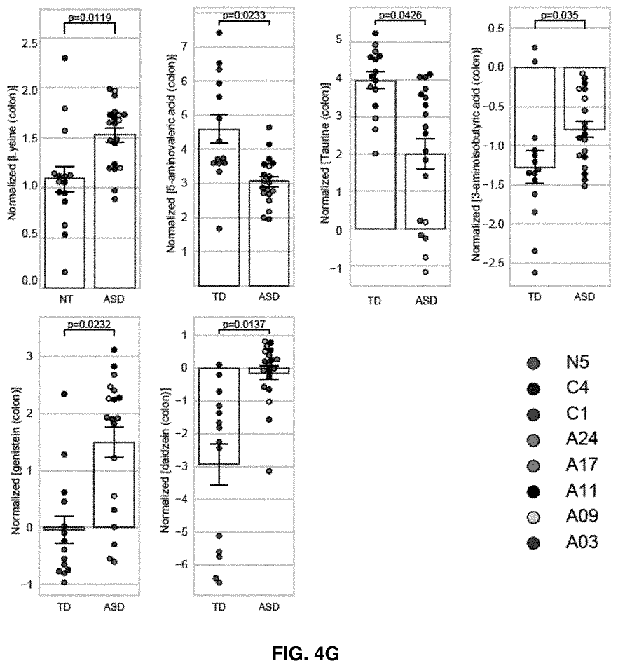

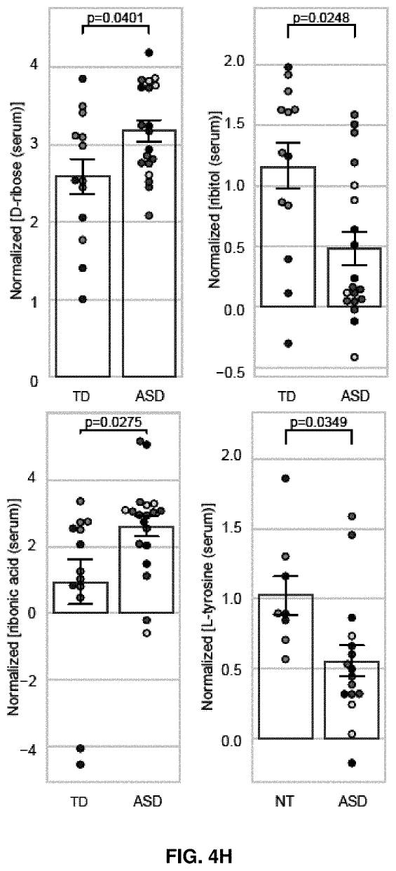

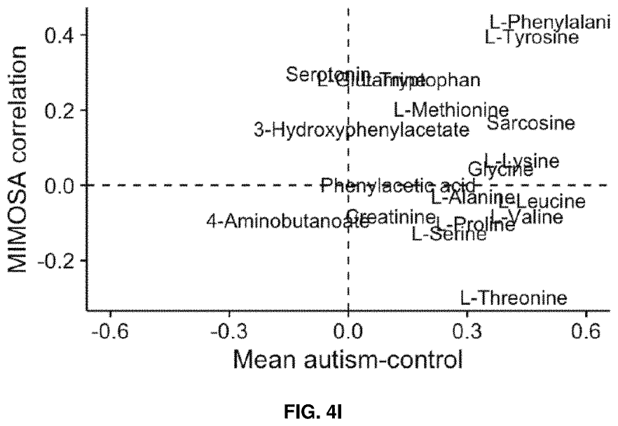

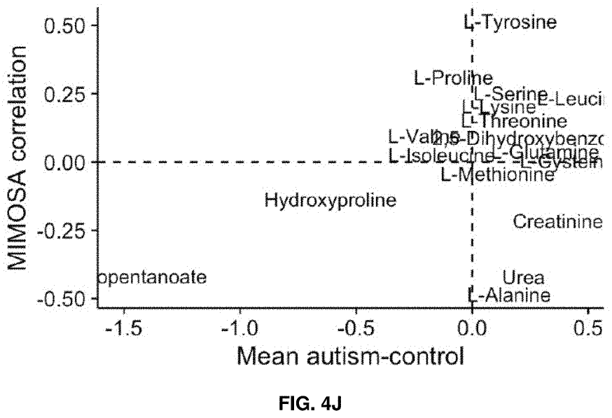

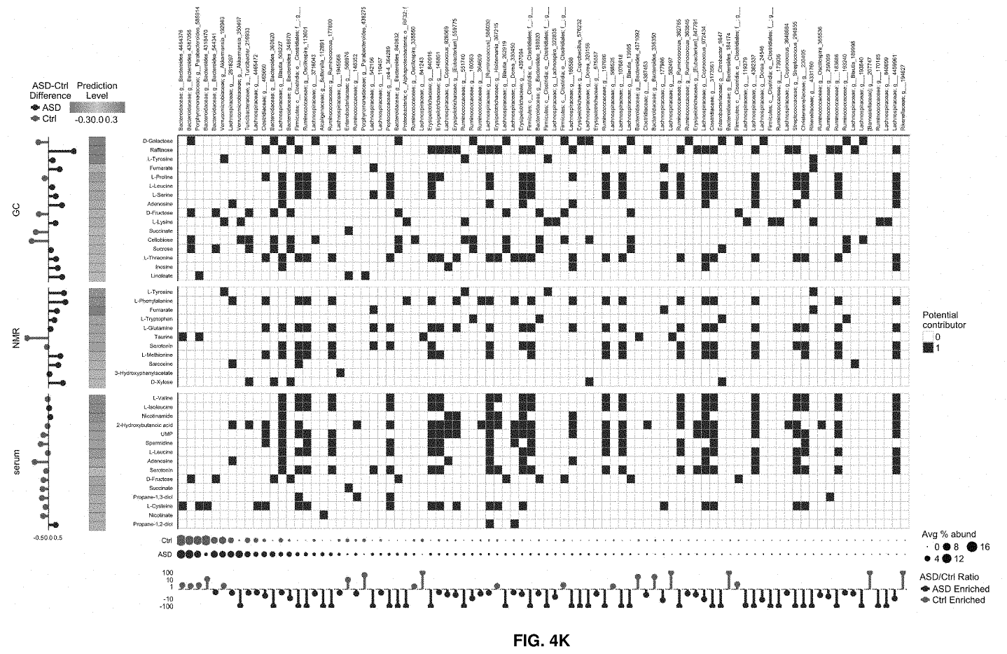

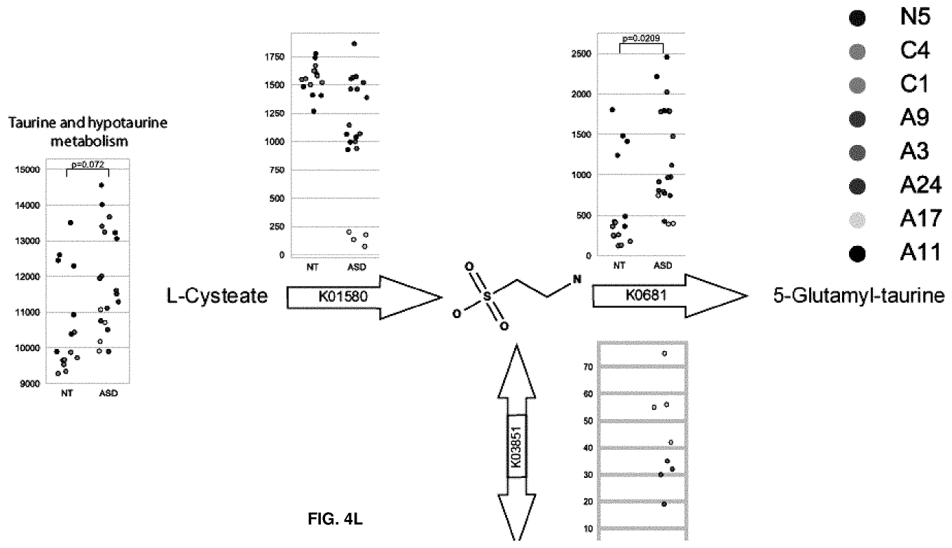

Regarding (b), it is observed herein that levels of certain metabolite-producing and/or degrading bacteria are altered in colon contents of subjects that exhibit symptoms of ASD, or a risk of developing these symptoms (See Example 4). Moreover, it is observed that some of the different bacteria are involved in production and/or degradation of the metabolites that differ between ASD individuals and TD controls (See FIGS. 41-4L). Accordingly, it is contemplated that in accordance with some embodiments herein, certain gut bacteria (comprising suitable gene products for producing and/or degrading metabolites) are indicative of symptoms of ASD, and/or a risk of developing the same. For example, it is observed herein that increased levels of a gut microbiota gene that is the ortholog of KEGG ortholog K0681 and/or decreased levels of the gene that is the ortholog of KEGG ortholog K01442 relative to levels present in a control subject are indicative of symptoms of ASD, for example defective social behavior (&e Example 4). Accordingly, in some embodiments, determining the profile comprises detecting (b), in which the sample comprises gut and/or feces material of the mother of the subject, and in which an elevated risk of ASD is indicated by increased levels of the gut microbiota gene that is the ortholog of KEGG ortholog K0681 and/or decreased levels of the gene that is the ortholog of KEGG ortholog K01442 relative to levels present in a control subject. By way of example, in some embodiments, the presence of the KEGG ortholog can be detected by sequencing the bacterial genome (e.g., using high-throughput genomic sequencing such as ILLUMINA® sequencing), through qualitative and/or quantitative nucleic acid amplification (e.g., PCR, qPCR, and the like), or through microarray analysis. The “presence” of a substance such as a gene, gene product, and/or splice variant has its customary and ordinary meaning as understood by one of skill in the art in view of this disclosure. It refers to the substance (e.g., gene, gene product, and/or splice variant) being physically in the sample. For example, it will be understood that if a gene, gene product, or splice variant (or signal corresponding thereto) is above the level of detection (LOD) in a nucleic acid assay such as qualitative PCR, quantitative PCR, or microarray analysis. As used herein “ortholog” has its customary and ordinary meaning as understood by one of skill in the art in view of this disclosure. It refers to genes found in different species that arise from a single genomic locus in a common ancestor. Some orthologs can have similar function to each other. A number of bioinformatics tools are available to identify orthologs, for example the KO (KEGG Orthology) database (accessible on the world wide web), and the NCBI HomoloGene database, accessible on the world wide web. The relevant KEGG orthologs can be identified, for example, using database and/or analytical tools such as gProfileR with GO and KEGG annotations, or the KEGG: Kyoto Encyclopedia of Genes and Genomes (accessible on the world-wide-web).

Regarding (c), it is observed herein that levels of certain metabolites in the colon and/or sera of subjects or their mothers indicates a presence or risk of developing symptoms of ASD (See Example 4). Accordingly, it is contemplated that in accordance with some embodiments herein, a profile certain colon and/or serum metabolite levels indicate symptoms of ASD, severity of symptoms of ASD, or a risk of developing ASD in a subject after birth. In some embodiments, determining the profile comprises detecting (c), and the sample comprises colon contents of the subject and/or serum of the subject. Colon levels of taurine, 5AV, 5-aminopentanoate, or cellobiose, below a non-ASD control; serum levels of ribitol or L-tyrosine below a non-ASD control; colon levels of 3AIBA, lysine, glyceric acid, geristein, and/or daidzein above a non-ASD control; and/or serum levels of D ribose or ribonic acid above a non-ASD control can indicate an increased risk of developing and/or severity of ASD in the subject in accordance with some embodiments herein. In some embodiments, determining the profile comprises (c), the sample comprises colon contents of the subject, and colon levels of taurine and 5AV below a non-ASD control indicate an increased risk of developing and/or severity of ASD in the subject after birth. In some embodiments, determining the profile comprises (c), the sample comprises colon contents of the subject, and colon levels of taurine below a non-ASD control indicate an increased risk of developing and/or severity of ASD in the subject after birth. In some embodiments, determining the profile comprises (c), the sample comprises colon contents of the subject, and colon levels of 5AV below a non-ASD control indicate an increased risk of developing and/or severity of ASD in the subject after birth. In some embodiments, determining the profile comprises (c), the sample comprises colon contents of the subject, and colon levels of 5-aminopentanoate and/or cellobiose below a non-ASD control indicate an increased risk of developing ASD and/or severity of ASD in the subject after birth. In some embodiments, determining the profile comprises (c), the sample comprises colon contents of the subject, and colon levels of geristein, and/or daidzein above a non-ASD control indicate an increased risk of developing and/or severity of ASD in the subject after birth. In some embodiments, determining the profile comprises (c), the sample comprises colon contents of the subject, and colon levels of glyceric acid and/or geristein above a non-ASD control indicate an increased risk of developing and/or severity of ASD in the subject after birth. In some embodiments, determining the profile comprises (c), the sample comprises colon contents of the subject, and colon levels of glyceric acid and/or geristein above a non-ASD control indicate an increased risk of developing and/or severity of ASD in the subject after birth and colon levels of 3AIBA and/or daidzein above a non-ASD control indicate an increased risk of developing and/or severity of ASD in the subject after birth. In some embodiments, determining the profile comprises (c), the sample comprises colon contents of the subject, and colon levels of glyceric acid and/or geristein above a non-ASD control indicate an increased risk of developing and/or elevated severity of ASD in the subject after birth and colon levels of 3AIBA above a non-ASD control indicate an increased risk of developing and/or severity of ASD in the subject after birth. In some embodiments, determining the profile comprises detecting (c), and the sample comprises colon contents of the subject, and colon levels of taurine, 5AV, 5-aminopentanoate, or cellobiose below a non-ASD control, and/or colon levels of 3AIBA, lysine, glyceric acid, geristein, and/or daidzein above a non-ASD control indicate an increased risk of developing and/or severity of ASD in the subject after birth. In some embodiments, determining the profile comprises detecting (c), wherein the sample comprises serum of the subject, and wherein serum levels of ribitol or L-tyrosine below a non-ASD control, and/or serum levels of D ribose or ribonic acid above a non-ASD control indicate an increased risk of developing and/or severity of ASD in the subject after birth. As discussed herein, some bacteria can also affect the rate of degradation, and therefore the level of certain indicated metabolites, and thus a rate of degradation of the noted metabolites can also provide relevant information for the profile. In some embodiments, metabolite levels are detected by mass spectrometry, for example gas-chromatography-mass-spectrometry (GC-MS), mass spectrometry-mass spectrometry (MS/MS), or MALDI. In some embodiments, metabolite levels are detected by nuclear magnetic resonance (NMR). In some embodiments, metabolite levels are detected by an immunoassay, for example an ELISA, a lateral flow assay, or a no-wash assay comprising an antibody specific for the metabolite.

In some embodiments, the profile comprises detecting (c), and the sample comprises, consists of, or consists essentially of serum of the subject. In some embodiments, the profile comprises detecting (c), and the sample comprises, consists of, or consists essentially of serum of the subject's mother. In some embodiments, the profile comprises detecting (c), and the sample comprises, consists of, or consists essentially of a colon contents sample of the subject (e.g., fecal material). In some embodiments, the profile comprises detecting (c), and the sample comprises, consists of, or consists essentially of a colon contents sample of the subject's mother (e.g., fecal material). In some embodiments, the profile comprises detecting (c), and the sample comprises, consists of, or consists essentially of a first serum sample and a second colon contents sample of the subject (e.g., fecal material). In some embodiments, the profile comprises detecting (c), and the sample comprises, consists of, or consists essentially of a first serum sample and a second colon contents sample of the subject's mother (e.g., fecal material). In some embodiments, the sample for (a), (b), and/or (c) comprises a gut or fecal sample of the subject. In some embodiments, the sample comprises colon contents of the subject.

Regarding (d) and/or (e), it is observed herein that levels of gene products in central nervous system (CNS) samples of subjects can indicate a presence and/or a risk of ASD symptoms in the subject after birth. For example, it is observed herein that levels of particular RNAs in the striatum (STR) of a subject are indicative of ASD-colonized mice, including levels of Gm26944, Gm13016, Gm17259, 4930539E08Rik and/or Dag1b gene product (Example 3). Accordingly, it is contemplated that in some embodiments, a profile in a sample of a subject comprises a presence and/or levels of the identified genes, for example, Gm26944, Gm13016, Gm7259, 4930539E08Rik and/or Dag1b gene product (these genes and gene products are referred to using their mouse nomenclature, though it will be appreciated that human orthologs of these genes are also explicitly contemplated). By way of example, as identified using the NCBI Homologene annotation database (accessible on the world wide web at www.ncbi.nlm.nih.gov) the identified human (H. sapiens) ortholog of 4930539E08Rik is “C6orf222,” and the identified human (H. sapiens) ortholog of Dag1b is “diacylglycerol lipase beta” (aka “DAGLB”). It is noted that Gm26944, Gm13016, Gm17259 refer to genes encoding RNAs that are not known to encode proteins, and as such, “gene products” can refer to RNA and/or polypeptide, depending on the particular gene and context. Furthermore it is observed herein that levels of the mutually exclusive exon in Neurexin 2 above a non-ASD control; levels of the skipped exon of Ube3a below the non-ASD control; levels of the skipped exon of Finr1 below the non-ASD control; levels of the skipped exon of Rims) above the non-ASD control; levels of the mutually exclusive exon in Ankryin 2 below the non-ASD control; levels of the skipped exon of Cacna1c above the non-ASD control; levels of the retained intron of Ads1 above a non-ASD control; levels of the skipped exon of the pogo transferrable element pogz above a non-ASD control; and/or levels of the skipped exon of Trip12 above the non-ASD control indicate an increased risk of ASD; (See Example 3). Accordingly, it is contemplated that in some embodiments, a profile in a sample of a subject comprising a presence and/or particular levels of the identified splice variants indicates a presence, risk, and/or severity of certain symptoms of ASD after birth, for example, impaired sociability, repetitive behavior, and/or anxiety. In some embodiments, determining the profile comprises determining (e), and wherein levels of the mutually exclusive exon in Neurexin 2 above a non-ASD control; levels of the skipped exon of Ube3a below the non-ASD control; levels of the skipped exon of Fmr1 below the non-ASD control; levels of the skipped exon of Rims1 above the non-ASD control; levels of the mutually exclusive exon in Ankryin 2 below the non-ASD control; levels of the skipped exon of Cacna1c above the non-ASD control; levels of the retained intron of Ads1 above a non-ASD control; levels of the skipped exon of the pogo transferrable element above a non-ASD control; and/or levels of the skipped exon of Trip12 above the non-ASD control indicate an increased severity of ASD. In some embodiments, for any of (d) and/or (e), the sample comprises, consists essentially of, or consists of gene products of the CNS of the subject. In some embodiments, the sample for (d) and/or (e) comprises a CNS tissue sample, such as prefrontal cortex (PFC) and/or striatum (STR). In some embodiments, the sample for (d) and/or (e) comprises, consists essentially or, or consists of cerebrospinal fluid (CSF) of the subject. In some embodiments, the splice variants of the noted genes are identified using high-throughput transcriptome sequencing. In some embodiments, the splice variants of the noted genes are identified using nucleic acid amplification assays, for example qualitative and/or quantitative reverse-transcriptase-PCR. By way of examples, the splice variants can be identified using primers such as those shown in Table 2.3.

Regarding (a), it is observed that sOTUs b20cd_Bacteroides, 02b40_Lachnospiraceae, 29857_Lachnospiraceae, and 4ae7e_Parabacteroides in the gut can substantially distinguish ASD and TD subjects (See FIGS. 2A-C). These sOTUs correspond to Bacteroides ovatus, E. tayi, and Parabacteroides merdae. It is noted that E. tayi is a Lachnospiraceae. Accordingly, in some embodiments, with respect to (a), the Lachnospiraceae comprises, consists essentially of, or consists of E. tayi. By way of example, B. ovatus can be identified and/or detected by SEQ ID NO: 24, Parabacteroides merdae can be identified and/or detected by SEQ ID NO: 42, and E. tayi can be identified and/or detected by SEQ ID NO: 40 and/or 41. In some embodiments, Bacteroides ovatus, Lachnospiraceae (e.g., E. tayi), and Parabacteroides merdae are identified using nucleic acid assays, for example qualitative and/or quantitative reverse-transcriptase-PCR, nucleic acids sequencing (e.g., 16S sequencing), and/or microarray analysis.

In some embodiments, the method detects ASD or a symptom of ASD, predicts a risk of ASD and/or a symptom of ASD, and/or predicts the severity of ASD in the subject after birth. In some embodiments, the method further comprises determining the subject as having or being at risk of developing ASD based on (a), (b), (c), (d), and/or (e) after birth.

As described herein, it is further contemplated that subjects having a symptom and/or risk of a symptom of ASD based on a profile comprising (a), (b), (c), (d), and/or (e) can be responsive to metabolites (See Example 5) and/or gut colonization by particular bacteria (See Example 1). Accordingly, in some embodiments, the method further comprises prenatally increasing a level of taurine and/or 5AV in the subject, for example a subject determined to be at risk for one or more symptoms of ASD. In some embodiments, the level of taurine and/or 5AV is prenatally increased by administering taurine and/or 5AV to the mother of the subject. In some embodiments, the level of taurine and/or 5AV is prenatally increased by administering a composition comprising, consisting essentially of, or consisting of Bacteroides ovatus. Parabacteroides merdae, and/or Bacteroides thetaiotaomicron (e.g., Bacteroides ovatus and Bacteroides thetaiotaomicron, Parabacteroides merdae and Bacteroides thetaiotaomicron, Bacteroides ovatus and Parabacteroides merdae, or Bacteroides ovatus and Parabacteroides merdae and Bacteroides thetaiotaomicron) to the mother of the subject. In some embodiments, the level of taurine and/or 5AV is prenatally increased by administering a composition comprising, consisting essentially of, or consisting of Bacteroides ovatus, Parabacteroides merdae, and/or Bacteroides thetaiotaomicron (e.g., Bacteroides ovatus and Bacteroides thetaiotaomicron, Parabacteroides merdae and Bacteroides thetaiotaomicron, Bacteroides ovatus and Parabacteroides merdae, or Bacteroides ovatus and Parabacteroides merdae and Bacteroides thetaiotaomicron) to the subject. In some embodiments, the level of taurine and/or 5AV is prenatally increased by administering a composition comprising, consisting essentially of, or consisting of a precursor of taurine and/or 5AV to the subject, thereby increasing the level of taurine and/or 5AV in the subject. In some embodiments, the level of taurine and/or 5AV is prenatally increased by administering a composition comprising, consisting essentially of, or consisting of a precursor of taurine and/or 5AV to the mother of the subject, thereby increasing the level of taurine and/or 5AV in the subject.

### Additional Embodiments

Along with the disclosure above, in some embodiments, the following options are set forth:

1. A composition or product combination comprising:
a) bacteria selected from the group consisting of: Bacteroides ovatus, Parabacteroides merdae, and Bacteroides thetaiotaomicron, and a mixture of two or more of the listed bacteria; and
b) a taurine precursor and/or a 5-Aminovaleric acid (5AV) precursor, wherein components (a) and (b) are provided in the same formulation or are provided in separate formulations in a product combination.

2. The composition or product combination of option 1, comprising the taurine precursor and the 5AV precursor.

3. The composition or product combination of any one of options 1-2, wherein the composition or product combination does not comprise Eisenbergiela tayi.

4. The composition or product combination of any one of options 1-3, wherein the taurine precursor is selected from the group consisting of: cysteine, cysteine sulfinic acid, homocysteine, cystathionine, hypotaurine, and a mixture of two or more of the listed items.

5. The composition or product combination of any one of options 1-4, wherein the 5AV precursor is selected from the group consisting of: lysine, cadaverine, 1-piperideine, and a mixture of two or more of the listed items.

6. The composition or product combination of any one of options 1-5, wherein the composition or product combination comprises Bacteroides ovatus and Parabacteroides merdae.

7. The composition or product combination of any one of options 1-5, wherein the composition or product combination comprises Bacteroides ovatus and Bacteroides thetaiotaomicron.

8. The composition or product combination of any one of options 1-5, wherein the composition or product combination comprises Parabacteroides merdae and Bacteroides thetaiotaomicron.

9. The composition or product combination of any one of options 1-5, wherein the composition or product combination comprises Bacteroides ovatus, Parabacteroides merdae, and Bacteroides thetaiotaomicron.

10. The composition or product combination of any one of options 1-9, consisting essentially of:
the bacteria; and
the taurine precursor and/or the 5AV precursor.

11. The composition or product combination of any one of options 1-10, for use in reducing one or more symptoms of Autism Spectrum Disorder (ASD) in a subject after birth, wherein said composition or product combination is administered to said subject prenatally.

12. The composition or product combination for use according to option 11, wherein said composition or product combination is administered directly to said subject prenatally.

13. The composition or product combination for use according to option 11, wherein said composition or product combination is administered to the mother of said subject prenatally, thereby administering said composition or product combination prenatally.

14. The composition or product combination for use according to any one of options 11-14, wherein the one or more symptoms of ASD is selected from the group consisting of: repetitive behavior, hyperactivity, anxiety, and a communication disorder.

15. A method of reducing or preventing a symptom of ASD in a selected prenatal subject after birth, the method comprising administering a composition or product combination comprising an amount of taurine and/or 5AV to a subject prenatally, the amount being effective to reduce or prevent the symptom of ASD in the subject after birth.

16. The method of option 15, wherein said composition or product combination comprises the taurine and the 5AV.

17. The method of any one of options 15-16, wherein the symptom of ASD comprises a sociability disorder, anxiety, and/or a repetitive behavior.

18. The method of any one of options 15-17, wherein the prenatal subject is selected as one being at risk of developing ASD or a symptom of ASD.

19. The method of any one of options 15-18, wherein at the time of administration, the blood-brain barrier of the prenatal subject is permeable to the taurine and/or 5AV.

20. The method of any one of options 15-19, wherein the composition or product combination is administered to the mother of the subject.

21. The method of any one of options 15-20, wherein the prenatal subject is selected as being at risk of developing ASD or a symptom of ASD due to the mother of the prenatal subject having a sample, preferably a fecal sample, comprising:
a reduced level of taurine and/or 5AV compared to a sample from control mother of a non-ASD offspring; and/or
an elevated level of 3-aminoisobutyric acid (3AIBA) compared to a sample from the control mother of a non-ASD offspring.

22. The method of any one of options 15-21, wherein the prenatal subject or the mother of the prenatal subject is selected as having:
reduced levels of taurine and/or 5AV compared to a non-ASD control or a mother of a non-ASD control; and/or
elevated levels of 3-aminoisobutyric acid (3AIBA) compared to a non-ASD control or a mother of a non-ASD control.

23. A method of reducing a symptom of ASD in a selected subject, the method comprising administering a composition or product combination (e.g., more than one composition) comprising bacteria selected from the group consisting of; Bacteroides ovatus, Parabacteroides merdae, and Bacteroides thetaiotaomicron, and a mixture of two or more of the listed bacteria to the subject, whereby the symptom of ASD is reduced in the subject after birth.

24. The method of option 23, wherein the composition or product combination is substantially free of Eisenbergiela tayi.

25. The method of option 23 or 24, wherein the composition or product combination further comprises a taurine precursor and/or a 5-Aminovaleric acid (5AV) precursor.

26. The method of any one of options 22-25, wherein the administering comprises colonizing a region of the subject's gastrointestinal tract.

27. The method of any one of options 23-26, wherein the administering comprises one or more fecal transplants.

28. The method of any one of options 23-27, wherein said composition or product combination is stabilized,
(for example, the composition can comprise at least one of a buffer, and insulation, and/or a sealant, or the composition can comprise at least one of a buffer, an insulation, a sealant, a cryoprotectant, an anti-oxidant, and/or a coating; and/or the composition can comprise live bacteria that are not in a logarithmic growth phase).

29. The method of any one of options 23-28, wherein said composition or product combination is administered prenatally.

30. The method of option 29, wherein said composition or product combination is administered directly to the selected subject prenatally.

31. The method of option 29, wherein said composition or product combination is administered to the mother of the selected subject prenatally, thereby administering said composition or product combination to the selected subject prenatally.

32. The method of any one of options 23-31, wherein said subject is selected as one being at risk of developing ASD or a symptom of ASD.

33. The method of option 32, wherein said subject has a colon sample showing at least:
reduced levels of taurine and/or 5AV compared to a non-ASD control; and/or
elevated levels of 3-aminoisobutyric acid (3AIBA) compared to the non-ASD control, and
is thereby selected as being at risk of developing ASD or a symptom of ASD.

34. The method of option 32 or 33, wherein the mother of the subject has a colon sample showing at least:
reduced levels of taurine and/or 5AV compared to a control mother of a non-ASD offspring; and/or
elevated levels of 3-aminoisobutyric acid (3AIBA) compared to the control mother of a non-ASD offspring, and
the subject is thereby selected as being at risk of developing ASD or a symptom of ASD.

35. A method of determining a profile of a sample of a subject, the method comprising detecting at least one of:
(a) a presence and/or level of a gut bacterium selected from the group consisting of: Bacteroides ovatus, Parabacteroides merdae, Bacteroides thetaiotaomicron, or a combination of two or more of the listed bacteria; and a presence and/or level of Eisenbergiela tayi;
(b) a presence or gene product level of a gut microbiota gene that is an ortholog of KEGG ortholog K0681, and/or
a presence or gene product level of a gene that is an ortholog of KEGG ortholog K1442, wherein the sample comprises gut, feces, or gut and feces material of the subject;
(c) a level of colon taurine, 5-Aminovaleric acid (5AV), lysine, 3-aminoisobutyric acid (3-AIBA), genistein, daidzein, lysine, 5-aminopentanoate, cellobiose, glyceric acid,
a level of serum D ribose, ribitol, ribonic acid, or L-tyrosine,
or a rate of degradation thereof;
(d) a level of a product of a gene of the subject selected from the group consisting of: Gm26944 (or an ortholog thereof), Gm13016 (or an ortholog thereof), Gm17259 (or an ortholog thereof), 4930539E08Rik (or ortholog thereof), Dag1b (or an ortholog thereof), a human ortholog of any of the listed genes, or a combination of two or more of the listed genes; and/or
(e) a level of a splice variant of the subject selected from the group consisting of: a mutually exclusive exon in Neurexin 2; a mutually exclusive exon in Ankryin 2; a skipped exon of Cacna1c; a retained intron of Ads1; or a skipped exon of a pogo transferrable element derived with ZNF domain Pogz,
wherein the profile comprises the detected presence and/or levels of (a), (b), (c),
(d), (e), or a combination of two or more of (a), (b), (c), (d), and (e).

36. The method of option 35, wherein determining the profile comprises determining (a), wherein the sample comprises gut and/or feces material of the subject, and wherein elevated risk of ASD is indicated by reduced levels of Bacteroides ovatus, Parabacteroides merdae, Bacteroides thetaiotaomicron; or increased levels of Eisenbergiela tayi, relative to levels present in a non-ASD control subject.

37. The method of any one of options 35-36, wherein determining the profile comprises detecting (b), wherein the sample comprises gut and/or feces material of the mother of the subject, and wherein elevated risk of ASD is indicated by increased levels of the gut microbiota gene that is the ortholog of KEGG ortholog K0681 and/or decreased levels of the gene that is the ortholog of KEGG ortholog K01442 relative to levels present in a non-ASD control subject.

38. The method of any one of options 35-37, wherein determining the profile comprises detecting (c), wherein the sample comprises colon contents of the subject and/or serum of the subject, and wherein colon levels of taurine, 5AV, 5-aminopentanoate, or cellobiose, below a non-ASD control, serum levels of ribitol or L-tyrosine below a non-ASD control, colon levels of 3AIBA, lysine, glyceric acid, genistein, and/or daidzein above a non-ASD control, and/or serum levels of D ribose or ribonic acid above a non-ASD control indicate an increased risk of developing and/or severity of ASD in the subject.

39. The method of any one of options 35-38, wherein determining the profile comprises detecting (c), wherein the sample comprises colon contents of the subject, and wherein colon levels of taurine, 5AV, 5-aminopentanoate, or cellobiose below a non-ASD control, and/or colon levels of 3AIBA, lysine, glyceric acid, genistein, and/or daidzein above a non-ASD control indicate an increased risk of developing and/or severity of ASD in the subject.

40. The method of any one of options 35-39, wherein determining the profile comprises detecting (c), wherein the sample comprises serum of the subject, and wherein serum levels of ribitol or L-tyrosine below a non-ASD control, and/or serum levels of D ribose or ribonic acid above a non-ASD control indicate an increased risk of developing and/or severity of ASD in the subject.

41. The method of any one of options 35-40, wherein determining the profile comprises determining (d), and wherein levels of Gm26944, Gm13016, and/or Gm17259 gene product greater than a non-ASD control, and/or levels of 4930539E08Rik and Dag1b, or a human ortholog of any of the listed genes below a non-ASD control indicate a presence or elevated risk of ASD.

42. The method of any one of options 35-41, wherein determining the profile comprises determining (e), and wherein levels of Gm26944, Gm13016, Gm17259, 4930539E08Rik, and Dag1b gene products or human orthologs thereof are determined.

43. The method of any one of options 35-42, wherein determining the profile comprises determining (f), and wherein levels of the mutually exclusive exon in Neurexin 2 above a non-ASD control; levels of the mutually exclusive exon in Ankryin 2 below the non-ASD control; levels of the skipped exon of Cacna1c above the non-ASD control; levels of the retained intron of Ads1 above a non-ASD control; and/or levels of the skipped exon of the pogo transferrable element above a non-ASD control indicate an increased risk of ASD.

44. The method of any one of options 35-43, wherein determining the profile comprises determining (f), and wherein levels of the mutually exclusive exon in Neurexin 2 above a non-ASD control; levels of the mutually exclusive exon in Ankryin 2 below the non-ASD control; levels of the skipped exon of Cacna1c above the non-ASD control; levels of the retained intron of Ads1 above a non-ASD control; and/or levels of the skipped exon of the pogo transferrable element above a non-ASD control indicate an increased severity of ASD.

45. The method of any one of options 35-44, wherein the method detects ASD or a symptom of ASD, predicts a risk of ASD and/or a symptom of ASD, and/or predicts the severity of ASD in the subject.

46. The method of any one of options 35-45, wherein the sample for (a), (b), and/or (c) comprises a gut or fecal sample of the subject.

47. The method of any one of options 35-46, wherein the sample for (d) and/or (e) comprises a cerebrospinal fluid (CSF) or central nervous system (CNS) tissue sample, such as prefrontal cortex (PFC) and/or striatum (STR).

48. The method of option 45, wherein the sample comprises colon contents of the subject.

49. The method of any one of options 35-48, further comprising determining the subject as having or being at risk of developing ASD based on (a), (b), (c), (d), and/or (e).

50. The method of option 49, further comprising prenatally increasing a level of taurine and/or 5AV in the subject.

51. The method of option 50, wherein the level of taurine and/or 5AV is prenatally increased by administering taurine and/or 5AV to the mother of the subject.

52. The method of option 50 or 51, wherein the level of taurine and/or 5AV is prenatally increased by administering a composition or product combination comprising Bacteroides ovatus, Parabacteroides merdae, and/or Bacteroides thetaiotaomicron to the mother of the subject.

53. The method of any one of options 50-52, wherein the level of taurine and/or 5AV is prenatally increased by administering a composition or product combination comprising Bacteroides ovatus, Parabacteroides merdae, and/or Bacteroides thetaiotaomicron to the subject.

54. The method of any one of options 50-53, further comprising prenatally administering a composition or product combination comprising a precursor of taurine and/or 5AV to the subject, thereby increasing the level of taurine and/or 5AV in the subject.

55. The method of any one of options 35-54, further comprising detecting a presence and/or a level of a metabolite, wherein the metabolite is a metabolite that is expressed differently in ASD and non-ASD subjects as shown in any of Tables 3-1, 3-2, and/or 3-3.

56. A composition or product combination comprising:
a) a bacteria that maps to an sOTU selected from the group consisting of: b20cd_Bacteroides, and 4ae7e_Parabacteroides; and
b) a taurine precursor and/or a 5-Aminovaleric acid (5AV) precursor, wherein components (a) and (b) are provided in the same formulation or are provided in separate formulations in a product combination.

57. The composition or product combination of option 56, comprising the taurine precursor and the 5AV precursor.

58. The composition or product combination of any one of options 56-57, wherein the composition or product combination does not comprise Eisenbergiela tayi.

59. The composition or product combination of any one of options 56-58, wherein the taurine precursor is selected from the group consisting of: cysteine, cysteine sulfinic acid, homocysteine, cystathionine, hypotaurine, and a mixture of two or more of the listed items.

60. The composition or product combination of any one of options 56-59, wherein the 5AV precursor is selected from the group consisting of: lysine, cadaverine, 1-piperideine, and a mixture of two or more of the listed items.

61. The composition or product combination of any one of options 56-60, comprising a bacteria that maps to the sOTU b20cd_Bacteroides.

62. The composition or product combination of any one of options 56-61, comprising a bacteria that maps to the sOTU 4ae7e_Parabacteroides.

63. The composition or product combination of any one of options 56-62, comprising a bacteria that maps to the sOTU b20cd_Bacteroides and a bacteria that maps to the sOTU 4ae7e_Parabacteroides.

64. The composition or product combination of any one of options 56-63, wherein a bacteria maps to an sOTU when the bacteria comprises a 16S rRNA sequence of at least 100 nucleotides that is least 97% identical to a reference 16S rRNA sequence of the sOTU.

65. The composition or product combination of any one of options 56-63, wherein a bacteria maps to an sOTU when the bacteria comprises a 16S rRNA sequence of at least 100 nucleotides that is least 99% identical to a reference 16S rRNA sequence of the sOTU.

66. A composition or product combination comprising:
a) bacteria selected from the group consisting of: Bacteroides ovatus, Parabacteroides merdae, and Bacteroides thetaiotaomicron, and a mixture of two or more of the listed bacteria; and
b) a metabolite that is expressed differently in ASD and non-ASD subjects as shown in any of Tables 3-1, 3-2, and/or 3-3, or a precursor of said metabolite, wherein components (a) and (b) are provided in the same formulation or are provided in separate formulations in a product combination.

67. A method of reducing or preventing a symptom of ASD in a selected prenatal subject after birth, the method comprising administering a composition or product combination comprising an amount a metabolite to a subject prenatally, the amount being effective to reduce or prevent the symptom of ASD in the subject after birth, wherein the metabolite is a metabolite that is expressed differently in ASD and non-ASD subjects as shown in any of Tables 3-1, 3-2, and/or 3-3.

68. The composition or product combination of any one of options 1-14 or 56-66, wherein the bacteria is in amount sufficient to establish a colony in the gut of a human subject when administered for microbiome transplant or probiotic treatment.

69. The composition or product combination of option 68, wherein the colony persists for at least 1, 2, 3, 4 or more weeks post-inoculation.

70. The method of any one of options 23-34 or 53-55, wherein the composition or product combination administered to the subject comprises bacteria in amount sufficient to establish a colony in the gut of the subject when administered for microbiome transplant or probiotic treatment. 71. The composition or product combination of option 68, wherein the colony persists for at least 1, 2, 3, 4 or more weeks post-inoculation.

### EXAMPLES

### Example 1: Microbiomes from Individuals with ASD are Sufficient to Induce Altered Behaviors in Mice

Gut microbiomes of individuals with ASD, and specifically that of ASD children with intestinal symptoms, have been observed to be significantly different from TD controls (De Angelis et al., 2013; Gondalia et al., 2012; Kang et al., 2013; Son et al., 2015; Strati et al., 2017). Herein, it was tested whether altered human microbiomes functionally contribute to ASD behaviors in mice. Fecal samples from male TD and ASD donors were selected based on Autism Diagnostic Observation Schedule (ADOS; (Gotham et al., 2007)) and gastrointestinal severity index scores (GSI) (Schneider et al., 2006) (Tables 1.1-1.4).

Human fecal samples were previously collected from typically developing children and children with autism spectrum disorders (ASD). (See the methods of Kang et al., 2013). All fecal samples and their metadata including gastrointestinal (GI)- and ASD-relevant clinical data were de-identified before being shared.

As autism is considered to be a neurodevelopmental disorder, with evidence for maternal effects on the fetus (Hallmayer et al., 2011; Lyall et al., 2014), pairs of male and female germ-free (GF) C57BL/6J weanlings were colonized with each of the TD or ASD donor samples and subsequently mated at seven to eight weeks of age (FIG. 1A).

Germ-free (GF) C57BL/6J weanlings (3-4 weeks of age) from the Mazmanian laboratory colony were colonized with fecal samples from human donor. Samples were collected by the Krajmalnik-Brown laboratory at the Arizona State University as part of a previous study (Kang et al., 2013), and kept at −80 C. Aliquots of 16 donor samples were sent to Caltech and used for colonization. To that end, frozen aliquots were thawed in an anaerobic chamber and resuspended in two volumes of reduced sodium bicarbonate solution (final concentration 5%). Subsequently, samples were vigorously vortexed and spun down. Supernatants were then used to colonize GF mice by gavage (100 ul/mouse; Instech, PA, USA). Colonized mice (4-6 females and 2-3 males per donor) were then allowed to rest for 3 weeks, and were subsequently mated. Pregnant dams were single-housed at E15.5-17.5, and offspring were weaned at 3 weeks of age. At weaning, different litters born within up to a week of each other were combined and housed in groups of 4-5 male or female mice per cage and used for subsequent analyses. Cages were assigned to either behavior testing or for tissue collection. Behavior testing started at 6 weeks of age, while tissue was collected at P45. Throughout the study, colonized animals were maintained in autoclaved microisolator cages with autoclaved water and chow (Laboratory Autoclavable Rodent Diet—5010, LabDiet, St. Louis, Mo.).

All mice were tested using the same battery of behavioral tests, starting six weeks of age, in the following order: Open field testing, marble burying, social behavior, USV (male-female contest). Mice were allowed to rest for at least two nights after cage changing before they were tested and tests were performed 4-7 days apart to allow mice to rest between tests. Mice were acclimated to the behavior testing room overnight to reduce stress and anxiety. Mice were tested during the light phase of the light cycle; to control for time of day effects, cages of different groups were alternated. During the initial discovery phase, the experimenter was blinded to the donor but not to the group. The experimenter as well as the person scoring videos were blinded in the subsequent validation phase. Each donor sample was tested 1-3 times and the aggregated data is presented.

Open field testing (OFT)—OFT, as a measure for locomotion and anxiety, was performed in 50×50 cm² white plexiglas arenas, recorded using an overhead camera, and tracked and analyzed using the EthoVision XT 10 software package (Noldus Information Technology). Prior to testing, the arena was disinfected using Rescue (formerly Accel) disinfectant (Virox technologies), followed by 70% ethanol and finally water. Mice were then introduced to the arena and allowed to explore 10 min while tracked. The distance traveled, as well as the number of entries and time spend in a 30×30 cm² center square, were analyzed by the EthoVision software.

Marble burying (MB)—MB, as a measure for repetitive behavior, was performed in a normal cage bottom (Lab Products) with floor area of 75 in² filled with 3-4 cm high deep fresh autoclaved wood chip bedding (Aspen chip bedding, Northeastern Products Corp, Warrensburg, N.Y.). Mice were first habituated to the cage for 10 minutes, and subsequently transferred to a holding cage while the bedding was leveled and 20 glass marbles (4×5) were placed on top. Mice were then returned to their own cage and removed after 10 minutes. The number of buried marbles (50% or more covered) was then recorded and photographed for reference. Bedding was replaced for each mouse, and marbles were soaked in 70% ethanol and dried in bedding in between each test.

Social Behavior (SI)—3-chamber sociability test—the 3-chamber sociability test was performed in a 60×40 cm² white plexiglass box divided in three chambers (20×40 cm²) by clear plexiglass dividers. Mice were first habituated to the full empty arena for 10 minutes. Subsequently, mice were confined to the center chamber and a stimulus mouse (sex-matched adult SPF C57B116) was placed in a small cage in one camber (social chamber) while a small object was placed in a cage on the other chamber (non-social chamber). Mice were then allowed to travel between chambers for 10 minutes, and the movement of mice was recorded by an overhead camera and tracked using the EthoVision XT 10 software package (Noldus Information Technology). A sociability index was calculated by the following:

$$SI = \frac{time in social chamber}{( time in social chamber ) - ( time in nonsocial chamber )} \times 50 - 100 \qquad ( I )$$

Prior to each test, arenas were disinfected using Rescue (formerly Accel) disinfectant (Virox technologies), followed by 70% ethanol and finally water.

Social Behavior (SI)—direct social interaction (DSI) test—As a more sensitive measure for sociability, the direct social interaction test was also used, where a mouse is allowed to interact with a stimulus mouse while the interaction is being recorded. Each mouse was introduced to a fresh empty cage and allowed to habituate for 10 minutes; grooming behavior was scored for the last five minutes of this period. Subsequently, a stimulus mouse (either juvenile or adult SPF C57BL/6J, depending on the experiment) was introduced to the cage for 6 additional minutes. A blinded observer scored videos for any social approach, aggression, or grooming behavior (ref) using the ETHOM software (Shih and Mok, 2000). A set of reference videos was used to ensure consistency over time.

Ultrasonic vocalization (USV)—to test deficits in communication, the male-female paradigm was used to test deficits in communication in male mice. Mice were single-housed and exposed to a new SPF C57BL/6 female for 10 minutes every day in the three days prior to the test. On the fourth day, mice were habituated to an empty cage (no bedding) with a filter soaked with a drop of fresh female urine for 10 minutes. Subsequently, the filter was removed and a novel female was introduced to the cage, and ultrasonic vocalizations were recorded using Avisoft UltraSoundGate 116Hme microphone and the Avisoft Sas-lab Recorder software. Total vocalization and vocalization counts were recorded during 3-minute sessions of male-female interaction.

Adult offspring mice that inherited donor-derived microbiota were either sampled (feces, serum, brains) or behavior tested (FIG. 1A). Donors were stratified using the standardized ADOS (StdADOS; Tables 1.1-1.4) score for disease severity (Gotham et al., 2009) and assigned samples to one of three groups, namely TD, Mild-ASD (StdADOS=4-5), or ASD (StdADOS=6-10). Mice colonized with samples from ASD donors (higher StdADOS scores) display behavioral deficits relevant to ASD, while mice colonized with samples from Mild-ASD donors behave as NT controls (FIG. 1F). Accordingly, further in-depth studies were performed on fecal samples from three NT and five ASD donors (Tables 1.1-1.4, in bold).

TABLE 1.1

| | ID in | | | | |
|---|---|---|---|---|---|
| Donor ID | Ms | Group | Gender | Age | GI score |
| C1-new | C1 | TD | M | 9 | 0 |
| C4-new | C4 | TD | M | 6 | 0 |
| C5-new | NA | TD | M | 9 | 2 |
| C11-new | NA | TD | M | 5 | 0 |
| N5-old | N5 | TD | M | 4 | 0 |
| A8-new | NA | ASD-Mild | M | 9 | 9 |
| A8-old | NA | ASD-Mild | M | 6 | 4 |
| A9-new | NA | ASD-Mild | M | 6 | 2 |
| A3-old | A3 | ASD | M | 8 | 2 |
| A7-old | NA | ASD | M | 6 | 3 |
| A9-old | A9 | ASD | M | 4 | 5 |
| A10-new | NA | ASD | M | 9 | 9 |
| A11-old | A11 | ASD | M | 5 | 5 |
| A15-old | NA | ASD | M | 3 | 7 |
| A17-old | A17 | ASD | M | 11 | 7 |
| A24-new | A24 | ASD | M | 4 | 8 |

TABLE 1.2

| | | | | | Play/ | |
|---|---|---|---|---|---|---|
| | | | | | imagi- | Stereo |
| | | | Communi- | | nation | behav/ |
| Donor | Std | ADOS | cation | Social | creativity | restric |
| ID | ADOS | totals | Sub | sub | total | Interests |
| C1-new | — | — | — | — | — | — |
| C4-new | — | — | — | — | — | — |
| C5-new | — | — | — | — | — | — |
| C11-new | — | — | — | — | — | — |
| N5-old | — | — | — | — | — | — |
| A8-new | 4 | 12 | 4 | 8 | 4 | 2 |
| A8-old | 4 | 8 | 3 | 5 | 0 | 2 |
| A9-new | 4 | 7 | 2 | 5 | 0 | 3 |
| A3-old | 6 | 19 | 6 | 13 | 4 | 6 |
| A7-old | 6 | 9 | 2 | 7 | 2 | 2 |
| A9-old | 6 | 12 | 6 | 6 | 1 | 1 |
| A10-new | NA | NA | NA | NA | NA | NA |
| A11-old | 7 | 16 | 5 | 11 | 1 | 2 |
| A15-old | 8 | 19 | 6 | 13 | 4 | 5 |
| A17-old | 9 | 15 | 4 | 11 | 2 | 0 |
| A24-new | 8 | 19 | 6 | 13 | 4 | 5 |

TABLE 1.3

| | | speech/lang/ | | sensory/ | health/ |
|---|---|---|---|---|---|
| | ATEC | comunication | sociability | cognition | physical |
| Donor ID | total | (ATEC1) | (ATEC2) | (ATEC3) | (ATEC4) |
| C1-new | — | — | — | — | — |
| C4-new | — | — | — | — | — |
| C5-new | — | — | — | — | — |
| C11-new | — | — | — | — | — |
| N5-old | — | — | — | — | — |
| A8-new | 72 | 22 | 10 | 20 | 20 |
| A8-old | 52 | 0 | 16 | 12 | 24 |
| A9-new | 24 | 2 | 5 | 6 | 11 |
| A3-old | 106 | 24 | 21 | 23 | 38 |
| A7-old | 58 | 3 | 14 | 12 | 29 |
| A9-old | 101 | 15 | 28 | 27 | 31 |
| A10-new | 100 | 6 | 26 | 19 | 49 |
| A11-old | 110 | 14 | 19 | 25 | 52 |
| A15-old | 86 | 23 | 14 | 11 | 38 |
| A17-old | 98 | 11 | 32 | 20 | 35 |
| A24-new | 127 | 20 | 17 | 26 | 64 |

TABLE 1.4

| | | Sensory | Ritual | SOCPP | | |
|---|---|---|---|---|---|---|
| | PDDBI | total (+) | total (+) | total (+) | SOCAPP (−) | Express (−) |
| Donor ID | total | (PDDBI1) | (PDDBI2) | (PDDBI3) | (PDDBI4) | (PDDBI5) |
| C1-new | — | — | — | — | — | — |
| C4-new | — | — | — | — | — | — |
| C5-new | — | — | — | — | — | — |
| C11-new | — | — | — | — | — | — |
| N5-old | — | — | — | — | — | — |
| A8-new | −7 | 16 | 19 | 10 | 39 | 13 |
| A8-old | −58 | 21 | 28 | 22 | 67 | 62 |
| A9-new | −156 | 6 | 2 | 5 | 76 | 93 |
| A3-old | 27 | 18 | 16 | 21 | 28 | 0 |
| A7-old | −127 | 8 | 17 | 12 | 74 | 90 |
| A9-old | 20 | 39 | 28 | 30 | 40 | 37 |
| A10-new | −55 | 32 | 16 | 20 | 66 | 57 |
| A11-old | −17 | 30 | 24 | 27 | 57 | 41 |
| A15-old | 27 | 45 | 19 | 7 | 34 | 10 |
| A17-old | 14 | 39 | 35 | 34 | 59 | 35 |
| A24-new | 49 | 43 | 31 | 18 | 29 | 14 |

Mice “humanized” with fecal microbiota from ASD donors show increased repetitive behavior (tested by marble burying), decreased sociability (tested by direct social interaction), and decreased communication (tested by ultrasonic vocalization (USV)) compared to mice colonized with fecal samples from TD controls (FIGS. 1B, 1F). Notably, behavioral effects by ASD microbiomes were more pronounced in male mice, and either weaker or absent in females (FIGS. 1B, 1F). These core features of ASD are not the result of anxiety or other locomotion deficits, as male mice spend the same amount of time in the center of an open-field arena and traveled similar distances (FIGS. 1B, IF). Female mice colonized with ASD microbiomes, however, travel shorter distances during open-field testing (FIGS. 1D, 1F). It was observed that the distance traveled in OFT negatively correlates with the donor age, indicating that some age related microbiome changes may affect locomotion (FIG. 1C). Additionally, marble burying is highly correlated with ADOS and GSI scores, and DSI negatively correlated with ADOS scores (FIG. 1C).

These data demonstrate that fecal transplantation from ASD donors into GF mice is sufficient to transfer ASD-relevant behavioral deficits in a sex-specific manner. Accordingly, it is contemplated that in some embodiments herein, transplant of compositions comprising fecal material, for example compositions comprising bacteria as described herein, can modulate symptoms of ASD.

### Example 2: Typically Developing TD and ASD Colonized Mice Harbor Different Bacterial Species that Correlated with Behaviors

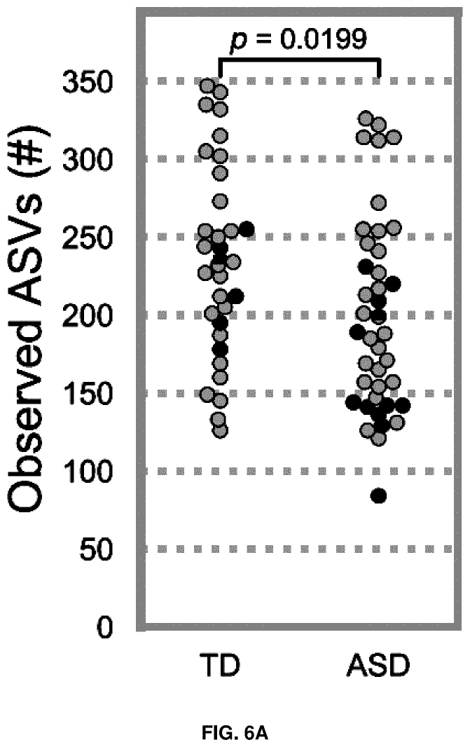

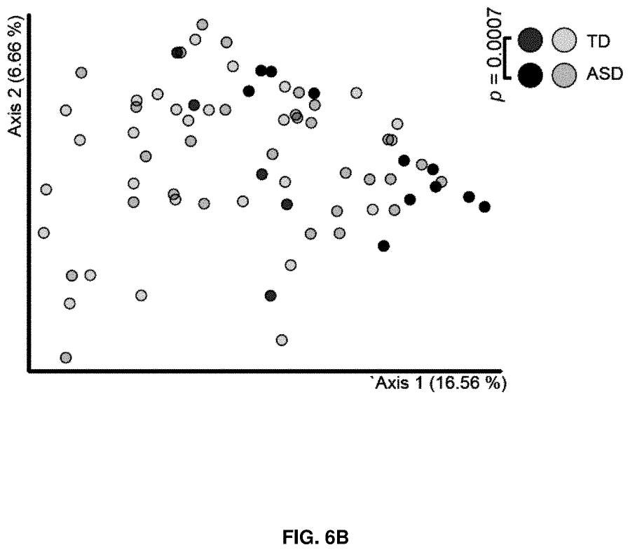

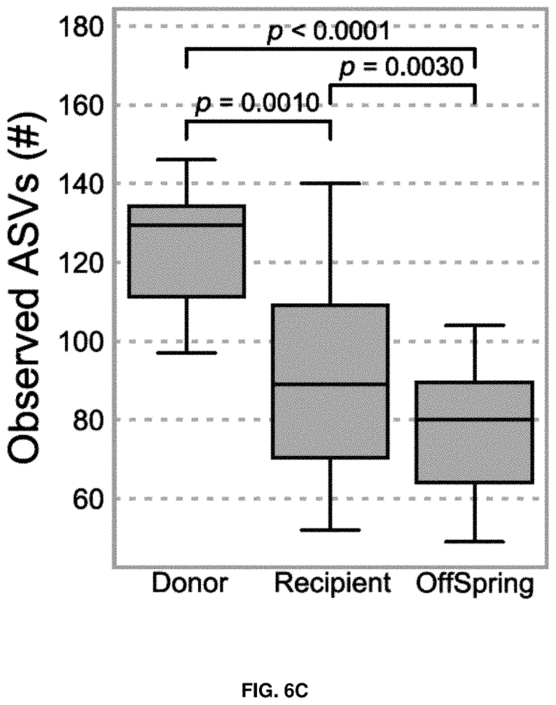

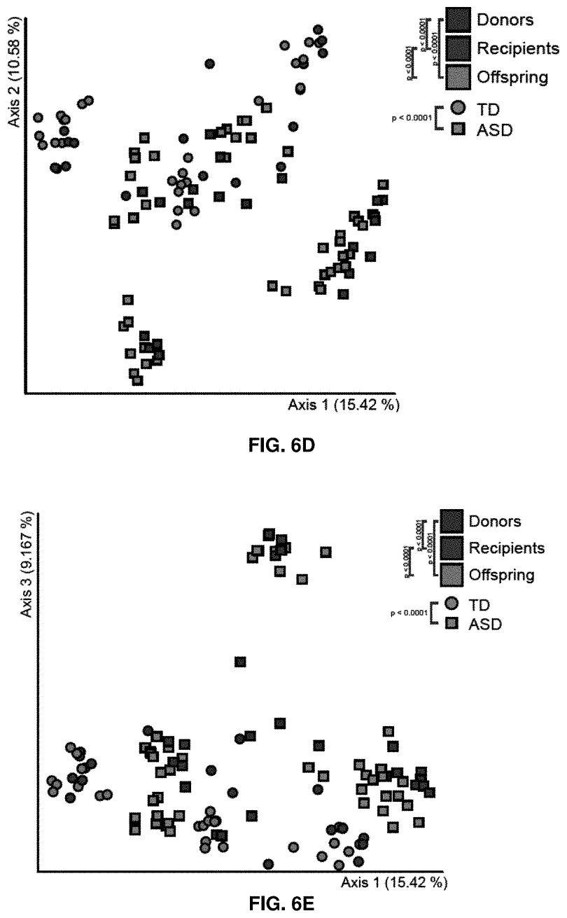

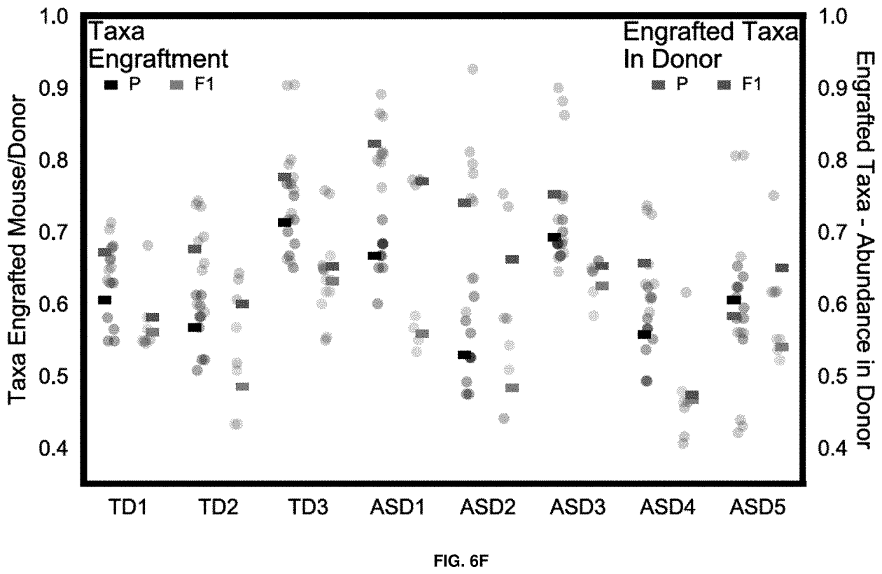

Transplantation of ASD microbiome in otherwise wild-type mice was shown to be sufficient to transfer relevant behavioral changes (FIGS. 1A-C). To validate microbiome transplant, feces were collected from the parental generation of mice directly colonized with human samples (P generation; Recipients) and colon contents of their offspring (F1 generation; Offspring). As previously reported in other systems (Seedorf et al., 2014), transplantation of gut bacteria from human fecal samples into mice results in a predictable shift three weeks after colonization (FIGS. 1G-I). Additional details on engraftment fidelity of colonization with human ASD microbiomes are shown in FIGS. 6A-F. Profiles from donor samples are significantly more similar to each other than to recipients or their offspring, by pairwise distance analysis (PERMANOVA) of unweighted Unifrac distances (FIG. 1G). A significant decrease in alpha diversity was observed upon colonization of mice, indicating loss of bacterial species due to sample processing or host-incompatibility (FIG. 1H). Only a slight shift in alpha and beta diversity was seen in F1 offspring compared to P recipients (FIGS. 1G-I). However, a clear separation between TD and ASD offspring is maintained, except for recipients of a single ASD-donor (A3) that clusters with TD donors (overall pairwise PERMANOVA, p-value=0.0010; FIG. 1D). For example, ASD subject exhibited significantly lower alpha diversity than TD subjects (p=0.0199). ASD samples exhibit different bacterial profiles from NT controls, even at the phylum level (FIG. 1E; See also FIGS. 1K-O). Bacteria that were significantly increased in TD gut microbiomes (relative to ASD) included Bacteriodetes, Bacteroidia, Bacteriodales, Bacteriodaceae, Rikenellaceae, Paraprevotellaceae, Odoribacteraceae, Barneciellaceae, Bacteroides, Paraprevotella, Butyricimonas, Holdemania, Pseudoramibacter eubacterium, Christensenella, Anaerotruncus, and Anaerofilum. Bacteria that were significantly increased in ASD gut microbiomes (relative to TD) were Betaproteobacteria, Burkholderiales, Enterococcaceae, Lactobacillales, Clostridiaceae, Eggerthella, Alistpes, Enterococcus, Clostridium, and Ruminococcus. Furthermore, ASD-colonized offspring exhibited ASD-relevant behaviors, including repetitive behaviors (as assessed by marble burying), impaired sociability (as measured by a sociability index), and anxiety (as measured by center duration an distance traveled) (FIG. 1F). Collectively, fecal transplantation from human donors to GF recipient mice maintains differences between TD and ASD microbiomes, which are vertically transmitted to offspring, contributing to differences in ASD behavior. Thus, it is contemplated that in accordance with some embodiments herein, the gut microbiome composition can induce ASD-behaviors, and further, that adjusting the gut microbiome can inhibit, ameliorate, reduce the likelihood of, or prevent ASD behaviors.

Effects on gastrointestinal physiology and gene expression in ASD offspring mice were also assessed (FIG. 7A-J). In the ASD mice, significant increases in intestinal transit time were observed, as measured by carmine-red gavage and detection in feces, (FIG. 7C), along with significant increases in serum Lipcalin-2 (LCN2) concentration as measured by ELISA (FIG. 7D). Also observed in the ASD population were significant increases in expression the tight-junction genes Occludin (Ocldn) in the distal ileum (FIG. 7E), and significant decreases in Zonula Occludens 2 (ZO2) in the proximal colon (FIG. 7J).

Cytokine expression was quantified in the distal ileum and the proximal colon of TD and ASD mice, as measured by Bioplex 23-plex assay. Measured cytokines included IL-1A, IL-1b, IL-2, IL-3, IL-4, IL-5, IL-6, IL-9, IL-10, IL-12p40, IL-12p70, IL-13, IL-17, Eotaxin, G-CSF, GM-CFS, IFN-g, KC, MCP-1, MIP-1a, MIP-1b, RANTES, and TNF-alpha. No significant differences between TD and ASD mice were observed for any of the cytokines measured. N_{TD}=15, N_{ASD}=20.

Previous microbiome analyses in ASD reported changes in specific bacterial taxa and genera, though there is little overlap in these reports (De Angelis et al., 2013; Gondalia et al., 2012: Kang et al., 2013; Son et al., 2015: Strati et al., 2017; Williams et al., 2011, 2012). To identify differential taxa from the microbiomes of TD versus ASD recipients in the cohort tested herein, DESeq2 analysis was performed (σ=0.001).

Frozen mouse fecal samples were shipped overnight on dry ice and stored in −80° C. until DNA extraction. Human feces that were used as donor samples for the mouse experiments were also shipped back to ASU in order to be processed for microbial DNA extraction and next-generation sequencing together with mouse fecal samples. At ASU, microbial genomic DNA was extracted from fecal samples using the PowerSoil® DNA Isolation Kit (Mobio Carlsbard, Calif.) with a modification based on the manufacturer protocol. Quality and quantity of genomic DNA was verified using a NanoDrop ND-1000 spectrophotometer (NanoDrop Technology, Rockland, Del.). Qualified genomic DNA samples were processed for 16S rRNA library preparation and next-generation sequencing at the Microbiome Analysis Laboratory in the Biodesign Swette Center for Environmental Biotechnology (accessible on the world-wide-web). The Earth Microbiome Project standard protocols (accessible on the world-wide-web) were employed with the barcoded primer set 515F-806R (515F: GTGCCAGCMGCCGCGGTAA (SEQ ID NO: 1), 2086R: GGACTACHVGGGTWTCTAAT (SEQ ID NO: 2)) that targets the V4 region of the bacterial (and archeal) 16S rRNA gene (Caporaso et al., 2012). Paired-end, 2×150 bp, next-generation sequencing was performed using MiSeq Illumina platform (MiSeq Reagent Kit v2; Illumina Inc., San Diego, Calif.) and microbiome sequencing data were analyzed using the Quantitative Insights Into Microbial Ecology (QIIME) software package.

Demultiplexed sequencing outputs were obtained from the ASU sequencing facility and analyzed using the QIIME 2 (version 2017.9) software package according to the suggested workflow (Caporaso et al., 2010). Since there was little overlap between forward and reverse reads, only forward reads (˜150 bp long) were used for subsequent analysis. Primers were first trimmed from the reads and sub-operational-taxonomic units (sOTUs) were obtained using the Deblur denoising plugin (Amir et al., 2017) on reads trimmed to 120 bp.

Subsequently, alignments were obtained using MAFFT (Yamada et al., 2016) and a phylogenetic tree was generated using FastTree (Price et al., 2009). Alpha and Beta diversities were analyzed using the core-metrics-phylogenetic for observed OTUs, Faith's phylogenetic diversity, and Pielou's evenness measures for alpha diversity and unweighted Unifrac and Bray-Curtis for beta diversity measures (Lozupone and Knight, 2005). Taxonomic analysis was performed using the q2-feature-classifier trained on GreenGenes 13_8 99% OTU table (McDonald et al., 2012). Differential abundance analysis was performed using the Phyloseq (1.20.0) and DESeq2 (1.16.1) R packages (Love et al., 2014; McMurdie and Holmes, 2013). To further analyze sOTUs that contribute to the discrimination between NT and ASD samples and to behavioral phenotypes, a RandomForest analysis (Liaw et al., 2002), as implemented in QIIME 2, was used. Per sample metagenomics prediction was done using PICRUSt. For compatibility with the PICRUSt package (Langille et al., 2013), closed-reference OTU tables were generated by clustering sOTUs with the GreenGenes 13_5 99% OTU map in QIIME. Predicted metagenomes were then generated according to the suggested workflow for PICRUSt 1.1.2.

In total, 31 sub-operational taxonomic units (sOTUs) are differentially abundant between groups (FIGS. 2A-2B). Those sOTUs belong predominantly of the Clostridia and Bacteroidia classes, as well as Verrucomicrobia, alpha and beta Proteobacteria with a single representative of each. sOTUs for Bacteroidia are associated with most controls. Specifically, Bacteroides ovatus, Parabacteroides merdae, and an sOTU closely related to Bacteroides thetaiotaomicron, are prevalent in all TD samples, and absent from ASD samples. Conversely, the Lachnospiraceum Eisenbergiela tayi is prevalent among all ASD recipients, and absent from TD groups (FIG. 2B). These observations were further corroborated by an unsupervised classification analysis using RandomForest. The trained classifier assigned all offspring samples correctly by group (TD/ASD; accuracy ratio over baseline:1.75). The trained model indicates 13 sOTUs contributing >1% to discrimination between TD and ASD samples (FIG. 2C), including E. tayi, B. ovatus, and P. merdae (FIG. 2D). The relative abundance of P. merdae and E. tayi in the original human cohorts is shown in FIGS. 2G and 2F, respectively. This differential abundance analysis indicates that several species of Bacteroides and Parabacteroides are exclusively present in TD samples, while different sOTUs that are present only in ASD belong to E. tayi (Lachnospiraceae).

Spearman correlations were performed to test whether discrete sOTUs positively or negatively co-vary with behavioral outcomes: The abundance of four bacterial sOTUs significantly correlates with both repetitive and social behaviors in mice (FIG. 2E). The Bacteroides species (b20cd _Bacteroides; maps to several different Bacteroides species) and P. merdae (4ae7e_Parabacteroides) both correlate with reduced repetitive behavior (less marbles buried) and increased social behavior (longer time socializing in both DSI and 3-chamber sociability). Conversely, the E. tayi sOTUs (02b40_Lacnospiraceae and 29857_Lacnospiraceae) show the opposite effects, as they correlate with increased repetitive behavior and social interaction deficits (FIG. 2E). The association of specific bacterial species (sOTUs) with TD or ASD samples that are also highly correlated with ASD-relevant behaviors supports the hypothesis that specific bacteria contribute to the etiology of ASD.

Accordingly, it is shown that the presence of Bacteroides species and P. merdae correlate with reduced repetitive behavior and increased social behavior. On the other hand, E. tayi sOTUs correlates with symptoms of ASD, including increased repetitive behavior and social interaction deficits. Accordingly, in some embodiments, ASD, or a risk or severity thereof can be identified based on a presence of E. tayi and/or an absence of Bacteroides species and/or P. merdae in a sample of a subject's gut (e.g. a fecal sample).

### Example 3: ASD Microbiomes Promote Extensive Alternative Splicing of ASD-Relevant Genes in the Brain

Social behavior is mediated by multiple brain regions including the prefrontal cortex (PFC) (Barak and Feng, 2016), and modulation of the excitatory/inhibitory balance in the PFC of an ASD mouse model is sufficient to restore normal social behavior (Selimbeyoglu et al., 2017). Evidence in both humans and animal models indicate that synaptic dysfunction and aberrant developmental trajectories in the striatum (STR) may result in increased repetitive behaviors (Langen et al., 2014; Rothwell et al., 2014). To explore the effects of gut bacteria on the central nervous system (CNS), RNA-seq analysis was performed on macro-dissected brain regions from offspring of TD or ASD microbiome colonized mice.

On P45, offspring mice were sacrificed by first administering 5% isoflurane by inhalation for 30 seconds followed by cervical dislocation. Subsequently, blood was collected by heart puncture into 1.1 ml z-gel serum collection tubes (Sarstedt AG $ Co, Germany) for serum collection. Serum was then collected according to manufacturer instructions and kept frozen in −80 C until analysis; brains were macro-dissected using a mouse brain slicer (1 mm coronal section slice intervals; Zivic instruments, Pittsburgh, Pa., USA) and sections of the prefrontal cortex and the striatum were collected into RNALater (Thermo-Fisher Scientific Inc., Waltham, Mass., USA) and kept frozen in −80 C until analysis; the intestines were dissected, colon and cecal contents were collected separately and flash frozen while the intestinal tissue (˜2 cm of the proximal colon and −2 cm of the terminal ileum) were rinsed in PBS and frozen in RNALater. To control for effects by the time of collection, mice from different groups were sacrificed in an alternated fashion. All samples were then assigned an identification number that prevented from direct identification of the groups to facilitate blinded analysis of samples downstream.

Brain tissue from prefrontal cortex and striatum was macro-dissected and flash frozen on dry ice. Approximately 30 mg of frozen brain tissue was then pulverized and RNA was extracted using Qiagen miRNAeasy kits according to the manufacturer's protocol. For each sample, RNA integrity number (RIN) values were quantified using an Agilent Bioanalyzer. RNA sequencing libraries were prepared using TruSeq Stranded mRNA Library Prep kits using polyA selection (Illumina). Libraries were barcoded and randomly pooled in sets of 24. Each pool was then sequenced twice on an Illumina HiSeq 4000 with standard chemistry and protocols for 69 base pair paired end reads (UCLA Neuroscience Genomics Core), to achieve an average depth of 56 million reads per sample. Demulitplexed fastq files were mapped to the mouse reference genome assembly (GRCm38/mm 11) using STAR with Gencode M11 annotations. Quality control was performed using PicardTools (CollectAlignmnetSummaryMetrics, CollectRnaSeqMetrics, CollectGcBiasMetrics, CollectInsertSizeMetrics, MarkDuplicates). To control for differences in RNA quality, read depth, and other sequencing-related technical artifacts across subjects, matrix of “sequencing statistics” was created, corresponding to the aggregate of above Picard Tools metrics for each sample. Two sequencing statistics, seqPC1 and seqPC2, were calculated as the first and second principal components of this matrix and were used as covariates in downstream analyses as previously published (Parikshak et al., 2016). Gene expression was quantified using featureCounts. Genes were filtered to retain only those (n=15695) with a minimum of 10 counts in at least half of the samples. Outlier samples (n=3) were identified from each brain region separately as those with standardized sample network connectivity Z scores <−2, as previously described (Oldham et al., 2012), and were removed. Count-level data then underwent TMM scale normalization, followed by voom transformation and differential gene expression (DGE) using the limma package (Law et al., 2014) in R using the following covariates: Group, Brain Region, RIN, seqPC1, and seqPC2. The limma::duplicateCorrelation function was used to account for non-independence of mice exposed to the same microbiome donor. DGE analysis was then repeated separately for each individual brain region. Test statistics were calculated for the group comparison and local FDR correction was applied to account for multiple comparisons using the fdrtool package in R (Strimmer, 2008). Genes with FDR <0.1 were identified as being differentially expressed. Pathway analysis was performed using gProfileR with GO and KEGG annotations, using a threshold of P<0.005 (uncorrected).

Analysis of event-level differential splicing was performed using rMATS v3.2.5 (Shen et al., 2014). BAM files from ASD- and NT groups were first merged. Percent spliced in (PSI) values were calculated for several classes of alternative splicing events, including skipped exon (SE), alternative 5′ splice site (A5SS), alternative 3′ splice site (A3SS), mutually exclusive exons (MXE), and retained introns (RI). Analyses were repeated separately for each individual brain region, to determine if there were regional differences in splicing patterns across groups. Events with FDR <0.05 were considered differentially spliced across groups.

Cell-type specific expression analysis of genes within each module was performed using the pSI package. (See Dougherty et al., 2010). Cell-type specific gene expression data was obtained from an RNAseq study of purified populations of neurons, astrocytes, oligodendrocytes, microglia, and endothelial cells derived from adult cerebral cortex (Zhang et al., 2016). Raw data (FPKM) was downloaded from GEO (GSE73721). Gene symbols were mapped to Ensembl gene identifiers using the biomaRt R package. Expression values were log 2 normalized and averaged across cell-type replicates. Specificity for the five CNS cell types was calculated with the specificity.index function. Significance was assessed using Fisher's exact test with a pSI threshold set to 0.05, followed by Bonferroni correction of p-values.

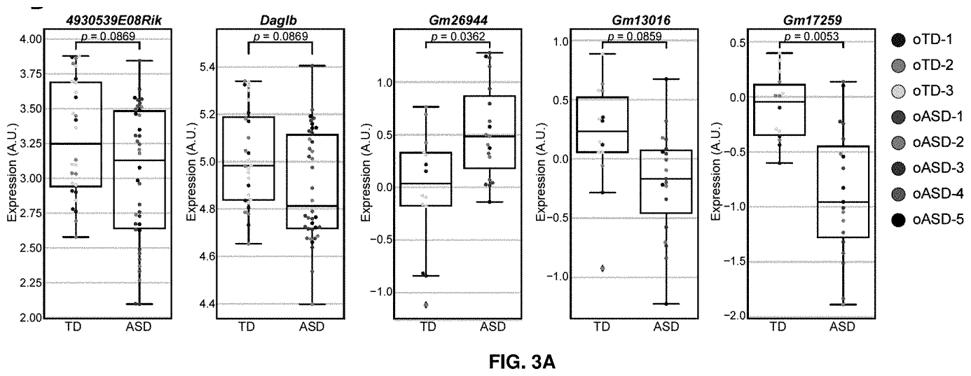

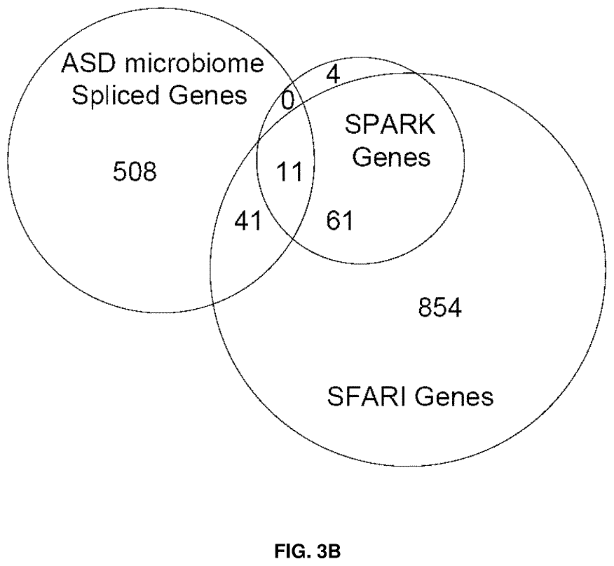

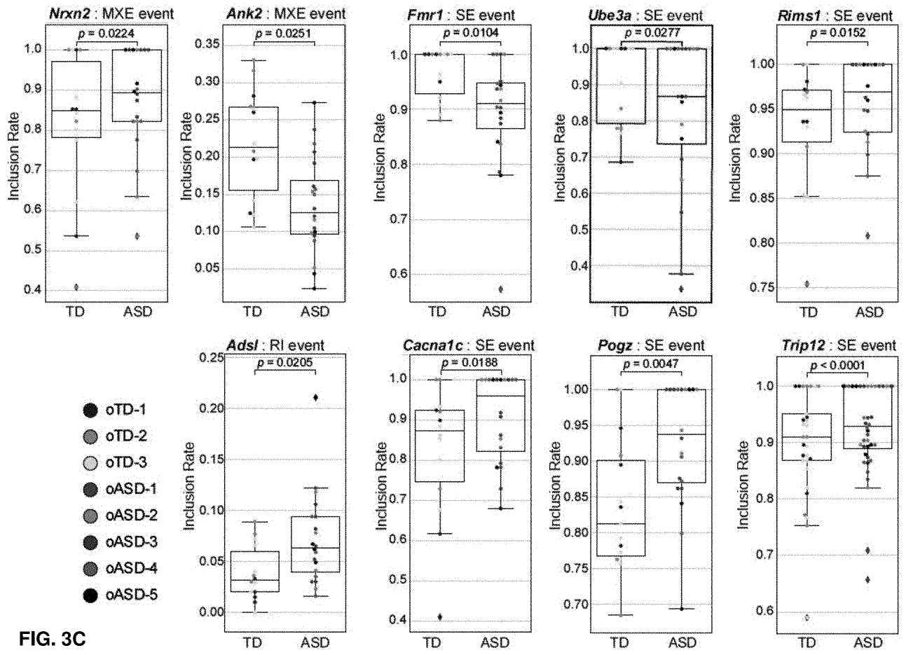

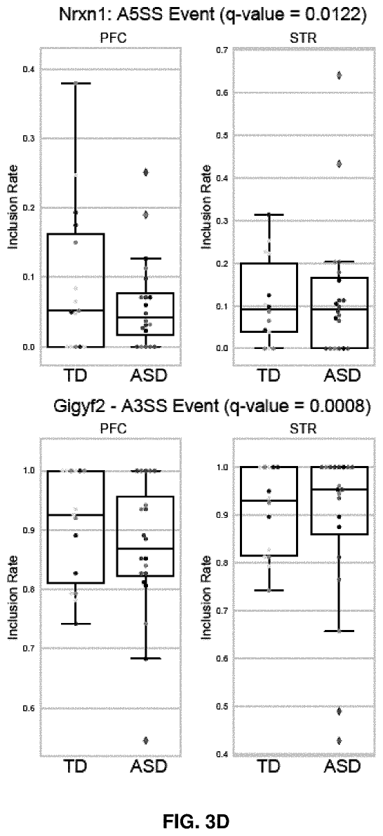

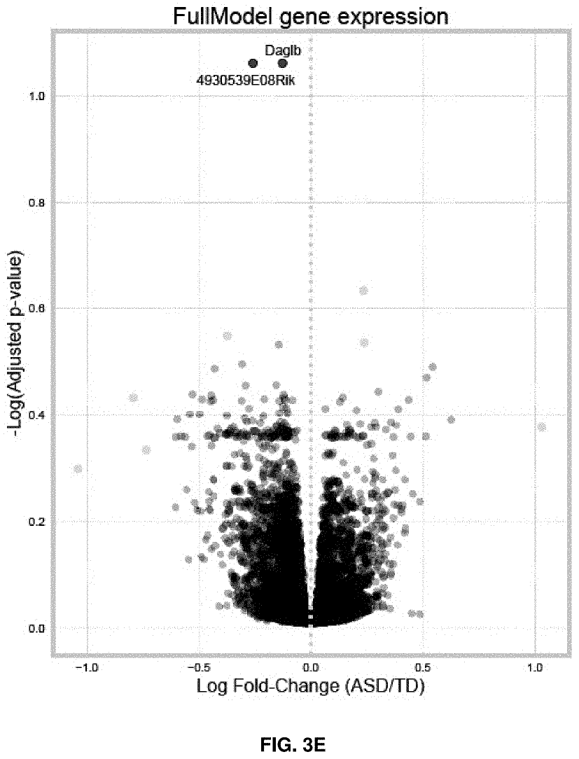

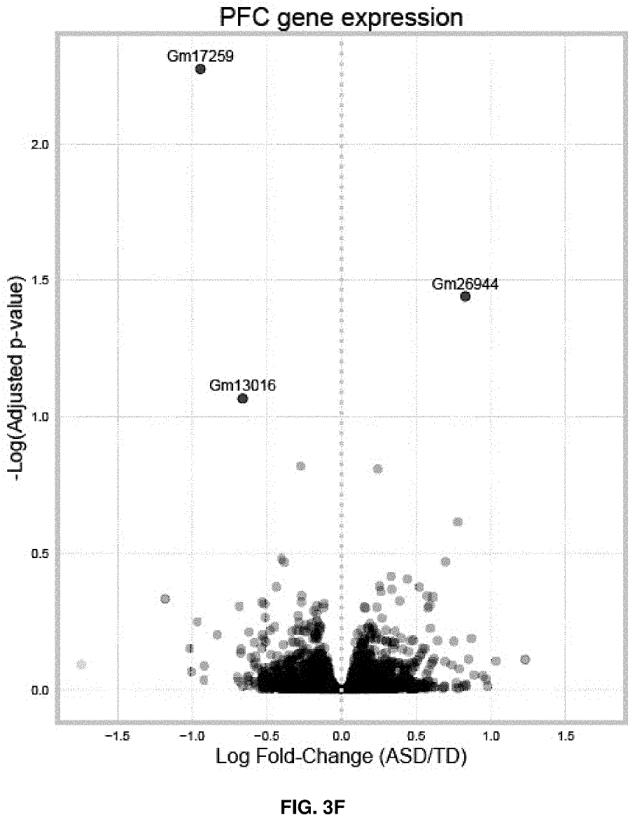

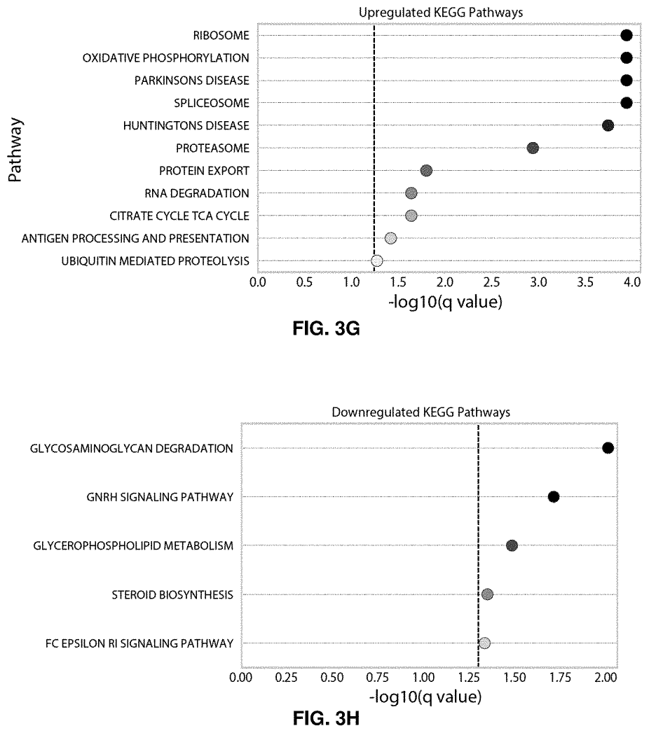

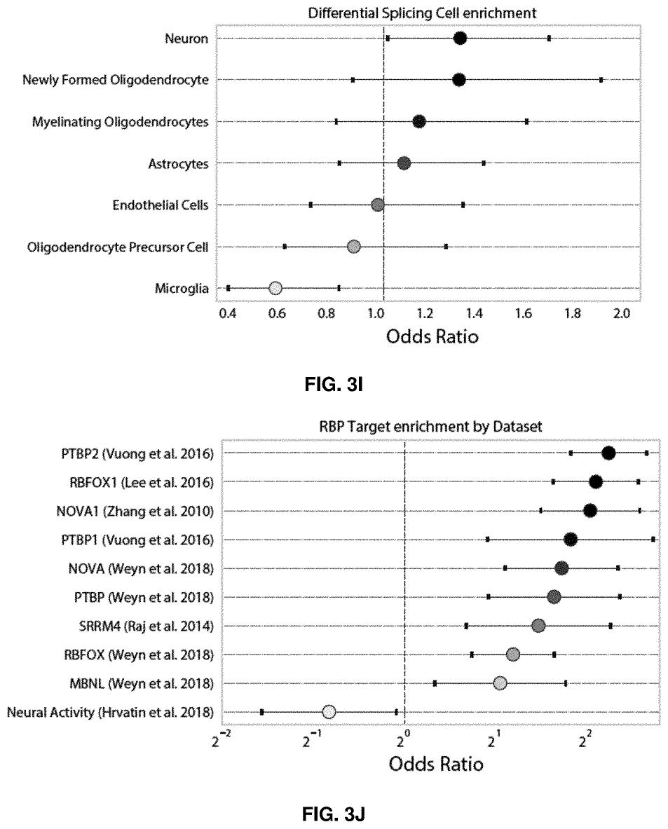

Surprisingly, very few genes show significant differential expression profiles between TD- and ASD-colonized mice (FIGS. 3E-F), with no difference in expression in the STR, three genes in the PFC, and two additional genes when analyzing the full dataset (FIG. 3A). In the PFC, three long-noncoding RNAs (lncRNAs) of unknown function are differentially expressed, with Gm26944 upregulated and Gm13016 and Gm17259 downregulated in ASD-colonized mice, compared to controls. The protein coding genes 4930539E08Rik and Dag/b are both downregulated in ASD-colonized mice. Diacylglycerol Lipase Beta (Dag1b) is reported to be involved in endocanabanoid production and affects axonal growth during development (Bisogno et al., 2003; Powell et al., 2015). Another layer of regulation on the transcriptome is alternative splicing (AS). An rMATS analysis (Shen et al., 2014) found a striking number of differences in alternatively spliced genes. In total, 498 genes are observed to be alternatively spliced in either brain regions: 123 with 3′ or 5′ alternative start sites (A3SS/A5SS) events, 78 with mutually exclusive exon (MXE) events, 315 with skipped exon (SE) events, and 8 events of retained introns (RI). All genes were compared in which splicing events were detected to curated lists of genes suspected or validated to play a role in ASD, from the SPARK for Autism gene list and the SFARJ Gene dataset (Abrahams et al., 2013) (FIG. 3B, Tables 2.1-2.3). 44 of the 498 events (8.8%) occur in genes associated with autism, and 8 in genes highly relevant to ASD (FIG. 3B). In the STR of ASD-colonized mice, the following were observed: an increased inclusion rate of a mutually exclusive exon (MXE) in Neurexin 2, a highly spliced presynaptic adhesion protein strongly linked to ASD (Dachtler et al., 2014; Sudhof, 2008) (FIG. 3C). Ankyrin 2, necessary for neuronal migration (Kordeli and Bennett, 1991; Willsey et al., 2013), exhibits decreased inclusion of a MXE in the STR of ASD-colonized mice, compared to TD controls (FIG. 3C). Other major contributors to human ASD that show aberrant splicing in the PFC of mice receiving ASD microbiota include the calcium voltage-gated channel subunit Cacna1c, required for neuronal survival (Lee et al., 2016); adnylsuccinate lyase Ads1 that is associated with infantile autism (Sivendran et al., 2004), and the pogo transposable element derived with zinc finger (ZNF) domain Pogz, associated with ASD and intellectual disability (Stessman et al., 2016). The protein encoded by Pogz has been described as a zinc finger protein containing a transposase domain at the C-terminus. When considering the full dataset, with samples from STR and PFC together, splicing events in two other ASD genes arise: A A3SS event in the GRB10 interacting GYF protein (Gigyf2), de novo mutations in which have been identified in ASD (Krumm et al., 2015), was detected in the brains of ASD-colonized mice (FIG. 3D). Additionally, an A5SS event in Neurexin 1, a paralog of Nrxn2, was detected (FIG. 3D). Cell-type enrichment analysis in the brains of ASD mice is shown in FIG. 3I. Differential splicing events were enriched (compared to non-ASD controls) in neurons, newly-formed oligodendrocytes, myelinating oligodendrocytes, and astrocytes. Differential splicing events were reduced (compared to non-ASD controls) in endothelial cells and oligodendrocyte precursor cells. Differential splicing of previously reported targets of specific RNA-binding proteins (RBPs) in ASD brains (compared to non-ASD controls) is shown in FIG. 3J. It is noted that differential splicing of targets of PTBP2, RBFOX1, NOVA1, PTBP1, NOVA, PTBP, SRRM4, RBFOX, and MBNL was enriched in ASD brains (compared to non-ASD controls), while differential splicing of neural activity targets was reduced in ASD brains (compared to non-ASD controls) (FIG. 3J).

TABLE 2.1

| AS | | | | | |
|---|---|---|---|---|---|
| Event | GeneID | Gene | | | |
| (Hits in SPARK) | | Symbol | PFC_FDR | Str_FDR | All_FDR |
| A3SS | ENSMUSG00000048000.15 | Gigyf2 | 0.9998 | 1.0000 | 0.0008 |
| A5SS | ENSMUSG00000024109.18 | Nrxn1 | 1.0000 | 1.0000 | 0.0122 |
| SE | ENSMUSG00000026219.14 | Trip12 | 1.0000 | 1.0000 | 0.0000 |
| SE | ENSMUSG00000051331.14 | Cacna1c | 1.0000 | 0.0188 | 0.0100 |
| SE | ENSMUSG00000038902.14 | Pogz | 0.0047 | 1.0000 | 1.0000 |
| MXE | ENSMUSG00000032826.17 | Ank2 | | 0.0251 | 0.1014 |
| MXE | ENSMUSG00000033768.16 | Nrxn2 | 1.0000 | 0.0224 | 1.0000 |
| RI | ENSMUSG00000022407.9 | Adsl | 1.0000 | 0.0205 | 0.0401 |

TABLE 2.2

| | | Coordinates (AS Exon, | | |
|---|---|---|---|---|
| AS | | Upstream Exon, | Transcript | GetPrime |
| Event | GeneID | Downstream Exon) | Name | ID |
| A3SS | ENSMUSG00000048000.15 | +chr1: 87407534-87407723, | gigyf2-217 | 2688828 |
| | | +chr1: 87407292-87407455 | gigyf2-203/210 | 2535936 |
| | | +chr1: 87407537-87407723, | | |
| | | +chr1: 87407292-87407455 | | |
| A5SS | ENSMUSG00000024109.18 | “−chr17: 90701966-90702011, | nrxn1-209 | 2668632 |
| | | −chr17: 90700743-90700905 | nrxn1-207/216 | 2668638 |
| | | −chr17: 90701987-90702011, | | |
| | | −chr17: 90700743-90700905” | | |
| SE | ENSMUSG00000026219.14 | “−chr1: 84777423-84777489, | Trip12- | 2538563 |
| | | −chr1: 84782826-84782963, | 207/201/206/208 | |
| | | −chr1: 84776316-84776412” | trip12-216 | 2691405 |
| SE | ENSMUSG00000051331.14 | “−chr6: 118637729-118637813, | | |
| | | −chr6: 118638426-118638510, | | |
| | | −chr6: 118630224-118630431” | | |
| SE | ENSMUSG00000038902.14 | “+chr3: 94856042-94856201, | pogz-205 | 2505354 |
| | | +chr3: 94853949-94854074, | pogz-203 | 2662340 |
| | | +chr3: 94860795-94860971” | | |
| MXE | ENSMUSG00000032826.17 | “−chr3: 127104760-127104862, | | NaN |
| | | −chr3: 127339131-127339192, | | |
| | | −chr3: 127079897-127079996 | | |
| | | −chr3: 127248133-127248232, | | |
| | | −chr3: 127339131-127339192, | | |
| | | −chr3: 127079897-127079996” | | |
| | | | | NaN |
| MXE | ENSMUSG00000033768.16 | “+chr19: 6448254-6448284, | nrxn2-204 | 2660375 |
| | | +chr19: 6443630-6443648, | nrxn2-209 | 2503392 |
| | | +chr19: 6472257-6472419 | | |
| | | +chr19: 6463823-6463868, | | |
| | | +chr19: 6443630-6443648, | | |
| | | +chr19: 6472257-6472419” | | |
| RI | ENSMUSG00000022407.9 | “+chr15: 80963659-80964027, | adsl-205 | 2608398 |
| | | +chr15: 80963659-80963729, | adsl-204/201 | 2608414 |
| | | +chr15: 80963879-80964027” | | |

TABLE 23

| | | | | SEQ | | SEQ |
|---|---|---|---|---|---|---|
| AS | | GetPrime | | ID | | ID |
| Event | GeneID | ID | Forward | NO: | Reverse | NO: |
| A3SS | ENSMUSG00000048000.15 | 2688828 | AATAACTTG | 3 | GGCCCTGGT | 4 |
| | | | GATGTTCCC | | ATAGTCATG | |
| | | | ACG | | G | |
| | | 2535936 | CAGCAGCC | 5 | TACTTCCTC | 6 |
| | | | ATAACGAA | | ACTAGCAGC | |
| | | | GAG | | C | |
| A5SS | ENSMUSG00000024109.18 | 2668632 | ACAAACTAC | 7 | AGCATGGTG | 8 |
| | | | ATTGTTCGG | | TAATCTTCT | |
| | | | TGAC | | TGC | |
| | | 2668638 | TAATGCCTG | 9 | ACTGATATT | 10 |
| | | | GCATGATGT | | GTCACCATA | |
| | | | G | | GCG | |
| SE | ENSMUSG00000026219.14 | 2538563 | — | | — | |
| | | 2691405 | | | — | |
| SE | ENSMUSG00000051331.14 | 2505354 | TACTTCCGT | 11 | GTTACTAAC | |
| | | | TTCTGTGAG | | AGTAGTGG | |
| | | | C | | ATGTAGAG | |
| SE | ENSMUSG00000038902.14 | 2662340 | AATCAGTGA | 13 | ACCAAAGT | 14 |
| | | | TGTCATTGA | | ATTACCAGC | |
| | | | GGAC | | GGA | |
| MXE | ENSMUSG00000032826.17 | NaN | | — | — | |
| | | NaN | — | — | | — |
| MXE | ENSMUSG00000033768.16 | 2660375 | TAAACAGC | 15 | TGTGATCTC | 16 |
| | | | GAAGGCAA | | GTCAGTGCT | |
| | | | GGA | | G | |
| | | 2503392 | AAGTTCTGC | 17 | GCAGAAGA | 18 |
| | | | AGTGAAGA | | ACTCATTGC | |
| | | | GGA | | CC | |
| RI | ENSMUSG00000022407.9 | 2608398 | GTGCACAA | 19 | TCACCAATC | 20 |
| | | | GATTTGCAC | | TGCTGTTTC | |
| | | | TG | | TC | |
| | | 2608414 | CTCCTGCTA | 21 | ACTCTAGCA | 22 |
| | | | TGTCGGAGA | | AGCTTTGGC | |
| | | | C | | A | |

KEGG pathways upregulated (FIG. 3G) and downregulated (FIG. 3H) in the brains of ASD mice were determined by Gene Set Enrichment Analysis (GSEA). In the brains of ASD mice, pathways associated with ribosomes, oxidative physphorylation, Parkinson's disease, spliceosome, Huntington's disease, proteasome, protein-export, RNA degradation, the citrate TCE cycle, antigen processing and presentation, and ubiquitin mediated proteolysis were identified as upregulated (FIG. 3G). In the brains of ASD mice, pathways associated with glycosaminoglycan degradation, GNRH signaling, glycerophospholipid metabolism, steroid biosynthesis, and FC epsilon RI signaling were identified as downregulated (FIG. 3H).

Therefore, deep sequencing of RNA has shown a substantial shift in alternative splicing patterns in both the STR and the PFC of mice harboring ASD relative to TD microbiomes, with a striking enrichment for ASD-related genes in the differentially spliced subset. Accordingly, in some embodiments a profile of a subject sample comprising detected splicing patterns in CNS tissues can indicate an ASD, and/or a risk or severity of developing ASD or symptoms thereof.

### Example 4: Products from Microbial Metabolism Differ Between TD and ASD-Colonized Mice

Gene expression, splicing, as well as the function of neurons in the enteric nervous system (ENS) or CNS can be impacted by small molecule metabolites (Nankova et al., 2014). An altered metabolomic profile has long been associated with ASD (Aldred et al., 2003; De Angelis et al., 2013; Evans et al., 2008; Naushad et al., 2013; Yap et al., 2010). Recently, metabolomes of ASD children and controls, as well as ASD animal models, have been surveyed to explore disease etiology and putative biomarkers, highlighting a role for amino acid metabolism (De Angelis et al., 2013), mitochondrial dysfunction (Rossignol and Frye, 2012), and production of neuroactive metabolites (Hsiao et al., 2013). Many of these differences have been hypothesized to originate from microbial processes in the intestine (De Angelis et al., 2015; Krajmalnik-Brown et al., 2015). To that end, untargeted proton nuclear magnetic resonance (1H NMR) and gas-chromatography-mass-spectrometry (GC-MS) analyses were performed on colon contents from TD- and ASD-colonized mice (FIG. 4A-B), and also surveyed the metabolome in mouse serum by GC-MS (FIG. 4C). In colon contents, a total of 122 metabolites were identified by GC-MS (out of a total of 246 detected), and 67 metabolites were detected and identified by NMR The raw NMR and mass spectrometry data on are provided as large Table 3-1 (NMR norm. data; submitted herewith as CALTE127PR-Table 3-1.txt), Table 3-2 (GC-MS_norm. data; CALTE127PR-Table 3-2.txt), and Table 3-3 (GC-MS serum data; CALTE127PR-Table 3-3.txt). These tables are also shown in an Appendix, below. In serum, a total of 130 metabolites were identified by GC-MS (out of a total of 255 detected). A deficit in amino acid degradation was observed in colon contents of ASD-colonized mice, remarkably similar to reported effects in feces from ASD children (De Angelis et al., 2013) (FIGS. 4I-J). A total of 27 metabolites are significantly different in the colon contents of TD-colonized offspring, compared to animals with ASD microbiomes (FIG. 4), with 24 detected by GC-MS (11 identified) and 4 by NMR (FIG. 4A-D). Specifically, this analysis identified metabolites of several different families: amino acids and their derivatives, isoflavonoids, carbohydrates and their derivatives, and fatty acids. It was observed that higher levels of lysine in ASD colons, compared to TD, while its breakdown product 5-aminovaleric acid (or 5-aminopentanoate; 5AV) is significantly lower, suggesting that lysine-degrading bacteria are missing from the ASD microbiome (FIGS. 4A-B, C-D, F). 5AV is a weak GABA receptor agonist (Callery and Geelhaar, 1985), and is significantly lower in children with ASD, compared to TD controls (Ming et al., 2012). Lower levels of another weak GABA agonist, taurine, are found in a subset of ASD subjects (Adams et al., 2011; Park et al., 2017; Tu et al., 2012). Additionally, mice colonized with ASD-microbiomes have 50% less taurine compared to TD-colonized mice (FIG. 4B, E, G). Without being limited by theory, it is contemplated that together, lower levels of 5AV and taurine suggest that gut microbes may affect behavior via GABA signaling. 3-aminoisobutyric acid (3AIBA), a degradation product of the amino acid thymine, is elevated in children with ASD (West et al., 2014); similarly, increased concentrations of 3AIBA were observed in ASD-colonized mice, compared to TD controls (FIGS. 4A, D, G). Another group of putatively bioactive molecules elevated in colons of ASD-colonized mice are the soy-derived isoflavonoids genistein and daidzein (FIGS. 4A, D, G). This finding indicates that the absence of dietary nutrient metabolizing bacteria in the ASD microbiomes compared to TD (Matthies et al., 2008, 2009). Although no direct effects by these isoflavonoids in autism have been demonstrated, various sexually-dimorphic effects on neurodevelopment and behavior are linked to genistein and daidzein, as well as hormonal effects (Ponti et al., 2017; Rodriguez-Gomez et al., 2014; Setchell and Cassidy, 1999). Furthermore, while 21 serum metabolites are differentially abundant (8 identified) between mice harboring TD vs. ASD microbiomes, only the isoflavone genistein is significantly different in both colon contents and serum (FIGS. 4C, F, G). Lastly, tyrosine is less abundant in ASD samples, compared to TD, as previously observed in individuals with ASD (Adams et al., 2011) (FIGS. 4C, H).

Without being limited by theory, it is contemplated that the gut microbiome can exert its effects on host behavior via multiple routes. Certain bacteria are highly correlated with hallmark ASD behaviors, namely repetitive behavior and deficits in sociability (See FIG. 2E). By correlating metabolites to patient metadata and behavioral outcomes following colonization with specific microbiomes, it is observed herein that several metabolites are associated with gastrointestinal dysfunction (FIG. 5A). This association also highlights the protective role of taurine, 5AV, and other molecules in ASD-like behaviors. In addition, it was predicted that isoflavones contribute to repetitive behavior effects while lysine, 3AIBA, and genistein are predicted to influence locomotion. Therefore, integration of microbiome and metabolome profiles emphasizes the potential contribution of microbial metabolites to behavioral outcomes in mice, and raises testable predictions that intestinal bacteria may contribute to gut-brain crosstalk in ASD via production and degradation of specific molecules.

In summary, these observations in “humanized” mice indicate that specific microbiome-derived or induced metabolites are dysregulated in ASD. Thus, in some embodiments, a profile of detected metabolites (and/or levels thereof) in a subject sample comprising colon contents (e.g. a fecal sample) and/or comprising serum can indicate presence of ASD or a symptom thereof, and/or a risk or severity of ASD or a symptom thereof in a subject.

### Example 5: Prenatal Administration of Microbiome-Regulated Metabolites Predictably Alters ASD-Like Behaviors

The microbiome directly impacts the metabolomic profile of its host (Wikoff et al., 2009). It was hypothesized that the availability of defined metabolites, either reduced in beneficial or enriched in harmful metabolites, underlies the ability of human ASD microbiomes to alter behavioral outcomes in mice. Taurine is the most abundant amino acid in the brain, and is required for proper brain development (Tochitani, 2017a). The developing fetus and neonate relies on the maternal supply of taurine (Reusens et al., 1995; Tochitani, 2017b).

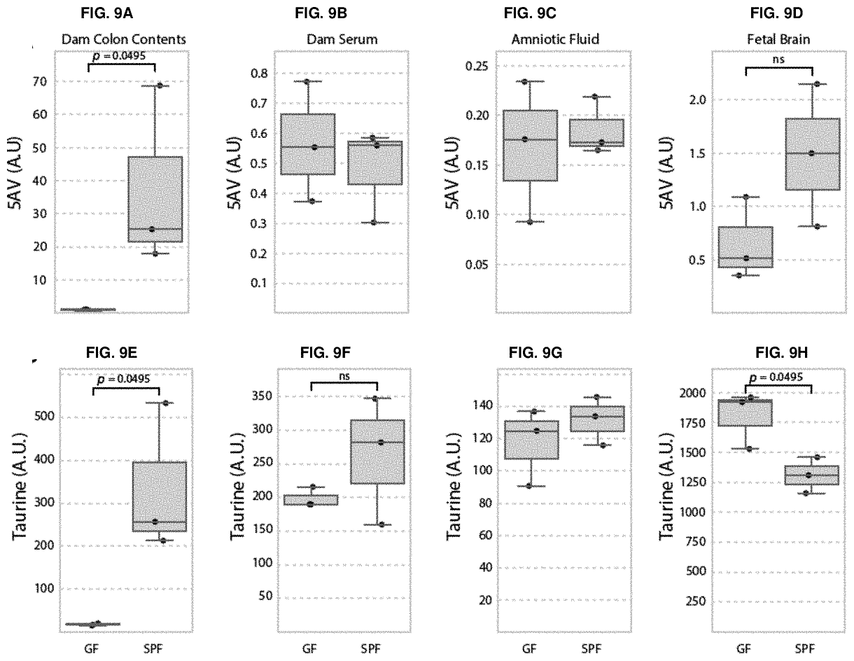

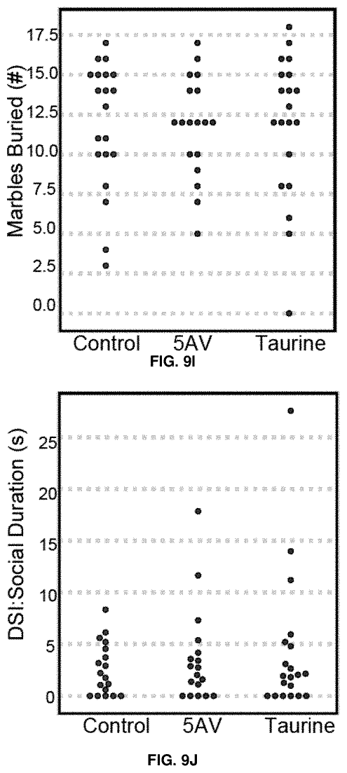

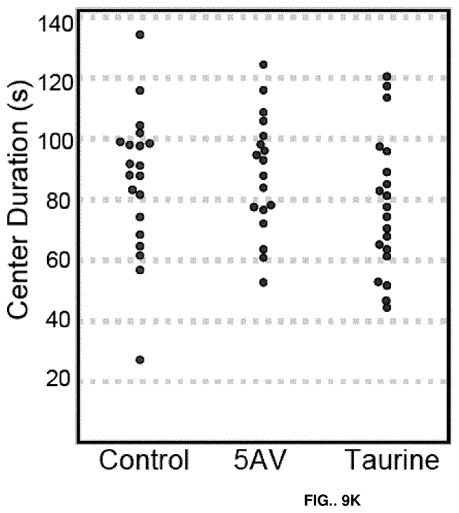

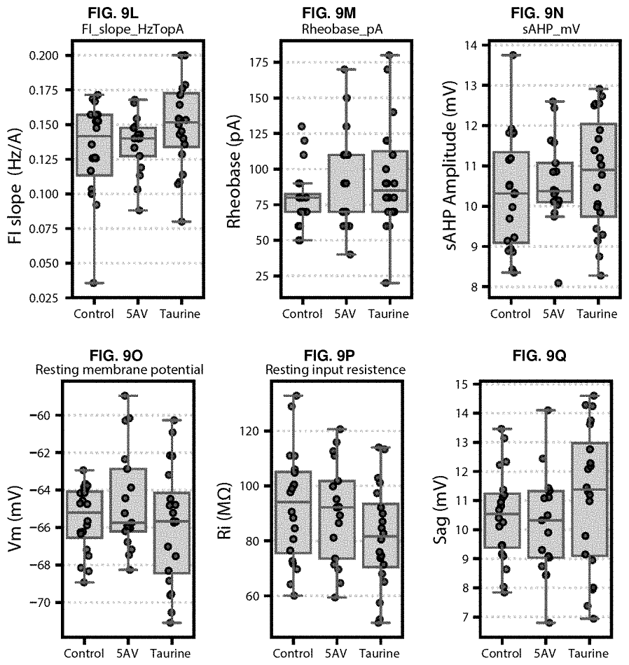

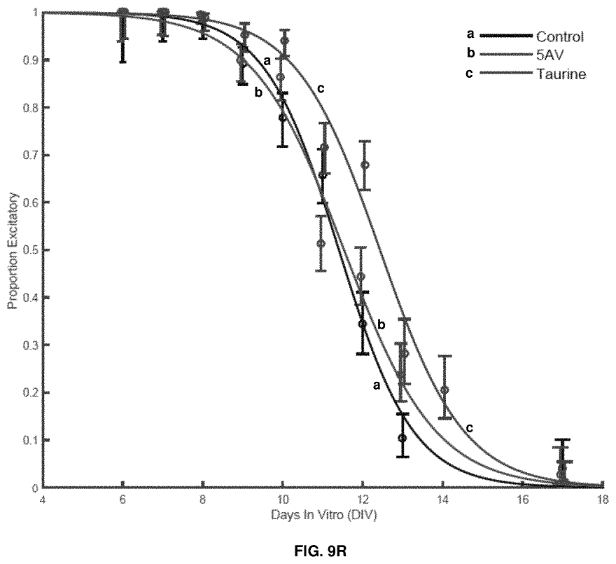

Taurine was orally administered to BTBR mice, a validated mouse model for ASD (McFarlane et al., 2008), from conception through adulthood to capture both prenatal and postnatal neurodevelopmental periods. Consistent with the hypothesis, BTBR mice have low levels of circulating taurine compared to C57BL/6 mice (Klein et al., 2016; McFarlane et al., 2008). It was predicted, based on correlation with behavior (See FIG. 5A), that taurine administration would decrease repetitive behavior and increase sociability. Indeed, taurine significantly reduces repetitive behavior in male mice, as measured by marble burying, with a trend toward increasing social duration in all mice (FIG. 5B). Additionally, offspring treated with taurine display reduced anxiety, as measured by center duration in the OFT. (FIG. 5D). 5AV is a product of lysine degradation by a bacterium that is likely to be absent from or rare in the ASD microbiome. It is will appreciated that the blood-brain barrier comprises endothelial cells, and selectively permits the movement of substances between circulating blood and the brain, for example permitting some molecules to pass from the blood to the brain, while preventing other molecules from passing from the blood to the brain. As such, it will be appreciated that at a given stage of development, some molecules may readily pass through the blood brain barrier, while others may not. In adult mice, only a 5AV precursor 1-piperideine (but not 5AV itself) can enter the brain, where it is rapidly converted to 5AV. In the developing brain, however, the blood-brain-barrier is reported to be more permissive until mid-gestation (Blanchette and Daneman, 2015). 5AV also reduced repetitive behavior, and increased sociability (FIGS. 5B-C). The timing of taurine and 5AV administration is significant. Unlike prenatal administration that showed significant improvement in ASD-related behaviors in BTBR mice, administration to juvenile mice starting 4 weeks of age and through adulthood did not rescue behavioral alterations (FIGS. 9A-S). These findings support the hypothesis that metabolites that can be metabolized or produced by gut bacteria contribute to ASD-relevant behavioral deficits. Furthermore, these data highlight the contribution of the gut microbiome and its metabolites in impacting behavior during particular developmental periods.

Accordingly, in some embodiments herein, taurine (for example, in a composition comprising taurine) is prenatally administered to a subject selected to be at risk of developing ASD and/or a symptom of ASD, so as to reduce the risk and/or severity of ASD or the symptom of ASD in the subject after birth. In some embodiments, a precursor of taurine is prenatally administered to the subject along with gut bacteria that contribute to the synthesis of taurine so as to prenatally increase taurine levels in the subject and reduce the risk and/or severity of ASD or the symptom of ASD in the subject after birth. In some embodiments, gut bacteria that contribute to the synthesis of taurine are prenatally administered to the subject so as to prenatally increase taurine levels in the subject and reduce the risk and/or severity of ASD or the symptom of ASD in the subject after birth.

### Example 6: Treatment with Metabolites Affects Gene Expression and Splicing in the Brain

In view of the results described herein, it was further hypothesized that 5AV and taurine exert their effects on behavioral outcomes by altering expression patterns in the brain. To address this hypothesis, brains were collected from adult metabolite-treated BTBR mice and relative expression was quantified for select genes and isoforms that are differentially expressed between TD- and ASD-colonized offspring (FIGS. 3A-J). Notably, the expression of the lncRNAs Gm17259 and Gm13016 is increased in the STR of BTBR mice administered with 5AV or taurine, but not their PFC (FIGS. 5E-H), suggesting they could compensate for their reduced expression in brains of mice colonized with a human ASD microbiota. To test whether the expression of splice variants containing exons highlighted by rMATs analysis (see FIG. 3B, Tables 2.1-2.3), isoforms of interest were identified, which contain or exclude exons implicated by the analysis. Transcripts of Neurexin 1, a highly-spliced gene expressed by neurons and astrocytes (Sudhof, 2008, 2017) are increased in expression in the STR as a result of 5AV administration (FIG. 51, FIG. 5J, FIG. 5N). Taurine increases the expression of isoforms containing a A3SS event in Gigyf2 (FIG. 5K-L), while 5AV has marginal effects on the expression of isoforms of Tip12 and Pogz (FIG. 5N). Therefore, treatment with 5AV and taurine according to some embodiments affects the expression of lncRNAs and of specific ASD-relevant isoforms in the brains of BTBR mice treated throughout their lives. This analysis indicates that bacterial metabolites directly or indirectly affect expression profiles in the brain as a mechanism to regulate complex behaviors in mice.

### Example 7: Effects of Metabolites on Brain Activity

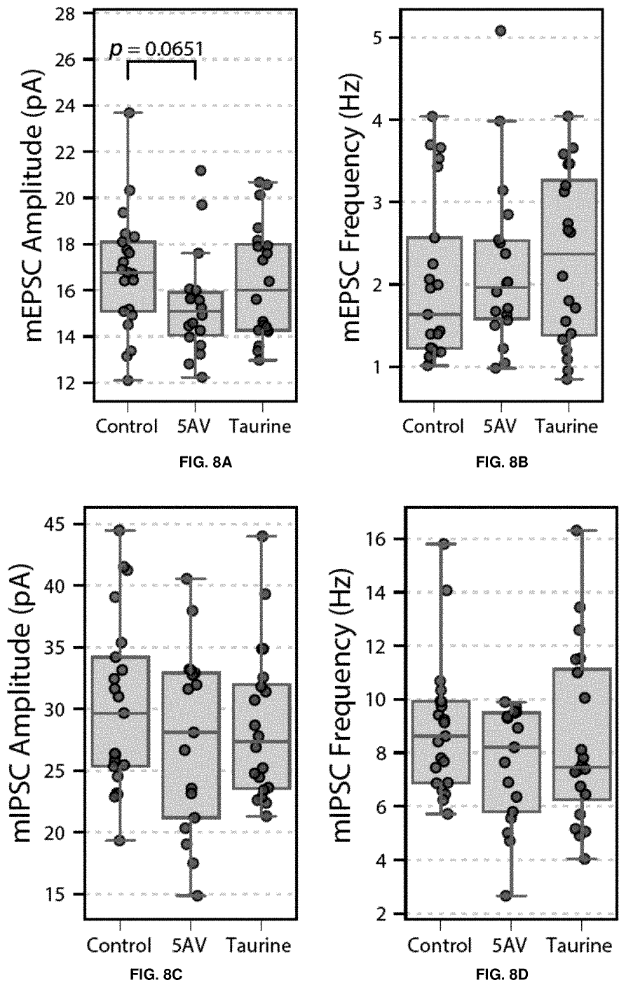

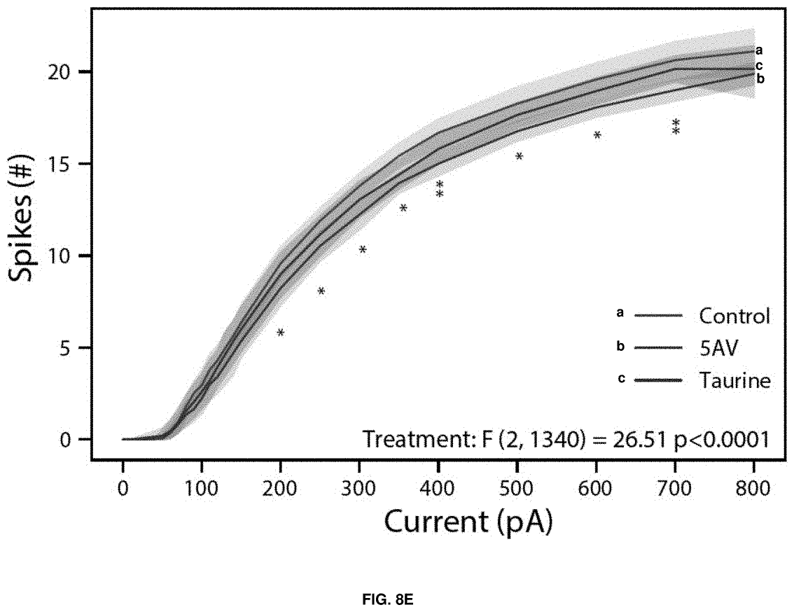

BTBR mice were orally administered 10 mM Taurine or 5AV in drinking water starting 3-4 weeks of age mating, and throughout their lifetime, and effects on the brain were observed. FIG. 8A illustrates effects on amplitude and frequency of mEPSCs in pyramidal neurons in the L5 of the mPFC in acute slices from BTBR mice treated with 5AV, Taurine, or control. FIG. 8B illustrates effects on amplitude and frequency of mIPSCs in pyramidal neurons in the L5 of the mPFC in acute slices from BTBR mice treated with 5AV, Taurine, or control. Hypotheses for FIGS. 8A-B were tested by ANOVA on trimmed means (10%) and subsequent post-hoc tests. FIG. 8C illustrates effects on excitability of pyramidal neurons in the L5 of the mPFC in acute slices from BTBR mice treated with 5AV, Taurine, or control in response to step-wise injection current, as measured by the number of action potential spikes. 2-way ANOVA.

While 5AV induced a decrease in mEPSC amplitude, it was not statistically significant (FIG. 8A). Statistically significant effects of 5AV and/or taurine on neuron activity were not otherwise observed in the assays that were performed (FIGS. 8B-E).

### REFERENCES

The following references are expressly incorporated herein by reference in their entireties for all purposes.
Abrahams, B. S., Arking, D. E., Campbell, D. B., Mefford, H. C., Morrow, E. M., Weiss, L. A., Menashe, I., Wadkins, T., Banerjee-Basu, S., and Packer, A. (2013). SFARI Gene 2.0: a community-driven knowledgebase for the autism spectrum disorders (ASDs). Mol. Autism 4, 36.
Adams, J. B., Audhya, T., McDonough-Means, S., Rubin, R. A., Quig, D., Geis, E., Gehn, E., Loresto, M., Mitchell, J., Atwood, S., et al. (2011). Nutritional and metabolic status of children with autism vs. neurotypical children, and the association with autism severity. Nutr. Metab. 8, 34.
Aldred, S., Moore, K. M., Fitzgerald, M., and Waring, R. H. (2003). Plasma amino acid levels in children with autism and their families. J. Autism Dev. Disord. 33, 93-97.
Amir, A., McDonald, D., Navas-Molina, J. A., Kopylova, E., Morton, J. T., Zech Xu, Z., Kightley, E. P., Thompson, L. R., Hyde, E R., Gonzalez, A., et al. (2017). Deblur Rapidly Resolves Single-Nucleotide Community Sequence Patterns. mSystems 2.
Barak, B., and Feng, G. (2016). Neurobiology of social behavior abnormalities in autism and Williams syndrome. Nat. Neurosci. 19, 647-655.
Barry, G., Briggs, J. A., Vanichkina, D. P., Poth, E. M., Beveridge, N. J., Ratnu, V. S., Nayler, S. P., Nones, K., Hu, J., Bredy, T. W., et al. (2014). The long non-coding RNA Gomafu is acutely regulated in response to neuronal activation and involved in schizophrenia-associated alternative splicing. Mol. Psychiatry 19, 486-494.
Bates, D., Machler, M., Bolker, B., and Walker, S. (2015). Fitting Linear Mixed-Effects Models Using 1me4. Journal of Statistical Software, Articles 67, 1-48.
Bisogno, T., Howell, F., Williams, G., Minassi, A., Cascio, M. G., Ligresti, A., Matias, I., Schiano-Moriello, A., Paul, P., Williams, E.-J., et al. (2003). Cloning of the first sn1-DAG lipases points to the spatial and temporal regulation of endocannabinoid signaling in the brain. J. Cell Biol. 163, 463-468.
Blanchette, M., and Daneman, R. (2015). Formation and maintenance of the BBB. Mech. Dev. 138 Pt 1, 8-16.
Bravo, J. A., Forsythe, P., Chew, M. V., Escaravage, E., Savignac, H. M., Dinan, T. G., Bienenstock, J., and Cryan, J. F. (2011). Ingestion of Lactobacillus strain regulates emotional behavior and central GABA receptor expression in a mouse via the vagus nerve. Proc. Natl. Acad. Sci. U.S.A 108, 16050-16055.
Buffington, S. A., Di Prisco, G. V., Auchtung, T. A., Ajami, N. J., Petrosino, J. F., and Costa-Mattioli, M. (2016). Microbial Reconstitution Reverses Maternal Diet-Induced Social and Synaptic Deficits in Offspring. Cell 165, 1762-1775.
Callery, P. S., and Geelhaar, L. A. (1985). 1-Piperideine as an in vivo precursor of the gamma-aminobutyric acid homologue 5-aminopentanoic acid. J. Neurochem. 45, 946-948.
Caporaso, J. G., Kuczynski, J., Stombaugh, J., Bittinger, K., Bushman, F. D., Costello, E. K., Fierer, N., Pefia, A. G., Goodrich, J. K., Gordon, J. I., et al. (2010). QIIME allows analysis of high-throughput community sequencing data. Nat. Methods 7, 335-336.
Caporaso, J. G., Lauber, C. L., Walters, W. A., Berg-Lyons, D., Huntley, J., Fierer, N., Owens, S. M., Betley, J., Fraser, L., Bauer, M., et al. (2012). Ultra-high-throughput microbial community analysis on the Illumina HiSeq and MiSeq platforms. ISME J. 6, 1621-1624.
Chaidez, V., Hansen, R. L., and Hertz-Picciotto, I. (2014). Gastrointestinal problems in children with autism, developmental delays or typical development. J. Autism Dev. Disord. 44, 1117-1127.
Chambers, J. M., and Hastie, T. (1992). Statistical Models in S (Wadsworth & Brooks/Cole Advanced Books & Software).
Cho, I., and Blaser, M. J. (2012). The human microbiome: at the interface of health and disease. Nat. Rev. Genet. 13, 260-270.
Christensen, D. L., Baio, J., Van Naarden Braun, K., Bilder, D., Charles, J., Constantino, J. N., Daniels, J., Durkin, M. S., Fitzgerald. R. T., Kurzius-Spencer, M., et al. (2016). Prevalence and Characteristics of Autism Spectrum Disorder Among Children Aged 8 Years—Autism and Developmental Disabilities Monitoring Network, 11 Sites, United States, 2012. MMWR Surveill. Summ. 65, 1-23.
Coe, B. P., Witherspoon, K., Rosenfeld, J. A., van Bon, B. W. M., Vulto-van Silfhout, A T., Bosco, P., Friend, K. L., Baker, C., Buono, S., Vissers, L. E. L. M., et al. (2014). Refining analyses of copy number variation identifies specific genes associated with developmental delay. Nat. Genet. 46, 1063-1071.
Coretti, L., Cristiano, C., Florio, E., Scala, G., Lama, A., Keller, S., Cuomo, M., Russo, R, Pero, R, Paciello, O., et al. (2017). Sex-related alterations of gut microbiota composition in the BTBR mouse model of autism spectrum disorder. Sci. Rep. 7, 45356.
Corominas, R, Yang, X., Lin, G. N., Kang, S., Shen, Y., Ghamsari, L., Broly, M., Rodriguez, M., Tam, S., Trigg, S. A., et al. (2014). Protein interaction network of alternatively spliced isoforms from brain links genetic risk factors for autism. Nat. Commun. 5, 3650.
Dachtler, J., Glasper, J., Cohen, R. N., Ivorra, J. L., Swiffen, D. J., Jackson, A. J., Harte, M. K., Rodgers, R. J., and Clapcote, S. J. (2014). Deletion of a-neurexin 11 results in autism-related behaviors in mice. Transl. Psychiatry 4, e484.
De Angelis, M., Piccolo, M., Vannini, L., Siragusa, S., De Giacomo, A., Serrazzanetti, D. I., Cristofori, F., Guerzoni, M. E., Gobbetti, M., and Francavilla, R. (2013). Fecal microbiota and metabolome of children with autism and pervasive developmental disorder not otherwise specified. PLoS One 8, e76993.
De Angelis, M., Francavilla, R., Piccolo, M., De Giacomo, A., and Gobbetti, M. (2015). Autism spectrum disorders and intestinal microbiota. Gut Microbes 6, 207-213.
De Palma, G., Lynch, M. D. J., Lu, J., Dang, V. T., Deng, Y., Jury, J., Umeh, G., Miranda, P. M., Pigrau Pastor, M., Sidani, S., et al. (2017). Transplantation of fecal microbiota from patients with irritable bowel syndrome alters gut function and behavior in recipient mice. Sci. Transl. Med. 9.
Dodd, D., Spitzer, M. H., Van Treuren, W., Merrill, B. D., Hryckowian, A. J., Higginbottom, S. K., Le, A., Cowan, T. M., Nolan, G. P., Fischbach, M. A., et al. (2017). A gut bacterial pathway metabolizes aromatic amino acids into nine circulating metabolites. Nature 551, 648-652.
Dougherty, J. D., Schmidt, E. F., Nakajima, M., and Heintz, N. (2010). Analytical approaches to RNA profiling data for the identification of genes enriched in specific cells. Nucleic Acids Res. 38, 4218-4230.
El Idrissi, A., Boukarrou, L., Heany, W., Malliaros, G., Sangdee, C., and Neuwirth, L. (2009). Effects of taurine on anxiety-like and locomotor behavior of mice. Adv. Exp. Med. Biol. 643, 207-215.
Erny, D., Hrabě Angelis, A. L., Jaitin, D., Wieghofer, P., Staszewski, O., David, E., Keren-Shaul, H., Mahlakoiv, T., Jakobshagen, K., Buch, T., et al. (2015). Host microbiota constantly control maturation and function of microglia in the CNS. Nat. Neurosci. 18, 965-977.
Evans, C., Dunstan, R. H., Rothkirch, T., Roberts, T. K., Reichelt, K. L., Cosford, R., Deed, G., Ellis, L. B., and Sparkes, D. L. (2008). Altered amino acid excretion in children with autism. Nutr. Neurosci. 11, 9-17.
Fernell, E., Karagiannakis, A., Edman, G., Bjerkenstedt, L., Wiesel, F.-A., and Venizelos, N. (2007). Aberrant amino acid transport in fibroblasts from children with autism. Neurosci. Lett. 418, 82-86.
Finegold, S. M., Molitoris, D., Song, Y., Liu, C., Vaisanen, M.-L., Bolte, E., McTeague, M., Sandier, R., Wexler, H., Marlowe, E. M., et al. (2002). Gastrointestinal microflora studies in late-onset autism. Clin. Infect. Dis. 35, S6-S16.
Fombonne, E. (2009). Epidemiology of pervasive developmental disorders. Pediatr. Res. 65, 591-598.
Gaugler, T., Klei, L., Sanders, S. J., Bodea, C. A., Goldberg, A. P., Lee, A. B., Mahajan, M., Manaa, D., Pawitan, Y., Reichert, J., et al. (2014). Most genetic risk for autism resides with common variation. Nat. Genet. 46, 881-885.
Gomez de Aguero, M., Ganal-Vonarburg, S. C., Fuhrer, T., Rupp, S., Uchimura, Y., Li, H., Steinert, A., Heikenwalder, M., Hapfelmeier, S., Sauer, U., et al. (2016). The maternal microbiota drives early postnatal innate immune development. Science 351, 1296-1302.
Gondalia, S. V., Palombo, E. A., Knowles, S R., Cox, S. B., Meyer, D., and Austin, D. W. (2012). Molecular characterisation of gastrointestinal microbiota of children with autism (with and without gastrointestinal dysfunction) and their neurotypical siblings. Autism Res. 5, 419-427.
Gonzalez, I., Munita, R., Agirre, E., Dittmer, T. A., Gysling, K., Misteli, T., and Luco, R. F. (2015). A lncRNA regulates alternative splicing via establishment of a splicing-specific chromatin signature. Nat. Struct. Mol. Biol. 22, 370-376.
Goodrich, J. K., Davenport, E. R., Clark, A. G., and Ley, R E. (2017). The Relationship Between the Human Genome and Microbiome Comes into View. Annu. Rev. Genet.
Gorrindo, P., Williams, K. C., Lee, E. B., Walker, L. S., McGrew, S. G., and Levitt, P. (2012). Gastrointestinal dysfunction in autism: parental report, clinical evaluation, and associated factors. Autism Res. 5, 101-108.
Gotham, K., Risi, S., Pickles, A., and Lord, C. (2007). The Autism Diagnostic Observation Schedule: revised algorithms for improved diagnostic validity. J. Autism Dev. Disord. 37, 613-627.
Gotham, K., Pickles, A., and Lord, C. (2009). Standardizing ADOS scores for a measure of severity in autism spectrum disorders. J. Autism Dev. Disord. 39, 693-705.
Hallmayer, J., Cleveland, S., Torres, A., Phillips, J., Cohen, B., Torigoe, T., Miller, J., Fedele, A., Collins, J., Smith, K., et al. (2011). Genetic heritability and shared environmental factors among twin pairs with autism. Arch. Gen. Psychiatry 68, 1095-1102.
Hiller, K., Hangebrauk, J., Jager, C., Spura, J., Schreiber, K., and Schomburg, D. (2009). MetaboliteDetector: comprehensive analysis tool for targeted and nontargeted G C/M S based metabolome analysis. Anal. Chem. 81, 3429-3439.
Hoban, A. E., Stilling, R. M., Ryan, F. J., Shanahan, F., Dinan, T. G., Claesson, M. J., Clarke, G., and Cryan, J. F. (2016). Regulation of prefrontal cortex myelination by the microbiota. Transl. Psychiatry 6, e774.
Hsiao, E. Y., McBride, S. W., Hsien, S., Sharon, G., Hyde, E R., McCue, T., Codelli, J. A., Chow, J., Reisman, S. E., Petrosino, J. F., et al. (2013). Microbiota modulate behavioral and physiological abnormalities associated with neurodevelopmental disorders. Cell 155, 1451-1463.
Irimia, M., Weatheritt, R J., Ellis, J. D., Parikshak, N. N., Gonatopoulos-Pournatzis, T., Babor, M., Quesnel-Vallibres, M., Tapial, J., Raj, B., O'Hanlon, D., et al. (2014). A highly conserved program of neuronal microexons is misregulated in autistic brains. Cell 159, 1511-1523.
Kang, D.-W., Park, J. G., Ilhan, Z. E., Wallstrom, G., Labaer, J., Adams, J. B., and Krajmalnik-Brown, R. (2013). Reduced incidence of Prevotella and other fermenters in intestinal microflora of autistic children. PLoS One 8, e68322.
Kang, D.-W., Adams, J. B., Gregory, A. C., Borody, T., Chittick, L., Fasano, A., Khoruts, A., Geis, E., Maldonado, J., McDonough-Means, S., et al. (2017). Microbiota Transfer Therapy alters gut ecosystem and improves gastrointestinal and autism symptoms: an open-label study. Microbiome 5, 10.
King, M., and Bearman, P. (2009). Diagnostic change and the increased prevalence of autism. Int. J. Epidemiol. 38, 1224-1234.
Klein, M. S., Newell, C., Bomhof, M R., Reimer, R A., Hittel, D. S., Rho, J. M., Vogel, H. J., and Shearer, J. (2016). Metabolomic Modeling To Monitor Host Responsiveness to Gut Microbiota Manipulation in the BTBRT+tf/j Mouse. J. Proteome Res. 15, 1143-1150.
Kordeli, E., and Bennett, V. (1991). Distinct ankyrin isoforms at neuron cell bodies and nodes of Ranvier resolved using erythrocyte ankyrin-deficient mice. J. Cell Biol. 114, 1243-1259.
Krajmalnik-Brown, R., Lozupone, C., Kang, D.-W., and Adams, J. B. (2015). Gut bacteria in children with autism spectrum disorders: challenges and promise of studying how a complex community influences a complex disease. Microb. Ecol. Health Dis. 26, 26914.
Kushak, R. I., Winter, H. S., Buie, T. M., Cox, S. B., Phillips, C. D., and Ward, N. L. (2016). Analysis of the Duodenal Microbiome in Autistic Individuals: Association with Carbohydrate Digestion. J. Pediatr. Gastroenterol. Nutr.
Langen, M., Bos, D., Noordermeer, S. D. S., Nederveen, H., van Engeland, H., and Durston, S. (2014). Changes in the development of striatum are involved in repetitive behavior in autism. Biol. Psychiatry 76, 405-411.
Langille, M. G. I., Zaneveld, J., Caporaso, J. G., McDonald, D., Knights, D., Reyes, J. A., Clemente, J. C., Burkepile, D. E., Vega Thurber, R. L., Knight, R, et al. (2013).
Predictive functional profiling of microbial communities using 16S rRNA marker gene sequences. Nat. Biotechnol. 31, 814-821.
Law, C. W., Chen, Y., Shi, W., and Smyth, G. K. (2014). voom: Precision weights unlock linear model analysis tools for RNA-seq read counts. Genome Biol. 15, R29.
Lee, A. S., De Jesús-Cortés, H., Kabir, Z. D., Knobbe, W., Orr, M., Burgdorf, C., Huntington, P., McDaniel, L., Britt, J. K., Hoffmann, F., et al. (2016). The Neuropsychiatric Disease-Associated Gene cacnac Mediates Survival of Young Hippocampal Neurons. eNeuro 3.
Liaw, A., Wiener, M., and Others (2002). Classification and regression by randomForest. R News 2, 18-22.
Lin, N., Chang, K.-Y., Li, Z., Gates, K., Rana, Z. A., Dang, J., Zhang, D., Han, T., Yang, C.-S., Cunningham, T. J., et al. (2014). An evolutionarily conserved long noncoding RNA TUNA controls pluripotency and neural lineage commitment. Mol. Cell 53, 1005-1019.
Love, M. I., Huber, W., and Anders, S. (2014). Moderated estimation of fold change and dispersion for RNA-seq data with DESeq2. Genome Biol. 15, 550.
Lozupone, C., and Knight, R. (2005). UniFrac: a new phylogenetic method for comparing microbial communities. Appl. Environ. Microbiol. 71, 8228-8235.
Luca, F., Kupfer, S. S., Knights, D., Khoruts, A., and Blekhman, R. (2017). Functional Genomics of Host-Microbiome Interactions in Humans. Trends Genet.
Lyall, K., Schmidt, R J., and Hertz-Picciotto, I. (2014). Maternal lifestyle and environmental risk factors for autism spectrum disorders. Int. J. Epidemiol. 43, 443-464.
Matthies, A., Clavel, T., Gutschow, M., Engst, W., Haller, D., Blaut, M., and Braune, A. (2008). Conversion of daidzein and genistein by an anaerobic bacterium newly isolated from the mouse intestine. Appl. Environ. Microbiol. 74, 4847-4852.
Matthies, A., Blaut, M., and Braune, A. (2009). Isolation of a human intestinal bacterium capable of daidzein and genistein conversion. Appl. Environ. Microbiol. 75, 1740-1744.
Matzke, M. M., Waters, K. M., Metz, T. O., Jacobs, J. M., Sims, A. C., Baric, R. S., Pounds, J. G., and Webb-Robertson, B.-J. M. (2011). Improved quality control processing of peptide-centric L C-M S proteomics data. Bioinformatics 27, 2866-2872.
Mazina, V., Gerdts, J., Trinh, S., Ankenman, K., Ward, T., Dennis, M. Y., Girirajan, S., Eichler, E. E., and Bernier, R (2015). Epigenetics of autism-related impairment: copy number variation and maternal infection. J. Dev. Behav. Pediatr. 36, 61-67.
McDonald, D., Price, M. N., Goodrich, J., Nawrocki, E. P., DeSantis, T. Z., Probst, A., Andersen, G. L., Knight, R, and Hugenholtz, P. (2012). An improved Greengenes taxonomy with explicit ranks for ecological and evolutionary analyses of bacteria and archaea. ISME J. 6, 610-618.
McElhanon, B. O., McCracken, C., Karpen, S., and Sharp, W. G. (2014). Gastrointestinal symptoms in autism spectrum disorder: a meta-analysis. Pediatrics 133, 872-883.
McFarlane, H. G., Kusek, G. K., Yang, M., Phoenix, J. L., Bolivar, V. J., and Crawley, J. N. (2008). Autism-like behavioral phenotypes in BTBR T+tf/J mice. Genes Brain Behav. 7, 152-163.
McMurdie, P. J., and Holmes, S. (2013). phyloseq: an R package for reproducible interactive analysis and graphics of microbiome census data. PLoS One 8, e61217.
Melnik, A. V., da Silva, R. R., Hyde, E R., Aksenov, A. A., Vargas, F., Bouslimani, A., Protsyuk, I., Jarmusch, A. K., Tripathi, A., Alexandrov, T., et al. (2017). Coupling Targeted and Untargeted Mass Spectrometry for Metabolome-Microbiome-Wide Association Studies of Human Fecal Samples. Anal. Chem. 89, 7549-7559.
Ming, X., Stein, T. P., Barnes, V., Rhodes, N., and Guo, L. (2012). Metabolic perturbance in autism spectrum disorders: a metabolomics study. J. Proteome Res. 11, 5856-5862.
Mussap, M., Noto, A., and Fanos, V. (2016). Metabolomics of autism spectrum disorders: early insights regarding mammalian-microbial cometabolites. Expert Rev. Mol. Diagn. 16, 869-881.
Nankova, B. B., Agarwal, R., MacFabe, D. F., and La Gamma, E. F. (2014). Enteric bacterial metabolites propionic and butyric acid modulate gene expression, including CREB-dependent catecholaminergic neurotransmission, in PC12 cells—possible relevance to autism spectrum disorders. PLoS One 9, e103740.
Naushad, S. M., Jain, J. M. N., Prasad, C. K., Naik, U., and Akella, R. R. D. (2013). Autistic children exhibit distinct plasma amino acid profile. Indian J. Biochem. Biophys. 50, 474-478.
Neufeld, K. M., Kang, N., Bienenstock, J., and Foster, J. A. (2011). Reduced anxiety-like behavior and central neurochemical change in germ-free mice. Neurogastroenterol. Motil. 23, 255-264, el 19.
Noecker, C., Eng, A., Srinivasan, S., Theriot, C. M., Young, V. B., Jansson, J. K., Fredricks, D. N., and Borenstein, E. (2016). Metabolic Model-Based Integration of Microbiome Taxonomic and Metabolomic Profiles Elucidates Mechanistic Links between Ecological and Metabolic Variation. mSystems 1.
Onore, C., Careaga, M., and Ashwood, P. (2012). The role of immune dysfunction in the pathophysiology of autism. Brain Behav. Immun. 26, 383-392.
Parikshak, N. N., Swarup, V., Belgard, T. G., Irimia, M., Ramaswami, G., Gandal, M. J., Hartl, C., Leppa, V., Ubieta, L. de la T., Huang, J., et al. (2016). Genome-wide changes in lncRNA, splicing, and regional gene expression patterns in autism. Nature 540, 423-427.
Park, E., Cohen, I., Gonzalez, M., Castellano, M. R., Flory, M., Jenkins, E. C., Brown, W. T., and Schuller-Levis, G. (2017). Is Taurine a Biomarker in Autistic Spectrum Disorder? Adv. Exp. Med. Biol. 975, 3-16.
Ponti, G., Rodriguez-Gomez, A., Farinetti, A., Marraudino, M., Filice, F., Foglio, B., Sciacca, G., Panzica, G. C., and Gotti, S. (2017). Early postnatal genistein administration permanently affects nitrergic and vasopressinergic systems in a sex-specific way. Neuroscience 346, 203-215.
Powell, D. R., Gay, J. P., Wilganowski, N., Doree, D., Savelieva, K. V., Lanthorn, T. H., Read, R., Vogel, P., Hansen, G. M., Brommage, R., et al. (2015). Diacylglycerol Lipase a Knockout Mice Demonstrate Metabolic and Behavioral Phenotypes Similar to Those of Cannabinoid Receptor 1 Knockout Mice. Front. Endocrinol. 6, 86.
Price, M. N., Dehal, P. S., and Arkin, A. P. (2009). FastTree: computing large minimum evolution trees with profiles instead of a distance matrix. Mol. Biol. Evol. 26, 1641-1650.
Reusens, B., Dahri, S., Snoeck, A., Bennis-Taleb, N., Remacle, C., and Hoet, J. J. (1995). Long-term consequences of diabetes and its complications may have a fetal origin: experimental and epidemiological evidence.
Rodriguez-Gomez, A., Filice, F., Gotti, S., and Panzica, G. (2014). Perinatal exposure to genistein affects the normal development of anxiety and aggressive behaviors and nitric oxide system in CD1 male mice. Physiol. Behav. 133, 107-114.
Rossignol, D. A., and Frye, R. E. (2012). Mitochondrial dysfunction in autism spectrum disorders: a systematic review and meta-analysis. Mol. Psychiatry 17, 290-314.
Rothwell, P. E., Fuccillo, M. V., Maxeiner, S., Hayton, S. J., Gokce, O., Lim, B. K., Fowler, S. C., Malenka, R C., and Sadhof, T. C. (2014). Autism-associated neuroligin-3 mutations commonly impair striatal circuits to boost repetitive behaviors. Cell 158, 198-212.
Roysommuti, S., and Michael Wyss, J. (2013). Perinatal Taurine Exposure on Infants. In Nutrition in Infancy, (Humana Press, Totowa, N.J.), pp. 393-408.
Sandin, S., Lichtenstein, P., Kuja-Halkola, R., Hultman, C., Larsson, H., and Reichenberg, A. (2017). The Heritability of Autism Spectrum Disorder. JAMA 318, 1182-1184.
Sandier, R. H., Finegold, S. M., Bolte, E. R., Buchanan, C. P., Maxwell, A. P., Vdisdnen, M. L., Nelson, M. N., and Wexler, H. M. (2000). Short-term benefit from oral vancomycin treatment of regressive-onset autism. J. Child Neurol. 15, 429-435.
Sandoval-Motta, S., Aldana, M., Martinez-Romero, E., and Frank, A. (2017). The Human Microbiome and the Missing Heritability Problem. Front. Genet. 8, 80.
Schaafsma, S. M., Gagnidze, K., Reyes, A., Norstedt, N., MAnsson, K., Francis, K., and Pfaff, D. W. (2017). Sex-specific gene-environment interactions underlying ASD-like behaviors. Proc. Natd. Acad. Sci. U.S.A 114, 1383-1388.
Schneider, C. K., Melmed, R. D., Barstow, L. E., Enriquez, F. J., Ranger-Moore, J., and Ostrem, J. A. (2006). Oral human immunoglobulin for children with autism and gastrointestinal dysfunction: a prospective, open-label study. J. Autism Dev. Disord. 36, 1053-1064.
Seedorf, H., Griffin, N. W., Ridaura, V. K., Reyes, A., Cheng, J., Rey, F. E., Smith, M. I., Simon, G. M., Scheffrahn, R. H., Woebken, D., et al. (2014). Bacteria from diverse habitats colonize and compete in the mouse gut. Cell 159, 253-266.
Selimbeyoglu, A., Kim, C. K., Inoue, M., Lee, S. Y., Hong, A. S. O., Kauvar, I., Ramakrishnan, C., Fenno, L. E., Davidson, T. J., Wright, M., et al. (2017). Modulation of prefrontal cortex excitation/inhibition balance rescues social behavior in CNTNAP2-deficient mice. Sci. Transl. Med. 9.
Setchell, K. D., and Cassidy, A. (1999). Dietary isoflavones: biological effects and relevance to human health. J. Nutr. 129, 758S-767S.
Sharon, G., Garg, N., Debelius, J., Knight, R., Dorrestein, P. C., and Mazmanian, S. K. (2014). Specialized metabolites from the microbiome in health and disease. Cell Metab. 20, 719-730.
Sharon, G., Sampson, T. R, Geschwind, D. H., and Mazmanian, S. K. (2016). The Central Nervous System and the Gut Microbiome. Cell 167, 915-932.
Shen, S., Park, J. W., Lu, Z.-X., Lin, L., Henry, M. D., Wu, Y. N., Zhou, Q., and Xing, Y. (2014). rMATS: robust and flexible detection of differential alternative splicing from replicate RNA-Seq data. Proc. Natl. Acad. Sci. U.S.A 111, E5593-E5601.
Sivendran, S., Patterson, D., Spiegel, E., McGown, I., Cowley, D., and Colman, R. F. (2004). Two novel mutant human adenylosuccinate lyases (ASLs) associated with autism and characterization of the equivalent mutant Bacillus subtilis ASL. J. Biol. Chem. 279, 53789-53797.
Snijders, A. M., Langley, S. A., Kim, Y.-M., Brislawn, C. J., Noecker, C., Zink, E. M., Fansler, S. J., Casey, C. P., Miller, D. R., Huang, Y., et al. (2016). Influence of early life exposure, host genetics and diet on the mouse gut microbiome and metabolome. Nat Microbiol 2, 16221.
Son, J. S., Zheng, L. J., Rowehl, L. M., Tian, X., Zhang, Y., Zhu, W., Litcher-Kelly, L., Gadow, K. D., Gathungu, G., Robertson, C. E., et al. (2015). Comparison of Fecal Microbiota in Children with Autism Spectrum Disorders and Neurotypical Siblings in the Simons Simplex Collection. PLoS One 10, e0137725.
Sonnenburg, E. D., Smits, S. A., Tikhonov, M., Higginbottom, S. K., Wingreen, N. S., and Sonnenburg, J. L. (2016). Diet-induced extinctions in the gut microbiota compound over generations. Nature 529, 212-215.
Stessman, H. A. F., Willemsen, M. H., Fenckova, M., Penn, O., Hoischen, A., Xiong, B., Wang, T., Hoekzema, K., Vives, L., Vogel, I., et al. (2016). Disruption of POGZ Is Associated with Intellectual Disability and Autism Spectrum Disorders. Am. J. Hum. Genet. 98, 541-552.
Stilling, R. M., Ryan, F. J., Hoban, A. E., Shanahan, F., Clarke, G., Claesson, M. J., Dinan, T. G., and Cryan, J. F. (2015). Microbes & neurodevelopment—Absence of microbiota during early life increases activity-related transcriptional pathways in the amygdala. Brain Behav. Immun. 50, 209-220.
Strati, F., Cavalieri, D., Albanese, D., De Felice, C., Donati, C., Hayek, J., Jousson, O., Leoncini, S., Renzi, D., Calabro, A., et al. (2017). New evidences on the altered gut microbiota in autism spectrum disorders. Microbiome 5, 24.
Strimmer, K. (2008). fdrtool: a versatile R package for estimating local and tail area-based false discovery rates. Bioinformatics 24, 1461-1462.
de Theije, C. G. M., Wopereis, H., Ramadan, M., van Eijndthoven, T., Lambert, J., Knol, J., Garssen, J., Kraneveld, A. D., and Oozeer, R. (2014). Altered gut microbiota and activity in a murine model of autism spectrum disorders. Brain Behav. Immun. 37, 197-206.
Tochitani, S. (2017a). Functions of Maternally-Derived Taurine in Fetal and Neonatal Brain Development. In Taurine 10, (Springer, Dordrecht), pp. 17-25.
Tochitani, S. (2017b). Functions of Maternally-Derived Taurine in Fetal and Neonatal Brain Development. Adv. Exp. Med. Biol. 975, 17-25.
Tsuiji, H., Yoshimoto, R., Hasegawa, Y., Furuno, M., Yoshida, M., and Nakagawa, S. (2011). Competition between a noncoding exon and introns: Gomafu contains tandem UACUAAC repeats and associates with splicing factor-1. Genes Cells 16, 479-490.
Tu, W.-J., Chen, H., and He, J. (2012). Application of L C-M S/M S analysis of plasma amino acids profiles in children with autism. J. Clin. Biochem. Nutr. 51, 248-249.
Turner, T. N., Coe, B. P., Dickel, D. E., Hoekzema, K., Nelson, B. J., Zody, M. C., Kronenberg, Z. N., Hormozdiari, F., Raja, A., Pennacchio, L. A., et al. (2017). Genomic Patterns of De Novo Mutation in Simplex Autism. Cell 171, 710-722.e12.
Ursell, L. K., Haiser, H. J., Van Treuren, W., Garg, N., Reddivari, L., Vanamala, J., Dorrestein, P. C., Turnbaugh, P. J., and Knight, R. (2014). The intestinal metabolome: an intersection between microbiota and host. Gastroenterology 146, 1470-1476.
Voineagu, I., Wang, X., Johnston, P., Lowe, J. K., Tian, Y., Horvath, S., Mill, J., Cantor, R. M., Blencowe, B. J., and Geschwind, D. H. (2011). Transcriptomic analysis of autistic brain reveals convergent molecular pathology. Nature 474, 380-384.
Webb-Robertson, B.-J. M., McCue, L. A., Waters, K. M., Matzke, M. M., Jacobs, J. M., Metz, T. O., Varnum, S. M., and Pounds, J. G. (2010). Combined statistical analyses of peptide intensities and peptide occurrences improves identification of significant peptides from M S-based proteomics data. J. Proteome Res. 9, 5748-5756.
West, P. R., Amaral, D. G., Bais, P., Smith, A. M., Egnash, L. A., Ross, M. E., Palmer, J. A., Fontaine, B. R, Conard, K. R., Corbett, B. A., et al. (2014). Metabolomics as a Tool for Discovery of Biomarkers of Autism Spectrum Disorder in the Blood Plasma of Children. PLoS One 9, el 12445.
Wikoff, W. R., Anfora, A T., Liu, J., Schultz, P. G., Lesley, S. A., Peters, E. C., and Siuzdak, G. (2009). Metabolomics analysis reveals large effects of gut microflora on mammalian blood metabolites. Proc. Natl. Acad. Sci. U.S.A 106, 3698-3703.
Williams, B. L., Hornig, M., Buie, T., Bauman, M. L., Cho Paik, M., Wick, I., Bennett, A., Jabado, O., Hirschberg, D. L., and Lipkin, W. I. (2011). Impaired carbohydrate digestion and transport and mucosal dysbiosis in the intestines of children with autism and gastrointestinal disturbances. PLoS One 6, e24585.
Williams, B. L., Hornig, M., Parekh, T., and Lipkin, W. I. (2012). Application of novel PCR-based methods for detection, quantitation, and phylogenetic characterization of Sutterella species in intestinal biopsy samples from children with autism and gastrointestinal disturbances. MBio 3.
Willsey, A. J., Sanders, S. J., Li, M., Dong, S., Tebbenkamp, A. T., Muhle, R. A., Reilly, S. K., Lin, L., Fertuzinhos, S., Miller, J. A., et al. (2013). Coexpression networks implicate human midfetal deep cortical projection neurons in the pathogenesis of autism. Cell 155, 997-1007.
Yamada, K. D., Tomii, K., and Katoh, K. (2016). Application of the MAFFT sequence alignment program to large data-reexamination of the usefulness of chained guide trees. Bioinformatics 32, 3246-3251.
Yap, I. K. S., Angley, M., Veselkov, K. A., Holmes, E., Lindon, J. C., and Nicholson, J. K. (2010). Urinary metabolic phenotyping differentiates children with autism from their unaffected siblings and age-matched controls. J. Proteome Res. 9, 2996-3004.
Yu, H., Guo, Z., Shen, S., and Shan, W. (2016). Effects of taurine on gut microbiota and metabolism in mice. Amino Acids 48, 1601-1617.
Zhang, Y., Sloan, S. A., Clarke, L. E., Caneda, C., Plaza, C. A., Blumenthal, P. D., Vogel, H., Steinberg, G. K., Edwards, M. S. B., Li, G., et al. (2016). Purification and Characterization of Progenitor and Mature Human Astrocytes Reveals Transcriptional and Functional Differences with Mouse. Neuron 89, 37-53.
Zheng, P., Zeng, B., Zhou, C., Liu, M., Fang, Z., Xu, X., Zeng, L., Chen, J., Fan, S., Du, X., et al. (2016). Gut microbiome remodeling induces depressive-like behaviors through a pathway mediated by the host's metabolism. Mol. Psychiatry 21, 786-796.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods, compositions, kits, and uses described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Wherever a method of using a composition (e.g., a composition comprising, consisting essentially of, or consisting of a bacteria, metabolite, metabolite precursor) is disclosed herein, the corresponding composition for use is also expressly contemplated. For example, for the disclosure of a method of reducing or preventing a symptom of ASD in a selected prenatal subject, comprising administering an amount of taurine and/or 5AV to the prenatal subject, the corresponding taurine and/or 5AV for use in reducing or preventing a symptom of ASD (via prenatal administration) is also contemplated.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

APPENDIX: TABLE 3-1

| Metabolit [symbol] | Sample ID | Diagnosis | 1,3-Dihyd [symbol] | 3-Hydrox [symbol] | 3-Phenylp [symbol] | 4-Aminob [symbol] | 4-Hydrox [symbol] | 4-Hydrox [symbol] |
|---|---|---|---|---|---|---|---|---|
| B71 | N5 | NT | −2.07795 | −2.3734 | −1.24787 | −0.06465 | NA | −2.51091 |
| B72 | C4 | NT | −3.16912 | −1.90609 | −2.58416 | −0.9992 | −0.56031 | −1.24312 |
| B73 | A17 | ASD | −2.85798 | −2.98351 | −1.58496 | −0.25312 | −0.5603 | −1.01668 |
| B74 | A09 | ASD | −3.5163 | −2.2268 | −2.3643 | −0.2268 | NA | 0.7484 |
| B75 | C1 | NT | −3.81589 | −1.87729 | −2.70041 | −2.94142 | −0.97459 | 1.14604 |
| B76 | A24 | ASD | −3.0565 | −2.77639 | −3.16341 | −1.54193 | NA | −2.00587 |
| B77 | A03 | ASD | −3.85022 | −2.85022 | −2.95714 | −2.01372 | NA | −0.70338 |
| B78 | MS | NT | −2.69986 | −2.14043 | −3.24735 | −0.66239 | NA | −0.69986 |
| B79 | A11 | ASD | −3.24101 | −3.37851 | −1.74624 | −1.49099 | NA | 0 |
| B80 | N5 | NT | −2.57997 | −3.28041 | −2.57997 | −2.57997 | −2.52552 | −2.88809 |
| B81 | C4 | NT | −3.31765 | −2.21073 | −2.43312 | −1.40338 | −1.66053 | −1.1112 |
| B82 | A17 | ASD | −2.9582 | −2.0957 | −1.9582 | 0.025313 | −1.51074 | −2.1697 |
| B83 | A09 | ASD | −3.848 | −2.36257 | −2.848 | −2.04064 | NA | 0.591626 |
| B84 | C1 | NT | −4.19265 | −3.09311 | −3.29956 | −2.75207 | −2.35614 | 0.247928 |
| B85 | A24 | ASD | −2.74521 | −2.02531 | −3.13223 | −2.37324 | NA | −1.16024 |
| B86 | A03 | ASD | −3 | −2.44057 | −3.54749 | −1.54749 | NA | −2.078 |
| B87 | N5 | NT | −2.93773 | −2.03726 | −3.14418 | −0.69487 | NA | −1.38519 |
| B88 | A11 | ASD | −2.6845 | −2.37638 | −0.98935 | −2.75489 | NA | NA |
| B89 | N5 | NT | −2.49525 | −1.96474 | −0.75829 | −1.14162 | NA | −0.8072 |
| B90 | C4 | NT | −4.10474 | −3.32713 | −2.35751 | −1.8677 | NA | −1.22559 |
| B91 | A17 | ASD | −2.41724 | −2.51034 | −2.24731 | −2.83227 | NA | −1.55925 |
| B92 | A09 | ASD | −2.08603 | −3.4318 | −3.55733 | −2.26188 | NA | −0.74998 |
| B93 | C1 | NT | −3.83918 | −3.68717 | −2.54967 | −2.0549 | −0.87982 | −0.7237 |
| B94 | A24 | ASD | −2.86129 | −3.44626 | −2.86129 | −1.6389 | NA | −2.0133 |
| B95 | A03 | ASD | −2.33087 | −3.6528 | −2.5153 | NA | NA | −2.21984 |
| B96 | N5 | NT | −3.87146 | −4.00896 | −2.87146 | −1.68703 | NA | −1.576 |
| B97 | A11 | ASD | −4.01748 | NA | −1.87998 | −1.81585 | NA | −1.16949 |
| B98 | N5 | NT | −3.37678 | −3.73935 | −1.59251 | −1.63982 | NA | −1.37678 |
| B99 | C4 | NT | −2.44846 | −3.44846 | −2.17544 | −1.54157 | NA | −1.94596 |
| B100 | A17 | ASD | −3.04828 | NA | −1.70035 | −2.70035 | −1.18578 | −1.46331 |
| B101 | A09 | ASD | −2.61783 | −2.61783 | −1.68821 | NA | −3.20279 | −0.11532 |
| B102 | C1 | NT | −3.73039 | NA | −2.66626 | NA | NA | −0.09179 |
| B103 | A24 | ASD | −2.69479 | −3.411 | −3.51792 | −0.91458 | NA | −1.97043 |
| B104 | A03 | ASD | −2.68307 | −2.68307 | −2.73554 | −3.3205 | NA | −1.42434 |
| B105 | A11 | ASD | −3.16993 | −2.38143 | −2.92892 | −1.34395 | NA | −1.01465 |
| B995 | GF | GF | −6.82718 | −3.01982 | −2.40091 | NA | NA | −1.76109 |
| B998 | GF | GF | −6.06609 | −0.49623 | −1.60666 | NA | NA | −1 |
| B1001 | GF | GF | −5.98271 | −1.98271 | −1.93832 | NA | NA | −1.31029 |

| 5-Aminop [symbol] | Acetate | Alanine | Arabinose | Arginine | Asparagin [symbol] | Aspartate | Betaine | Butyrate |
|---|---|---|---|---|---|---|---|---|
| −1.51091 | 5.943187 | 1.881411 | 0.737021 | −0.70355 | NA | 2.459487 | NA | 1.17279 |
| 2.763977 | 7.044953 | 1.857677 | −0.04811 | −0.36177 | −0.46868 | 2.564231 | NA | 2.569645 |
| −0.46566 | 7.701635 | 2.9558 | 2.511253 | −0.31366 | −0.95109 | 2.905785 | NA | 3.427421 |
| −1.07152 | 6.874343 | 2.33058 | −0.47678 | −0.55695 | −0.82825 | 2.14032 | NA | 2.610688 |
| −1.0433 | 5.844104 | 2.759017 | −0.00854 | −0.27157 | −1.0433 | 2.689258 | NA | 1.553342 |
| −1.5063 | 6.143874 | 2.739772 | −0.47154 | 0.667528 | 0.743476 | 2.400589 | −3.69393 | 1.090342 |
| −0.48765 | 6.455028 | 2.097313 | −0.50783 | −0.61318 | −0.50783 | 2.325419 | −3.85022 | 1.136841 |
| 1.365095 | 7.305321 | 1.817084 | −0.55547 | −0.1796 | −0.1023 | 1.881935 | −4.14043 | 2.46584 |
| −0.53051 | 6.024713 | 1.954196 | −0.05658 | 0.547488 | −1.11548 | 2.489385 | NA | 1.013806 |
| −1.11048 | 5.766037 | 1.602238 | 0.28945 | −0.03248 | −1.49905 | 2.266489 | −4.69544 | 0.962769 |
| 1.57437 | 5.630046 | 2.12402 | 0.57437 | 0.229841 | −0.19791 | 3.559342 | −7.01809 | 1.536502 |
| −0.86304 | 6.116429 | 2.839757 | 3.247705 | 0.337257 | −1.24771 | 2.989637 | −6.41763 | 2.91776 |
| −1 | 6.286686 | 2.4411 | 0 | 0.078003 | −1.46949 | 2.371172 | −6.16993 | 2.005001 |
| −0.27208 | 5.905575 | 2.348728 | −0.35614 | 0.247928 | −0.28575 | 2.162391 | NA | 1.055282 |
| −1.24771 | 5.871452 | 2.046915 | −0.4064 | 0.41526 | −0.60385 | 2.576723 | −4.24771 | 1.405737 |
| −1.1394 | 5.37664 | 2.70044 | 0.298967 | 0 | −0.69334 | 2.247928 | −4.24793 | 0.990477 |
| −0.93773 | 5.595214 | 2.431508 | 0.177751 | 0.709972 | 0.188806 | 2.37697 | −5.25965 | 1.813595 |
| −0.82889 | 4.957983 | 1.970308 | 0.39079 | 0 | −0.58496 | 2.894728 | −6.07682 | 0.523097 |
| 0.629442 | 5.76658 | 1.27146 | 1.411637 | −0.25579 | 0.422284 | 2.424934 | −4.66518 | 1.692374 |
| 2.907287 | 6.125882 | 2.233838 | 0.65396 | −0.66417 | 0.717263 | 1.958271 | −5.59017 | 1.068046 |
| −0.4865 | 5.97535 | 2.844859 | 3.235609 | −0.4865 | −1.77338 | 2.784888 | −3.83227 | 2.724872 |
| 0.038517 | 5.901353 | 2.822439 | 1.971919 | −0.74998 | −0.9944 | 3.117019 | −4.84684 | 2.818496 |
| 0.514462 | 6.100731 | 2.280919 | −1.25421 | 0.238827 | −0.21468 | 2.442111 | −1.76117 | 1.417165 |
| −0.4644 | 4.863902 | 2.355937 | 1.10218 | 0.479743 | −0.12433 | 3.131173 | −3.30875 | 0.489204 |
| 0.484701 | 5.526111 | 1.324477 | 0.739515 | 0.273197 | −3.06784 | 1.200195 | −3.06784 | 1.180087 |
| 0.383356 | 6.803377 | 1.795815 | 0.917038 | −0.08296 | −2.37669 | 2.120322 | −2.576 | 2.026663 |
| 0.50608 | 6.937877 | 1.753347 | −0.53206 | 1.297215 | −1.29502 | 2.336547 | −4.01748 | 1.518571 |
| 0.298781 | 7.218059 | 2.10075 | −0.71382 | 0.701219 | −1.08728 | 1.887919 | −4.22478 | 1.892083 |
| 2.304199 | 6.996554 | 1.621213 | 0.799467 | 0.521166 | −0.74802 | 3.282859 | NA | 2.163794 |
| −0.24092 | 6.36996 | 2.626719 | 3.276927 | −1.33778 | −2.60082 | 2.71388 | −4.18578 | 2.807442 |
| −1.10325 | 5.563504 | 2.787519 | 1.347409 | 0.544446 | −1.03286 | 2.855591 | −5.01014 | 1.787519 |
| 1.591535 | 4.491414 | 2.30603 | 0.141092 | 0.364764 | −1.23563 | 2.56172 | −4.0199 | 0.261387 |
| −1.31147 | 5.217835 | 2.053107 | 0.44698 | 0.336233 | −0.60365 | 2.427303 | −4.21836 | 1.411001 |
| −1.18665 | 5.361322 | 2.18553 | 1.449152 | 0.064162 | −0.25202 | 1.73116 | −3.031 | 1.167785 |
| −0.72247 | 5.186827 | 2.0466 | 0.628031 | −1.72247 | −1.10579 | 2.274525 | NA | 0.415037 |
| 0.085711 | −0.92029 | 1.65058 | 0.954182 | −0.27259 | 1.838158 | 1.394409 | −3.65725 | −2.33533 |
| 0.425764 | −0.36565 | 0.052852 | 1.098818 | −0.0774 | 2.358077 | 1.522625 | −1.45138 | −1.5115 |
| 0.293413 | −0.41286 | 0.451916 | 1.464371 | −0.368 | 2.279383 | 0.930178 | −3.39775 | −1.85343 |

| Choline | Citrate | Creatine | Creatine [symbol] | Creatinin [symbol] | Desamino [symbol] | Dimethyl [symbol] | DSS-d6 (C [symbol] | Ethanol |
|---|---|---|---|---|---|---|---|---|
| −1.78844 | −0.9021 | −0.97485 | −3.3734 | −4.24787 | −1.02548 | −5.24787 | 5.321667 | 2.794699 |
| −1.79061 | −0.20565 | −2.09873 | −2.6837 | −3.6837 | 0.821832 | −4.16912 | 5.663451 | 3.657425 |
| −0.85798 | −2.19502 | −0.61005 | −2.53605 | −4.85798 | 0.016488 | −4.12102 | 5.711558 | 4.108765 |
| −2.3643 | −2.04237 | −0.08632 | −2.68623 | −5.10127 | 0.028017 | −4.3643 | 5.468273 | 5.273773 |
| −1.70041 | −1.00854 | −0.78614 | −3.23093 | −5.40085 | 0.70767 | −4.07893 | 5.753648 | 4.443067 |
| −1.00587 | −1.69393 | 1.10193 | −1.57845 | −3.27889 | 0.030964 | −4.86385 | 5.290648 | 3.994904 |
| −2.13401 | −1.26526 | −2.57011 | NA | −6.65757 | −1.75068 | −4.33565 | 5.496927 | 4.741169 |
| −1.1149 | −2.0409 | 0.085635 | NA | −5.94779 | 0.527946 | −4.62586 | 5.206714 | 6.027881 |
| −2.30812 | −1.61298 | −2.24101 | NA | −3.89308 | 0.194378 | −5.11548 | 5.454062 | 2.993047 |
| −0.85414 | −3.47305 | 0.050512 | NA | −4.11048 | −1.21432 | −4.69544 | 4.874096 | 2.938763 |
| −1.77016 | −1.8888 | −2.16011 | NA | −3.43312 | −0.93062 | −4.69616 | 5.136415 | 2.713232 |
| −1.41763 | −1.66274 | −1.83267 | NA | −3.0957 | −0.06008 | −4.83267 | 5.736871 | 4.581665 |
| −2.16993 | −2.46949 | −1.58496 | NA | −3.71049 | −0.02018 | −4.58496 | 4.984577 | 5.222661 |
| −1.91254 | −1.83007 | −1.35614 | NA | −5.41504 | −0.20558 | −5 | 5.154502 | 5.505563 |
| −1.30911 | −1.70338 | −1.97469 | NA | −5.83267 | 0.025313 | −4.83267 | 5.321834 | 3.8643 |
| −1.24793 | −2.72437 | −0.25924 | −3.16046 | −3.7885 | −2.03847 | −3.7885 | 4.906574 | 6.311808 |
| −1.52269 | −2.67469 | −1.03726 | NA | −3.84462 | −0.20076 | −4.84462 | 5.309885 | 3.207951 |
| −1.90689 | −1.61738 | 0.122857 | −2.32193 | NA | −0.4769 | −4.49185 | 5.081163 | 3.444785 |
| −1.17333 | −1.75829 | −1.71098 | −2.34325 | −2.96474 | −0.43636 | −3.66518 | 5.4928 | 2.199008 |
| −2.91209 | −2.48583 | −2.02945 | −3.51978 | −3.74217 | −1.46915 | −4.91209 | 4.242408 | 3.352935 |
| −1.20778 | −1.46304 | −2.60988 | 0.042196 | −2.7168 | −1.55925 | −3.83227 | 5.737266 | 3.110241 |
| −1.90824 | −2.08603 | −1.80731 | −3.62445 | −4.84684 | −0.91873 | −3.4932 | 4.137736 | 1.699197 |
| −1.96471 | −2.0549 | −0.56616 | −2.61678 | −3.83918 | 0.180724 | −4.68717 | 5.145402 | 4.742444 |
| −2.12433 | −3.06774 | −2.24462 | −2.37587 | −3.76818 | −0.59826 | −4.44626 | 5.386317 | 2.27894 |
| −1.19337 | −2.88727 | 0.525115 | −3.5153 | −3.06784 | −2.7268 | −3.97473 | 5.179771 | 3.712645 |
| −1.44825 | −2.63045 | −3.16096 | −3.74593 | −4.33089 | −2.08296 | −5.00896 | 4.823613 | 3.433983 |
| −1.16949 | −1.58452 | −0.91315 | −2.16949 | −3.33941 | −1.43252 | −4.01748 | 5.815092 | 5.016492 |
| −0.96175 | −1.59251 | −0.59251 | −2.96175 | −3.54671 | −0.90285 | −3.96175 | 5.607793 | 3.949146 |
| −1.33298 | −2.44846 | −1.67587 | NA | −4.03342 | −0.71149 | −3.71149 | 5.121079 | 4.301409 |
| −1.64973 | −1.25978 | −1.80727 | NA | −3.50771 | −1.04828 | −4.92275 | 5.646794 | 5.132085 |
| −1.45555 | −2.61783 | −1.36629 | NA | −2.76222 | −1.25526 | −4.20279 | 5.144359 | 4.068008 |
| −1.66626 | −1.98037 | −0.23563 | NA | −4.18982 | −1.10236 | −4.60486 | 4.964677 | 5.843598 |
| −1.38547 | −2.63339 | −1.04843 | NA | NA | −0.97043 | −4.89643 | 4.936146 | 3.62006 |
| −1.3006 | −2.44603 | −0.39239 | NA | NA | −5.49043 | −4.49043 | 4.664075 | 4.08464 |
| −2.16993 | −1.4199 | −2.30743 | NA | −5.04439 | −1.01465 | −5.62936 | 5.525145 | 2.955606 |
| −0.31938 | NA | 0.652602 | NA | −2.33533 | −3.92029 | NA | 4.327324 | 8.849771 |
| 1.291463 | NA | −0.72624 | NA | 0.16273 | −0.39366 | NA | 5.088413 | 7.773459 |
| 0.418168 | NA | −0.55645 | NA | −0.02852 | −1.31029 | NA | 5.17179 | 8.94502 |

| Formate | Fructose | Fumarate | Galactose | Glucose | Glutamat [symbol] | Giutamin [symbol] | Glycerol | Glycine | Homocitr [symbol] |
|---|---|---|---|---|---|---|---|---|---|
| −0.23292 | −0.07795 | −1.97485 | 2.03753 | 3.269141 | 3.560556 | 1.233255 | 2.100856 | 1.792874 | −0.38989 |
| −0.9992 | 1.063538 | −2.6837 | 0.048108 | 3.218033 | 3.397692 | 1.964276 | 1.108862 | 1.195449 | 0.108862 |
| 0 | 0.451874 | −2.98351 | −0.17616 | 2.138257 | 4.378033 | 0.644519 | 0 | 2.964324 | −1.19502 |
| −1.16267 | 0.876014 | −1.59877 | NA | 1.894972 | 3.530517 | 2.196415 | −0.04237 | 1.45843 | −0.17843 |
| −0.54287 | 0.169001 | −4.40085 | −0.1914 | 2.180346 | 4.124667 | 1.969833 | 0.243002 | 2.337238 | −0.78614 |
| −1.24914 | 0.570774 | −2.54193 | −0.38812 | 3.605788 | 3.722048 | 2.030964 | 0.595578 | 2.25249 | −0.88657 |
| −1.01372 | −1.65757 | −1.23131 | 0.717465 | 2.592724 | 3.712023 | 1.090618 | 0.45095 | 2.117212 | −0.04286 |
| −0.27536 | 0.470065 | −2.94779 | 0.786922 | 3.675179 | 3.217119 | 0.800405 | −0.30393 | 0.8071 | −0.8817 |
| −0.72316 | 0.928917 | −3.11548 | 0.027481 | 2.607899 | 3.606761 | 1.580331 | −0.45251 | 1.982555 | −0.86755 |
| −0.71055 | −1.19294 | −1.57997 | 1.207435 | 3.078246 | 3.662842 | 0.211448 | 0.507497 | 1.83073 | −0.83746 |
| −0.30384 | 1.664908 | −2.43312 | 0.689272 | 3.510367 | 3.48375 | 1.836782 | 0.111196 | 2.566876 | −1.52623 |
| −0.06008 | 1.961748 | −2.41763 | NA | 2.360447 | 4.634258 | 0.721921 | 1.576723 | 2.696112 | −0.37324 |
| −0.45568 | 1.25214 | −1.96047 | −0.0614 | 2.946419 | 3.217015 | 1.684943 | 0.345775 | 2.176589 | −1.922 |
| −0.87072 | 0.651052 | −3.83007 | −0.28575 | 2 | 3.16365 | 1.098032 | 0.826548 | 1.97728 | −0.93391 |
| −0.95002 | −0.21796 | −4.51074 | 0.874691 | 4.057596 | 3.759789 | 1.265364 | −0.18881 | 1.825544 | 0.109847 |
| −1 | −1.01911 | −0.2145 | 2.78136 | 3.659714 | 3.754888 | 0.850105 | 0.860597 | 2.789619 | −1.1394 |
| −0.29003 | 0.199777 | −2.67469 | 1.78474 | 3.626042 | 3.9579 | 1.389003 | −0.20076 | 1.834863 | 0.393788 |
| −0.60108 | 0.609685 | −3.16993 | 1.328326 | 3.048598 | 3.976432 | 1.127756 | 1.289507 | 2.388751 | −0.55325 |
| 0.217465 | 0.474373 | −2.49525 | 2.205186 | 3.698956 | 4.146196 | 1.539393 | 1.294824 | 1.042181 | 0.949531 |
| −1.02945 | 1.311908 | −3.15721 | 0.115812 | 3.392828 | 2.779649 | −0.04791 | 0.121329 | 2.313113 | −0.59017 |
| −0.0422 | 1.175222 | −2.41724 | 0.364124 | 2.386895 | 4.298726 | 1.525279 | 1.114146 | 2.83543 | 0.548549 |
| −0.42431 | 1.349557 | −3.69484 | 0.634286 | 3.066714 | 4.457194 | 1.000043 | −0.48538 | 2.593337 | −1.9944 |
| −0.07836 | 1.594526 | −4.42414 | 0.078363 | 2.150771 | 3.258857 | 1.579615 | 1.088932 | 2.016039 | −0.74231 |
| −0.44626 | −0.52026 | −3.59826 | 1.376474 | 3.732658 | 3.712606 | 1.096002 | 1.120559 | 2.168454 | −0.52026 |
| −1.21984 | 0.88325 | −1.61718 | 2.503027 | 4.055246 | 3.579858 | 0.889455 | 0.610232 | 1.254088 | −0.58241 |
| −0.52353 | 0.37647 | −2.37669 | 1.671926 | 2.433983 | 3.527092 | 1.128543 | 0.893113 | 1.637778 | 0.128543 |
| 0.318801 | 0.035629 | −2.75445 | NA | 2.451753 | 3.499794 | 1.543233 | 1.144406 | 1.89421 | −0.36213 |
| −0.99212 | −0.29878 | −3.73935 | 1.194758 | 2.722418 | 3.500415 | 1.286181 | 1.395806 | 1.497685 | 0.335934 |
| −0.361 | 0.559034 | −2.17544 | 0.204982 | 2.702979 | 3.951707 | 1.064609 | 1.151452 | 2.332899 | 0.466423 |
| 0.24718 | −0.42024 | −2.50771 | 0.299647 | 3.629284 | 4.255505 | 1.590325 | −0.86385 | 2.682117 | 0.046881 |
| −0.41023 | 0.771217 | −2.68821 | 1.767934 | 3.042425 | 3.937494 | 1.952753 | 1.810036 | 2.630102 | −0.76222 |
| −0.58991 | 0.104796 | −3.28293 | 0 | 2.120788 | 3.367639 | 0.908208 | 0.468387 | 2.25519 | −0.54597 |
| −0.66377 | 0.403696 | −3.89643 | −0.04843 | 2.952821 | 3.815067 | 0.048431 | 1.173962 | 1.273497 | 0.45407 |
| −0.51315 | −0.06416 | −1.20502 | 3.346413 | 3.709246 | 3.553967 | 1.425453 | 0.831501 | 2.124283 | −1.93584 |
| −0.54189 | 0 | −2.30743 | 1.665264 | 3.352211 | 3.613817 | 0.870489 | 0.854459 | 2.165059 | 0.265461 |
| −1.28802 | 4.71416 | NA | NA | 1.47203 | 3.459957 | 2.670674 | NA | 2.2416 | −1.18332 |
| −0.55829 | 5.389495 | NA | NA | 0.834778 | 4.257529 | 2.813494 | NA | 0.393342 | −0.42223 |
| −0.47492 | 4.481834 | NA | NA | 0.924179 | 3.673713 | 2.064412 | 3.192214 | 0.366016 | 0.028515 |

| Isobutyra [symbol] | Isoleucin [symbol] | Isopropan [symbol] | Lactate | Leucine | Lysine | Malonate | Methanol | Methioni [symbol] | Methyla [symbol] |
|---|---|---|---|---|---|---|---|---|---|
| NA | 0.642899 | −2.3734 | −0.01266 | 1.144445 | 0.948525 | 2.074056 | 0.706324 | 0.012656 | −1.44052 |
| NA | 0.236869 | −2.32113 | −0.51377 | 0.80357 | 0.62789 | −0.6837 | 0.439686 | 0.223194 | 0.078804 |
| NA | 2.060882 | −2.63559 | 1.229482 | 2.254024 | 1.675998 | 2.052912 | 1.351472 | 0.776225 | −1.27302 |
| NA | 1.369054 | −2.2268 | 1.36362 | 1.626654 | 1.760855 | −1.47678 | 1.244S09 | 0.422296 | −0.02802 |
| NA | 1.702434 | −2.15293 | 1.54166 | 1.912029 | 1.793903 | 2.303049 | −0.02581 | 1.042089 | −0.84627 |
| NA | 1.805031 | 2.690735 | 1.659708 | 1.917506 | 1.643941 | −2.22 | −0.03096 | 1.285893 | −1.57845 |
| NA | 1.471708 | 1.077135 | 0.627827 | 1.659838 | 1.487083 | −1.10299 | 0.273163 | 0.571244 | −1.85022 |
| NA | −0.0409 | 1.023756 | 0.666922 | 0.575774 | 0.959103 | −2.42423 | 0.365095 | −0.15337 | −1.30393 |
| NA | 0.861803 | NA | 0.310788 | 0.91427 | 1.444219 | 0.157541 | 0.106915 | −0.04223 | −1.94555 |
| NA | 0.28945 | −3.11048 | 0.440694 | 0.041523 | 0.864253 | 0.763989 | 0.068323 | −0.44752 | −0.80467 |
| NA | 0.716623 | −3.31765 | −1.43312 | 0.769816 | 1.126571 | −1.62577 | 1.647249 | 0.161822 | −1.40338 |
| NA | 1.659185 | −2.71719 | 1.226226 | 2.016998 | 1.727028 | 1.204421 | 0.680402 | 0.701311 | −0.35154 |
| NA | 1.481127 | −3.46949 | 0.06848 | 1.888067 | 0.974733 | −2.08246 | 0.178803 | 0.376969 | −0.12553 |
| NA | 1.546894 | −3.29956 | 3.415742 | 1.622052 | 1.144658 | 1.290019 | −0.32757 | 0.577429 | −0.50815 |
| NA | 0.610276 | 1.793041 | 0.200755 | 0.774662 | 1.1897 | −1.97469 | −0.02531 | 0.41526 | −0.95002 |
| NA | 1.804641 | 0.018859 | 0.881356 | 1.966392 | 1.473172 | −1.72437 | 0.366782 | 0.956644 | −0.85561 |
| NA | 1.294935 | 0.547701 | −1.20076 | 1.445402 | 2.29749 | −1.98664 | −0.0502 | 0.936743 | −0.29003 |
| NA | 0.774933 | −1.49185 | −2.07682 | 0.945552 | 1.730539 | −0.46211 | 1.315502 | 0.357813 | −1.09954 |
| NA | −0.39839 | 0.701144 | −0.88382 | −0.11059 | 0.534494 | −0.6879 | 0.494693 | −0.32533 | −0.66518 |
| NA | 0.423296 | −3.15721 | 3.619287 | 0.952092 | 0.159368 | NA | 0.047908 | 0.164722 | 0.262832 |
| −3.24731 | 1.957804 | −1.89367 | 0.536961 | 2.062545 | 1.970782 | −0.53459 | 0.670227 | 0.975082 | −0.18842 |
| −4.4318 | 1.405299 | −3.4932 | 0.331963 | 1.112518 | 1.731428 | −2.01677 | 0.011141 | 0.825585 | 0.132982 |
| NA | 1.32629 | −2.25421 | 3.893275 | 1.627525 | 1.062362 | 0.711999 | −0.66925 | 0.705145 | −0.42414 |
| NA | 0.598138 | −2.12433 | 0.150679 | 1.191818 | 1.1976 | −1.52026 | 0.150679 | 0.329848 | −0.65966 |
| −2.45117 | 0.391591 | 3.670927 | 0.697694 | 0.205178 | 0.889455 | −0.80481 | 0.365119 | −0.1S455 | −0.74591 |
| −3.00896 | −0.00896 | 5.310937 | −0.3422 | 0.383356 | 1.057128 | −1.68703 | 0.761868 | −0.24343 | −2.24343 |
| −2.25195 | 0.860262 | −1.81585 | 0.136323 | 1.168385 | 1.238019 | −0.03563 | 0.136323 | 0.215179 | −1.16949 |
| −2.63982 | 0.775219 | −0.31789 | 1.961086 | 1.060622 | 1.118627 | −1.02315 | 1.097147 | 0.323656 | −0.39696 |
| −1.90414 | −0.40407 | −3.44846 | −0.60716 | 0.116324 | 1.116324 | −1.74802 | 1.821445 | −0.05614 | −1.44846 |
| −3.50771 | 1.458077 | −2.92275 | 0.286708 | 1.44066 | 1.199652 | −0.48534 | 0.662217 | 0.178793 | 0.015854 |
| NA | 1.77776 | −1.22878 | 0.505557 | 2.014997 | 1.989857 | −2.36629 | 0.18953 | 0.932372 | −0.39543 |
| −4.18982 | 1.35707 | −2.48938 | 2.019629 | 1.486132 | 1.571727 | −2.0199 | −0.76356 | 0.591535 | −1.28293 |
| −3.411 | 0.313026 | 3.532351 | −0.22967 | 0.688535 | 1.923751 | −1.49044 | −0.75892 | 0.26546 | −0.36037 |
| −4.49043 | 0.985307 | 2.180229 | 0.46377 | 1.18553 | 1.65168 | −1.09811 | 0.526381 | 0.290933 | −0.68307 |
| NA | 1.007268 | −3.45943 | −0.74671 | 1.190822 | 1.70156 | −0.79647 | 0.32484 | 0.216133 | −0.50007 |
| NA | 1.076704 | NA | −0.08571 | 0.848779 | 1.262934 | −3.24222 | −3.07229 | 0.900742 | NA |
| −1.93681 | 0 | NA | 0.246794 | 0.66862 | 2.076018 | NA | −2.67377 | −0.35184 | NA |
| −1.05197 | −0.14982 | NA | 0.330171 | 0.602251 | 1.945066 | NA | −2.66078 | −0.55645 | NA |

| Nicotinat [symbol] | p-Cresol | Phenylac [symbol] | Phenylala [symbol] | Proline | Propionat [symbol] | Pyruvate | Ribose | Sarcosine |
|---|---|---|---|---|---|---|---|---|
| −0.32504 | −2.92594 | −2.74537 | 0.767078 | 0.968065 | 3.873662 | −1.40657 | 2.31437 | −2.44052 |
| −1.44666 | −3.03162 | −1.44666 | 0.638232 | 1.160001 | 4.731744 | −1.63307 | 0.486229 | NA |
| −0.48875 | −2.63559 | −0.48875 | 1.82852 | 2.164387 | 1.957936 | −1.58496 | 1.804984 | −3.85798 |
| −1.77934 | −3.2268 | −0.62014 | 1.727399 | 0.964823 | 4.381878 | −1.73203 | 1.957628 | −3.5163 |
| −0.35646 | −2.5935 | −1.00854 | 1.84232 | 1.670608 | 3.872941 | −1.0433 | 1.599146 | −4.07893 |
| −1.372 | −3.16341 | −0.95696 | 1.772771 | 1.524163 | 3.698389 | −1.90966 | NA | NA |
| −1.61318 | −2.75068 | −1.61318 | 1.375848 | 1.181629 | 4.0881 | −1.37217 | NA | NA |
| −1.42423 | −3.14043 | −1.3932 | 0.453092 | 0.383129 | 4.184712 | −2.24735 | 0.511644 | NA |
| −0.41504 | −3.37851 | −1.14585 | 0.921612 | 1.206451 | 3.91978 | −1.65605 | 0.418501 | −2.89308 |
| 0.032478 | −3.28041 | −2.03248 | 0.281837 | 0.514011 | 3.801744 | −1.60798 | 1.879466 | −5.69544 |
| −0.1112 | −3.55866 | −1.8888 | 0.763273 | 1.465729 | 3.6428 | −1.04081 | 0.981913 | −3.43312 |
| −0.02531 | −1.89407 | −1.37324 | 1.495259 | 1.767245 | 0.615793 | −0.58474 | 2.618543 | −3.41763 |
| −−0.67807 | −3.36257 | −1.58496 | 1.678698 | 1.601564 | 3.724893 | −1.74366 | 0.544321 | −3.71049 |
| −1.79055 | −4 | −1.64245 | 1.67948 | 1.577429 | 3.267957 | −2.35614 | 1 | −5 |
| 0.074223 | −3.02531 | −2.92578 | 0.789384 | 1.254795 | 3.793041 | −0.72414 | 1.520479 | NA |
| −1.03847 | −2.38995 | −2.078 | 1.788246 | 2.042091 | 3.413851 | −1.46657 | 1.351985 | −3.926 |
| −0.06326 | −2.20076 | −2.03726 | 1.32029 | 1.450004 | 3.680904 | −1.17219 | 1.155383 | −2.84462 |
| −0.09954 | −2.32193 | −3.07682 | 0.973033 | 1.328326 | 3.166358 | −1.01073 | 1.891851 | −2.6845 |
| 0.300606 | −2.49525 | −1.37978 | −0.47535 | 0.993033 | 4.15978 | −0.96474 | 2.539393 | −2.34325 |
| −1.70264 | −2.8677 | −2.91209 | 0.423296 | 1.646327 | 2.981966 | −1.64531 | 0.698931 | −2.02945 |
| −0.30871 | −1.7168 | −1.41724 | 1.863535 | 2.390119 | 1.009029 | −0.58435 | 2.193789 | −2.32977 |
| 0.774398 | −2.37291 | −3.01677 | 1.612591 | 1.761312 | 3.988508 | −1.07425 | 2.282443 | −2.76884 |
| −0.78028 | −2.83918 | −2.0091 | 1.312828 | 1.32629 | 3.645536 | −1.65155 | 1.601925 | −3.54967 |
| 0.114459 | −2.59826 | −2.59826 | 1.026232 | 1.558245 | 3.11293 | −0.88554 | 1.662268 | −2.76818 |
| −0.30231 | −2.6528 | −0.54847 | 0.154552 | 0.780157 | 3.740373 | −1.38977 | 1.647321 | −3.6528 |
| −0.63045 | −2.80733 | −2.47291 | 0.144844 | 1.124438 | 4.441013 | −0.87146 | 1.280136 | −5.00896 |
| −1.38521 | −3.01748 | −1.16949 | 0.800141 | 1.136323 | 4.730711 | −1.38521 | −0.61149 | −4.75445 |
| −0.99212 | −2.37678 | −1.68873 | 0.613163 | 0.430571 | 4.857901 | −1.45925 | 1.201484 | −4.54671 |
| 0.324129 | −2.71149 | −2.33298 | 0.05404 | 1.731449 | 4.84271 | −0.67587 | 0.836942 | −4.44846 |
| 0.062148 | −2.33778 | −2.42024 | 1.393159 | 1.893172 | 1.458077 | −0.48534 | 1.492292 | −3.70035 |
| 0.055946 | −3.42518 | −1.61783 | 1.860222 | 1.938224 | 3.618303 | −1.33772 | 1.644493 | −2.68821 |
| −0.56047 | −2.8679 | −2.14543 | 1.428561 | 1.538096 | 2.45042 | −2.38247 | 1.333737 | −3.8679 |
| 0.216272 | −2.08907 | −3.13089 | 0.826038 | 0.946551 | 3.666578 | −1.54593 | 0.781644 | NA |
| −0.19581 | −2.44603 | −1.99857 | 1.025273 | 1.599686 | 3.665024 | −0.43514 | 2.274445 | −3.68307 |
| 0.11211 | −2.54189 | −2.54189 | 0.846377 | 1.359328 | 3.274525 | −1.2718 | 1.370643 | −2.92892 |
| NA | −3.50525 | −1.57925 | 1.451271 | 1.803999 | NA | −2.15475 | NA | NA |
| NA | −2.74416 | −1 | 0.773115 | NA | NA | −1.39366 | NA | NA |
| −2.59039 | −1.85343 | −1.02852 | 0.646645 | 0.988832 | NA | −2.33886 | NA | NA |

| Serine | Serotonin | Succinate | Sucrose | Taurine | Threonin [symbol] | Trehalose | Trimethyl |
|---|---|---|---|---|---|---|---|
| 1.286107 | −1.83283 | 3.861523 | NA | 4.082298 | 0.789217 | −0.81047 | −3.51091 |
| 0.91834 | −0.79061 | 5.385466 | NA | 2.96016 | 0.223194 | −1.96749 | −1.2816 |
| 0.215268 | −1.27302 | −1.44294 | NA | 0.171766 | 2.134485 | −2.35548 | −2.27302 |
| 0.159262 | −1.98579 | 3.650278 | NA | 2.729513 | 0.797587 | NA | −1.68623 |
| 1.553342 | −1.70041 | 1.947874 | NA | 4.93621 | 1.457127 | NA | −1.23093 |
| 2.147373 | −0.08249 | 4.164743 | NA | 4.071311 | 1.541288 | NA | −2.27889 |
| 1.646206 | −2.85022 | 5.343833 | NA | 1.408514 | 1.231168 | NA | −1.57011 |
| 0.591371 | −2.14043 | 5.503681 | NA | 3.976283 | 0.040897 | NA | −1.3932 |
| 1.469485 | −2.05658 | 3.072699 | NA | 3.319151 | 0.97904 | NA | −2.61298 |
| 0.943596 | −1.95848 | 4.605291 | NA | 4.542962 | 0.494382 | −1.55248 | −1.85414 |
| 1.544156 | −1.69616 | 3.650798 | NA | 2.661393 | 1.262684 | NA | −2.01809 |
| 1.806371 | −0.68971 | −2.1697 | NA | −1.1697 | 1.913286 | −0.41763 | −1.33017 |
| 1.630975 | −1.922 | 3.378897 | NA | 1.844095 | 1.415037 | NA | −2.04064 |
| 1.915879 | −0.81018 | 3.188589 | NA | 3.805744 | 1.321928 | NA | −1.38529 |
| 0.981113 | −0.90193 | 3.000222 | NA | 3.514953 | −1.34081 | NA | −1.58474 |
| 2.239913 | −1.60407 | 5.297037 | NA | 0.211504 | 1.889064 | −1.63322 | −1.75607 |
| 1.32029 | −0.63516 | 3.553058 | NA | 3.294935 | 1.126927 | −3.52269 | −1.63516 |
| 2.199309 | −1.58496 | 1.236067 | NA | 2.550718 | 1.240597 | −2.32193 | −2.16993 |
| 0.718526 | −2.34325 | 2.427579 | NA | 4.608034 | −0.47535 | NA | −1.34325 |
| 1.581761 | −1.15721 | 4.040283 | NA | 2.015684 | 1.377925 | −3.74217 | −0.10474 |
| 2.337652 | −1.60988 | −1.9578 | NA | −0.77338 | 2.163965 | −1.51034 | −1.55925 |
| 1.960515 | −2.40206 | 0.054697 | NA | 2.07995 | 1.293848 | −1.82694 | −2.08603 |
| 0.909763 | −1.17621 | 3.910508 | NA | 4.752867 | 1.280919 | −3.83918 | −1.51725 |
| 1.687143 | −1.52026 | 1.707549 | NA | 4.065496 | 0.809245 | −3.59826 | −2.18322 |
| 0.347197 | −2.58241 | 3.66461 | −1.84545 | −0.26049 | 0.282657 | −1.80481 | −1.54847 |
| 1.04415 | −2.80733 | 3.668406 | NA | 4.632007 | 0.551754 | −2.16096 | −2.20161 |
| 1.044294 | −1.53206 | 4.254915 | NA | 4.142389 | 0.936714 | −4.33941 | −2.87998 |
| 1.372154 | −2.08728 | 5.170111 | NA | 5.241602 | 0.828331 | −2.02315 | −1.12044 |
| 1.131484 | −1.44846 | 3.012337 | NA | 4.207964 | 0.261198 | −3.12653 | −1.8635 |
| 2.309276 | −2.11539 | −2.04828 | NA | NA | 1.777695 | −1.86385 | −1.86385 |
| 2.209026 | −1.42518 | 1.275259 | NA | 3.066673 | 1.923548 | −1.33772 | −1.45555 |
| 1.517535 | −1.60486 | 1.333737 | NA | 3.737953 | 1.439532 | −2.43494 | −1.73039 |
| 0.403696 | −1.33571 | 1.080852 | NA | 3.752469 | 0.595425 | NA | −1.66377 |
| 1.523594 | −1.60778 | 1.257766 | NA | −0.17754 | 1.434386 | −0.73554 | −2.36114 |
| 1.24716 | −2.16993 | 2.872481 | NA | 3.603064 | 1.307281 | −1.72247 | −2.72247 |
| 2.729328 | NA | NA | NA | 0.547861 | 2.953362 | −1.08571 | NA |
| 2.345422 | NA | NA | NA | NA | 3.562356 | −0.2206 | NA |
| 1.521114 | −1.89525 | NA | 3.229177 | NA | 3.079334 | −0.45915 | NA |

| Tryptoph [symbol] | Tyrosine | Uracil | Uridine | Valerate | Valine | Xylose |
|---|---|---|---|---|---|---|
| −0.60402 | 0.698547 | 0.706324 | NA | −0.70355 | 1.352041 | 0.376619 |
| −1.36177 | 0.992765 | −2.32113 | −0.24312 | −0.36177 | 1.209389 | −0.3413 |
| 0.338416 | 1.781058 | −1.44294 | −0.68806 | 1.103951 | 2.719448 | 0.451874 |
| −0.5163 | 1.688811 | −1.73203 | −1.19438 | 1.000272 | 1.93948 | 0.095131 |
| 0.008537 | 1.995751 | −1.15293 | −0.27157 | 0.327066 | 2.217531 | −0.54287 |
| −0.294 | 2.066883 | −1.372 | −0.77639 | −0.372 | 2.25249 | −1.00587 |
| 0.042865 | 1.375848 | NA | −0.87622 | 0.682275 | 2.01131 | −0.10299 |
| −2.0409 | 0.674264 | −1.99359 | −1.42423 | 1.276214 | 0.922577 | −1.08981 |
| −0.74624 | 0.906891 | −1.57116 | −0.7697 | −0.22471 | 1.439112 | −0.34289 |
| −1.37351 | 0.085917 | 0.447515 | NA | −0.53894 | 0.953214 | −0.65105 |
| −0.86834 | 0.900776 | −0.35988 | −1.59182 | −2.06389 | 1.428996 | 0.870656 |
| −0.39526 | 1.542371 | −1.06008 | −0.77377 | 0.477187 | 2.50421 | 0.811188 |
| −0.29956 | 1.754888 | −2.26303 | −1.4975 | 0.724893 | 2.20294 | −0.54057 |
| 0.108524 | 1.675957 | −2.14202 | −1.16711 | 0 | 2.052568 | −0.79055 |
| −1.30911 | 1.068199 | −1.02531 | −0.58474 | −0.52889 | 1.357157 | 0.357157 |
| −0.20353 | 1.857981 | −0.94415 | 0.055853 | −0.17111 | 2.263825 | 0.152952 |
| −0.22991 | 1.869629 | −0.82225 | −0.27476 | 0.050201 | 1.913606 | −0.08973 |
| −0.90689 | 1.122857 | −0.16993 | −1.29546 | −0.6845 | 1.39079 | 0.161589 |
| −0.75829 | −0.02132 | 0.744213 | −1.85782 | 0.021322 | 0.089709 | 0.674672 |
| −0.62669 | 0.888806 | −2.74217 | −1.35751 | −3.51978 | 1.642495 | −1.22559 |
| 0.337652 | 1.722316 | −0.99097 | −0.9578 | 0.536961 | 2.629888 | 0.801933 |
| −0.31633 | 1.729749 | −0.01114 | −0.14025 | 0.502871 | 1.911013 | −0.75938 |
| −0.18892 | 1.680898 | −2.10221 | −0.59971 | 0.442111 | 2.018806 | −0.68717 |
| −1.12433 | 1.503279 | 0 | −0.55873 | −0.65966 | 1.79787 | 1.65808 |
| −1.06784 | 0.492875 | −0.20655 | −1.76528 | −0.499 | 0.78682 | 0.382821 |
| −1.60297 | 0.37647 | 0.052815 | −1.40015 | 0.008961 | 1.140786 | 0.362598 |
| −1.29502 | 0.991507 | −2.43252 | −0.63897 | 0.103533 | 1.374835 | 0.671817 |
| 0.360182 | 0.784208 | −2.37678 | −0.81879 | 1.118627 | 1.53544 | −0.68873 |
| −0.65838 | 0.136502 | 0 | NA | −2.22607 | 0.680823 | −1.01106 |
| −0.01585 | 1.253844 | −1.42024 | −0.55351 | 0.178793 | 2.206538 | 0.38711 |
| −0.05595 | 1.998286 | −0.94405 | −0.70636 | −0.11532 | 2.218676 | −0.16465 |
| 0.019629 | 1.61753 | −1.25909 | −0.31946 | −0.29501 | 2.067563 | −0.88604 |
| −0.98954 | 1.30128 | −0.66377 | −0.80896 | −0.32354 | 1.30128 | −0.61844 |
| −0.65754 | 1.109486 | 0.223819 | −0.41361 | −0.59561 | 1.567565 | 1.28765 |
| 0.071083 | 1.14543 | −1.20309 | −0.67516 | −1.1375 | 1.442106 | −0.54189 |
| −0.5234 | 1.299526 | NA | −1.92029 | −3.01982 | 1.800356 | −0.61772 |
| −0.67377 | 1.355976 | NA | −0.89616 | −2.06609 | 1.492332 | 0 |
| −1.62516 | 0.571877 | NA | −1.22782 | −2.22782 | 1.000282 | NA |

[symbol]

indicates data missing or illegible when filed

APPENDIX: TABLE 3-2

| Tube # | Sample ID | Diagnosis | 11-trans- [symbol] | 1-monoli [symbol] | 2,5-dihyd [symbol] | 2-hydroxy [symbol] | 2-linoleo [symbol] | 2-palmito [symbol] | 3-(3-hydr [symbol] |
|---|---|---|---|---|---|---|---|---|---|
| B3 | C4 | NT | 1.064639 | 0.065716 | −3.24084 | 2.155411 | 2.243494 | 0.774297 | 0.397475 |
| B5 | A17 | ASD | 1.580548 | 0.562208 | −5.00783 | 3.530679 | 2.393495 | 1.512131 | NA |
| B7 | A09 | ASD | 1.297375 | 2.135245 | −2.34665 | 3.618977 | 2.379294 | 0.572655 | 0.903617 |
| B9 | C1 | NT | 1.449447 | 0.369015 | −2.75087 | 2.400211 | 2.352934 | 0.538915 | −0.43403 |
| B11 | A24 | ASD | 1.736555 | 1.202903 | −3.42869 | 2.845256 | 2.786305 | 1.804516 | 0.331925 |
| B13 | A03 | ASD | 1.390292 | 1.584274 | −4.16823 | 3.378288 | 1.539332 | 0.400286 | −2.45203 |
| B15 | N5 | NT | 1.189338 | 0.501093 | −4.0302 | 2.592045 | 3.352121 | 1.234002 | 0.76977 |
| B17 | A11 | ASD | 1.732148 | 2.221837 | −4.20326 | 3.508895 | 1.132805 | 0.473525 | 0.63764 |
| B19 | N5 | NT | 2.066602 | 1.599976 | −4.0561 | 4.01393 | 3.015705 | 1.502111 | −1.8321 |
| B21 | C4 | NT | 1.231565 | 1.610751 | −3.75089 | 3.520461 | 2.653946 | 1.034403 | 0.939252 |
| B23 | A17 | ASD | 1.820649 | 0.235989 | −4.35847 | 2.338229 | 1.20783 | 0.982979 | −3.66794 |
| B25 | A09 | ASD | 0.663228 | 0.706541 | −3.97064 | 2.458534 | 2.37347 | 1.548059 | 1.169322 |
| B27 | C1 | NT | 1.330684 | 1.119807 | −2.92709 | 3.519523 | 2.854882 | 1.247776 | −0.24063 |
| B29 | A24 | ASD | 1.703431 | 1.285261 | −3.93751 | 2.864037 | 2.88309 | 1.821011 | 0.845776 |
| B31 | A03 | ASD | 0.635464 | −0.38622 | −4.02807 | 2.292382 | 1.81931 | 0.100451 | −1.70422 |
| B33 | N5 | NT | 1.361813 | 1.368319 | −4.89511 | 3.476083 | 3.222194 | 1.78835 | 1.370397 |
| B35 | A11 | ASD | 1.91183 | 1.389349 | −2.6986 | 2.251825 | 2.528858 | 1.341105 | 0.387347 |
| B37 | N5 | NT | 1.788769 | 0.449428 | −3.43322 | 2.453976 | 3.090868 | 1.210648 | −1.31981 |
| B39 | C4 | NT | 0.964241 | 0.496135 | −4.11005 | 3.634272 | 1.570458 | 1.127612 | −0.69298 |
| B41 | A17 | ASD | 1.643179 | 1.129915 | −5.05222 | 3.373563 | 1.274107 | 0.750007 | NA |
| B43 | A09 | ASD | 0.945242 | 0.949682 | −3.91321 | 2.329803 | 2.851012 | 1.111022 | 0.865549 |
| B45 | C1 | NT | 1.648866 | 0.968153 | −4.3375 | 3.575098 | 2.740019 | 1.283126 | 0.057045 |
| B49 | A03 | ASD | 1.36032 | 0.390969 | −2.83844 | 1.745132 | 2.74513 | 1.21744 | −3.4149 |
| B51 | N5 | NT | 1.598381 | 0.859822 | −3.25847 | 2.608192 | 2.520436 | 1.443595 | 0.585735 |
| B53 | A11 | ASD | 1.837604 | 1.488547 | −4.88772 | 3.668477 | 2.30705 | 0.978148 | 0.232932 |
| B55 | N5 | NT | 1.573365 | 1.076826 | −2.089 | 3.99945 | 2.097446 | 0.849778 | −1.21418 |
| B57 | C4 | NT | 1.659914 | 0.313643 | −3.78164 | 2.428672 | 2.032385 | 1.35312 | NA |
| B59 | A17 | ASD | 1.98039 | 1.484171 | −4.71161 | 3.923263 | 2.80664 | 2.064881 | NA |
| B61 | A09 | ASD | 1.244661 | 0.747006 | −3.81896 | 2.420483 | 2.528712 | 0.533378 | 0.803094 |
| B63 | C1 | NT | 1.123538 | 0.248498 | −4.02245 | 2.712958 | 2.084726 | 0.217502 | −0.47308 |
| B65 | A24 | ASD | 1.489634 | 0.262512 | −4.21707 | 3.497852 | 1.573588 | 0.901042 | 0.151734 |
| B67 | A03 | ASD | 1.500234 | 1.121901 | −4.42054 | 2.553408 | 3.142406 | 1.218088 | 0.102891 |
| B69 | A11 | ASD | 2.130042 | 1.253956 | −4.63058 | 3.724629 | 2.80705 | 1.793734 | 0.493365 |
| B996 | GF | GF | −2.06596 | 0.381516 | −0.67779 | 1.215043 | 0.941267 | 0.713474 | −2.75327 |
| B999 | GF | GF | −1.85618 | −0.00728 | −2.10486 | 2.871865 | 0.537383 | 0.574875 | −2.58221 |
| B1002 | GF | GF | −1.82568 | 0.35491 | −1.55731 | 2.633637 | 0.687947 | 0.816002 | −2.71565 |

| 3-(4-hydr [symbol] | 3,4-dihyd [symbol] | 3-aminois [symbol] | 3-deoxyh [symbol] | 3-hydroxy [symbol] | 3-hydroxy [symbol] | 3-methyl [symbol] | 4-hydroxy [symbol] | 4-hydroxy [symbol] | 5-aminov [symbol] |
|---|---|---|---|---|---|---|---|---|---|
| 1.290961 | 0.401898 | −1.20472 | NA | −0.8094 | 0.278263 | −3.138 | −5.1504 | −0.02716 | 5.535864 |
| −1.13432 | 0.851051 | −1.28097 | NA | −1.2131 | −1.94878 | −2.87185 | 2.343961 | 0.712396 | 2.690824 |
| 3.00617 | −0.4605 | −0.77723 | NA | −1.38486 | 1.584736 | −3.1246 | 0.192669 | 0.447841 | 3.209295 |
| 0.624623 | 0.314388 | −1.30216 | NA | 0.137244 | 1.243423 | −2.64224 | 1.577351 | 1.945831 | 1.67966 |
| 0.901571 | −1.05715 | −1.51125 | NA | −0.74502 | 1.759305 | −2.56553 | −0.31438 | −0.67405 | 2.817607 |
| −1.98412 | −0.28563 | −1.43501 | NA | 0.394855 | −1.70046 | −3.26736 | −5.47571 | 1.447761 | 4.642388 |
| 1.852229 | −3.18336 | −1.61735 | NA | −1.05303 | 1.351206 | −2.9662 | −4.1215 | −1.9266 | 4.235433 |
| 2.317443 | −0.98726 | −1.14182 | NA | 0.380094 | −2.11812 | −3.22024 | −4.35294 | 0.32628 | 3.718559 |
| −1.83995 | −0.33955 | −2.61872 | NA | −1.14227 | −1.61973 | −2.56674 | −4.35036 | −0.12086 | 3.739433 |
| 1.645624 | 0.205024 | −1.11918 | NA | −1.15125 | −1.58008 | −3.19379 | −4.54181 | −1.79991 | 6.518614 |
| −0.8758 | 0.997817 | −0.92199 | NA | −0.42985 | −1.69252 | −3.41833 | 2.441018 | 0.201972 | 2.500382 |
| 3.44587 | 0.625369 | −0.27218 | NA | −0.28108 | −1.72476 | −3.54509 | 0.966359 | 0.996904 | 3.07906 |
| 1.591488 | 1.188867 | −1.05678 | NA | 0.519933 | 1.188476 | −3.18872 | −7.18922 | 2.603403 | 4.905965 |
| 1.989981 | −0.63894 | −1.12412 | NA | −1.02171 | 1.733224 | −3.06718 | −0.45075 | −1.02845 | 2.781293 |
| −0.86832 | 0.279898 | −0.27459 | NA | −0.46751 | −1.07441 | −2.12887 | −4.82315 | 1.978752 | 3.258642 |
| 2.712873 | −0.63134 | −1.3495 | NA | −0.63752 | −1.89057 | −3.04413 | NA | 0.450815 | 3.593485 |
| 2.10198 | 0.012502 | −0.85282 | NA | 0.234583 | −1.51878 | −2.4111 | −5.36121 | −0.82558 | 3.59695 |
| −0.90855 | −0.90279 | −1.847 | NA | −0.83085 | 1.519484 | −3.231 | −4.51837 | −2.23452 | 3.349473 |
| −1.18997 | 0.572434 | −1.34636 | NA | 0.15886 | 1.057336 | −5.59484 | −4.27207 | 1.823134 | 7.408526 |
| 0.404115 | 0.388326 | −0.55052 | 0.164275 | −1.55962 | −1.71142 | −2.53501 | 1.833558 | −1.09918 | 1.996772 |
| 2.965195 | −0.4042 | −0.39706 | NA | −0.75965 | 1.317196 | −3.92678 | −2.92385 | 1.050334 | 3.035456 |
| 1.61352 | 1.129458 | −0.89909 | NA | 0.558312 | −1.7626 | −2.99336 | 2.09361 | 3.000574 | 3.604283 |
| −1.20588 | −0.02889 | −0.13585 | NA | −0.68241 | −0.90528 | −3.30519 | 1.142143 | −1.3155 | 1.954755 |
| 1.960864 | −1.65668 | −2.34112 | NA | −1.40604 | 1.556967 | −2.74799 | −4.22389 | −2.59083 | 3.597675 |
| 2.211441 | −0.94885 | −1.35752 | NA | 0.160996 | −1.69363 | −3.49834 | −4.94396 | −0.61099 | 3.56791 |
| −1.22105 | −0.08047 | 0.249704 | NA | 0.157879 | −1.39869 | −3.01244 | −5.01255 | 1.918146 | 3.71163 |
| −1.08965 | 0.065065 | −1.43687 | NA | −0.87701 | 0.563286 | −2.50489 | −4.53455 | −1.15602 | 5.932368 |
| −0.96401 | 1.486078 | −0.71182 | NA | −0.4397 | −1.55476 | −3.02666 | 2.41247 | 1.534958 | 2.718926 |
| 2.853186 | −0.70535 | −0.07925 | NA | −0.94535 | 1.504802 | −2.35093 | −4.55067 | −0.17933 | 3.510063 |
| 0.951582 | 0.713818 | 0.07459 | NA | 0.235007 | −3.14741 | −5.53591 | NA | 2.737434 | 6.337562 |
| 1.167247 | −2.15617 | −1.06725 | NA | −2.11143 | 1.781316 | −4.009 | −0.54961 | −0.52833 | 2.175191 |
| 2.099613 | −1.88241 | −0.89949 | NA | −0.87494 | 1.195508 | −3.10404 | −5.55137 | −1.50495 | 2.876965 |
| 2.337694 | −1.06359 | −0.2015 | NA | 0.502611 | −2.15925 | −3.03576 | −4.58009 | −1.49053 | 4.14394 |
| −0.37687 | −1.80362 | −1.94922 | 0.616833 | −0.77844 | −2.8365 | −3.48783 | −2.40123 | −0.78109 | −2.18617 |
| −0.72325 | −2.46957 | −1.69981 | 0.515115 | −1.08249 | 1.181152 | −2.45976 | −3.1935 | −0.99509 | −2.10996 |
| 0.475535 | −1.87628 | 0.273586 | 0.520585 | −0.8541 | −2.54314 | −2.94384 | −0.58154 | −0.78937 | −4.17634 |

| 5-hydroxy [symbol] | 5-hydroxy [symbol] | adenine | adenosin [symbol] | alpha-hy [symbol] | alpha-toc [symbol] | arachidic | behenic a [symbol] | benzoic a [symbol] | beta-cyan [symbol] |
|---|---|---|---|---|---|---|---|---|---|
| 3.978019 | 0.554611 | 0.028902 | −0.56791 | −0.92753 | 0.501709 | 0.590576 | −2.75595 | 1.041665 | −0.22507 |
| 3.873708 | −1.74095 | 0.722868 | −0.21078 | −1.06031 | 0.62551 | 0.546459 | −1.90447 | 1.338284 | −0.81991 |
| 3.825957 | −0.70647 | 0.672568 | −0.74941 | 1.544046 | 0.53121 | 1.294288 | −1.357 | 1.523075 | 0.1887 |
| 4.362761 | 1.209595 | 1.022484 | −0.31966 | 0.590986 | 0.651884 | 0.638062 | −3.63863 | 0.614392 | −0.33673 |
| 4.199038 | −1.92855 | −0.35272 | −0.17816 | −0.42722 | 1.244309 | 1.371151 | −2.99147 | 0.934139 | 0.046736 |
| 4.209278 | 0.453275 | 1.303937 | −1.23168 | 0.402254 | −0.02401 | 1.111524 | −1.09426 | 1.81002 | −0.1825 |
| 4.29272 | −1.42551 | 0.083379 | 0.028339 | 0.863188 | 0.53353 | −0.04784 | −2.57028 | 0.302009 | −0.53778 |
| 3.927726 | −0.4273 | 0.014281 | −0.62491 | −0.15505 | 0.53194 | 1.435054 | −1.59033 | 1.249108 | 0.0105 |
| 3.978635 | −1.06452 | −1.41503 | −1.48883 | 0.435837 | 0.917697 | 1.212542 | −1.15957 | 2.388825 | −0.18598 |
| 3.620072 | 0.609134 | 0.509794 | −1.33479 | −0.07885 | 0.964128 | 1.201722 | −1.7503 | 1.208484 | −0.55685 |
| 3.890463 | −0.99126 | 1.118372 | −0.49534 | 0.636393 | 0.645774 | 0.560571 | −1.7595 | 1.046712 | −0.2187 |
| 3.557104 | 0.483119 | 1.035891 | −1.4165 | −0.14317 | −0.00047 | −0.18191 | −1.83955 | 1.373314 | −1.7311 |
| 4.081225 | 0.81666 | 0.144568 | −1.1869 | 0.604291 | 0.950169 | 1.31551 | −4.45833 | 1.100615 | −0.81105 |
| 4.140515 | −0.91831 | −1.53034 | 0.035685 | −0.29227 | 0.805046 | 1.201532 | −3.77129 | 0.488855 | 0.132559 |
| 3.297951 | 0.591959 | NA | −0.85538 | 0.952059 | −0.03858 | 0.039289 | −1.09569 | 1.498733 | −0.14373 |
| 4.100213 | 0.391719 | −1.65262 | −0.41194 | 0.636698 | 1.091208 | 0.010023 | −2.33792 | 1.071877 | −0.39872 |
| 3.975694 | −0.70194 | 0.441175 | −0.03796 | 0.298988 | 0.902001 | 1.097635 | −1.38033 | 1.030978 | −0.39983 |
| 4.141538 | 0.527403 | 0.127638 | 0.035648 | 0.346939 | 0.462555 | 0.533044 | −2.31749 | 0.849017 | −0.61219 |
| 3.444893 | 0.358926 | 0.96518 | −1.65405 | −1.41498 | 0.537632 | 1.385192 | −2.25572 | 2.31289 | −0.8449 |
| 3.001759 | −1.15847 | 0.777783 | −0.79394 | 0.723324 | 0.449395 | 0.351253 | −2.45375 | 0.990098 | −0.23267 |
| 3.827374 | −0.10759 | 0.768576 | −0.4558 | 0.28011 | 0.365519 | 0.465841 | −2.06895 | 0.873439 | −1.85799 |
| 4.312365 | 1.188445 | 1.14538 | −0.9011 | 1.051902 | 0.89354 | 0.776144 | −2.88014 | 1.211046 | −0.14854 |
| 3.594991 | 0.851249 | −1.07444 | −0.82551 | 0.453797 | 0.965609 | 0.427161 | −3.01557 | 1.696805 | −0.468 |
| 4.226498 | −2.05603 | −1.06882 | 0.387645 | 0.947573 | 1.045896 | 0.997954 | −1.85298 | 0.117002 | −0.84017 |
| 3.717375 | −2.05501 | −3.48899 | 0.294672 | −0.00377 | 0.635546 | 1.252221 | −1.59891 | 1.170195 | −0.2036 |
| 3.31545 | −0.12946 | 0.506124 | −2.03009 | 1.094182 | 0.604309 | 0.454206 | −1.97723 | 2.170726 | 0.462436 |
| 3.990378 | 0.493042 | 1.082963 | −0.85664 | 0.32024 | 1.072144 | 0.09629 | NA | 1.043032 | −1.09087 |
| 4.105595 | −2.03837 | 0.514762 | 0.004965 | 0.96007 | 1.18985 | 1.257428 | −1.2484 | 1.555036 | −0.33312 |
| 4.162057 | −1.40788 | 1.580988 | −0.15374 | −1.30983 | 0.007873 | −0.22085 | −1.90326 | 0.770379 | −0.74722 |
| 3.62377 | 1.952022 | 0.403218 | −1.45545 | 0.781781 | 0.590703 | 0.719559 | −3.33888 | 0.641155 | −1.17209 |
| 3.712055 | −0.46531 | −2.08514 | 0.169148 | 0.258596 | 0.385564 | 0.695823 | −2.59837 | 0.402458 | −0.26111 |
| 4.333321 | −2.00322 | −1.25749 | −0.02119 | 0 | 1.132389 | 1.00458 | −4.31256 | 0.528392 | −1.06242 |
| 4.360549 | −1.08306 | −1.31335 | −0.17591 | −1.43009 | 0.663795 | 1.053745 | −1.89644 | 1.573137 | 0.590622 |
| −0.45444 | −0.34255 | 1.027409 | −3.54479 | 2.177635 | −0.85651 | −1.66857 | −4.9727 | 0.178385 | 1.284876 |
| −1.0318 | −1.9045 | 1.190574 | −1.66905 | 2.301205 | −0.78853 | −2.23295 | −5.00755 | −0.59217 | 1.228905 |
| −0.73012 | −1.22233 | 0.973844 | −3.40715 | −0.17858 | −0.50044 | −0.89057 | −3.42627 | 0.082634 | 0.41248 |

| beta-sito [symbol] | campeste [symbol] | carbonate | catechol | cellobios [symbol] | cholest-7- [symbol] | cholestan [symbol] | cholester [symbol] | cholic aci [symbol] | citraconic [symbol] |
|---|---|---|---|---|---|---|---|---|---|
| 1.888513 | 0.018758 | 4.858511 | 0.345734 | −2.13049 | −4.87984 | −2.72594 | 2.707492 | −1.18259 | −0.18132 |
| 2.288183 | 0.558095 | 4.753048 | 0.874911 | −3.29753 | −2.53587 | −2.93092 | 3.144175 | 2.027736 | −0.7462 |
| 2.450379 | 0.54005 | 4.801345 | −0.09082 | −1.16719 | −3.12785 | −2.83623 | 2.985328 | 0.071027 | −0.93717 |
| 2.136174 | 0.275586 | 4.422425 | 0.410096 | −1.72469 | −3.06324 | −3.18726 | 2.901331 | −0.41549 | −0.28237 |
| 2.657405 | 1.025468 | 4.647594 | −0.63238 | −2.51606 | −2.3836 | −2.16944 | 3.894352 | 1.048427 | −0.79287 |
| 2.139924 | 0.333223 | 4.972805 | −0.13068 | −2.88006 | −4.0953 | −3.04303 | 3.106514 | 2.00461 | −0.67396 |
| 2.050102 | 0.277858 | 4.532819 | −0.74312 | −1.35721 | −2.89979 | −2.8713 | 2.818187 | 0.70412 | −1.27363 |
| 2.288859 | 0.639372 | 4.703801 | −0.16584 | −1.2575 | −2.18204 | −2.183 | 3.133038 | 0.845603 | −0.85799 |
| 2.682416 | 0.929141 | 5.028501 | 0.147745 | −1.31171 | −2.07838 | −2.32182 | 3.903183 | 1.195791 | −0.1879 |
| 2.245018 | 0.55929 | 5.131256 | −0.41251 | −1.11473 | −2.39725 | −2.34288 | 3.137142 | 1.027112 | −1.28647 |
| 2.24574 | 0.512251 | 4.932934 | 0.903298 | −3.55748 | −3.03557 | −2.41428 | 3.039076 | 2.02858 | −0.4619 |
| 2.061572 | 0.241778 | 5.056232 | 0.534138 | −3.17189 | −6.6842 | −1.9213 | 2.363509 | −0.44375 | −0.2475 |
| 2.417274 | 0.801881 | 4.293694 | −0.54527 | −1.39088 | −2.24089 | −2.29924 | 3.545023 | 1.12199 | −1.29471 |
| 2.256123 | 0.54295 | 4.587898 | −0.70436 | −2.38707 | −2.07473 | −2.87748 | 3.299589 | 1.511715 | −0.78992 |
| 1.927996 | 0.081574 | 5.206368 | 0.247296 | −3.65829 | −5.80638 | −3.33706 | 3.3383 | 0.915712 | −0.04198 |
| 2.405619 | 0.747426 | 4.808671 | −0.19589 | −1.94836 | −3.48308 | −2.67934 | 3.263354 | 1.156348 | −0.73396 |
| 2.49309 | 0.860709 | 4.871352 | −0.06811 | −1.95698 | −1.76355 | −1.75312 | 3.530332 | 0.392906 | −0.0972 |
| 2.084061 | 0.531826 | 4.566412 | −0.6115 | −1.5202 | −2.17218 | −2.14771 | 3.451795 | 1.188008 | −0.64769 |
| 1.957786 | −0.02544 | 5.151499 | −0.25061 | −2.85249 | −4.1416 | −2.34923 | 2.491905 | 0.082814 | −0.97042 |
| 2.185606 | 0.410543 | 4.98933 | −0.35497 | −2.70787 | −1.56448 | −3.17529 | 3.106245 | 2.101308 | −0.96405 |
| 2.309155 | 0.487827 | 4.780761 | 1.745509 | −2.51263 | −3.10957 | −3.14612 | 2.721123 | 0.144952 | −0.70784 |
| 2.382021 | 0.719603 | 4.582571 | −0.18121 | −2.27806 | −3.17714 | −2.58651 | 3.361022 | 0.578888 | −0.74138 |
| 2.408206 | 0.527935 | 5.323538 | 0.172764 | −1.46381 | −3.18631 | −2.92999 | 3.544132 | 1.555205 | 0.091094 |
| 2.548003 | 0.907008 | 4.4201 | −0.77107 | −1.51795 | −1.13353 | −2.38598 | 3.721017 | 1.61279 | −1.24069 |
| 2.51331 | 0.948342 | 4.975245 | −0.14987 | −2.18908 | −1.68155 | −1.71032 | 3.582453 | 1.022684 | −1.00667 |
| 2.352914 | 0.283599 | 5.304441 | 0.323386 | −1.05153 | −2.77415 | −2.50012 | 3.344081 | 0.958639 | −0.169 |
| 2.359189 | 0.375105 | 5.051926 | 0.280548 | −3.13797 | −3.29174 | −3.13185 | 2.715686 | 0.732526 | −0.33211 |
| 2.895023 | 1.242465 | 5.141702 | 0.558075 | −2.17111 | −1.44701 | −1.6423 | 3.500459 | 3.191754 | −0.63517 |
| 1.755926 | −0.14945 | 4.53582 | 1.724139 | −3.23335 | −4.60477 | −3.62595 | 2.202915 | 0.437534 | −0.53278 |
| 1.998475 | 0.385167 | 3.566246 | 1.882048 | −1.15118 | −3.02498 | −2.40521 | 2.972484 | 1.003357 | −1.10967 |
| 1.972279 | 0.130827 | 4.1.30632 | −0.80665 | −3.26974 | −2.72592 | −2.43097 | 3.056801 | 1.215559 | −1.86012 |
| 2.409438 | 0.606541 | 4.759259 | 1.589304 | −0.92636 | −2.28851 | −1.88484 | 3.701478 | 2.443093 | −1.14138 |
| 2.598905 | 0.978019 | 4.494609 | −0.01268 | −2.49592 | −2.53929 | −1.84531 | 3.362905 | 0.904774 | −0.62242 |
| 1.393228 | −0.35349 | 3.935882 | −0.85911 | 0.788294 | −5.09751 | −2.77094 | 1.515934 | −0.26043 | −1.1485 |
| 1.672034 | 0.007277 | 3.652074 | −0.96741 | 1.13135 | −5.62052 | −2.85551 | 2.062917 | −1.06009 | −1.56265 |
| 1.958793 | 0.270025 | 4.018329 | −1.03574 | 1.142633 | −4.53403 | −2.51528 | 2.049303 | −0.04288 | −1.42003 |

| citric acid | creatinin [symbol] | [symbol] | [symbol] zein | dehydrol [symbol] | D-fructos [symbol] | D-galacto [symbol] | D-galacto [symbol] | D-gluconi [symbol] | D-glucose [symbol] |
|---|---|---|---|---|---|---|---|---|---|
| NA | 0.606831 | 1.701093 | −0.19499 | −1.1309 | 3.31734 | NA | 3.358536 | NA | 1.946058 |
| NA | −0.7383 | 1.322204 | 0.403651 | −1.34419 | 1.893475 | NA | 2.856723 | NA | 0.967103 |
| NA | −0.50243 | 1.972494 | 0.51726 | −0.90754 | −0.06184 | NA | 0.398576 | NA | 1.045764 |
| NA | −0.35781 | 2.764882 | −1.13839 | −1.9108 | 2.168534 | NA | 0.366686 | NA | 1.234652 |
| NA | −0.71917 | 0.499412 | 0.272169 | −0.83919 | −2.42828 | NA | 4.308427 | NA | 0.083429 |
| NA | −0.65756 | 5.300061 | −1.56396 | −1.5452 | 1.411522 | NA | −0.07025 | NA | 1.094048 |
| NA | −0.92119 | −0.06944 | −5.11296 | −1.70841 | 3.016093 | NA | 4.022048 | NA | 0.907772 |
| NA | −0.43621 | 1.234427 | −0.07506 | −1.48679 | −0.07478 | NA | 2.144252 | NA | 0.796381 |
| NA | 0.188314 | 2.225165 | −6.41125 | −1.35567 | 1.072874 | NA | 2.418878 | NA | 2.256358 |
| NA | −0.61567 | 4.389185 | 0.105325 | −0.70094 | 1.507795 | NA | 2.424716 | NA | 1.126983 |
| NA | −0.40508 | 1.627518 | 0.106434 | −1.06191 | 1.85687 | NA | 0.133165 | NA | 0.799273 |
| NA | 0.537339 | 1.911491 | 0.574683 | −0.83538 | 1.18984 | NA | 0.254534 | NA | 1.908302 |
| NA | −1.9175 | 0.831008 | −1.65747 | −1.39658 | 1.347244 | NA | −0.37259 | NA | 0.793877 |
| NA | −0.30562 | 4.299171 | −0.0641 | −0.71413 | 2.888071 | NA | 3.929759 | NA | −0.14563 |
| NA | 0.646577 | 1.750239 | −0.63896 | −1.09497 | 1.674444 | NA | 1.291966 | NA | 1.627036 |
| NA | −0.71122 | 0.852449 | −2.43211 | −1.30509 | 1.619831 | NA | 2.647646 | NA | 1.965894 |
| NA | 0.29928 | 1.429177 | 0.555107 | −1.30979 | 0.163175 | NA | 0.514492 | NA | 1.484687 |
| NA | −2.07346 | 1.143595 | −6.54125 | −1.5628 | 3.416186 | NA | 4.316349 | NA | 1.263976 |
| NA | −0.78555 | 1.617929 | −0.70547 | −0.97129 | 0.494778 | NA | 2.915706 | NA | 1.613154 |
| NA | −0.49617 | 1.16078 | −0.56749 | −1.16742 | 0.507156 | NA | 1.733045 | NA | 0.463656 |
| NA | 0.048507 | 0.090991 | 0.824009 | −0.67847 | −0.16349 | NA | 0.323714 | NA | 1.05975 |
| NA | −1.1306 | 0.767535 | −1.81675 | −1.47248 | 2.177301 | NA | 0.054621 | NA | 1.762604 |
| NA | 0.318116 | 2.073501 | −0.24735 | −1.89528 | 2.907513 | NA | 1.916523 | NA | 2.10992 |
| NA | −0.77332 | 0.195464 | −5.59767 | −1.42433 | 3.246001 | NA | 3.808301 | NA | −0.07057 |
| NA | −0.56652 | 1.695763 | 0.788124 | −1.48355 | 1.276098 | NA | 0.217427 | NA | 1.086165 |
| NA | −0.03969 | 2.175711 | −5.75606 | −0.56907 | −0.09888 | NA | 1.60736 | NA | 1.846213 |
| NA | −1.85093 | 1.689237 | −1.36282 | −1.55981 | 2.990438 | NA | 0.342895 | NA | 2.0187 |
| NA | −0.65314 | 1.474317 | −0.00496 | −0.90515 | 1.638693 | NA | 2.890344 | NA | 1.060204 |
| NA | −0.27352 | 2.548012 | −1.01857 | −0.87744 | −0.13562 | NA | 0.379102 | NA | 0.310752 |
| NA | −1.52723 | 2.193308 | −2.24962 | −1.92408 | 1.297829 | NA | 1.972052 | NA | 0.320621 |
| NA | −0.88532 | 3.209767 | −3.1375 | −0.8076 | 3.249794 | NA | 0.594272 | NA | 0.996133 |
| NA | −4.14911 | 1.449474 | 0.209925 | −1.81181 | 4.209755 | NA | 2.061294 | NA | 1.576324 |
| NA | −0.19231 | 1.50863 | 0.251108 | −1.10721 | 2.260948 | NA | 0.114843 | NA | 1.455971 |
| −0.47397 | −3.04207 | 1.337253 | −2.17988 | −0.39658 | 2.511966 | 1.238391 | 0.861613 | 2.30268 | 1.970121 |
| −0.51837 | −4.01517 | 0.999398 | −2.6902 | −0.25202 | 2.431126 | 1.417367 | 1.60303 | 2.641571 | 1.675436 |
| NA | −3.05394 | 1.350527 | −3.02807 | −0.4328 | 2.60359 | 1.14331 | 1.029722 | 2.19248 | 1.535102 |

| D-glucose [symbol] | D-malic a [symbol] | D-mannit [symbol] | D-manno [symbol] | D-pinitol* | D-ribose | D-xylose | fumaric a [symbol] | galactinol | gallic acid |
|---|---|---|---|---|---|---|---|---|---|
| −1.97748 | −1.23469 | 0.997348 | 2.069151 | −0.01912 | 1.867024 | 3.601889 | −4.29219 | −1.33614 | −8.70521 |
| −2.23461 | −2.5946 | 1.097918 | 1.362098 | 0.044119 | 1.740897 | 3.44216 | −0.61795 | 0.095272 | 1.181866 |
| −2.78533 | −0.91759 | 0.978608 | 1.558882 | 0.367916 | 2.03901 | 1.994897 | 0.260313 | 0.811425 | −2.53533 |
| −0.81717 | −1.27083 | 0.295511 | 1.56423 | −1.18574 | 1.944312 | 0.483741 | 1.521348 | −3.12556 | −2.22691 |
| −0.69369 | −2.19225 | 1.089844 | 3.288938 | −2.31576 | 1.333612 | 4.151257 | −0.52256 | −2.91326 | −2.19321 |
| −3.50279 | −1.60673 | 0.129549 | 3.168944 | −1.2617 | 1.871889 | 1.774288 | 0.380741 | −1.57561 | 1.948862 |
| 0.47986 | −1.35493 | −0.00021 | 4.072223 | −0.83774 | 1.076811 | 2.109445 | −0.97362 | −0.72154 | −1.0615 |
| −3.61538 | −1.49765 | 0.475534 | 1.063352 | −1.25266 | 2.227393 | 1.836581 | 0.169835 | −0.86792 | −4.12066 |
| −2.33526 | −1.08473 | 0.643455 | 3.591699 | −2.05308 | 1.535291 | 3.274242 | −0.59681 | −3.91286 | −2.09139 |
| −2.5559 | −1.32713 | 0.193335 | 1.329514 | −1.74831 | 1.885673 | 3.531112 | −0.57369 | −0.26409 | −2.14556 |
| −1.9925 | −1.76059 | 1.204569 | 1.352777 | −0.04169 | 2.237989 | 3.732151 | −1.46529 | −0.30782 | −1.80864 |
| −2.57679 | −0.153 | 1.357573 | 1.451947 | 0.53074 | 1.972903 | 2.279588 | 1.846623 | −0.05132 | 1.330028 |
| −1.63327 | −1.64459 | 1.145699 | 0.873282 | −0.47606 | 1.183975 | −1.65357 | −0.07696 | −1.4494 | 1.396066 |
| −0.32615 | −1.50856 | −1.31066 | 3.065807 | −2.25034 | 1.739905 | 2.153604 | −0.61406 | −0.46618 | −1.77477 |
| NA | −0.95543 | −1.70526 | 2.450669 | −0.59808 | 2.261613 | 1.870967 | 1.800953 | −4.2251 | 3.064256 |
| −1.44509 | −1.27036 | −0.32282 | 3.815306 | −1.12632 | 1.471165 | 3.358025 | 0.172058 | −1.47801 | 1.281291 |
| −1.95608 | −1.01328 | 0.349281 | 1.769287 | −1.62132 | 2.005477 | 1.879442 | 1.640024 | −0.25078 | −4.45024 |
| −1.31513 | −1.26286 | −0.354 | 4.431538 | −2.2786 | 1.985015 | 3.718251 | 0.909686 | −3.11411 | 3.753534 |
| −1.38402 | −1.7797 | −0.06281 | 0.677992 | 0.253734 | 1.121419 | 2.959153 | 1.250363 | −0.58988 | 2.345375 |
| −2.07498 | −1.94914 | 4.296319 | 0.876518 | −1.10662 | 1.297837 | 3.503608 | 0.169822 | −0.17823 | −0.33685 |
| −1.33173 | −0.57315 | 0.497217 | 1.459268 | 0.235887 | 2.206003 | 2.110146 | −0.7072 | 0.339995 | −2.01018 |
| −0.57349 | −1.36595 | 0.411387 | 1.188364 | −0.41753 | 1.876755 | −1.55599 | −1.34495 | −2.53724 | 0.870614 |
| −5.84109 | −1.50523 | −0.77797 | 3.070035 | −1.6616 | 0.937496 | 1.715109 | 1.629749 | −2.94338 | 2.078437 |
| −0.1599 | −2.32572 | 0.359376 | 3.167518 | −1.89706 | 1.111659 | 0.436578 | −0.64004 | −0.22297 | −1.12981 |
| −1.77654 | −1.27233 | 1.090756 | 1.438255 | −1.60784 | 2.040466 | 1.419259 | 1.083088 | −0.62715 | −3.65632 |
| −4.12909 | −1.93141 | 0.360922 | 2.816996 | −2.97819 | 1.460788 | 1.353709 | 0.12128 | −1.96859 | 0.076009 |
| −2.40359 | −1.52714 | 0.645986 | 1.543286 | −0.79373 | 1.420653 | 1.706406 | 1.111802 | −2.97925 | 1.828315 |
| −1.74659 | −2.53953 | 1.377173 | 1.124985 | 0.251101 | 1.78732 | 3.403031 | −0.54133 | −0.0461 | −0.55367 |
| −0.92916 | −0.80632 | 0.98853 | 1.457783 | 0.224857 | 2.783401 | 2.650584 | 1.627168 | 0.21038 | −1.96501 |
| −1.92443 | −2.85529 | 1.355716 | −0.20223 | −2.05576 | 1.379105 | 0.890729 | −5.15995 | −2.6524 | −1.23008 |
| −0.41142 | −1.66353 | 1.290784 | 1.711566 | −2.65287 | 1.976859 | 1.606284 | 1.165394 | −3.3322 | −2.39477 |
| −2.04125 | −1.39496 | 0.431173 | 3.137486 | −0.44266 | 2.009135 | 1.346701 | −1.81084 | −1.25615 | −0.14344 |
| −0.80091 | −1.45546 | 0.635325 | 1.556244 | −1.30725 | 1.960829 | 1.155044 | 0.159767 | −0.16792 | −4.11598 |
| −5.76593 | −2.87245 | 4.997305 | 1.066541 | 6.770024 | 2.638242 | 2.503288 | −0.45517 | 7.108064 | −0.71009 |
| −6.81302 | −1.74298 | 5.111494 | 1.540268 | 6.807486 | 2.689896 | 2.615649 | 0.514977 | 7.221215 | −0.81996 |
| −5.59298 | −1.05105 | 4.995277 | 1.032202 | 6.792382 | 2.858524 | 2.763293 | 0.40204 | 7.278975 | −1.28009 |

| gamma-t [symbol] | genistein | gentisic a [symbol] | glucosyl g [symbol] | glyceric a [symbol] | glycerol | glycerol 3 [symbol] | glycolic a [symbol] | [symbol] | hexanoic |
|---|---|---|---|---|---|---|---|---|---|
| −2.08568 | 0.61949 | 0.74152 | NA | −2.28172 | 4.213957 | 2.050542 | 0.73788 | 0.252301 | −1.55602 |
| −0.79869 | 2.409253 | 1.227806 | NA | −2.07215 | 4.2.30123 | 2.219945 | 0.150504 | 0.098776 | −0.89688 |
| −1.0459 | 2.471317 | 0.886952 | NA | −0.98437 | 3.987997 | 2.558697 | 0.770182 | 0.66403 | −0.88871 |
| −1.12359 | −0.54581 | 3.207557 | NA | −2.23742 | 3.439144 | 2.273987 | 0.15459 | 0.410774 | −2.2235 |
| −0.96708 | −0.54507 | 3.491851 | NA | −2.45509 | 3.989409 | 2.612523 | −0.06746 | 0.793951 | −1.61265 |
| −1.63812 | 0.306953 | 0.360745 | NA | −1.26295 | 4.250743 | 2.118995 | 0.389527 | 0.626977 | −0.71757 |
| −1.69668 | −0.64804 | 3.24933 | NA | −2.48022 | 3.500654 | 2.317846 | −0.06707 | 0.202303 | −2.27532 |
| −1.0063 | 2.279228 | 3.112904 | NA | −0.74939 | 4.013807 | 2.1442.12 | 0.513096 | 0.737863 | −0.90451 |
| −1.09585 | −0.96144 | 0.068156 | NA | −1.77132 | 4.773024 | 2.491388 | 0.80491 | 0.868062 | −0.39834 |
| −1.02147 | 2.344606 | 0.059837 | NA | −4.67196 | 4.121486 | 2.112347 | 0.244638 | 0.115514 | −1.27752 |
| −1.46156 | 1.920445 | 0.813354 | NA | −2.14922 | 4.284188 | 2.176945 | 0.490979 | 0.003315 | −0.75748 |
| −1.8822 | 1.223319 | 1.332728 | NA | −1.02099 | 4.106133 | 2.141517 | 1.277206 | −0.17507 | −0.85421 |
| −1.29153 | 1.282911 | 3.237894 | NA | −1.79198 | 3.794633 | 2.311155 | −0.21097 | 0.444151 | −1.6181 |
| −0.92718 | −0.5995 | 3.443559 | NA | −2.01389 | 3.718916 | 2.514474 | 0.426263 | 0.623831 | −1.68381 |
| −2.87786 | −0.29787 | 0.958471 | NA | 0 | 4.499326 | 1.590741 | 1.535255 | 0.097019 | −0.76258 |
| −0.94774 | −0.38765 | 0.800994 | NA | 1.94379 | 3.885288 | 2.034125 | 0.914547 | 0.321062 | −1.36697 |
| −1.33868 | 2.247988 | 0.219984 | NA | −1.38166 | 4.274032 | 2.2.36506 | 0.682067 | 0.90796 | −1.17003 |
| −1.9222 | −0.80129 | 2.997511 | NA | −2.31232 | 3.778211 | 2.05616 | 0.308368 | 0.28518 | −1.21243 |
| −1.47319 | −0.23664 | 0.501543 | NA | −3.80713 | 4.592572 | 1.948804 | 0.654683 | 0.078606 | −0.58416 |
| −1.00882 | 1.933616 | 2.694166 | NA | −1.59254 | 3.803855 | 1.755401 | 0.466535 | −0.25172 | −1.08899 |
| −1.88219 | 2.263968 | 0.775005 | NA | −1.54679 | 3.903381 | 2.445261 | 0.757784 | −0.06936 | −1.1781 |
| −1.07318 | 0.457508 | 1.064327 | NA | −1.60635 | 4.250968 | 2.358992 | −0.04471 | 0.401214 | −1.97247 |
| −1.66249 | 1.823292 | 0.012645 | NA | −0.91906 | 4.472158 | 1.87731 | 0.775999 | 0.64854 | −1.00312 |
| −0.7729 | −0.09132 | 3.277083 | NA | −2.31487 | 3.334607 | 2.374029 | −0.18404 | 0.280666 | −2.08155 |
| −0.94564 | 3.119913 | 3.127525 | NA | −1.28806 | 3.92018 | 2.188512 | 0.478853 | 0.397438 | −1.30224 |
| −1.90713 | −0.76729 | 1.488917 | NA | −4.09198 | 4.687989 | 2.290641 | 0.957429 | 0.218796 | −0.27985 |
| −1.04628 | −0.74059 | 0.305742 | NA | −2.84355 | 4.149498 | 1.934049 | 0.34634 | −0.06713 | −1.20578 |
| −0.90029 | 2.683541 | 3.550001 | NA | −1.65849 | 5.609897 | 2.604841 | 0.635346 | 0.415087 | −0.67133 |
| −1.88584 | 0.553886 | 0.854795 | NA | −1.17183 | 3.1504616 | 2.775177 | 1.009439 | −0.31301 | −1.50515 |
| −1.44209 | 0.025994 | −0.09206 | NA | −3.09031 | 3.074372 | 2.145304 | 0.028646 | 0.335249 | −1.54048 |
| −1.33552 | 0.01095 | 3.50204 | NA | −2.03771 | 4.455645 | 2.58181 | 0.070643 | −0.14089 | −1.50563 |
| −1.2611 | 1.904816 | −0.32143 | NA | −2.41898 | 3.712757 | 1.888309 | −0.40829 | 0.740249 | −1.83175 |
| −1.16127 | 2.828843 | 3.419516 | NA | −1.33368 | 4.510716 | 2.478462 | 0.523192 | 0.652125 | −1.126 |
| −3.27566 | 0.776205 | 2.052372 | 3.381765 | −1.72936 | 3.631912 | 1.388426 | −0.36015 | −0.43913 | −1.64563 |
| −3.12872 | 0.645272 | 2.637134 | 3.392412 | −2.09998 | 3.549213 | 1.627475 | −0.79201 | −0.08898 | −1.93907 |
| −4.23363 | 0.477786 | 2.327361 | 3.435886 | −2.05551 | 3.781309 | 1.248359 | −0.72293 | −0.08327 | −1.69862 |

| hydrocinn [symbol] | hydroxyla [symbol] | inosine | inositol p [symbol] | L-2-amino [symbol] | lactic acid | L-alanine | L-cystein [symbol] | L-glutami [symbol] | L-glutami [symbol] |
|---|---|---|---|---|---|---|---|---|---|
| −0.81835 | 3.55802 | −0.64427 | −0.74323 | 1.898615 | 1.507875 | 1.589429 | 2.708037 | 3.930965 | 0.539883 |
| 2.495417 | 3.735788 | −0.70669 | NA | 0.92608 | 2.23704 | 1.883522 | 2.245987 | 5.175427 | 1.249374 |
| −3.79178 | 3.95886 | −0.44086 | −0.43138 | −0.49927 | 0.933181 | 1.773365 | 2.834298 | 5.173249 | 1.178272 |
| −3.88333 | 2.959174 | −0.85127 | −0.71279 | −1.14362 | 3.350891 | 0.950234 | 2.059892 | 4.518029 | 0.905969 |
| −2.35678 | 3.811117 | −0.35411 | 0.495169 | 1.507795 | 0.950447 | 1.451202 | 2.795997 | 4.679551 | 0.989237 |
| 2.227225 | 3.143619 | −0.68315 | −0.35204 | 0.572695 | 0.823257 | 1.643255 | 1.570012 | 4.649318 | 0.886458 |
| 1.893153 | 3.855346 | −0.30238 | −0.00184 | 2.095384 | 1.132359 | 1.541597 | 2.14031 | 5.536945 | 1.417356 |
| −2.36174 | 3.570489 | −0.21938 | −0.55197 | −3.07551 | 1.621137 | 1.60576 | 2.252193 | 4.925799 | 0.844873 |
| 2.215289 | 4.225028 | −0.75201 | 0.249601 | 1.692526 | 2.292754 | 2.220997 | 1.727256 | 4.944783 | −0.21551 |
| −0.0602 | 3.200837 | −0.00571 | −0.48921 | 1.998457 | 1.79545 | 1.341939 | 2.507231 | 3.655312 | 0.356623 |
| 2.21533 | 4.629247 | −0.45901 | −5.53397 | 1.75934 | 1.230538 | 1.95521 | 2.527905 | 4.867376 | 0.997903 |
| 2.46728 | 4.262242 | −0.99917 | −1.76714 | −0.73732 | 2.557132 | 1.986993 | 2.81651 | 4.336408 | 1.014888 |
| −3.78545 | 3.518338 | −0.84644 | −1.03861 | −0.76515 | 4.905353 | 1.267932 | 1.905146 | 4.480785 | 0.937857 |
| −2.6508 | 3.758161 | −0.27214 | 0.104196 | 1.760498 | 1.797814 | 1.267922 | 3.045732 | 4.780958 | 1.022527 |
| 1.812694 | 4.552145 | −1.34533 | −1.63508 | 1.63181 | 1.491651 | 1.97529 | 2.611863 | 4.976123 | 0.149724 |
| 2.291668 | 3.767609 | −0.65199 | −0.72103 | 1.905222 | 0.501235 | 1.544352 | 2.015873 | 4.697478 | 0.853098 |
| −2.86888 | 3.980263 | −0.34699 | −0.26343 | −1.69886 | 1.069545 | 1.664565 | 2.603128 | 4.438544 | 0.122817 |
| 2.029306 | 3.712518 | −0.33623 | −0.46184 | 1.987067 | 1.228141 | 1.215578 | 2.515548 | 4.172184 | 0.274148 |
| 2.037827 | 4.165836 | −1.10147 | −1.4541 | 1.39655 | 5.044559 | 1.758334 | 1.882308 | 3.579507 | 0.194806 |
| 1.255094 | 3.536644 | −0.58984 | NA | 1.415796 | 1.241087 | 1.5913876 | 2.52014 | 4.655877 | 0.814027 |
| −0.70251 | 4.004648 | −0.44595 | −0.94092 | −0.03184 | 1.847965 | 1.6559 | 2.976094 | 4.827407 | 1.236294 |
| 2.230179 | 2.977704 | −1.02909 | −0.915 | −1.08711 | 5.49533 | 1.368269 | 2.000709 | 4.880879 | 0.958795 |
| 2.216721 | 4.9150014 | −1.10118 | −1.40525 | 0.163667 | 1.225324 | 2.018383 | 1.585828 | 2.847362 | −0.86894 |
| −0.95813 | 3.621877 | −0.22065 | 0.410967 | 2.314346 | 0.276521 | 1.050055 | 2.474052 | 5.084512 | 0.765354 |
| −3.49506 | 3.908274 | −0.09171 | −0.35238 | −1.56401 | 1.153416 | 1.774185 | 2.057475 | 4.520135 | 0.181902 |
| 0.673983 | 4.797518 | −0.91846 | −0.41085 | 2.458691 | 1.050671 | 2.343659 | 2.042539 | 3.774524 | −0.63596 |
| 2.644514 | 4.336555 | −0.94641 | −0.96232 | 1.266158 | 1.811754 | 1.499819 | 1.747538 | 3.481558 | 0.121915 |
| 2.375979 | 3.952155 | −0.35947 | −5.30273 | 1.821314 | 1.76717 | 1.951532 | 2.808621 | 5.158392 | 1.36785 |
| −2.95935 | 3.466921 | −0.01617 | −0.05753 | 0.436828 | 1.577553 | 1.428991 | 3.310732 | 5.759199 | 1.064026 |
| 0.250675 | 4.445441 | −0.81447 | −0.6167 | −0.15822 | 3.700158 | 1.300748 | 1.765077 | 3.80918 | −0.04285 |
| −2.6488 | 3.689517 | −0.25028 | 0.547306 | 1.971409 | 1.161339 | 1.261425 | 2.803817 | 5.083399 | 0.995801 |
| −0.44982 | 3.537997 | −0.56764 | −0.80157 | −1.33485 | 1.562466 | 1.016156 | 2.17243 | 4.273172 | 0.917858 |
| −1.82111 | 4.113985 | −0.40479 | −0.00099 | −1.35129 | 0.830479 | 1.742001 | 2.340829 | 5.400141 | 0.581035 |
| −1.72912 | 4.135295 | −0.77541 | −3.99081 | −2.40695 | 2.819152 | 1.441911 | 2.812894 | 5.73842 | 5.109944 |
| −3.35707 | 3.061672 | −0.43861 | −4.12805 | −2.6382 | 2.929974 | 0.792231 | 2.891144 | 6.086555 | 5.398551 |
| −3.19694 | 3.279849 | −0.6371 | −3.80113 | −2.55308 | 2.742532 | 0.890434 | 2.702747 | 5.584906 | 4.685955 |

| linoleic a [symbol] | L-isoleuci [symbol] | L-leucine | L-lysine | L-methio [symbol] | L-ornithin [symbol] | L-proline | L-pyroglu [symbol] | L-serine | L-threoni [symbol] |
|---|---|---|---|---|---|---|---|---|---|
| 4.759865 | −0.95626 | 3.217028 | 3.480834 | 2.052531 | 3.405456 | 3.940973 | 5.495399 | 1.1441.87 | 0.893312 |
| 4.918865 | −0.15197 | 4.626829 | 3.403062 | 1.872472 | 1.133826 | 3.482905 | 5.415358 | 0.43847 | 1.780059 |
| 5.410865 | −1.17619 | 3.473145 | 4.063404 | 1.620256 | 3.25784 | 3.772552 | 5.448089 | 1.208642 | 0.52688 |
| 4.407303 | −0.03957 | 3.795275 | 3.855666 | −0.40527 | 3.769299 | 3.563014 | 5.603838 | 0.365519 | 0.628479 |
| 4.856877 | −1.1287 | 3.071894 | 3.960498 | 1.647983 | 3.17065 | 3.593175 | 5.271299 | 0.861765 | 0.99966 |
| 5.071925 | −1.43876 | 3.277075 | 3.9411.74 | 1.554938 | 4.568442 | 3.240147 | 5.506369 | 1.064586 | 1.116568 |
| 4.125199 | −1.06653 | 2.016205 | 4.006538 | 1.41355 | 3.08703 | 4.022063 | 5.727639 | 1.638045 | 0.282372 |
| 5.347262 | −1.26657 | 0.867364 | 4.092736 | 1.068865 | 3.19852 | 3.483413 | 5.405884 | 1.283368 | 0.948863 |
| 5.141069 | −2.49813 | −0.80213 | 3.481093 | 1.294802 | 2.857394 | 3.19677 | 5.398784 | 0.446838 | 0.359266 |
| 5.047768 | −1.7244 | 2.528369 | 3.56533 | 0.432492 | 2.909509 | 2.9426 | 4.377747 | 1.171661 | 1.542358 |
| 4.836429 | 0.108112 | 4.671816 | 3.666934 | 2.196001 | 0.637641 | 3.445914 | 5.543493 | 0.621825 | 1.978621 |
| 5.342429 | −0.27209 | 4.577732 | 3.691535 | 1.864115 | 1.99256 | 3.683131 | 5.51089 | 1.679691 | 0.860434 |
| 5.102347 | −0.23383 | 4.36158 | 3.637907 | 2.219271 | 3.721016 | 4.157145 | 5.185915 | 0.768625 | 1.813214 |
| 5.184573 | −1.44221 | 2.656688 | 4.24565 | 1.636356 | 3.56904 | 3.355914 | 5.415724 | 1.213157 | 1.035084 |
| 4.937199 | −2.24005 | −1.91818 | 3.508107 | 1.38458 | 2.625785 | 3.43037 | 5.952872 | 1.672211 | 2.073128 |
| 5.138477 | −0.97556 | −0.9728 | 4.011831 | 1.457798 | 1.495196 | 3.69881 | 5.105959 | 1.47189 | 1.058098 |
| 5.253758 | −2.47976 | −1.85202 | 3.790243 | 1.152399 | 3.001151 | 3.293768 | 5.514473 | 0.590249 | −0.30928 |
| 4.527365 | −2.19867 | −1.32273 | 3.606953 | 1.158775 | 2.897728 | 3.43308 | 4.98263 | 0.641871 | 1.935632 |
| 4.767777 | −1.62705 | 1.262.792 | 3.251998 | 1.582416 | −0.00067 | 2.765758 | 4.687111 | 0.865373 | −1.82726 |
| 4.809597 | −0.22859 | 4.142671 | 3.586081 | 1.888399 | 0.106971 | 3.039232 | 5.157597 | 0.780386 | −0.04031 |
| 5.013743 | −1.27521 | 3.011977 | 4.002603 | 1.632145 | 2.696875 | 3.994995 | 5.320207 | 1.367803 | 1.01507 |
| 5.254389 | −0.32847 | 4.448844 | 3.791733 | 2.821282 | 4.255651 | 3.875625 | 5.496891. | 0.929552 | 0.840989 |
| 5.334909 | −2.28647 | 0.619218 | 2.956394 | −0.05251 | 1.646859 | 2.599701 | 4.750539 | 0.108153 | −0.58749 |
| 4.614544 | −1.74698 | −0.28554 | 4.147405 | 1.284534 | 3.293934 | 3.491438 | 5.264256 | 1.062501 | 1.641604 |
| 4.8946 | −2.35584 | −2.08177 | 3.869424 | 0.846746 | 3.097786 | 3.159997 | 5.187624 | 0.905974 | 0.442536 |
| 4.716251 | −0.04072 | −1.10695 | 3.881458 | 1.046506 | 1.852809 | 3.176513 | 4.998498 | 0.437438 | 1.78208 |
| 4.813347 | −1.85932 | −1.14898 | 3.363548 | 1.026906 | 1.295834 | 3.635041 | 4.868615 | 0.26773 | 1.524194 |
| 5.494449 | −0.7293 | 4.002605 | 3.745387 | 1.560187 | 0.513691 | 3.180551 | 5.53533 | 0.482545 | 0.556243 |
| 4.433297 | −1.04734 | 2.800306 | 4.331145 | 2.054859 | 4.160893 | 4.520776 | 5.98227 | 1.726742 | 0.289704 |
| 4.702381 | −1.30134 | 2.79687 | 3.705855 | −1.22548 | 3.958802 | 4.042507 | 5.168281 | 0.38515 | 1.328535 |
| 4.284434 | −1.5693 | 2.520595 | 4.347198 | 1.649089 | 4.211638 | 3.629753 | 5.61865 | 1.263877 | 1.088451 |
| 5.172504 | −1.83573 | −2.29414 | 3.34388 | −2.66342 | 1.916514 | 3.486847 | 4.68702 | 0.671245 | 0.282879 |
| 4.97747 | −2.18883 | −1.79087 | 3.941449 | 1.122542 | 4.141021 | 3.859064 | 5.765992 | 0.975882 | 1.031506 |
| 3.458197 | 0.267442 | 4.413918 | 4.01674 | 2.704743 | −1.70649 | 5.982232 | 6.102159 | 5.430389 | 1.585706 |
| 3.080979 | −0.1732 | 4.295694 | 4.206811 | 2.780619 | −1.6116 | 3.767972 | 6.221582 | 5.306705 | 1.540502 |
| 3.643146 | 1.412383 | 3.946184 | 3.984357 | −1.63029 | −1.62367 | 6.332442 | 5.794658 | 4.659222 | 1.261868 |

| L-threoni [symbol] | L-tyrosine | L-valine | maleic aci [symbol] | malonic a [symbol] | maltose | melibiose | methyl 16 [symbol] | methyl li [symbol] | methyl ol [symbol] |
|---|---|---|---|---|---|---|---|---|---|
| 2.127985 | 2.374457 | 4.115844 | −1.76706 | −1.04479 | 0.799712 | −3.3411 | 2.426431 | 2.377278 | 3.388559 |
| 2.214247 | 2.83973 | 5.087501 | −0.47326 | 0.434997 | 0.20086 | −2.36287 | 2.715914 | 1.749027 | 2.746924 |
| 1.936362 | 3.526712 | 4.17762 | −2.40738 | −0.48782 | 1.124933 | −1.45252 | 2.133109 | 0.553408 | 1.784362 |
| 1.724896 | 3.879414 | 4.643988 | −2.26504 | 0.179264 | 1.235385 | NA | 2.307723 | 1.653374 | 2.508884 |
| 2.187855 | 3.044814 | 4.047575 | −2.07285 | −0.83188 | 1.754839 | −3.44641 | 2.983485 | 2.740017 | 3.646547 |
| 1.590051 | 2.703431 | 4.027493 | −2.35871 | −0.78593 | 0.630555 | −0.57978 | 2.30685 | −0.24855 | 0.446214 |
| 2.188624 | 2.260557 | 3.486099 | −2.74195 | 0.615489 | 1.755415 | −2.83607 | 2.666171 | 2.321079 | 3.080208 |
| 1.493451 | 2.513583 | 2.415199 | 1.734885 | −0.44248 | 0.138941 | NA | 1.657576 | 0.394817 | 1.435137 |
| 0.970395 | 2.104803 | 2.982777 | 3.524448 | 0 | 0.18456 | −2.5512 | 3.584687 | 1.431948 | 2.482381 |
| 1.490406 | 1.664819 | 3.427935 | −2.51571 | −1.05087 | −0.12828 | −2.4888 | 1.537797 | 0.356178 | 1.822877 |
| 2.266651 | 2.801175 | 5.117494 | −2.0408 | −0.11599 | −0.02807 | −3.42443 | 2.593253 | 0.93536 | 1.960319 |
| 2.048339 | 3.686549 | 4.87568 | −1.11094 | −0.5297 | 1.097618 | −1.18477 | 1.528023 | 1.542657 | 2.214979 |
| 1.801441 | 3.849474 | 4.919732 | −2.18928 | 0.551911 | 0.533452 | NA | 2.193125 | 1.45708 | 2.180659 |
| 1.977328 | 2.740583 | 3.829096 | −1.73507 | −1.07257 | 1.557911 | −1.65544 | 2.91293 | 2.128342 | 3.144565 |
| 1.297125 | 1.915431 | 3.018322 | −2.68063 | −0.19233 | −0.58781 | −5.56345 | 1.740684 | 1.643657 | 2.022222 |
| 1.769809 | 1.446685 | 3.88657 | 5.435561 | 1.074357 | 1.321591 | −2.3079 | 1.98713 | 1.889877 | 2.535775 |
| 1.231495 | 1.931259 | 2.216288 | −1.85983 | −1.21947 | 0.393382 | −2.36448 | 2.35556 | 1.684145 | 2.573125 |
| 1.15997 | 1.910544 | 2.941545 | −2.32839 | 0 | 1.371157 | −0.67675 | 2.983632 | 1.640363 | 2.440967 |
| 1.908209 | 1.965077 | 4.061757 | 5.008435 | 0.146709 | 1.020551 | −2.9851 | 2.251022 | −0.70388 | 0.279131 |
| 1.865929 | 2.432357 | 4.694817 | −0.77634 | 1.088566 | 0.305551 | −0.7715 | 1.782748 | 0.880295 | 1.854434 |
| 1.954514 | 3.337602 | 3.809016 | −2.23044 | −0.72122 | 0.613661 | −2.84389 | −0.52155 | 1.047178 | 1.832689 |
| 2.065877 | 3.730611 | 4.88532 | −2.37301 | 0.77428 | 1.433048 | −2.11248 | 2.235553 | 1.669307 | 2.578203 |
| 0.554989 | 1.02391 | 2.255937 | 1.196417 | −2.75925 | 1.147935 | −2.42927 | 2.734035 | 1.507007 | 2.433489 |
| 1.388109 | 1.622334 | 2.867976 | −2.77025 | 1.311191 | 1.989109 | −1.21421 | 2.288737 | 1.705184 | 2.291222 |
| 1.418848 | 2.00457 | 2.17791 | 3.703769 | −1.30549 | 0.059905 | −1.81558 | 2.100599 | 0.429885 | 1.533971 |
| 0.595284 | 2.530409 | 3.681769 | 3.984101 | 0.227986 | 0.770489 | −3.41386 | 2.505462 | 0.129777 | 0.973803 |
| 1.385234 | 1.726049 | 3.255814 | −2.14932 | −1.89113 | 1.057754 | −2.56425 | 2.232 | 1.591594 | 2.294067 |
| 2.135084 | 2.464611 | 4.637801 | −0.00842 | 1.196177 | 0.70824 | −1.56289 | 2.443504 | 1.455844 | 2.375693 |
| 2.167007 | 3.371459 | 3.600548 | −1.60585 | −1.02392 | 0 | −2.56325 | 1.745164 | 0.531602 | 1.923562 |
| 1.499282 | 3.086725 | 4.153034 | 0.970958 | −0.02587 | 0.581974 | −6.1255 | 2.080728 | 0.86894 | 1.632176 |
| 2.161538 | 2.957513 | 3.632145 | −1.33705 | −1.91221 | 0.973592 | NA | 1.807849 | 0.722623 | 2.088931 |
| 1.179277 | 1.71614 | 2.375404 | 3.037172 | −2.07303 | 1.102524 | −1.53835 | 2.870852 | 2.348065 | 3.016055 |
| 1.72818 | 2.473651 | 2.445688 | 2.413029 | −0.58724 | 0.000987 | NA | 3.008438 | 1.262353 | 2.364249 |
| 6.166946 | 4.673046 | 5.429131 | −1.7504 | 1.719304 | 0.535414 | 2.632643 | 1.351046 | −0.15438 | 0.435183 |
| 6.290158 | 4.551205 | 5.095939 | −1.85068 | 1.84845 | 0.521975 | 2.755343 | 0.438334 | 0.151204 | 0.757402 |
| 5.06329 | 4.069245 | 4.674419 | −2.32771 | −0.56713 | 0.678902 | 2.867029 | 0.992166 | −0.11428 | 0.534414 |

| methyl p [symbol] | methylsu [symbol] | monoste [symbol] | myo-inos [symbol] | myristic a [symbol] | N-methyl | nonanoic | oleanitril [symbol] | oleic acid | oxalic aci [symbol] |
|---|---|---|---|---|---|---|---|---|---|
| −0.50114 | −0.83371 | −2.11396 | 3.757482 | 3.093371 | 1.760222 | −0.04415 | 4.574658 | 4.140252 | −2.5687 |
| −1.9885 | −1.40287 | −0.24006 | 2.938692 | 3.180599 | −3.24269 | 0.811343 | 5.335127 | 4.2761.1 | −2.88198 |
| −1.0427 | −1.24464 | −0.42396 | 2.616945 | 3.182643 | −3.44858 | 0.631809 | 5.73315 | 4.730159 | −2.89542 |
| −0.1992 | −1.03743 | −1.81038 | 3.182812 | 2.752976 | 1.267458 | −0.57175 | 4.896088 | 3.802382 | −2.83725 |
| −0.03142 | −1.66833 | −0.15099 | 4.275739 | 3.224822 | 1.666453 | −0.22584 | 4.645308 | 4.356582 | −3.03598 |
| −0.60851 | −1.50522 | 0.468601 | 1.902779 | 3.544652 | −0.68023 | 0.304098 | 5.858702 | 4.544286 | −2.52504 |
| 0.312916 | −1.18574 | −1.04251 | 3.679524 | 2.723218 | 2.159339 | −0.48811 | 4.491797 | 3.49403 | −3.45748 |
| −1.24471 | −1.62759 | 0.198223 | 2.117953 | 3.105437 | −3.37599 | 0.381887 | 5.028144 | 4.759904 | −2.94167 |
| −1.44874 | −1.20226 | −0.06101 | 3.200972 | 3.496429 | −2.91741 | 1.400493 | 6.670418 | 4.54616 | −2.25613 |
| 1.526664 | −2.29881 | −0.23918 | 2.452733 | 2.884828 | −0.03489 | −0.21566 | 3.839167 | 4.456788 | −3.12371 |
| −1.27342 | −1.55489 | −2.20783 | 2.300466 | 3.308483 | −3.08677 | 0.602565 | 5.461641 | 4.226967 | −2.59302 |
| −0.59708 | −0.71074 | −1.64633 | 2.631928 | 3.086765 | 0.498296 | 0.567473 | 4.909487 | 4.541472 | −2.69629 |
| −0.20146 | −1.88387 | 0.204331 | 2.807194 | 3.00416 | −3.75547 | 0.00059 | 4.912191 | 4.516481 | −3.37412 |
| −0.14085 | −1.5084 | −0.9921 | 3.428719 | 3.302679 | 1.330126 | −0.46283 | 4.974801 | 5.02.3701 | −3.11025 |
| 0.370515 | −0.47922 | −1.96778 | 2.474349 | 2.845035 | −2.61175 | 0.49023 | 4.426194 | 4.617726 | −2.31394 |
| −0.21202 | −1.6957 | −0.43267 | 3.148173 | 3.120318 | 1.43829 | −0.33118 | 4.834791 | 4.326953 | −3.07518 |
| −0.49746 | −0.59413 | 0.038442 | 2.76162 | 3.10011 | −2.65481 | 0.252692 | 4.684121 | 4.68706 | −2.62796 |
| −0.76884 | −0.96189 | −1.37223 | 3.081646 | 2.853383 | 0.884776 | −0.04144 | 5.435133 | 4.039203 | −3.06982 |
| 2.130596 | −2.37517 | −0.25375 | 1.835292 | 3.064107 | −3.59053 | 0.568905 | 5.938912 | 4.165302 | −2.53691 |
| 2.746575 | −1.62714 | −0.1232 | 1.960383 | 2.712825 | −2.94313 | 0.317814 | 4.551167 | 4.091605 | −3.0297 |
| −0.8853 | −1.40338 | −1.74749 | 2.389325 | 2.851721 | 0.599834 | 0.01803 | 3.639007 | 4.272763 | −3.00315 |
| −0.23925 | −1.31879 | −0.70967 | 3.094856 | 3.233841 | −2.69325 | 0.344957 | 4.84568 | 4.611634 | −2.91977 |
| 1.977372 | −0.65486 | −1.3467 | 2.903973 | 3.181416 | −4.2211 | 0.649591 | 5.619841. | 4.52313 | −2.35442 |
| 0.051244 | −1.46697 | −1.3184 | 3.000409 | 2.776547 | 1.233485 | −0.53044 | 4.633922 | 3.972593 | −3.55256 |
| −0.76703 | −2.18401 | 0.179625 | 2.128574 | 2.84496 | −2.55642 | 0.438118 | 5.172297 | 4.562338 | −2.98833 |
| 2.414854 | −1.04631 | −0.03089 | 1.446523 | 3.377085 | −2.89947 | −1.28998 | 6.131715 | 4.344818 | −2.0487 |
| 0.809017 | −1.08217 | −1.03619 | 2.672816 | 2.966179 | 0.55174 | 0.2.47719 | 5.477398 | 4.154978 | −3.03295 |
| −0.84343 | −0.6624 | 0.877611 | 2.68027 | 3.556691 | −2.24704 | 0.839936 | 5.303513 | 4.839133 | −2.52471 |
| 0.026246 | −1.1186 | −1.79311 | 2.646412 | 2.677879 | 0.840011 | −0.15716 | 5.149547 | 3.82289 | −3.01083 |
| 2.260985 | −1.11629 | −0.83104 | 1.947972 | 3.039819 | −0.1734 | −0.93995 | 5.337596 | 4.214284 | −2.85253 |
| 0.677506 | −2.51216 | −0.71011 | 2.870268 | 2.514108 | 1.577218 | −1.05725 | 3.542139 | 3.886527 | −3.27526 |
| −0.67104 | −1.65183 | −0.88524 | 3.417367 | 2.87098 | 0.167956 | −0.44432 | 4.677812 | 4.252552 | −3.44263 |
| 0.231411 | −1.12746 | 0.056973 | 3.115517 | 3.094253 | −4.34626 | 0.536945 | 5.507355 | 4.585989 | −2.65011 |
| −0.79216 | −2.23704 | −1.46419 | 3.185505 | 1.654401 | −4.21695 | −0.59127 | 4.866795 | 2.90948 | −3.41723 |
| 0.483205 | −3.27195 | −1.25876 | 4.940211 | 1.071128 | −4.16352 | −1.1075 | 3.579464 | 2.472691 | −3.6731 |
| −0.83227 | −3.1011 | −0.21727 | 3.050128 | 1.617569 | −3.62532 | −1.16122 | 4.444689 | 3.112294 | −3.29704 |

| palatinos [symbol] | palmitic a [symbol] | palmitole | pentadec [symbol] | pentaned [symbol] | phosphat [symbol] | phytosphi [symbol] | porphine | pseudo u [symbol] | pyrogallo [symbol] |
|---|---|---|---|---|---|---|---|---|---|
| NA | 6.543708 | 0.933004 | 3.743339 | −1.2832 | 6.600635 | 0.183632 | −1.96638 | NA | −1.42467 |
| NA | 6.486906 | 0.905331 | 0.690106 | −2.03606 | 5.524887 | 1.394754 | 2.134022 | NA | −0.72268 |
| NA | 6.683028 | 1.447539 | 3.108737 | −1.96927 | 5.625008 | 0.161118 | 3.511015 | NA | −2.26609 |
| NA | 6.202815 | 0.586395 | 3.251281 | −1.70147 | 6.363802 | 0.078976 | −2.48782 | NA | −0.36742 |
| NA | 6.65372 | 1.236749 | 3.620132 | −1.25997 | 6.003502 | 1.235716 | 2.673765 | NA | −3.22374 |
| NA | 6.555664 | 1.170271 | 3.36706 | −1.20757 | 5.96919 | 1.382425 | 1.640737 | NA | −3.2283 |
| NA | 5.908905 | 0.75739 | 3.419473 | −0.70774 | 5.815475 | −0.17778 | 2.233916 | NA | −2.28282 |
| NA | 6.649812 | 1.609918 | 3.447325 | −1.57758 | 5.42986 | 1.62984 | 3.197595 | NA | −2.77761 |
| NA | 6.865421 | 1.336662 | 3.773746 | −0.64421 | 6.659811 | 2.000576 | 2.663543 | NA | −2.55072 |
| NA | 6.398424 | 1.092935 | 3.51213 | −1.70215 | 5.287146 | 2.496829 | 3.22209 | NA | −4.36699 |
| NA | 6.584847 | 1.026733 | 1.387958 | −1.86101 | 5.804335 | 2.436509 | 2.743248 | NA | −0.49415 |
| NA | 6.60693 | 1.398205 | 3.115469 | −0.91852 | 6.408264 | 0.057244 | 2.605166 | NA | −1.44999 |
| NA | 6.541197 | 1.105389 | 3.303899 | −2.11008 | 5.61673 | 0.307588 | 2.648084 | NA | −2.88711 |
| NA | 6.65544 | 1.680216 | 3.381351 | −1.29594 | 5.729321 | 1.464166 | 2.250726 | NA | −2.49565 |
| NA | 6.70151 | 0.990598 | 3.164452 | −0.04167 | 6.834333 | 1.978465 | −1.50333 | NA | −1.64036 |
| NA | 6.347148 | 1.4684 | 3.046803 | −0.48985 | 5.519974 | 0.545388 | 2.448271 | NA | −2.34477 |
| NA | 6.739792 | 1.524749 | 3.849431 | −1.11203 | 6.452454 | 2.882875 | −6.71731 | NA | −1.90986 |
| NA | 6.232391 | 0.853328 | 3.499744 | −1.02652 | 6.038301 | 2.503873 | 1.711334 | NA | −3.54983 |
| NA | 6.427306 | 0.684013 | 2.906579 | −1.69581 | 5.461425 | 1.727505 | 4.158251 | NA | −2.60053 |
| NA | 6.223413 | 0.898718 | 0.92103 | −2.2856 | 5.098631 | 2.287512 | 2.897328 | NA | −2.77883 |
| NA | 6.204572 | 1.164435 | 3.123823 | −1.75712 | 5.891695 | 0.481619 | 3.362771 | NA | −0.96997 |
| NA | 6.812252 | 1.349845 | 3.452313 | −1.75293 | 6.051751 | 0.619165 | 1.638128 | NA | −2.23031 |
| NA | 6.759988 | 1.4521.9 | 3.469237 | −1.17739 | 6.556299 | 0.702718 | 2.910499 | NA | −1.99672 |
| NA | 6.044157 | 1.008931 | 3.457377 | −1.39213 | 5.669977 | 1.549187 | 2.212921 | NA | −3.02126 |
| NA | 6.530616 | 1.250353 | 3.730672 | −1.5459 | 5.510291 | 2.963896 | 3.729864 | NA | −2.43171 |
| NA | 6.293505 | 0.902971 | 3.52482 | −1.17119 | 6.323125 | 1.143612 | 3.217934 | NA | −2.85237 |
| NA | 6.295177 | 0.91.866 | 3.567339 | −1.33601 | 6.167971 | 1.656842 | 2.445021 | NA | −1.6344 |
| NA | 6.948786 | 1.447629 | 0.878293 | −1.54132 | 5.489953 | 2.579495 | 3.598358 | NA | −1.73765 |
| NA | 5.865973 | 1.111137 | 3.358797 | −1.94053 | 6.182317 | 0.491405 | 1.379835 | NA | −1.68807 |
| NA | 6.411344 | 0.932359 | 3.176604 | −2.6652 | 5.516715 | 1.003889 | 1.635399 | NA | −3.73706 |
| NA | 5.93457 | 1.04156 | 3.688587 | −2.22605 | 5.45753 | 1.208204 | 2.578036 | NA | −3.18977 |
| NA | 6.570071 | 1.218835 | 3.529786 | −1.14466 | 5.494232 | 2.494637 | 2.903186 | NA | −1.50341 |
| NA | 6.580455 | 1.368261 | 3.945077 | −1.55229 | 6.044132 | 2.167386 | 2.567365 | NA | −1.47011 |
| 2.620623 | 5.13398 | 0.64306 | 0.480962 | −2.97324 | 5.104748 | −1.42982 | 2.021262 | 2.038913 | −4.04624 |
| 3.198306 | 4.780218 | −0.28334 | 0.934067 | −3.45053 | 4.985275 | −1.58741 | 1.417794 | 2.22209 | −3.27008 |
| 3.391893 | 5.194478 | −0.43112 | 0.587525 | −3.26455 | 4.712371 | 0.764775 | 1.049481 | 2.158893 | −4.0763 |

| pyruvic a [symbol] | quinic aci [symbol] | raffinose | sebacic a [symbol] | shkimic a [symbol] | stearic ac [symbol] | stigmaste [symbol] | succinic a [symbol] | sucrose | syringic a [symbol] |
|---|---|---|---|---|---|---|---|---|---|
| −0.4562 | NA | NA | 1.158269 | 0.996348 | 5.353363 | −0.2453 | 5.138901 | −1.20554 | 1.650299 |
| −1.11031 | NA | −1.2661 | 1.005038 | −2.52912 | 5.297301 | 1.40164 | 1.649807 | −0.08262 | −0.20022 |
| 0.426123 | NA | NA | −0.08514 | −2.00459 | 5.522289 | 0.42598 | 4.186039 | −0.59878 | 1.409593 |
| −0.57503 | NA | NA | 0.924811 | 1.427225 | 5.212942 | −0.05696 | 3.845654 | −1.11671 | 0.971937 |
| 0.4221 | NA | NA | 1.382692 | −2.54171 | 5.939787 | 1.333656 | 5.113635 | 1.1585 | 3.372618 |
| 0.000896 | NA | NA | 0.400628 | 0.430096 | 5.625324 | 0.297471 | 6.441632 | −0.31824 | 1.563861 |
| −0.45901 | NA | −1.91865 | 0.848192 | −1.43917 | 4.941444 | 0.517817 | 5.707652 | −0.36723 | 2.302714 |
| −0.0105 | NA | NA | 1.059418 | 0.573939 | 5.493381 | 1.412808 | 3.585861 | −0.79367 | 1.14587 |
| −0.7888 | −2.46343 | NA | 1.565301 | −1.14226 | 5.972535 | 1.295249 | 5.230085 | −1.4811 | 2.385727 |
| −0.05092 | NA | NA | 0.645464 | 0.452393 | 5.358359 | 2.317244 | 5.481832 | −1.83222 | 1.847557 |
| −0.46794 | NA | NA | 1.117226 | −1.80983 | 5.567734 | 2.860411 | 1.576993 | −0.56058 | 0.945427 |
| 0.000466 | NA | NA | −0.37973 | −2.05955 | 5.118437 | −0.34294 | 5.467393 | −1.03241 | 0.504289 |
| −0.73407 | NA | NA | 0.371911 | 0.895299 | 5.545617 | 0.310321 | 5.206074 | −0.87674 | 1.024895 |
| −0.40875 | NA | NA | 0.748285 | −1.94823 | 5.575444 | 1.781394 | 5.478351 | −1.39514 | 1.442149 |
| −1.14247 | NA | NA | 0.117075 | 1.021858 | 5.644664 | −0.96684 | 7.700437 | −1.29048 | 1.595932 |
| −0.49653 | NA | NA | −0.2394 | −1.69076 | 5.199597 | 1.199376 | 7.053045 | −0.7917 | 1.825583 |
| −0.50314 | NA | NA | 1.059555 | 0.054347 | 5.587422 | 1.828392 | 4.884376 | −1.72066 | 1.277529 |
| −0.48843 | NA | NA | 0.914967 | −2.08179 | 5.40094 | 1.542192 | 5.187691 | −1.01688 | 3.061997 |
| −0.04422 | NA | NA | 0.074879 | −0.39404 | 5.392516 | −0.26734 | 6.693985 | −0.28364 | 1.485021 |
| −0.36832 | NA | −1.82993 | 1.220469 | −1.79232 | 5.02023 | 3.031926 | 1.320988 | 0.06467 | −2.35142 |
| 0.175701 | NA | NA | 0.246498 | −1.56429 | 5.054081 | 0.64556 | 4.48122 | −1.32011 | 1.237049 |
| −0.16085 | NA | NA | 1.002106 | 1.274219 | 5.608455 | 0.109144 | 5.38022 | −1.07195 | 1.082511 |
| −0.67688 | NA | NA | 1.075428 | 0.804977 | 5.557317 | 0.944848 | 2.951916 | −1.11537 | 1.792323 |
| −0.70122 | NA | −2.36688 | 0.472259 | −1.30234 | 5.168504 | 1.734258 | 4.53766 | −0.54913 | 2.244869 |
| −0.30528 | NA | NA | 1.393734 | 0.183032 | 5.470158 | 2.020962 | 5.41131 | −1.29411 | 1.383064 |
| −0.75746 | NA | NA | 0.41107 | −1.96025 | 5.259034 | 1.202427 | 5.673528 | −1.11553 | 2.303233 |
| −0.37654 | NA | NA | 0.141026 | −0.46239 | 4.95654 | 2.049683 | 4.280022 | −1.73094 | 1.969783 |
| −0.15344 | NA | NA | 1.469412 | −1.58752 | 5.781642 | 2.444659 | 2.062604 | −0.00545 | 1.160683 |
| 0.17168 | NA | NA | −0.47917 | −1.29903 | 4.787159 | 0.419804 | 3.613471 | −1.20063 | 1.606739 |
| 0.093044 | NA | NA | 0.351761 | 1.322908 | 5.397919 | 0.462448 | 2.888768 | −0.89767 | 1.286882 |
| −0.39784 | NA | NA | 0.421544 | −2.55379 | 4.941884 | 0.713022 | 4.291159 | −1.83587 | 2.013988 |
| −0.73544 | NA | NA | 1.262579 | 3.449346 | 5.627766 | 0.40291 | 4.754353 | −1.21873 | 2.33714 |
| −0.55192 | NA | NA | 1.68192 | 1.082349 | 5.553413 | 1.923497 | 4.149034 | −1.34288 | 1.680496 |
| −1.14664 | 2.139967 | 6.601546 | −2.21815 | 2.361941 | 4.448555 | −0.0064 | 1.570783 | 1.163098 | 0.300532 |
| −1.2618 | 1.844079 | 6.754505 | −1.84062 | 2.511755 | 4.337523 | 1.21372 | 0.691583 | 1.233174 | 0.335115 |
| −1.11383 | 2.101496 | 6.72978 | −2.06164 | 2.526375 | 4.584125 | −0.9384 | 1.625927 | 1.169566 | 0.344638 |

| thymine | trans-4-h [symbol] | uracil | urea | Unknown | Unknown | Unknown | Unknown | Unknown | Unknown |
|---|---|---|---|---|---|---|---|---|---|
| −0.3688 | 1.225113 | 4.750287 | −0.26861 | −1.7015 | 1.527692 | 1.157726 | 0.434564 | −1.95724 | −1.59638 |
| −0.13666 | 1.993731 | 3.37946 | 1.427615 | −1.94384 | 1.234491 | 1.646679 | −0.23511 | −1.86004 | −1.64343 |
| −0.24624 | 2.33439 | 2.702023 | 1.551096 | −1.61221 | 2.380351 | 1.497095 | −0.37035 | −2.96961 | −1.45434 |
| −0.89731 | 2.920284 | 4.514724 | 0.604018 | −2.87441 | 2.093838 | 0.881819 | −0.55709 | −3.5783 | 1.986017 |
| −0.36852 | 0.894854 | 3.500913 | −0.08112 | −1.66272 | 2.196619 | 1.951558 | −0.30039 | −3.09398 | −1.79599 |
| −0.53822 | 0.994039 | 3.441409 | 0.224145 | −2.09182 | 2.700613 | 1.912917 | 0 | −3.66194 | −2.41174 |
| −0.3517 | 2.322061 | 3.010723 | 0.199008 | −1.60591 | −0.14677 | 1.347037 | −1.35986 | −3.2556 | −1.51039 |
| −0.19203 | 1.02546 | 2.748057 | 0.698139 | −1.96631 | 0.943646 | 1.65915 | −0.47828 | −3.0058 | −1.46052 |
| 0.056695 | 1.662075 | 3.999678 | 0.565948 | −1.45569 | 0.711973 | 0.700897 | 0.541335 | −3.08835 | −1.25037 |
| −0.30484 | 1.672839 | 2.431467 | 0.492894 | −1.75736 | 1.613415 | 1.40642 | −0.56406 | −3.64727 | −2.10327 |
| −0.4499 | 1.951247 | 3.870972 | 1.511361 | −1.0456 | 1.027041 | 1.391804 | −0.00498 | −0.8703 | −1.04935 |
| −0.47126 | 2.491082 | 4.504262 | 0.948507 | −1.07307 | 2.103444 | 1.255873 | 0.617886 | −3.80751 | −1.16524 |
| −0.70975 | 2.922827 | 2.618448 | 0.368792 | −1.88226 | 1.762857 | 1.192927 | −0.85653 | −3.54084 | −1.21324 |
| −0.51836 | 0.997609 | 3.411436 | 0.44748 | −1.87789 | 2.533557 | 1.989429 | −0.40343 | −2.63189 | −1.77677 |
| −0.31529 | 1.453944 | 4.950606 | −0.11651 | −1.01886 | 2.133874 | 1.268646 | 1.102464 | −3.06137 | −1.57899 |
| −0.6262 | 1.951603 | 2.679898 | 0.401467 | −1.68663 | 0.095352 | 1.667133 | −0.3735 | −2.7968 | −1.74116 |
| −0.28611 | 0.881683 | 4.665758 | −0.25995 | −1.90156 | 1.717238 | 1.571117 | 0.170227 | −2.69371 | −1.91906 |
| −0.21843 | 1.767759 | 3.933471 | 0.374578 | −1.83531 | −0.14001 | 0.718692 | −0.16652 | −3.41027 | −1.63865 |
| −0.34216 | 2.686914 | 2.634081 | 0.958288 | −1.26762 | 1.543466 | 1.557781 | −0.16717 | −3.5087 | −1.38815 |
| −0.22251 | 1.243427 | 2.867915 | 1.191049 | −1.60788 | 0.517756 | 1.608263 | −0.56867 | −3.58956 | −1.27192 |
| −0.158 | 2.249896 | 3.583837 | 0.532805 | −1.44499 | 2.622964 | 1.665853 | −0.05026 | −3.40383 | −1.57267 |
| −0.66284 | 2.717717 | 3.545472 | 0.020962 | −2.03414 | 2.22388 | 0.387924 | −0.35912 | −3.9328 | 2.204978 |
| 0.198031 | 0.6243 | 4.634396 | −0.69251 | −0.98662 | 0.953433 | 0.759341 | 0.606239 | −3.35682 | −0.01579 |
| −0.67265 | 1.82003 | 2.923155 | 0.238762 | −2.0633 | −0.5077 | 1.52593 | −0.92304 | −2.99223 | −1.57173 |
| −0.15608 | 0.727271 | 2.611931 | 1.482012 | −1.82088 | 1.544516 | 1.713519 | −0.37958 | −3.18988 | −1.78253 |
| 0.646658 | 0.816669 | 3.731235 | 1.367602 | −1.04341 | 1.098036 | 1.749935 | 0.627835 | −1.92747 | 1.161417 |
| −0.20011 | 1.76355 | 4.385902 | −0.00316 | −1.2313 | 1.501148 | 0.802298 | 0.06426 | −2.25095 | −1.76475 |
| −0.12396 | 1.65898 | 3.161775 | 1.640944 | −1.42701 | 1.557897 | 1.753368 | −0.05631 | −2.9252 | −1.51632 |
| −0.51924 | 2.425629 | 4.164708 | −0.24326 | −1.88628 | 3.226605 | 1.364293 | −1.64626 | −3.20479 | −1.52178 |
| −1.53221 | 2.759556 | 3.404478 | 0.242481 | −2.14364 | 1.851188 | 1.062108 | −0.51177 | −1.80739 | 1.49954 |
| −0.39069 | 0.897642 | 2.391167 | 0.332538 | −1.6821 | 2.246872 | 2.112586 | −0.61248 | −3.47512 | −0.96129 |
| −0.7706 | 1.669858 | 3.087322 | 0.802371 | −1.81217 | 3.175364 | 1.275568 | −0.72583 | −3.36591 | −1.59287 |
| 0.168141 | 1.268603 | 3.364693 | 0.368672 | −1.76322 | 2.524576 | 1.813937 | −0.18125 | −3.21543 | −0.63559 |
| −0.22173 | 3.063882 | 3.407118 | 7.598364 | −2.13944 | −0.97275 | 2.505104 | 0.92972 | −4.00648 | −1.00745 |
| −0.06015 | 2.348334 | 2.701904 | 7.613924 | −2.78742 | −1.21464 | 2.498563 | 1.56598 | −2.01574 | −2.02806 |
| −0.11011 | 3.019499 | 2.961763 | 6.718807 | −3.01685 | −0.81056 | 2.352415 | −1.07833 | −2.33583 | −1.91085 |

| Unknown | Unknown | Unknown | Unknown | Unknown | Unknown | Unknown | Unknown | Unknown | Unknown |
|---|---|---|---|---|---|---|---|---|---|
| 0.607786 | −2.60335 | −0.1913 | 0.851188 | 0.709819 | 0.333957 | −2.93292 | −2.42959 | 0.377268 | 1.212564 |
| 0.277315 | −1.3317 | 3.696042 | 1.44166 | −0.63757 | −0.58438 | −4.70501 | −0.79498 | −0.04412 | 0.589809 |
| −1.03399 | −1.19301 | −0.54755 | 0.948521 | −0.57492 | −1.02031 | −3.49574 | −3.13202 | 0.310052 | 0.42728 |
| −0.13784 | −3.54856 | 3.283554 | −0.04622 | −0.54003 | 0 | −3.36147 | −4.11273 | −0.22846 | 1.007455 |
| 0.412856 | −1.8063 | −1.12297 | 1.636309 | −0.38206 | −0.66127 | −2.68776 | −0.50045 | 0.816007 | 0.344285 |
| 0.650651 | −1.53876 | 2.961414 | 0.879907 | −0.11267 | −0.40879 | −3.50198 | −0.99025 | −0.17718 | −0.59044 |
| 0.16784 | −1.98749 | 0.255444 | 1.852899 | −0.37002 | −1.34944 | −3.28232 | −0.18099 | −0.45196 | −0.69376 |
| 0.051887 | −1.46362 | 1.115359 | 0.181678 | −0.65943 | −0.88194 | −3.15048 | −3.31112 | 0.84637 | 0.036612 |
| 0.332821 | −0.77251 | 1.868779 | 0.286572 | −0.49719 | 0.281212 | −2.91301 | −0.76274 | 0.412903 | 0.855329 |
| 0.552666 | −0.92166 | 2.282287 | 0.409445 | 0.659751 | −1.06522 | −2.98821 | −2.44894 | 0.644225 | −0.30509 |
| 0.652509 | −1.82293 | 3.932261 | 1.960765 | −0.52392 | −0.19815 | −2.77572 | −0.78073 | 1.569612 | 0.901675 |
| 0.557913 | −1.46065 | −0.43954 | 1.817554 | −0.71066 | 0.274382 | −2.42003 | −3.07195 | 1.402108 | 0.922343 |
| 0.144839 | −1.3025 | 3.849162 | 0.327454 | −0.79934 | −1.20372 | −3.10354 | −2.43118 | −0.034 | −0.06911 |
| 0.301902 | −1.68604 | 2.564582 | 2.058712 | −0.2882 | −0.75811 | −2.90332 | −0.58777 | 0.666992 | 0.290641 |
| 1.163652 | −0.87112 | 2.452666 | 0.665013 | −1.27331 | 0.702785 | −2.88677 | −0.71247 | 0.398112 | 0.744702 |
| 2.648816 | −1.31839 | 2.343498 | 1.958354 | −0.16422 | −1.15549 | −2.7909 | −0.7094 | 0.738266 | −0.47382 |
| 0.805554 | −3.12204 | 0.693176 | 0.189919 | −0.17332 | 0.518193 | −2.93476 | −1.53497 | −0.06552 | 0.884862 |
| 0.35255 | −1.97331 | 2.0432.55 | 1.18225 | −0.73133 | −0.27368 | −2.42091 | −0.30664 | 0.324677 | 0.903371 |
| 0.000669 | −0.84841 | 1.802912 | 1.333407 | −0.30949 | −0.74333 | −3.71411 | −1.29162 | 0.317255 | 0.028288 |
| −0.007 | −1.36879 | 3.662512 | 0.661891 | 0.109237 | −0.84949 | −3.52893 | −1.71554 | 0.739962 | 0.108284 |
| 0.283429 | −1.77488 | 0.39394 | 1.632763 | −1.1294 | −0.6005 | −3.28794 | −3.5242 | 1.06443 | 0.451244 |
| −0.03401 | −1.09984 | 3.537371 | 0.985653 | −0.55073 | −0.42883 | −3.53511 | −2.05172 | 1.168572 | 0.61282 |
| 1.083355 | −1.95653 | 1.90209 | 1.025679 | −1.34431 | 0.551061 | −2.27081 | −0.43435 | 1.106879 | 0.304598 |
| −0.16532 | −2.38864 | 0.289587 | 1.131731 | −0.30869 | −1.30834 | −2.82508 | 0.067282 | −1.60269 | −0.59266 |
| −0.11635 | −1.74381 | 1.754701 | 0.003771 | −0.81438 | −0.80661 | −2.91443 | −2.72342 | −0.48437 | 0.043364 |
| 0.478397 | −0.655 | 1.891365 | 0.662357 | −0.37499 | 0.085871 | −2.46516 | −0.56444 | 1.665731 | 0.834674 |
| 0.878657 | −1.91774 | 2.395942 | 0.923465 | −0.64558 | −0.00891 | −3.21222 | −3.28514 | −0.07948 | 0.839634 |
| 0.341194 | −0.99257 | 2.933913 | 1.387495 | −0.5336 | −0.55569 | −4.4687 | −0.38804 | 1.307117 | 0.758931 |
| 0.229077 | −1.90321 | 2.062635 | 2.170375 | −0.0856 | −0.35522 | −3.02457 | −3.17268 | 0.955793 | 0.790952 |
| −5.17273 | −2.37855 | 2.103385 | 0.747696 | −0.5309 | −1.17914 | −3.6662 | −0.75343 | 0.6768 | −0.4358 |
| 0.257942 | −0.84878 | 2.95446 | 1.629365 | −0.61441 | −1.53926 | −2.70944 | −0.66506 | 0.204497 | −0.28337 |
| 0.297033 | −1.69514 | 2.472549 | 1.169249 | −1.00332 | −1.03384 | −3.65662 | −0.08776 | −0.81664 | −0.35913 |
| 0.148646 | −1.2319 | 2.876716 | 0.426316 | −0.61996 | −0.51549 | −1.78992 | −2.82152 | 1.043929 | 0.323295 |
| −0.4321 | −2.4419 | 5.021874 | 0.125422 | −0.92485 | −1.30769 | −3.66384 | −3.33034 | 0.388543 | −0.95632 |
| −0.15271 | −2.34618 | 3.769399 | 0.096241 | −1.87559 | −5.05064 | −3.73121 | −3.25436 | −0.06298 | −1.50563 |
| −0.62883 | −2.28213 | 2.985231 | −0.03138 | −0.61912 | −1.96976 | −3.9108 | −4.16156 | −0.1733 | −1.53012 |

| Unknown | Unknown | Unknown | Unknown | Unknown | Unknown | Unknown | Unknown | Unknown | Unknown |
|---|---|---|---|---|---|---|---|---|---|
| −1.74555 | 0.098704 | 1.650255 | 2.30376 | −0.71553 | −1.1121 | −1.50167 | −1.68763 | −2.813 | −0.39067 |
| −0.68947 | 0.207789 | 0.705023 | 1.934548 | −0.42868 | −1.0012 | −0.93385 | −2.49572 | −2.95449 | −0.51616 |
| −0.96087 | 0.565112 | 0.894903 | 2.191626 | −0.35262 | −0.42219 | −0.52393 | −2.015S48 | −2.80596 | −0.3278 |
| −1.7762 | −0.37625 | 1.315342 | 1.833693 | −0.30655 | 0.48505 | −1.85118 | −1.9439 | −2.41346 | −0.77349 |
| −0.85639 | 0.846943 | 0.158164 | 3.039814 | −0.4156 | −0.19685 | −1.57715 | −2.00601 | −2.78168 | −0.10818 |
| −2.00163 | −5.82854 | 0.759438 | 3.530721 | −0.29312 | 0.351388 | −0.98318 | −2.50075 | −2.58499 | 0.330577 |
| −1.0645 | 0.516319 | 0.54973 | 1.829121 | −0.59252 | 0.431236 | −1.58779 | −2.06108 | −2.31743 | 0.1258 |
| −1.49396 | 0.506852 | 0.108871 | 2.796594 | −0.1923 | 0.566151 | −0.97983 | −2.84778 | −3.28578 | −0.01786 |
| −1.03264 | 0.306354 | 1.492177 | −0.3152 | 0.050957 | −0.81653 | 0.07202 | −2.02805 | −2.72032 | 0.122918 |
| −1.70315 | 0.901001 | 0.261723 | 2.637959 | −0.79653 | −0.54306 | −1.97939 | −2.50271 | −2.85233 | 0 |
| −0.60833 | 0.610347 | 0.8779 | 1.744064 | −0.53075 | −0.5143 | −0.81542 | −2.79297 | −3.84082 | 0.675599 |
| −0.98198 | 0.465297 | 1.429263 | 1.962998 | −0.03874 | −0.59056 | −0.74053 | −2.36028 | −2.62362 | −1.03459 |
| −1.56958 | 0.568285 | −0.13661 | 2.067752 | −0.1955 | 0.499031 | −1.07966 | −2.18048 | −2.55652 | −1.16199 |
| −0.93909 | 0.527423 | 1.39541 | 3.475059 | −0.91642 | 0.566272 | −1.55554 | −2.25203 | −2.75888 | −0.1103 |
| −0.96713 | 0.527249 | 1.719761 | 2.43326 | 0.083565 | 0.847785 | −0.61597 | −2.57267 | −3.41217 | 0.870342 |
| −1.40418 | 0.363098 | 1.05376 | 2.067112 | −1.51509 | 0.745524 | −1.12121 | −3.05473 | −3.5209 | 0.10538 |
| −1.09079 | 0.128256 | 1.328846 | 2.345948 | −0.03279 | −0.30909 | −1.52583 | −2.29718 | −3.09087 | 0.704184 |
| −0.74783 | 0.079134 | 0.87547 | 1.337655 | −0.64919 | −0.40218 | −0.7376 | −2.61502 | −3.56757 | 0.869911 |
| −1.35217 | 1.012089 | 0.277784 | 2.5344 | −1.13441 | 0.753039 | −1.13824 | −2.94076 | −4.34355 | −0.36627 |
| −0.95368 | 0.452461 | 0.504713 | 2.086521 | −0.85487 | 0.190101 | −1.0117 | −2.54267 | −3.48343 | 0.760267 |
| −0.98378 | 0.515034 | 1.003806 | 2.093731 | −0.53214 | −0.66344 | −1.87586 | −2.23831 | −3.1454 | −0.20528 |
| −1.35027 | 0.31892 | 0.412173 | 1.893952 | 0.04599 | 0.240703 | −0.96849 | −2.19468 | −2.58432 | −0.5922 |
| −0.74154 | 0.659424 | 1.512176 | 1.371699 | −0.22709 | 0.496917 | −0.27241 | −2.1658 | −3.38658 | −0.22605 |
| −1.30752 | 0.416364 | 0.196713 | 2.017069 | −0.92202 | 0.493799 | −1.31869 | −1.8471 | −3.28063 | 0.38231 |
| −1.34282 | 0.693955 | 0.384879 | 2.260672 | −0.25417 | −0.2733 | −1.2281 | −2.42069 | −3.09896 | 0.476381 |
| −1.11728 | 0.834288 | 1.438918 | 1.483794 | −0.47 | 0.599824 | −0.74269 | −2.31884 | −2.40769 | 0.611421 |
| −0.90397 | 0.451859 | 1.121596 | 1.604273 | −0.33383 | −0.81565 | −0.86255 | −2.17916 | −2.90902 | −0.44979 |
| −0.60132 | 0.692795 | 0.771796 | 2.152191 | −0.39951 | −0.18966 | −0.91327 | −2.59578 | −3.10795 | −3.14976 |
| −0.8858 | 0.302543 | 1.346124 | 2.417167 | −0.56682 | −0.32428 | −1.55074 | −2.45437 | −3.47984 | −0.27135 |
| −2.09538 | 0.084536 | −0.01806 | 1.60603 | −1.73608 | 0.836309 | −1.64465 | −4.2175 | −5.07085 | 0.695404 |
| −1.06997 | 0.448396 | 0.888028 | 3.38995 | −1.33423 | −0.60195 | −2.29597 | −2.92989 | −2.77584 | −2.04351 |
| −0.80488 | 0.470132 | 0.418226 | 1.753925 | −0.76108 | 1.415059 | −1.0129 | −2.6192 | −4.07122 | −0.07554 |
| −1.05039 | 0.521739 | 0.455664 | 2.560554 | 0.176685 | −1.17924 | −0.76757 | −2.58185 | −2.78514 | −0.07328 |
| −3.03374 | 0.662708 | −0.06734 | 4.831724 | −2.33547 | 0.787438 | −1.46499 | −2.58413 | −3.10493 | −0.28711 |
| −3.52095 | 0.171372 | −0.3745 | 4.823774 | −2.13707 | 0.52988 | −2.11878 | −2.96352 | −3.45541 | −1.39438 |
| −2.87189 | −1.38727 | −0.1186 | 4.510708 | −3.37583 | 0.031383 | −2.74213 | −3.69299 | −3.89711 | −0.73877 |

| Unknown | Unknown | Unknown | Unknown | Unknown | Unknown | Unknown | Unknown | Unknown | Unknown |
|---|---|---|---|---|---|---|---|---|---|
| 0.190888 | 1.105833 | −2.88896 | 1.737036 | 0.090048 | −1.04187 | 0.238057 | −2.39831 | −1.12433 | −1.13914 |
| 0.640743 | 0.626956 | −0.64362 | 1.160643 | −0.40432 | 0.186325 | −0.279 | −1.51817 | −0.15962 | 1.623796 |
| 1.024254 | 1.022065 | −3.25082 | 0.216143 | −0.20438 | 0.173227 | −0.36759 | −1.03895 | −1.61302 | 0.334856 |
| −0.50656 | 2.005105 | −1.01654 | 0.991566 | −2.71177 | 0.247401 | −2.14888 | −1.82788 | −1.5284 | 0.518514 |
| 0.412949 | 1.372852 | −4.73619 | −0.20236 | −0.36698 | −0.44455 | 0.571564 | 1.50745 | 0.750207 | 1.132929 |
| 1.835539 | −0.01759 | −1.90136 | 1.723486 | −0.71847 | −0.67111 | −1.2895 | −1.92167 | −1.19405 | −1.13566 |
| −0.36966 | 0.777726 | −4.28478 | 0.716964 | −1.59597 | −1.10913 | −1.86491 | −2.03405 | −0.7702 | 1.361429 |
| 1.047283 | −1.07824 | −0.90975 | 0.901097 | −0.49584 | −0.3441 | −1.27691 | −1.38609 | −0.81449 | 0.949175 |
| 0.247504 | 0.793899 | −0.2631 | 1.17884 | −0.25946 | 0.030436 | 0.073889 | −1.08583 | −1.15679 | −0.54656 |
| 0.372804 | 1.335896 | −1.61725 | 1.972483 | −1.65677 | −0.98518 | −0.98391 | −1.73444 | −1.35727 | −1.23002 |
| 1.298545 | −0.40263 | −1.22217 | −0.01144 | −1.06028 | 0.120908 | 0.1493 | −1.53321 | −0.54482 | −0.2574 |
| 0.165455 | 0.803015 | −4.66037 | 1.416552 | −0.91253 | −0.24385 | −0.12039 | −1.80662 | −2.33076 | 0.971875 |
| −0.34657 | 0.81592 | −1.44915 | 0.926864 | −0.97952 | −1.50479 | −2.34599 | −1.56999 | −2.34702 | 0.765557 |
| 0 | −0.85922 | −4.61292 | 0.82998 | −0.59654 | −0.52261 | −1.20644 | −1.56952 | 0.114008 | 0.726578 |
| 1.619947 | 0.68482 | 0.64279 | 2.575319 | −0.83637 | 0.238166 | 0.16576 | 1.54887 | −1.05961 | 1.436118 |
| 0.524241 | −0.20861 | −2.40352 | −0.04731 | −1.30265 | −0.73547 | −0.30409 | −1.43369 | 0.116733 | 0.286425 |
| 1.902726 | 0.995761 | −0.24558 | 1.301721 | −0.97195 | −0.70162 | 0.657762 | −1.1633 | −0.67441 | 0.423754 |
| 0.696493 | 1.20326 | −1.61432 | −0.07491 | −1.66504 | −0.85409 | 1.107643 | −2.63744 | −2.3922 | −0.24643 |
| −0.04808 | 0.87162 | −2.23369 | 2.734727 | −1.0209 | 0.201981 | 0.068572 | −1.41763 | −2.31123 | 0.180426 |
| 1.284892 | 0.485377 | −2.43637 | 0 | −1.11432 | −0.5859 | −1.17396 | −1.51271 | −5.84002 | −0.93949 |
| 0.844754 | 0.307434 | −3.66478 | 0.641.101 | −0.88146 | 0.236485 | −0.75554 | −1.78672 | −0.85116 | 0.910109 |
| 0.023152 | 0.655148 | −1.71965 | 1.217946 | −1.90586 | −1.00008 | −1.82031 | −6.08007 | −2.0381 | −0.02753 |
| 0.345689 | 0.147439 | 0.011501 | 1.46225 | −0.25999 | 0.09029 | −1.01728 | −1.59083 | −1.22391 | −1.41031 |
| 0.054814 | 0.788968 | −4.54879 | 0.309044 | −1.63374 | −1.35231 | −0.40886 | −2.19955 | −2.68301 | 1.376551 |
| 0.91112 | 1.021173 | −0.30993 | −0.31151 | −1.21523 | −0.97649 | −0.87533 | −0.71737 | −1.03182 | 0.721517 |
| 1.183183 | −1.33928 | −1.83562 | 0.322244 | −0.57729 | −0.07487 | −0.75777 | −1.71734 | −3.10021 | 0.729818 |
| 0.08953 | 1.052149 | −1.77524 | 3.478341 | −1.50515 | −0.65561 | −0.28644 | −2.02924 | −3.3116 | 0.090362 |
| −0.09123 | −0.68387 | −1.2416 | 1.314882 | −0.50331 | −0.14646 | −0.13927 | −1.37119 | −0.99513 | −0.02829 |
| 0.304343 | 0.646873 | −4.22615 | 0.728796 | −1.66473 | −0.85854 | −1.27401 | −1.94071 | −1.30111 | 1.586962 |
| 1.649336 | 0.901926 | −1.01551 | 1.104217 | −1.44542 | −1.9703 | −1.48266 | −2.08532 | −3.13936 | 0.923686 |
| 0.057531 | 0.593169 | −3.34547 | 1.129828 | −1.18736 | −1.57259 | −1.55324 | −1.1981 | −0.3228 | −0.09075 |
| 0.217651 | 1.641431 | −1.61215 | 0.401.677 | −1.64733 | −1.42855 | −0.01666 | −2.13398 | −0.28249 | 0.569615 |
| 0.849807 | −0.42086 | −1.2644 | 0.353441 | −1.30199 | −0.44147 | −0.87763 | −0.70334 | −1.47377 | 0.622497 |
| −0.00327 | 0.003273 | −2.63356 | 3.53246 | −0.00493 | −2.52197 | −2.316 | −2.33129 | −0.64181 | 2.314428 |
| −1.48998 | −0.05124 | −1.94754 | 3.808289 | 0.009004 | −2.90583 | −2.41482 | −2.46847 | −1.15133 | 2.429976 |
| −0.46167 | −0.88977 | −1.93925 | 3.670346 | −0.11145 | −2.5136 | −2.0386 | −2.55611 | −0.49539 | 2.26534 |

| Unknown | Unknown | Unknown | Unknown | Unknown | Unknown | Unknown | Unknown | Unknown | Unknown |
|---|---|---|---|---|---|---|---|---|---|
| 0.999946 | 2.63892 | 0.306337 | 0.070965 | −0.10575 | −1.04389 | −0.00279 | −1.14964 | 0 | 1.199626 |
| 1.088574 | −1.54324 | 0.34178 | −0.16426 | −0.29517 | −1.26006 | −1.57103 | −0.86995 | 0.316512 | 0.45168 |
| 2.658325 | 2.642642 | 0.028003 | −0.18158 | −0.78652 | −1.87336 | −1.46556 | −1.14333 | −0.11181 | 0.699646 |
| −0.15591 | 3.186195 | 0.946019 | −0.18598 | 1.00591 | −0.74635 | −0.09176 | −1.39751 | −0.29773 | 1.816511 |
| 1.552279 | 2.855016 | 0.132508 | −0.19995 | −0.25551 | −1.19402 | −1.40788 | −0.42565 | 0.449115 | 1.387178 |
| 3.077216 | 2.846071 | 0.28359 | −0.62817 | −1.16417 | −1.63349 | −0.04224 | −1.35733 | −0.45565 | 1.187383 |
| 1.29477 | 2.808462 | 0.747211 | −0.35739 | −1.02209 | −1.8139 | −1.98312 | −0.75432 | 0.751864 | 0.502847 |
| 2.832755 | 2.654989 | 0.448868 | −0.55499 | −0.36941 | −1.54629 | −1.73052 | −1.23032 | −0.55283 | 1.4574809 |
| 1.630055 | 2.336313 | −0.96509 | −0.63042 | −1.13046 | −0.49599 | −1.50618 | −0.49183 | 0.376242 | 0.335132 |
| 0.695764 | 2.350457 | 0.052017 | −0.77104 | −0.62007 | −1.71827 | −2.10881 | −1.53472 | 0.686784 | 0.415504 |
| 0.387048 | NA | 0.463444 | −0.03876 | −0.14717 | −1.71662 | −0.82526 | −0.32641 | 0.811749 | 1.003076 |
| 0.403951 | 2.216989 | 0.375909 | 0.037376 | −0.1114 | −0.98029 | −1.27919 | −0.78661 | −0.25001 | 0.467695 |
| −0.30542 | 2.781737 | 0.507558 | −0.95374 | 0.033385 | −1.91752 | −0.68571 | −0.83006 | −1.16126 | 1.46664 |
| 1.510046 | 3.278826 | 0.29971 | −0.29737 | −0.32304 | −1.57322 | −4.19822 | −0.43763 | 0.925952 | 0.372889 |
| 2.261292 | 1.91572 | 0.321399 | −2.18657 | 0.981086 | −0.80688 | 0.403532 | −0.65308 | 0.173595 | 1.584195 |
| −4.43838 | 2.496125 | 0.654247 | −0.45466 | −1.85451 | −2.03238 | −2.16685 | −0.89528 | 0.863458 | 0.423543 |
| 0.339584 | 3.006433 | 0.448983 | 0.028634 | −0.31329 | −0.48592 | −1.5614 | −1.1833 | −0.15068 | 0.918761 |
| 1.535863 | 2.702705 | −0.39811 | 0.012862 | −0.86376 | −0.81294 | −0.66599 | −1.07198 | 0.810283 | 0.852582 |
| 2.151971 | 2.046079 | 0.26331 | −0.5937 | −1.64059 | −2.82527 | −2.07793 | −1.17577 | −0.88008 | 1.502201 |
| 0.500864 | 2.290946 | 0.649887 | −0.17951 | −0.61902 | −4.35751 | −1.04746 | −0.99932 | 0.179515 | 3.13847 |
| 1.467976 | 2.47644 | 0.312631 | −0.20198 | −0.42343 | −2.11149 | −1.95026 | −0.96262 | 0.366976 | 0.208903 |
| −0.1874 | 2.96674 | 0.869182 | −0.3122 | 0.396285 | −1.49501 | −0.31164 | −0.83374 | −1.37355 | 1.585658 |
| −1.66518 | 1.923734 | −0.94407 | 0.071424 | 0.581057 | −0.62653 | 0.0574571 | −0.55126 | −0.40704 | 0.199996 |
| 1.228629 | 3.195881 | 0.674812 | −0.64884 | −0.83735 | −2.03708 | −2.83278 | −1.02438 | 0.536141 | 0.620752 |
| 3.004828 | 2.800946 | 0.479111 | −1.16834 | −0.1033 | −1.19247 | −1.78213 | −1.16439 | −0.47426 | 1.462975 |
| 3.093792 | 2.924719 | −0.52899 | −1.7774 | 1.557287 | NA | −1.5297 | −0.12842 | 0.145997 | 0.941237 |
| 0.348261 | 2.48432 | 0.163433 | −0.23264 | −1.58753 | −1.39305 | −1.06248 | −0.98472 | −0.59857 | 1.507103 |
| 3.851987 | 2.822599 | 0.758067 | −0.2711 | −0.24462 | −3.94459 | −0.87813 | −0.31731 | 0.470363 | 1.010003 |
| −0.05892 | 2.90193 | 0.474254 | 0.5741 | −0.1563 | −1.54885 | −1.28611 | −0.65309 | 0.008355 | 0.619159 |
| 2.699761 | 2.584482 | 0.398661 | −0.99405 | 0.041939 | −1.74359 | −0.49795 | −0.76283 | −2.82298 | 2.179719 |
| 3.480038 | 3.165486 | −0.01095 | −0.35378 | −0.39583 | −2.0737 | −0.30013 | −0.63907 | 0.545216 | 0.310074 |
| 2.047388 | 2.410752 | −0.39323 | 0.317605 | 0.37431.8 | −1.91813 | 3.870028 | −0.71561 | 0.296998 | 1.326139 |
| −0.08005 | 2.993182 | 0.433254 | −0.46909 | −0.11389 | −1.7038 | −1.4772 | −0.96053 | −0.24045 | 1.119031 |
| 2.313609 | 1.768556 | −0.84936 | −1.22372 | 0.693656 | −4.42572 | −0.84768 | −1.03392 | 0.377163 | 1.696881 |
| 2.267195 | 1.835104 | −1.5849 | −1.26715 | 0.637688 | −4.9341 | −0.49011 | −0.86834 | 0.430012 | 1.66932 |
| 2.143151 | 1.338903 | −1.49989 | −1.61272 | 0.335088 | −4.41951 | −0.83871 | −1.05453 | 0.285017 | 1.691556 |

| Unknown | Unknown | Unknown | Unknown | Unknown | Unknown | Unknown | Unknown | Unknown | Unknown |
|---|---|---|---|---|---|---|---|---|---|
| −0.74635 | 2.287053 | −0.12815 | 1.536587 | −0.12617 | −0.9705 | 2.054836 | 0.827579 | −0.80474 | 3.555507 |
| −1.00453 | 1.913666 | 0.169999 | 0.252138 | −1.19107 | −3.26757 | 1.990821 | 0.604573 | −0.68903 | 0.414385 |
| 0.008902 | 2.850192 | 0.182571 | 0.265779 | −0.46656 | −2.54834 | −0.50625 | 0.929355 | −2.90108 | 0.42366 |
| −0.94686 | 2.614697 | 0.48624 | 1.39526 | −0.38879 | −1.02819 | 1.908955 | 1.160958 | −0.42317 | 2.251018 |
| −1.80817 | 2.430165 | −0.73701 | 0.82144 | 0.132973 | −1.90137 | 0.375021 | 0.494031 | −2.45826 | 2.520218 |
| −1.04811 | NA | −1.3214 | 0.865115 | −0.38756 | −1.0775 | 2.129205 | −0.59568 | −3.41203 | 0.593379 |
| −1.22244 | 3.038393 | 0.025822 | 1.37389 | −0.00722 | −1.49348 | 0.808941 | 0.732536 | −2.40564 | 4.860852 |
| −2.45778 | 1.958725 | 0.154358 | 0.798468 | −0.50577 | −1.65995 | 2.020381 | 0.913024 | −2.90944 | 0.150539 |
| −0.81707 | 2.435904 | 0.351255 | 1.004085 | −0.02046 | −2.04497 | 1.743716 | 0.874508 | −4.13881 | 3.211544 |
| −1.61809 | 2.279918 | −1.19953 | 0.855482 | −0.41452 | −1.5832 | 1.865944 | 1.470891 | −3.20095 | 1.31988 |
| −0.95242 | 2.085184 | 0.525178 | 0.604227 | 0 | −2.5996 | −0.03311 | 1.042519 | −0.86894 | 0.48283 |
| 0.408296 | 0.586928 | 0.204249 | 1.171157 | −0.301 | −2.88583 | −3.1057 | 0.723544 | −2.45275 | 0.841904 |
| −1.39349 | 2.4345 | 0.183257 | 1.181529 | −0.18861 | −1.39985 | 2.120336 | 0.800439 | −1.17795 | 0.454698 |
| −1.75362 | 2.525528 | −0.79521 | 0.195365 | −0.17335 | −1.23604 | −3.10445 | 0.736805 | −2.24599 | 2.697328 |
| −0.03416 | NA | −0.44021 | 1.133778 | 0.037005 | −1.58851 | −0.0776 | −0.51338 | −2.5093 | 1.651111 |
| −0.39755 | 2.347813 | −0.12718 | 0.868203 | −0.01002 | −1.60317 | 0.99176 | 1.350907 | −2.97196 | 3.205838 |
| −0.57188 | −4.92341 | −0.2808 | 1.530037 | −0.07583 | −1.39123 | 2.056096 | −0.04863 | −2.50776 | 2.110138 |
| −0.97071 | 2.381598 | 0.297174 | 0.923857 | −0.38087 | −1.935 | 0.644274 | 1.013323 | −2.55506 | 4.737389 |
| −0.9932 | 2.203969 | −0.90233 | 0.829188 | −0.42678 | −2.11285 | 2.251324 | 1.751494 | −0.43147 | 0.207539 |
| −1.47855 | 2.218 | 0.319501 | −0.21533 | −0.46786 | −2.44337 | −0.23088 | 0.333288 | −1.53569 | −0.08545 |
| −0.11554 | 1.655279 | 0.230825 | 0.196818 | −0.35438 | −2.38127 | 1.904264 | 1.038809 | −3.24423 | 1.065798 |
| −1.53799 | 2.507688 | 0.302094 | 1.243162 | −0.18635 | −1.13285 | 2.117436 | 1.128919 | −1.07913 | 1.513816 |
| −0.60143 | 1.615305 | −0.4401 | 1.312135 | −0.1871 | −2.01508 | 1.996959 | −0.35248 | −2.31259 | 3.258404 |
| −1.14763 | −5.63284 | −0.18937 | 1.608979 | 0.037782 | −1.0517 | 0.912711 | 0.202361 | −2.11878 | 4.023367 |
| −0.9092 | 2.352131 | −0.23803 | 0.552409 | −0.22735 | −1.66552 | 1.842978 | 0.870204 | −3.1003 | 2.136739 |
| −0.85327 | NA | −0.70296 | 0.153239 | 0.007115 | −2.2195 | −0.30956 | 0.798085 | −1.57839 | 1.005141 |
| −0.68071 | 2.134529 | 0.358808 | 0.911468 | 0 | −1.98792 | 0.18S529 | 1.238057 | −1.59715 | 2.035017 |
| −0.63277 | 2.806312 | 0.501434 | 0.621027 | −0.52027 | −2.48411 | −0.28414 | 0.767184 | −0.51787 | −0.10549 |
| −0.12913 | 3.188112 | 0.202339 | 0.379547 | −0.20708 | −1.58518 | 2.036252 | 1.414416 | −2.45048 | 1.291067 |
| −1.49176 | 2.198814 | −0.44538 | 1.409076 | −0.27858 | −1.19966 | 2.126802 | 1.571054 | −2.44074 | −0.43135 |
| −1.48042 | 2.392244 | −1.28694 | 0.971081 | 0.05457 | −1.31502 | 0.784955 | 0.974355 | −2.53302 | 2.559651 |
| −0.65987 | 1.925545 | −0.38983 | 3.634099 | −0.539 | −0.06035 | 3.509127 | 0.877227 | −3.22942 | 3.855185 |
| −0.82167 | 2.358275 | 0.255474 | 1.087957 | 0.165745 | −1.22952 | 2.124739 | 1.323481 | −2.45481 | 1.987192 |
| −1.44199 | 1.430493 | 1.103726 | −0.92749 | −1.29493 | −0.01632 | 2.071765 | 0.805554 | 5.893228 | 0.449529 |
| −1.76674 | 1.430945 | 1.082421 | −1.12555 | −1.06454 | 0.189405 | 2.043581 | 0.892094 | 5.9568 | 0.5058 |
| −1.72405 | 1.568916 | 1.010575 | −0.94214 | −1.5052 | 0.157358 | 2.081593 | 0.751485 | 5.956408 | 0.42353 |

| Unknown | Unknown | Unknown | Unknown | Unknown | Unknown | Unknown | Unknown | Unknown | Unknown |
|---|---|---|---|---|---|---|---|---|---|
| 1.301334 | 2.85093 | −2.12153 | 0.580543 | −3.19746 | NA | −1.38672 | 1.181896 | 0.045902 | NA |
| −0.25223 | 0.794034 | 1.114404 | 2.723307 | −1.87987 | NA | 0.489233 | −1.30433 | −0.31965 | NA |
| −0.37207 | 0.130285 | 1.751074 | 1.297212 | −2.18293 | NA | −1.24912 | −0.09376 | −0.11361 | NA |
| 2.431029 | 3.070434 | 1.741307 | 0.479378 | −1.18573 | NA | −0.19994 | 1.80532 | −0.1453 | NA |
| 0.403692 | 0.125364 | 2.50517 | 0.912986 | −2.41338 | NA | −0.3934 | −2.30471 | 0.124804 | NA |
| 1.712405 | 2.15728 | −1.94674 | 1.425111 | 1.502203 | NA | 1.471197 | 2.090566 | −0.0513 | NA |
| 0.0465 | 0.887283 | 1.865548 | 1.283261 | −1.17087 | NA | 0.196022 | −0.51036 | −0.00319 | NA |
| 0.82555 | 2.812015 | 1.547846 | 0.93382 | −2.32021 | NA | −0.23566 | −0.57601 | −0.025 | NA |
| −0.23228 | 1.027355 | 2.773293 | 0.886215 | −1.26042 | NA | 0.392434 | −1.94422 | −0.18343 | NA |
| 0.665423 | 2.052717 | −2.20908 | 1.053397 | −1.75302 | NA | −0.50928 | 0.920017 | −0.11102 | NA |
| −0.10179 | 0.832029 | 1.562537 | 0.958259 | −2.5556 | NA | −0.42523 | −0.99 | 0.108248 | NA |
| −0.23956 | 1.298751 | −1.9911 | 0.774677 | 0.658126 | NA | 0.595126 | 0.207729 | −0.09381 | NA |
| 2.039829 | 2.688974 | 1.861342 | 1.158024 | 1.059022 | NA | 1.081987 | 2.557523 | −0.00059 | NA |
| 0.319825 | 1.5986 | −1.55316 | 1.137947 | −2.14781 | NA | −0.07018 | −1.90296 | 0.295223 | NA |
| 1.28318 | 3.385844 | −2.51839 | 0.240582 | 2.631623 | NA | 2.33037 | 2.913604 | −0.03857 | NA |
| −0.55166 | 1.332533 | 1.577436 | 1.374799 | 0.961969 | NA | 1.154782 | −0.8779 | −0.25553 | NA |
| 0.811747 | 2.619306 | −1.77098 | 1.097853 | −2.4281 | NA | −0.15751 | −0.88221 | −0.82702 | NA |
| 0.304751 | 1.333291 | 2.150288 | 1.740218 | 3.379143 | NA | 3.093769 | −1.21365 | −0.35094 | NA |
| 0.236352 | 2.2335 | 0.674488 | 1.39066 | 1.923284 | NA | 1.575821 | −0.02517 | −0.25434 | NA |
| 0.171455 | 0.442448 | 0.939294 | 1.405502 | −1.2156 | 0.49295 | −0.70845 | −1.31051 | −0.5161 | NA |
| 0.24658 | 1.355979 | 1.975078 | 0.803962 | −1.61226 | NA | −1.04975 | 0.225264 | −0.51437 | NA |
| 2.369399 | 3.024844 | 2.495227 | 0.769774 | 0.755582 | NA | 1.087637 | 2.415095 | −0.05411 | NA |
| 1.561953 | 2.847846 | 2.801666 | −0.51309 | 1.736053 | NA | 1.857345 | 1.993044 | −0.0115 | NA |
| −0.35944 | 1.321411 | 2.52648 | 1.641767 | −2.49775 | NA | −0.00173 | −1.77187 | −0.06309 | NA |
| 0.498884 | 2.446027 | 1.749923 | 1.10805 | −2.51409 | NA | −0.31183 | −0.75283 | 0.122498 | NA |
| −0.03632 | 1.477886 | −1.70155 | 1.470037 | −0.77939 | NA | −0.15536 | −0.70996 | −0.35585 | NA |
| 0.819741 | 1.7831 | −2.38086 | 0.544305 | 1.832949 | NA | 1.536203 | −0.51416 | 0.37212 | NA |
| 0.328536 | 1.604454 | 1.735418 | 2.352714 | −1.55709 | NA | 0.193207 | −0.41351 | 0.185751 | NA |
| 0.003399 | 1.16858 | 1.439687 | 1.170035 | −5.49869 | NA | −2.67102 | 0.353557 | −0.43688 | NA |
| 2.421001 | 2.474585 | 1.474238 | 1.235995 | −1.01894 | NA | −0.26829 | 1.228099 | −0.03846 | NA |
| 2.156283 | 3.133873 | 1.770539 | 1.185311 | −2.55447 | NA | −0.31246 | −1.94801 | 0.095912 | NA |
| 2.981719 | 4.937318 | 2.213092 | 1.212969 | −0.31866 | NA | 1.581837 | 2.637647 | 0.462367 | NA |
| 0.889688 | 2.993193 | 1.95184 | 1.069216 | −2.49587 | NA | 0.258788 | −0.76972 | −0.03255 | NA |
| 1.734271 | 4.327212 | −0.35902 | −1.41949 | −2.97538 | 1.845059 | 1.447869 | 0.980726 | 1.476119 | 1.153362 |
| 1.881778 | 3.846829 | −0.73418 | −0.89463 | −2.46753 | 2.097824 | 1.527514 | 1.233782 | 1.758941 | −0.83672 |
| 1.819795 | 4.319798 | −1.24457 | −1.17848 | −3.28926 | 1.961038 | 1.484382 | 1.088458 | 1.697093 | −1.47487 |

| Unknown | Unknown | Unknown | Unknown | Unknown | Unknown | Unknown | Unknown | Unknown | Unknown |
|---|---|---|---|---|---|---|---|---|---|
| 0.072877 | −1.89339 | −0.96484 | −1.15643 | −1.72545 | −1.42296 | NA | NA | −1.90871 | −0.78959 |
| 0.82974 | 2.188637 | −1.22438 | −0.43017 | −1.4669 | −0.95797 | 2.568738 | −2.1154 | −2.39815 | −0.80814 |
| 0.68765 | −1.179 | −0.20466 | 0.072082 | −0.1106 | −1.91111 | 2.957718 | NA | 0.449329 | 0.469691 |
| 1.125661 | −2.05252 | −0.05526 | −0.8122 | −1.05414 | −1.85332 | 1.603147 | −0.98102 | −0.52543 | −0.07502 |
| 1.119177 | −0.69145 | −0.67707 | −0.9345 | −0.5666 | 1.65443 | NA | MA | −2.17279 | 0.471578 |
| 0.945192 | −1.41958 | −1.21128 | 0.249263 | −1.41029 | −1.1712 | 2.857362 | −3.04387 | −1.92084 | 0.187159 |
| 0.11659 | 2.723745 | −1.54714 | −1.10908 | −1.7385 | −1.9918 | NA | NA | −0.68357 | 1.122751 |
| 0.3.33111 | −2.38815 | 0.114624 | −0.62961 | −0.68562 | −1.95658 | NA | NA | −1.81427 | 0.273543 |
| 1.755744 | −1.18006 | −0.23779 | 1.092674 | −0.69779 | −0.20909 | 3.602294 | NA | −1.00148 | −0.91382 |
| 0.171178 | −2.09326 | −0.65991 | −2.17889 | −1.42669 | −0.96722 | NA | −1.61787 | −2.34937 | −0.08937 |
| 0.650528 | 2.51736 | 0.141741 | −0.04688 | −1.07321 | −1.7545 | NA | −1.48179 | −2.91884 | −0.63003 |
| −0.58546 | 1.981395 | 0.183428 | −0.75743 | −1.25546 | −1.5356 | NA | −1.4351 | −0.20701 | −0.17758 |
| 1.078083 | −2.54385 | −0.17013 | −0.73468 | −2.10381 | −1.90991 | NA | −1.22903 | −3.61469 | 0.045974 |
| 0.645197 | −0.72873 | −0.92086 | −0.63478 | −0.38658 | −1.45332 | 2.455307 | NA | −2.25168 | 1.035052 |
| 0.895218 | −1.52947 | −0.2222 | −1.2994 | −2.19679 | −1.99463 | NA | NA | −2.95221 | −3.58172 |
| 0.167468 | 2.645786 | −0.87675 | −0.81124 | −1.09243 | −1.72964 | NA | −4.53951 | −1.85251 | 0.791924 |
| 0.166892 | −2.42447 | −0.54176 | −0.88312 | −1.0622 | −0.5367 | NA | NA | −1.981 | −0.46333 |
| 0.90075 | −1.77481 | −1.55763 | −0.17548 | −0.48011 | −0.39863 | 2.64886 | NA | −1.11327 | −0.29027 |
| 0.215305 | 2.36141 | −2.78202 | 0.256572 | −2.38224 | −2.02073 | 3.074611 | −5.92901 | −2.36665 | −0.24941 |
| 0.31687 | 2.392868 | 0.441457 | −1.57495 | −2.92819 | −2.82998 | NA | −1.92073 | −3.34936 | −0.63071 |
| −0.32099 | 1.812603 | −0.09039 | −2.36344 | 0.25524 | −0.60994 | NA | −1.47106 | −2.41011 | 0.442356 |
| 1.004969 | −1.5466 | 0.385084 | −0.95506 | −1.28744 | −2.78558 | NA | −0.77692 | −0.10611 | −0.19285 |
| 0.245876 | 2.254016 | −0.05379 | −0.17997 | −3.47895 | −3.0999 | NA | −6.67403 | −2.05337 | −4.69191 |
| 0.019294 | 2.806391 | −1.79925 | −1.19199 | −1.32779 | −1.44243 | NA | −7.5283 | 0.001727 | 0.858401 |
| 0.078122 | 1.898144 | 0.276875 | −0.54875 | −1.20945 | −0.93102 | NA | −5.24302 | 0.353048 | −0.34784 |
| 1.460329 | 2.397377 | −0.19139 | 0.512933 | −1.43231 | −1.7182 | NA | −7.02556 | −1.73315 | −0.76212 |
| 0.393919 | 2.672398 | 0.58392 | −0.04527 | −1.11637 | −1.20785 | NA | 1.50884 | −2.43944 | −0.45948 |
| 0.99882 | −0.54402 | −3.80582 | −0.50316 | −0.02425 | −0.56236 | NA | 1.37743 | −2.92786 | −0.14339 |
| 0.322003 | 2.123378 | −0.38333 | −0.37637 | 0.027208 | −1.4411 | 2.422776 | 1.46925 | −2.05171 | 0.898298 |
| 1.039324 | −2.60168 | −0.16068 | −0.28318 | −1.6567 | −1.92253 | NA | −1.22695 | −2.74055 | −0.0965 |
| 0.158855 | 2.500763 | −3.87634 | −4.14907 | −1.07547 | −2.52157 | NA | NA | −2.16564 | 0.784473 |
| 0.274798 | −1.67462 | −3.07758 | −0.90638 | −1.07741 | −1.49604 | NA | −7.8793 | −0.80332 | −3.8422 |
| 0.529118 | −1.61559 | −0.24735 | −0.16572 | −0.47334 | −0.37212 | NA | NA | −1.29938 | 0.052221 |
| −0.70088 | −1.27969 | −0.60422 | −1.15218 | −0.70527 | −4.86221 | 1.820604 | −0.67666 | −0.79519 | 0.234612 |
| −0.55734 | −2.23004 | −2.10438 | NA | −1.33237 | −2.23187 | NA | −1.37556 | −0.47507 | 0.374352 |
| −0.81098 | 0.790382 | −0.8564 | −1.72114 | −0.49495 | −4.75318 | NA | −0.95746 | −0.61407 | 0.033593 |

| Unknown | Unknown | Unknown | Unknown | Unknown | Unknown | Unknown | Unknown | Unknown | Unknown |
|---|---|---|---|---|---|---|---|---|---|
| −0.60219 | −2.71786 | 0.083648 | −0.19787 | −1.71785 | −1.31937 | −0.43777 | 0.212006 | −0.43252 | 0.877956 |
| −0.45183 | −3.77111 | −0.19228 | 0.962204 | −0.49772 | −2.90442 | −0.96932 | −0.06963 | −0.14854 | 1.046462 |
| −0.61956 | −2.52891 | −0.31997 | −0.12566 | −2.07801 | −1.59762 | −0.0089 | 0.50385 | −0.96796 | 0.904972 |
| −0.1794 | −2.2385 | −0.33297 | −1.87885 | −2.55559 | −4.24139 | −2.13932 | −0.1848 | 0.007405 | 1.327324 |
| 0.296573 | −2.32867 | −0.32287 | −0.25628 | −1.24727 | 1.22647 | −0.72962 | −0.35673 | −0.07615 | 0.024267 |
| −1.55255 | −3.14215 | −0.65431 | 0.759965 | −0.03913 | −2.37917 | 0.282719 | −0.42544 | −1.8242 | 0.090871 |
| −0.77886 | −2.81879 | −0.36585 | −0.32666 | −1.91707 | −1.78627 | −2.48509 | 0.163767 | 0.044653 | 1.514513 |
| 0.090956 | −2.50399 | −0.54226 | 0.41563 | −2.03344 | −1.93256 | 0.760376 | −0.69997 | −1.56828 | 0.397053 |
| −1.3827 | −3.84793 | 0.240674 | −0.14982 | −1.96045 | −0.73372 | −0.56711 | 0.528102 | −0.05805 | 0.237041 |
| −0.35831 | −2.81538 | −0.54436 | 0.858988 | −2.57317 | −3.93933 | 0.144238 | 0.922592 | −1.23435 | 1.269594 |
| −0.57404 | −2.96732 | −0.2356 | 1.030531 | −0.63635 | −1.19853 | −1.26297 | 0.430022 | 0.175295 | 0.956572 |
| −1.24856 | −2.98936 | −0.58946 | 0.640102 | −2.5857 | −2.89167 | −0.71636 | −0.71669 | −0.00055 | −0.65091 |
| −0.52524 | −2.69535 | −0.28905 | −1.03598 | −2.98594 | −0.00388 | −0.96801 | 0.203587 | −0.21094 | 0.047626 |
| −0.42617 | −2.34694 | −0.10183 | 0.453142 | −1.55484 | −1.43087 | −0.3933 | 0.46406 | 0.345811 | 1.136445 |
| −1.44325 | −2.64982 | −0.88407 | 1.321479 | 0.363475 | −2.44134 | −0.38325 | −3.0082 | −2.27884 | −1.01864 |
| 0.029962 | −2.96115 | −0.37132 | 1.33233 | −2.33742 | −1.72213 | −1.82815 | 0.290039 | 0.145003 | 0.716732 |
| −0.56277 | −3.22637 | −0.5616 | 0.834274 | 0.198098 | −1.83015 | 0.590926 | −0.22914 | −1.36728 | 1.544481 |
| −0.03618 | −3.20575 | −0.33311 | 1.193879 | −2.32846 | −1.04887 | −1.34946 | 0.28727 | −0.08315 | 0.105994 |
| −0.19988 | −2.48959 | −0.84553 | 0.01438 | −2.47529 | −1.95472 | −0.4549 | 0.374457 | −1.14781 | −0.4562 |
| 0.440435 | −2.42415 | −0.87454 | 0.172457 | −1.36139 | −0.97406 | −0.92898 | −0.47475 | −0.26941 | 0.046096 |
| 1.12815 | −3.4088 | −0.44733 | −0.20005 | −2.4286 | 1.89753 | −0.98156 | 0.005896 | −0.38651 | −1.00047 |
| −0.25476 | −2.36253 | −0.03256 | −0.7108 | −2.73729 | −2.18643 | −0.99384 | 0.544654 | −0.05891 | −0.52901 |
| −0.55692 | −3.94398 | −0.1371 | −0.6913 | −1.77594 | −1.64148 | −0.34319 | −0.29903 | −2.43447 | 0.609024 |
| −0.34716 | −3.05514 | −0.4216 | −0.17477 | −1.95313 | −1.20706 | −1.48871 | 0.769453 | 0.41696 | 1.287094 |
| −0.19243 | −2.55608 | −0.58565 | 1.113176 | −2.37325 | −2.07715 | 0.351303 | 0.498213 | −1.37985 | 0.364372 |
| −0.43897 | −2.67787 | −0.58128 | 0.49782 | −2.48114 | −1.15858 | −1.0315 | −0.00711 | −0.99236 | −0.62436 |
| 0.312338 | −3.11504 | −0.35663 | −0.50784 | −2.93654 | −1.68075 | −0.67146 | 0.554354 | −1.06061 | 1.297225 |
| −0.10555 | −2.43303 | −0.13146 | 0.518158 | −0.45562 | −0.62125 | −0.87168 | 0.500072 | 0.446845 | 1.101666 |
| 0.477559 | −2.29128 | −0.49349 | 0.127645 | −1.63267 | −1.37785 | −1.68719 | 0.31194 | 0.265523 | −0.06063 |
| −0.30423 | −2.49686 | −0.4807 | −1.02111 | −2.5162 | −0.32202 | −0.39077 | 0.120357 | −0.43716 | −1.19863 |
| −0.5191 | −2.37414 | −0.64923 | −0.27415 | −2.49273 | −1.44409 | −1.25181 | −0.37205 | 0.253533 | 0.82273 |
| −1.50124 | −2.90785 | −0.37775 | 0.877329 | −1.35542 | −0.92864 | −0.83702 | 0.101795 | −0.94007 | 1.546458 |
| 0.11744 | −1.98948 | −0.22097 | −0.03769 | −1.20188 | −1.39777 | −0.16014 | −0.0231 | −0.04311 | 1.451863 |
| −2.65753 | −3.55724 | −4.23502 | −0.86474 | −4.20397 | −1.55671 | −4.23594 | −1.35924 | −2.91644 | 0.493827 |
| −2.08431 | −3.42421 | −2.96415 | −0.0725 | −3.06551 | 1.16148 | −4.76588 | −1.98228 | −3.00536 | 0.541472 |
| −2.31205 | −3.39418 | −2.91321 | 0.396194 | −3.13611 | −0.75265 | −3.39588 | −1.50032 | −3.12282 | 0.942473 |

| Unknown | Unknown | Unknown | Unknown | Unknown | Unknown | Unknown | Unknown | Unknown | Unknown |
|---|---|---|---|---|---|---|---|---|---|
| 1.468798 | 1.207846 | 3.792078 | NA | NA | 1.151748 | −2.23962 | −3.07369 | −4.46776 | 0.562963 |
| 0.179839 | 0.757162 | 3.540534 | NA | NA | −1.30063 | −3.22361 | −2.42541 | −3.11751 | 1.347055 |
| −2.09611 | −1.36743 | 3.596235 | NA | NA | 1.493281 | −1.39401 | −4.14633 | −3.54379 | 1.983267 |
| 0.540915 | 1.020764 | 4.244458 | NA | NA | 1.323401 | −1.62247 | −4.09324 | −4.13522 | 0.871074 |
| 1.703853 | −0.51878 | 4.099121 | NA | NA | 1.779983 | −1.81187 | 0.145132 | −4.77217 | 1.387216 |
| −2.48013 | −1.56501 | 3.910607 | NA | NA | 1.289627 | −1.18313 | −3.67071 | −4.81513 | 1.631824 |
| 1.147678 | 0.363384 | 3.878515 | NA | NA | 0.316263 | −2.07699 | 0.544061 | −4.14927 | 0.592973 |
| −1.8411 | −1.65995 | 3.681114 | NA | NA | 1.399641 | −1.4727 | −3.65651 | −4.76041 | 2.102439 |
| 0.855933 | −1.20022 | 3.834381 | NA | NA | 0.482507 | −3.17933 | 0.91701 | −3.97655 | 1.993051 |
| −0.88449 | 0.672119 | 3.417505 | NA | NA | 1.968638 | −1.77407 | −2.64261 | −3.71527 | 1.050691 |
| −1.07122 | 0.442098 | 3.755481 | NA | NA | 0.970793 | −2.26922 | −1.98269 | −5.40877 | 1.219032 |
| −2.12575 | −2.34913 | 3.453944 | −4.62178 | NA | −0.67512 | −2.46585 | −4.85211 | −4.67903 | 0.160766 |
| −0.72389 | −1.19943 | 3.649918 | NA | NA | −0.88552 | −1.49879 | −2.97766 | −4.28336 | 1.566234 |
| 0.190466 | −1.00001 | 4.045885 | NA | NA | 1.765093 | −1.03651 | 0.594288 | −4.49962 | 1.503062 |
| −1.83605 | −1.75876 | 3.219003 | NA | NA | 0.594289 | −2.14592 | −3.68665 | −4.0129 | 0.564034 |
| −1.25495 | 0.19026 | 3.803855 | NA | NA | 0.164586 | −1.91448 | 0.773559 | −3.44721 | 1.195994 |
| −0.27679 | −0.74339 | 4.257957 | NA | NA | 1.376458 | −1.30874 | −3.8851 | −2.88699 | 1.553209 |
| 0.087079 | −1.28257 | 4.003631 | NA | NA | −0.52991 | −2.87028 | 0.951468 | −5.99887 | 1.19886 |
| −2.88169 | −2.10365 | 3.445366 | NA | NA | −0.31004 | −2.42863 | −1.38855 | −3.67572 | 1.577333 |
| −0.91792 | −0.14289 | 3.468521 | NA | NA | 1.006348 | −1.9565 | −2.5584 | −2.97933 | 1.458608 |
| −1.59852 | −2.08815 | 3.711608 | NA | NA | 1.046205 | −1.66184 | −3.70631 | −4.23319 | 0.831131 |
| −0.27547 | −1.57219 | 4.017615 | NA | NA | −0.75539 | −1.67548 | −3.2245 | −4.28621 | 1.160591 |
| 0.685551 | −1.40567 | 3.528728 | NA | NA | 1.132233 | NA | −1.89263 | −4.20464 | 1.412507 |
| −0.35825 | 0.577753 | 4.019784 | NA | NA | 0.236933 | −1.56554 | −0.36519 | −3.06997 | 1.309385 |
| −0.49502 | −1.28235 | 3.673771 | NA | NA | 1.394S21 | −1.39035 | −5.53144 | −3.10201 | 1.931178 |
| −4.35191 | −1.53832 | 3.876675 | NA | NA | 1.338107 | −1.52732 | −0.85528 | −3.03307 | 1.476246 |
| −0.55441 | 0.31525 | 3.973904 | NA | NA | 0.245693 | −3.5055 | −3.43396 | −6.28375 | 0.609643 |
| −0.61834 | 1.363135 | 3.971956 | NA | NA | −0.72948 | −2.0739 | −2.42632 | −4.30935 | 2.000048 |
| −2.04848 | −1.56802 | 4.119788 | NA | NA | 1.891667 | −1.02017 | −4.16216 | −3.95752 | 0.847918 |
| −1.49777 | −2.16752 | 2.893287 | NA | NA | 1.299045 | −1.25658 | −3.85624 | −6.18146 | 1.345944 |
| −0.1169 | 0.836576 | 3.67811 | NA | NA | 1.751493 | −0.7627 | −0.03278 | −5.25751 | 0.716243 |
| 1.218546 | −1.16763 | 3.822854 | NA | NA | 1.321187 | −2.99783 | −3.03306 | −4.46547 | 1.479755 |
| 0.532374 | −0.86409 | 4.079774 | NA | NA | 1.560118 | −1.11824 | −3.695 | −3.46928 | 1.913927 |
| −1.15518 | 0.291976 | −2.61748 | 1.802655 | 2.2577 | 0.078216 | −1.27798 | −6.29574 | 2.441541 | 0.204358 |
| −0.42716 | 0.443314 | −2.7329 | 2.018028 | 2.13988 | −0.01654 | −1.17974 | NA | 2.621252 | −0.42056 |
| −1.29515 | 0.779798 | −3.52792 | 1.800334 | 2.130854 | 0.166842 | −1.19717 | NA | 2.512141 | 0.364073 |

| Unknown | Unknown | Unknown | Unknown | Unknown | Unknown | Unknown | Unknown | Unknown | Unknown |
|---|---|---|---|---|---|---|---|---|---|
| 0.287877 | −1.60473 | NA | −4.4257 | −0.78898 | −1.76224 | −3.58839 | NA | −2.9149 | −1.26424 |
| −6.21815 | −2.81366 | −4.08371 | −4.33667 | −1.94332 | −0.81109 | −6.75401 | NA | −3.47137 | −1.98054 |
| −4.69661 | −1.31804 | NA | −2.26943 | −1.54339 | 0.17939 | −1.74056 | NA | −2.9942 | −0.79119 |
| −5.45321 | −0.89278 | NA | −1.99567 | −1.27466 | 0.265442 | −2.88823 | NA | −2.74135 | 0.191369 |
| 1.189754 | −1.08222 | NA | −2.94969 | 3.96912 | 0.682723 | −1.39971 | NA | NA | −2.16861 |
| −2.89649 | −1.36482 | −3.14267 | −3.2834 | −1.04014 | 0.213989 | −1.73453 | NA | −4.35622 | −0.10802 |
| −1.53008 | −2.11627 | NA | −2.43733 | 3.861994 | 0.500204 | −2.05339 | NA | −2.9788 | −2.34612 |
| −4.69717 | −1.93429 | NA | −3.89655 | −0.7621 | 0.426701 | −1.60126 | NA | −4.44325 | −1.19774 |
| −4.9333 | −2.25986 | NA | −3.9809 | 3.821136 | 0.937016 | −3.75186 | NA | −3.56697 | −1.85657 |
| 1.337327 | −0.99124 | NA | −1.79511 | 0.548496 | 0.281022 | −1.05549 | NA | −2.46096 | −0.70321 |
| −4.94791 | −2.42089 | NA | −2.39962 | −1.70337 | 1.222364 | −2.33839 | NA | −3.33222 | −2.24895 |
| NA | −1.93602 | NA | −3.24397 | −3.49889 | −0.61049 | −4.2174 | 0.408394 | −8.79798 | −0.99527 |
| −4.08874 | −1.09574 | −0.50033 | −0.47811 | −1.72168 | 0.484619 | −1.78166 | NA | −3.33542 | 0.183888 |
| 2.018894 | −5.27503 | NA | −3.66847 | 3.650553 | 0.797829 | −1.14862 | NA | NA | −2.07189 |
| NA | −1.4347 | NA | −5.57606 | −1.48915 | −1.15411 | −3.25278 | NA | −4.66522 | −1.68195 |
| −7.06498 | −1.62165 | NA | −3.56013 | 4.079124 | −0.31544 | −2.58089 | NA | −4.04964 | −1.60841 |
| NA | −0.95656 | NA | −4.93414 | 0.557261 | 1.003562 | −2.05973 | NA | −2.93989 | −1.50524 |
| −6.34469 | −1.63827 | NA | −3.58838 | 4.248576 | 1.117324 | NA | NA | −2.50102 | −2.5666 |
| 0.667393 | −1.93022 | −0.38339 | −0.03875 | 1.270221 | −0.5625 | −3.20442 | NA | −4.27651 | −1.33237 |
| −6.00828 | −0.76271 | NA | −2.19795 | −1.59166 | 1.747107 | NA | NA | −2.15069 | −1.72444 |
| 0.203172 | −1.2123 | NA | −2.69961 | −2.01115 | 0.116915 | −2.09749 | 0.432031 | −3.47939 | −1.32955 |
| −5.79368 | −1.09057 | NA | −1.57466 | −1.84683 | −1.20025 | −3.0453 | 1.434596 | −2.61388 | −0.08089 |
| −6.94933 | −1.96307 | NA | −6.59507 | −0.26114 | −1.0576 | NA | NA | −4.63118 | −1.82722 |
| −0.3141 | −1.77661 | NA | −2.62613 | 3.34958 | 1.078031 | −0.98813 | NA | −1.89436 | −1.76597 |
| −3.89915 | −1.09106 | NA | −2.57748 | 0.477853 | 0.4493 | −1.72597 | NA | −3.11458 | −1.64547 |
| NA | −2.76274 | NA | NA | 2.345087 | −0.63173 | −2.7854 | NA | −3.00542 | −1.92102 |
| 0.034717 | −1.81699 | NA | −5.93628 | 0.560274 | −1.07635 | NA | NA | −2.91262 | −1.93588 |
| −5.64216 | −1.35181 | −4.21365 | −2.07608 | −0.86399 | 1.673753 | −1.86468 | NA | −2.75678 | −1.26035 |
| 0.077284 | −0.89095 | NA | −1.78037 | −2.70089 | 0.558908 | −1.45326 | 0.663588 | −2.68557 | −1.30067 |
| 0.32005 | −1.14102 | NA | −1.55737 | −1.17917 | 1.401707 | −1.41839 | 1.519579 | −2.73301 | 0.2126 |
| 1.637105 | −4.2854 | NA | −4.27118 | 3.090018 | −0.29215 | −1.64572 | NA | −3.14577 | −2.8151 |
| −4.38893 | −0.52644 | NA | −3.19111 | 1.222528 | 1.781293 | −2.54937 | NA | −1.95025 | −1.10943 |
| −3.86415 | −1.81205 | NA | −2.59453 | −0.30377 | 0.898091 | −2.04546 | NA | −2.33068 | −1.8738 |
| 0.396146 | −2.31217 | 0.477224 | 1.349582 | 1.504623 | 0.073831 | −2.99963 | 2.513947 | −3.18896 | 1.091567 |
| 0.520667 | −1.73261 | 0.745643 | 1.661695 | 1.532091 | 1.414707 | −2.73454 | 2.620544 | −2.64623 | 1.042048 |
| 0.655683 | −2.30453 | 0.713129 | 1.599787 | 1.653008 | 1.314783 | −2.38522 | 2.808343 | −3.23635 | 1.44044 |

| | | | | | | | | |
|---|---|---|---|---|---|---|---|---|
| −3.2112 | −0.70196 | −0.04644 | −2.48456 | −1.15343 | −0.99477 | −1.10801 | −2.05549 | −0.14996 |
| −0.10141 | 0.407989 | 0.39748 | −2.81414 | −0.01638 | −1.30406 | −1.3946 | 2.872459 | −0.99216 |
| −0.862 | −1.43069 | 0.582087 | −3.47048 | −2.29363 | −0.10522 | −3.0478 | −0.92216 | −1.28473 |
| 0.313264 | −0.94124 | 0.312229 | −2.96281 | −0.50659 | −0.59978 | −1.59157 | 0.753899 | −0.44684 |
| 0.667361 | 0.23048 | 0.597041 | −2.83588 | 0.215578 | −0.44241 | 0.019836 | 2.462042 | −0.51973 |
| −1.43891 | 0.799768 | 0.486579 | −2.44016 | 0.443574 | −1.2661 | −0.85448 | −0.08018 | −0.25687 |
| −1.1162 | −0.54719 | −0.13172 | −4.17046 | −1.60527 | −0.8153 | −4.17523 | 1.30249 | −1.56412 |
| −2.86244 | −1.33508 | −1.69691 | −2.72999 | −1.50005 | −0.35441 | −0.01978 | −1.42891 | −0.44132 |
| −1.99696 | −0.85995 | 0.573111 | −2.77537 | −0.28623 | −2.59902 | −3.06009 | 0.260486 | 0.252004 |
| −1.90669 | −1.37164 | 0.399795 | −3.50319 | 0.151016 | −0.643 | −0.61093 | −0.5755 | −0.28781 |
| −0.97923 | 0.087995 | −0.4274 | −2.56107 | −0.2281 | 0.161491 | −2.19033 | 1.031326 | −1.00614 |
| −0.7671 | −1.81647 | 0.283369 | −2.61647 | −0.47181 | 0.484733 | −2.15999 | −1.04217 | −1.62361 |
| 0.148411 | −1.20432 | 0.353479 | −2.50175 | −0.32279 | −0.9426 | −1.42987 | 2.134371 | −0.69882 |
| 0.504089 | 0.248666 | −0.3659 | −2.2417 | −0.17084 | −0.65757 | −0.05244 | 1.960599 | −0.37344 |
| −1.35195 | −0.58854 | −1.90838 | −2.33964 | −1.10811 | −0.68734 | −3.07147 | 1.368858 | −0.78644 |
| −1.9183 | −0.12824 | 0.738251 | −3.06915 | 0.51757 | −0.1389 | 0.051893 | 0.14417 | 0.005313 |
| −1.1804 | −1.01562 | 0.726466 | −2.83896 | −0.23471 | −0.96799 | −1.75302 | −0.93043 | −0.75803 |
| −2.50885 | −2.14946 | −1.02562 | −2.22213 | −1.79204 | −0.51833 | −0.85534 | −1.99427 | −0.62593 |
| −0.78913 | −0.12361 | −0.29786 | −2.75315 | −0.8817 | −1.12775 | −1.80714 | −1.29788 | −1.44492 |
| −0.34192 | −0.24554 | 0.540435 | −2.88013 | −0.54444 | 0.433943 | −2.57956 | 0.305891 | −0.48224 |
| 0.448076 | −1.14113 | 0.229708 | −3.13783 | −0.16685 | −1.02674 | −1.26954 | 2.526858 | −1.13196 |
| 0.787063 | 0.266857 | 0.246685 | −5.5353 | 0.019673 | −0.94273 | −0.05703 | 2.36378 | −0.99233 |
| −0.38435 | 0.415483 | 0.573309 | −3.48484 | 0.535746 | −1.68662 | −1.13721 | −1.5627 | −0.70167 |
| 0.144899 | −0.26965 | 0.265508 | −3.53065 | −0.09076 | −0.79086 | −1.15872 | 2.675464 | −0.52612 |
| −1.04531 | −0.98886 | 0.998313 | −2.49328 | −0.97596 | −0.93684 | −2.43863 | −0.71838 | −0.65599 |
| 1.696491 | 0.578935 | 2.010264 | −5.07385 | 1.169515 | 1.095608 | 1.013409 | 2.200701 | −0.41032 |
| 1.755006 | 0.909839 | 2.709594 | −4.36812 | 1.244459 | 1.390492 | 1.229962 | 2.145856 | −0.44507 |
| 1.973114 | 0.841636 | 2.494596 | −3.67162 | 1.164113 | 1.351768 | 1.214039 | 2.229237 | −0.3643 |

| | | | | | | | |
|---|---|---|---|---|---|---|---|
| NA | −0.4046 | −2.51996 | −1.11319 | −4.95952 | −1.89426 | −1.04473 | NA |
| NA | 0.317655 | −1.18726 | −0.97884 | −0.47883 | −2.97731 | −1.3795 | NA |
| NA | −0.81194 | −2.30572 | −2.46217 | −0.74083 | −1.52919 | −1.70492 | NA |
| NA | 0.719521 | −2.09341 | −3.14216 | −1.02706 | −3.75501 | −1.86703 | NA |
| NA | −0.29533 | −0.95083 | −1.32352 | −3.4553 | −1.96378 | −0.42502 | NA |
| NA | 0.743261 | −2.41668 | −1.02546 | −0.32091 | −3.20097 | −0.40141 | NA |
| NA | −1.43421 | −2.98116 | −2.01063 | −2.99694 | −2.51479 | −2.25886 | NA |
| NA | 0.629798 | −2.25772 | −1.82468 | −4.30362 | −5.41803 | −1.11485 | NA |
| NA | −0.0125 | −1.47174 | −1.16947 | −1.1149 | −1.00326 | −1.60397 | NA |
| NA | 0.677022 | −2.73704 | −1.27186 | −1.27835 | −2.73153 | −0.75088 | NA |
| NA | 0.39733 | −2.10865 | −2.19538 | −2.83223 | −4.85802 | −1.29028 | NA |
| NA | 0.471652 | −2.34697 | −2.79281 | −0.50311 | −3.477 | −1.12168 | NA |
| NA | −0.0059 | −0.77686 | −2.10042 | −1.02489 | −3.17145 | −1.22198 | NA |
| NA | 0 | −2.55499 | −1.50154 | −3.34499 | −1.94383 | −0.7308 | NA |
| NA | −0.86735 | −1.89408 | −1.8695 | −0.85282 | −2.3625 | −0.54535 | NA |
| NA | 1.447519 | −1.19909 | −0.8482 | −0.51639 | −1.50555 | 0.180186 | NA |
| NA | −0.72719 | −2.15986 | −1.1597 | −0.72057 | −3.86949 | −0.96116 | NA |
| NA | 0.863532 | −2.9259 | −1.6853 | NA | −3.37713 | −2.14065 | NA |
| NA | 0.680012 | −1.8774 | −1.57446 | −3.71732 | −3.7706 | −0.77222 | NA |
| NA | 1.056993 | −1.32999 | −1.96556 | −0.41277 | −1.67299 | −0.79764 | NA |
| NA | −1.44232 | −2.57856 | −2.07759 | −0.80585 | −1.39589 | −2.17078 | NA |
| NA | −0.24161 | −3.19121 | −1.53563 | −0.97302 | −3.43053 | −1.51767 | NA |
| NA | 0.50215 | −3.20927 | −1.02779 | 0.143447 | −4.66447 | −2.1497 | NA |
| NA | 0.451756 | 0.116964 | −1.36026 | −0.03233 | −2.92024 | 0.278693 | NA |
| NA | −0.33114 | −1.15969 | −1.31326 | −0.58516 | −0.94452 | −1.507 | NA |
| 7.42967 | 1.168598 | 5.300618 | −2.06243 | 0.259113 | −0.38989 | −3.26521 | 0.789602 |
| 7.531022 | 1.94982 | 5.347735 | −1.94649 | 0.623202 | 0.463683 | −3.75964 | 1.206895 |
| 7.588999 | 1.396251 | 5.401059 | −1.71131 | 0.748009 | 0.185629 | −3.01859 | 1.213151 |

| | | | | | | | | | | |
|---|---|---|---|---|---|---|---|---|---|---|
| −1.09658 | −3.45631 | −2.09933 | −0.76431 | −1.76279 | −3.84712 | −3.43662 | NA | NA | 2.768821 | NA |
| −1.48603 | NA | −2.09669 | −1.23444 | −0.49653 | −0.60707 | −3.23375 | NA | NA | 1.915305 | NA |
| −0.36538 | −0.00301 | −0.66969 | −1.31111 | 0.002243 | −1.11509 | −1.65993 | NA | NA | 2.555293 | NA |
| −2.0536 | −3.92478 | −2.66147 | −1.6956 | −3.00741 | −2.6391 | −3.18749 | NA | 2.799633 | 0.441648 | NA |
| −0.32592 | NA | −1.20723 | −1.08548 | −1.58005 | −2.03985 | −2.67447 | NA | −0.61504 | 0.266313 | NA |
| −0.84217 | −3.16641 | −1.34538 | −1.19209 | −0.47516 | −0.09034 | −0.70321 | NA | NA | 0.673632 | NA |
| −1.84961 | −1.28056 | −0.52116 | −1.72469 | −1.98526 | −1.96288 | −2.8642 | NA | NA | 1.51168 | NA |
| −0.90617 | NA | −2.45691 | −1.25472 | −1.991 | −2.8333 | −3.89871 | NA | NA | 1.950935 | NA |
| −1.3336 | −0.81269 | 1.077721 | −1.06389 | 0.081654 | −0.22039 | −0.77444 | NA | NA | 0.948808 | NA |
| −1.04159 | −0.53118 | −0.55901 | −1.39242 | −0.33808 | −1.47308 | −2.94233 | NA | NA | 2.175026 | NA |
| −0.91054 | −2.89497 | −1.56246 | −1.37123 | −1.99927 | −2.42133 | −2.96832 | NA | NA | 1.90023 | NA |
| −0.36362 | 0.017185 | −1.35149 | −1.35352 | 0.150991 | −0.71607 | −4.32192 | NA | NA | 1.302736 | 7.616509 |
| −0.95447 | −2.90932 | −2.72381 | −1.33635 | −1.63106 | −1.02863 | −1.38059 | NA | 3.321543 | −0.31368 | NA |
| −0.54289 | NA | −2.05294 | −1.12933 | −2.23762 | −2.53319 | −3.24839 | NA | −0.39645 | −0.32821 | NA |
| −0.47857 | −1.4687 | −1.69573 | −0.92904 | −1.43204 | −1.51404 | −1.5289 | NA | NA | 2.825395 | NA |
| −0.32814 | −4.00202 | −1.38692 | −1.06179 | −1.02422 | −1.50296 | −2.01869 | NA | NA | .1.545516 | 6.540785 |
| −1.85983 | −0.51433 | 1.346016 | −1.06375 | 0.400468 | 0.242864 | −0.2976 | NA | NA | 0.715137 | NA |
| −2.02194 | NA | −1.06076 | −1.24786 | −1.54508 | −2.80149 | −3.93198 | NA | NA | 1.403653 | NA |
| −0.93323 | NA | −1.64284 | −0.96888 | −0.96565 | −1.08252 | −1.28454 | −4.98466 | NA | 2.712127 | NA |
| −0.78153 | −0.231 | −2.24321 | −0.55451 | −0.36526 | −0.71402 | −0.8483 | NA | NA | 2.399105 | NA |
| −2.14145 | −2.37077 | −1.93434 | −1.54106 | −1.62456 | −0.40358 | −1.00613 | NA | 2.932054 | −1.22992 | NA |
| −1.15881 | −0.65372 | 0.018057 | −1.4283 | −0.27019 | −1.37698 | −2.06565 | NA | NA | −1.25954 | NA |
| −1.78515 | −3.04023 | −3.00432 | −1.50209 | −1.64182 | −0.82916 | −2.27313 | NA | NA | −0.96161 | NA |
| −0.40381 | −0.69342 | −0.3093 | −1.10053 | −1.27659 | −1.38585 | −1.60072 | NA | NA | 3.084482 | −2.87178 |
| −1.07053 | −1.88857 | −1.53114 | −0.84116 | −0.89515 | 0.216873 | −0.55526 | NA | NA | 1.636245 | NA |
| −2.75469 | −9.45635 | −3.91801 | −2.02444 | −2.42616 | −2.97755 | −2.25937 | 4.081404 | NA | NA | NA |
| −3.56542 | NA | −5.77889 | −2.01039 | −3.44743 | −3.27371 | −3.16405 | 4.212216 | NA | NA | NA |
| −2.79709 | −7.95623 | −3.88196 | −1.70644 | −3.23115 | −3.36957 | −3.27433 | 4.204966 | NA | −4.59659 | NA |

[symbol]

indicates data missing or illegible when filed

APPENDIX: TABLE 3-3

| Metabolit | Sample ID | Diagnosis | 1,3,5-pen [symbol] | 1,3-dihyd [symbol] | 1,3-propa [symbol] | 1,5-anhyd [symbol] | 1-hexade [symbol] | 1-monoli [symbol] | 1-monop [symbol] |
|---|---|---|---|---|---|---|---|---|---|
| B851 | NS | NT | 0.374296 | NA | −1.00583 | 2.242986 | NA | 0 | 0.663894 |
| B854 | C4 | NT | 0.187691 | NA | −0.68141 | 1.788965 | NA | 0.621914 | 0.604689 |
| B859 | A17 | ASD | 0.622963 | NA | −0.76155 | 1.941167 | NA | 1.242084 | 0.616677 |
| B864 | A09 | ASD | 0.010531 | NA | −0.77596 | 1.973399 | NA | −0.01001 | 0.782099 |
| B869 | C1 | NT | 0.645945 | −0.98399 | 4.910697 | 2.342994 | NA | 0.022923 | 0.391671 |
| B873 | A24 | ASD | 0.283471 | 1.334809 | −0.87095 | 2.456735 | NA | 0.556426 | 0.443872 |
| B876 | A11 | ASD | 0.408015 | NA | −1.26262 | 2.184205 | NA | 0.4498 | 0.8053 |
| B881 | N5 | NT | 0.424118 | NA | 4.463933 | 2.110167 | NA | 0.102815 | 0.301123 |
| B884 | C4 | NT | 0.407149 | MA | −0.60876 | 2.207428 | NA | 0.990969 | 0.76738 |
| B889 | A17 | ASD | 0.536352 | NA | −0.46069 | 1.573478 | NA | 0.647115 | 0.535791 |
| B894 | A09 | ASD | 0.485638 | NA | −0.40986 | 2.237036 | NA | 0.550627 | 1.023256 |
| B899 | C1 | NT | 0.444864 | NA | −0.63217 | 2.797356 | NA | 0.378482 | 0.463694 |
| B904 | A24 | ASD | 0.202097 | NA | −1.09387 | 2.261506 | NA | 0.291465 | 0.088489 |
| B907 | A03 | ASD | 0.48251 | 0.855369 | −0.93594 | 2.404556 | −2.10772 | 0.879142 | 0.544679 |
| B912 | A11 | ASD | 0.229034 | NA | 4.732727 | 2.410245 | NA | 0.25148 | 0.330607 |
| B917 | NS | NT | 0.31164 | NA | −0.86691 | 2.321942 | NA | 0.30376 | 0.367601 |
| B922 | C4 | NT | 0.386678 | NA | −0.88163 | 1.804392 | NA | 0.287394 | 0.0538 |
| B927 | A17 | ASD | 0.678353 | NA | 4.124037 | 2.199489 | NA | 0.874791 | 0.65011 |
| B932 | A09 | ASD | 0.181049 | NA | −0.93124 | 2.061192 | NA | 0.487566 | 0.046038 |
| B937 | C1 | NT | 0.507303 | −1.71147 | 4.83554 | 2.462224 | NA | −0.12263 | 0.254869 |
| B941 | A24 | ASD | 0.095842 | NA | 0.034225 | 2.471793 | NA | 1.202134 | 1.136361 |
| B946 | A03 | ASD | −0.11755 | 0.006152 | 0.815341 | 1.875004 | NA | 0.251744 | 1.177748 |
| B947 | A11 | ASD | 0.309542 | NA | −0.47062 | 2.238488 | NA | 0.846861 | 0.537502 |
| B952 | NS | NT | 0.221888 | NA | −1.05611 | 1.153089 | NA | −0.34864 | 0.499347 |
| B957 | C4 | NT | 0.356374 | NA | −0.87035 | 2.22124 | NA | 0.206907 | 0.769498 |
| B962 | A17 | ASD | 0.177209 | −3.1031 | −0.04274 | 1.7922 | NA | 0.362779 | 0.863352 |
| B965 | A09 | ASD | 0.09042 | NA | −0.49304 | 2.271692 | NA | 0.599909 | 0.770177 |
| B970 | C1 | NT | 0.386887 | −3.19131 | 0.528013 | 2.097225 | NA | 0.524402 | 1.607262 |
| B973 | A03 | ASD | 0.544821 | NA | −0.94346 | 2.610822 | −1.36432 | 1.25865 | 0.880599 |
| B978 | A11 | ASD | −0.01364 | NA | −1.24785 | 1.943289 | NA | 0.49285 | 0.753175 |
| B983 | A03 | ASD | 0.360467 | 0.061979 | −0.07408 | 2.274256 | NA | −0.2079 | 0.773627 |
| B984 | NS | NT | −0.125S2 | 0.480514 | 0.269623 | 1.976771 | NA | 0.416027 | 1.022222 |
| B1007 | GF | GF | −0.23791 | NA | 4.728313 | 2.602676 | NA | −1.21573 | 0.708423 |
| B1010 | GF | GF | 0.159585 | NA | 4.818796 | 2.590533 | NA | −1.22016 | 0.056189 |
| B1013 | GF | GF | −0.03204 | NA | −0.80417 | 2.798385 | NA | −0.94994 | 0.929387 |
| Chow_1 | Chow | Chow | −0.23522 | NA | 0.303337 | −0.7041 | 1.880044 | 3.63748 | 3.428218 |
| Chow_2 | Chow | Chow | 0.13817 | NA | 0.022138 | −0.34379 | 1.624623 | 3.193933 | 2.947773 |
| Chow_3 | Chow | Chow | 0.285494 | NA | 0.405563 | −0.67197 | 1.685885 | 3.457163 | 3.247898 |

| 2-amino [symbol] | 2-ethylhe [symbol] | 2-hydroxy [symbol] | 2-hydroxy [symbol] | 2-hydroxy [symbol] | 2-ketoiso [symbol] | 2-linoleo [symbol] | 2-monop [symbol] | 2-piperid [symbol] |
|---|---|---|---|---|---|---|---|---|
| 2.331399 | NA | −1.28875 | 0.958907 | 0.142995 | −1.17635 | −0.50131 | −1.29893 | −0.22926 |
| 2.040975 | NA | −0.34368 | 1.065582 | −0.08308 | −1.14754 | 0.162105 | 0.107736 | −0.6048 |
| 2.023662 | −1.00695 | −0.63231 | 1.239696 | 0.423086 | −1.4087 | 0.438814 | 0.182883 | −0.71442 |
| 1.941268 | NA | 0.341786 | 1.025868 | 0.231425 | −1.80476 | −0.0661 | −0.74139 | −0.15311 |
| 2.472993 | NA | 0.524677 | 1.580805 | 0.991337 | −1.9107 | −0.24027 | −1.72878 | −2.35954 |
| 2.010336 | NA | −0.2661 | 1.07622 | 0.541968 | −1.49543 | −0.63233 | −1.11409 | −1.34356 |
| 2.292603 | NA | −1.02972 | 1.458944 | −0.10987 | −1.59042 | 0.379926 | −0.83613 | −0.68839 |
| 1.635297 | NA | −1.08775 | 1.163749 | 0.013395 | −1.27028 | −0.56736 | −0.8937 | −2.3094 |
| 2.452374 | 0.009246 | −1.09861 | 1.186516 | 0.088692 | −0.82518 | 0.552037 | −0.45457 | −1.41045 |
| 2.431278 | −0.17419 | −1.15386 | 1.274187 | 0.064128 | −1.3556 | 0.035097 | −0.31414 | −2.00763 |
| 2.099542 | NA | 0.02548 | 1.398844 | 0.008519 | −1.32957 | −0.43964 | −0.72922 | −1.02522 |
| 2.749853 | NA | −0.37049 | 1.509797 | 0.390508 | −0.89292 | −0.69289 | −1.36636 | −0.84515 |
| 1.945073 | NA | −0.22709 | 0.966785 | 0.528074 | −1.48765 | −0.28723 | −1.19466 | −1.67702 |
| 2.093044 | NA | −0.86687 | 1.179594 | 0.427368 | −2.55425 | 0.57483 | −0.20536 | −1.06214 |
| 2.452798 | −1.14875 | −1.24707 | 1.514625 | 0.313981 | −1.55572 | 0.005037 | −0.76193 | −1.65291 |
| 1.934947 | −0.46507 | −0.48869 | 1.176223 | 0.560493 | −1.25844 | −0.45963 | −1.56959 | −1.20139 |
| 2.150923 | NA | −1.57144 | 0.924442 | −0.40909 | −1.42533 | −0.29176 | −0.65161 | −1.12671 |
| 7.719989 | NA | −0.78209 | 1.364104 | 0.146591 | −1.2486 | 0.434917 | −0.22914 | −0.51924 |
| 1.752768 | NA | −0.21872 | 1.264347 | −0.22483 | −1.04289 | −0.31547 | −1.36864 | −1.03484 |
| 2.711045 | NA | −0.3193 | 1.14097 | 1.131919 | −1.22629 | −0.4323 | −1.1941 | −2.02418 |
| 2.085474 | −0.0127 | −0.3801 | 1.39054 | 0.779 | −1.3213 | −0.25615 | −0.38853 | −5.88298 |
| 0.399149 | 1.807046 | −0.74926 | 1.774759 | 1.396364 | −3.53919 | −1.45249 | −1.5144 | −0.45489 |
| 2.608905 | NA | −0.56556 | 1.388465 | 0.047192 | −1.68442 | −0.04415 | −0.4879 | −0.9894 |
| 2.706852 | NA | 0.797249 | 1.195151 | 0.234016 | −1.76804 | −1.58655 | −1.75605 | −0.69866 |
| 2.358687 | NA | −0.05624 | 1.228831 | 0.775568 | −0.62754 | −0.28776 | −1.07439 | −1.31415 |
| 1.21387 | 0.118239 | −1.03054 | 1.477336 | 0.411929 | −1.68003 | −0.98816 | −1.18134 | −0.05126 |
| 1.891127 | NA | −0.19859 | 1.362012 | 0.220439 | −0.74953 | −0.09972 | −0.46059 | −0.17324 |
| 1.236126 | NA | −0.50898 | 1.809056 | 0.656257 | −5.76997 | −1.47198 | −1.05543 | 0.245942 |
| 2.342801 | NA | −0.3837 | 1.231805 | 0.865942 | −1.84484 | 0.400789 | −0.86379 | −0.76003 |
| 2.435262 | NA | −0.27017 | 1.263789 | −0.04182 | −1.97852 | −0.93635 | −0.94532 | −0.56004 |
| 0.566192 | 1.662075 | −0.41275 | 1.554209 | 0.3952 | −2.04368 | −1.07625 | −1.33196 | 0.26503 |
| 0.37085 | 1.099782 | −0.65185 | 1.585004 | 0.597878 | −1.97558 | −0.87804 | −0.55282 | 0.319688 |
| 1.931375 | NA | 1.019363 | 0.73936 | 1.244024 | −4.75638 | −2.7943 | −1.687 | −0.47785 |
| 2.406492 | NA | 1.096239 | 0.873366 | 1.276722 | −4.68052 | −1.73352 | −1.85207 | −1.51747 |
| 2.588283 | NA | 1.266215 | 0.917796 | 1.23745 | −3.68597 | −1.65394 | −1.56279 | −6.14952 |
| NA | NA | −0.22095 | 0.485584 | 1.143874 | −3.89615 | −1.26594 | 0.360425 | 0.161303 |
| −1.87546 | NA | −1.71229 | −0.12515 | 1.022918 | −3.74565 | −1.63446 | −0.00613 | −0.24863 |
| −2.32481 | NA | −1.66555 | 0.133486 | 1.232162 | −4.1313 | −1.38475 | 0.137327 | 0.046199 |

| 3-(4-hydr [symbol] | 3,5-dimet [symbol] | 3-amino- [symbol] | 3-aminois [symbol] | 3-hydroxy [symbol] | 3-hydroxy [symbol] | 3-phosph [symbol] | 4-guanidi [symbol] | 4-hydroxy [symbol] | 4-hydroxy [symbol] |
|---|---|---|---|---|---|---|---|---|---|
| NA | NA | 0.870529 | NA | NA | 0.854061 | 0.805686 | NA | NA | 3.269171 |
| NA | NA | 1.445837 | NA | NA | 1.036586 | 1.320465 | NA | NA | 2.954963 |
| NA | NA | 1.321047 | NA | NA | 0.373512 | 0.969044 | NA | NA | 3.11811 |
| NA | NA | 1.012514 | NA | NA | 0.565062 | −1.11734 | NA | NA | 2.386117 |
| NA | NA | 2.050034 | NA | NA | 2.382998 | −0.8794 | NA | NA | 3.025887 |
| NA | NA | 1.792524 | NA | NA | 1.060095 | −1.62617 | NA | NA | 3.039684 |
| NA | NA | 1.301717 | NA | NA | 1.620578 | 2.03572 | NA | NA | 3.139932 |
| NA | NA | 1.853858 | NA | NA | 1.373732 | 2.483559 | NA | NA | 3.223662 |
| NA | NA | 1.832595 | NA | NA | 0.272819 | 1.665867 | NA | NA | 3.346168 |
| NA | NA | 1.151638 | NA | NA | −0.17121 | 1.788893 | NA | NA | 3.745411 |
| NA | NA | 1.879874 | NA | NA. | 0.653389 | 0.238576 | NA | NA | 2.715963 |
| NA | NA | 1.791149 | NA | NA | 1.006397 | −1.34948 | NA | NA | 3.191517 |
| NA | NA | 1.778173 | NA | NA | 1.144654 | −0.58628 | NA | NA | 2.79995 |
| NA | NA | 1.283875 | NA | NA | 0.824254 | −1.63627 | NA | NA | 3.020412 |
| NA | NA | 2.001036 | NA | NA | 0.189315 | −1.10296 | NA | NA | 3.236861 |
| NA | NA | 1.476127 | NA | NA | 0.580818 | −0.28363 | NA | NA | 3.278783 |
| NA | NA | 1.0075 | NA | NA | 0.622244 | 2.300482 | NA | NA | 3.437038 |
| NA | NA | 1.640611 | NA | NA | 0.946225 | 1.273 | NA | NA | 3.375149 |
| NA | NA | 1.393102 | NA | NA | 0.606341 | 1.269674 | NA | NA | 2.671804 |
| NA | NA | 2.633442 | NA | NA | 1.670755 | −1.65345 | NA | NA | 3.276888 |
| NA | NA | 1.563205 | NA | NA | 0.542567 | −1.65378 | NA | NA | 2.851378 |
| NA | NA | 0.069683 | NA | NA | 0.348055 | −2.50433 | NA | NA | 1.947551 |
| NA | NA | 1.965561 | NA | NA | 0.595222 | 0.578884 | NA | NA | 3.484864 |
| NA | NA | 1.205628 | NA | NA | 1.112457 | −0.69096 | NA | NA | 3.22902 |
| NA | NA | 1.495318 | NA | NA | 1.490986 | −1.81492 | NA | NA | 3.241845 |
| NA | NA | 0.566766 | NA | NA | 0.31665 | −1.47945 | NA | NA | 3.248701 |
| NA | NA | 1.263742 | NA | NA. | 1.166467 | 0.542996 | NA | NA | 2.870015 |
| NA | NA | 1.394807 | NA | NA | 1.190865 | −3.18122 | NA | NA | 2.782251 |
| NA | NA | 0.988163 | NA | NA | 2.028024 | −1.474 | NA | NA | 2.951264 |
| NA | NA | 1.056334 | NA | NA | 0.494556 | 0.494732 | NA | NA | 3.092829 |
| NA | NA | 1.157807 | NA | NA | 0.575376 | −3.00078 | NA | NA | 2.490901 |
| NA | NA | 0.490755 | NA | NA | 1.047392 | −0.14384 | NA | NA | 2.422326 |
| NA | NA | 1.148567 | NA | NA | 0.895064 | −2.17642 | NA | NA | 2.145353 |
| NA | NA | 1.75436 | NA | NA | 0.658411 | −1.50305 | NA | NA | 2.539767 |
| NA | NA | 1.290299 | NA | NA. | 1.03162 | −1.68797 | NA | NA | 2.391007 |
| −2.29181 | −1.64422 | −0.86442 | −3.30888 | 0.938942 | NA | −5.01167 | 2.682019 | −0.98908 | −2.38441 |
| −1.64245 | −2.23641 | −0.45098 | −2.83131 | 0.758649 | −4.5946 | −4.15794 | 3.323933 | −0.23129 | −1.30305 |
| −1.28055 | −2.15471 | 0.07723 | −2.39885 | 0.586202 | −4.95164 | −4.68922 | 3.395473 | −0.4245 | −2.30671 |

| 4-hydroxy [symbol] | 4-methyl- [symbol] | 5-aminov [symbol] | aconitic a [symbol] | adenine | adenosin [symbol] | adenosin [symbol] | allantoin | alpha ket [symbol] |
|---|---|---|---|---|---|---|---|---|
| NA | NA | −1.59694 | −0.88183 | −3.48713 | −0.0606 | 2.122141 | −0.0663 | 0.906424 |
| NA | −2.05207 | −0.15583 | −0.82291 | NA | −2.78657 | NA | −0.96204 | 0.985069 |
| NA | NA | −1.17398 | −0.71197 | NA | −4.1005 | NA | −1.3614 | 1.24152b |
| NA | NA | −1.11907 | −0.62785 | NA | −1.80533 | −2.59362 | −2.27902 | 1.509825 |
| NA | NA | −0.95316 | 0.023694 | NA | −0.08934 | −0.07627 | −0.34 | 2.289686 |
| NA | NA | −1.01089 | −1.57346 | NA | −4.66692 | NA | −0.15538 | 1.33663 |
| NA | NA | −0.14644 | −0.97015 | −3.5105 | −1.11677 | −0.46548 | −0.26592 | 0.854352 |
| NA | NA | −1.22143 | −0.58711 | NA | −0.3411 | 0.533705 | −0.09969 | 0.828776 |
| NA | −2.13257 | −0.44309 | −0.69791 | NA | −1.68683 | −1.99457 | −0.90991 | 1.203659 |
| NA | NA | −1.13269 | −0.94316 | NA | −1.249 | −0.84381 | −0.14019 | 0.844784 |
| NA | NA | −0.8507 | −0.58896 | NA | −1.02486 | −0.59253 | −1.41523 | 1.450726 |
| NA | NA | −0.89474 | −0.79155 | NA | −0.50895 | −0.87831 | −0.41932 | 1.958318 |
| NA | NA | −1.33567 | −1.53454 | NA | NA | NA | −0.52942 | 1.339796 |
| NA | NA | −1.49149 | −1.38182 | NA | −5.96718 | NA | −0.62023 | 1.063172 |
| NA | NA | −1.03455 | −0.53723 | NA | 0.425923 | 0.353722 | −0.92293 | 1.263637 |
| NA | NA | −1.33314 | −0.51473 | NA | 0.374829 | 1.578191 | −1.00745 | 1.087498 |
| NA | −2.2729 | −0.79999 | −0.93245 | NA | −0.99015 | 0.41743 | −0.56142 | 0.398102 |
| NA | NA | −1.1968 | −0.92606 | NA | −1.55628 | −1.21971 | −0.95858 | 0.870081 |
| NA | NA | −1.01448 | −0.94074 | −3.56676 | −1.30605 | −0.73066 | −1.11062 | 0.928897 |
| NA | NA | −0.90678 | 0.016589 | NA | 0.733907 | 0.112759 | −0.51768 | 1.614228 |
| NA | NA | −0.99388 | −1.4191 | NA | NA | NA | −4.19526 | 1.200986 |
| −1.5108 | NA | −1.92687 | −2.11562 | NA | NA | −2.51235 | NA | −0.17367 |
| NA | NA | −0.1874 | −0.73175 | NA | −0.19251 | 0.283493 | −0.24638 | 1.167327 |
| NA | NA | −0.01167 | −0.76942 | NA | 0.210436 | 0.75628 | −0.21127 | 2.560645 |
| NA | −1.87412 | −0.22558 | −0.35143 | NA | 0.58081 | 0.327357 | −2.02496 | 1.320193 |
| NA | NA | −1.42193 | −3.61405 | NA | NA | NA | −1.28135 | 0.663809 |
| NA | NA | −0.89213 | −0.82854 | NA | −0.38651 | 0.673254 | −0.97284 | 1.081545 |
| NA | NA | −0.82396 | −1.81649 | NA | −0.63744 | NA | NA | 0.79759 |
| NA | NA | −1.33634 | −0.29279 | NA | −6.11651 | NA | −0.78174 | 1.302564 |
| NA | NA | −1.08496 | −0.79991 | NA | −0.30588 | 0.632165 | −1.50418 | 0.693229 |
| NA | NA | −0.94031 | −1.46851 | NA | NA | NA | −0.98904 | 0.563262 |
| NA | NA | −1.15757 | −1.35397 | NA | −3.23456 | NA | −3.3203 | 0.213586 |
| NA | NA | −1.58427 | −0.35348 | NA | 0.989449 | 1.869688 | −4.94649 | 2.024359 |
| NA | NA | −1.48567 | −0.31623 | NA | 1.235026 | 1.77285 | −2.78783 | 1.681468 |
| −2.25375 | NA | −1.49111 | −0.61299 | −3.61529 | 0.357552 | −0.91799 | −3.44968 | 1.531847 |
| 0.005237 | 0.55956 | 1.054742 | −1.91125 | 0.796835 | 1.273875 | NA | NA | −0.95574 |
| 0.319786 | 1.043964 | 1.806539 | −1.66681 | 1.280627 | 2.041984 | −2.97093 | −6.07545 | −0.80454 |
| −3.89305 | 0.467824 | 1.833341 | −1.38233 | 1.49802 | 2.047091 | −3.251 | −4.55287 | −0.65435 |

| alpha-toc [symbol] | arabitol | arachidic [symbol] | arachidon [symbol] | behenic a [symbol] | beta-sitos [symbol] | campeste | capric aci [symbol] | carbonate [symbol] | cellobiose |
|---|---|---|---|---|---|---|---|---|---|
| −1.98782 | NA | −1.00795 | 0.942809 | −1.25133 | −2.15648 | −1.76432 | 1.101926 | 6.325546 | NA |
| −1.75359 | NA | −0.53436 | 0.88379 | −1.31555 | −2.04831 | −1.16969 | 0.790466 | 6.828573 | NA |
| −1.05103 | NA | 0.023253 | 0.957828 | −2.50164 | −1.84594 | −0.94871 | −0.63483 | 6.495825 | NA |
| −0.4015 | NA | 0.345474 | 0.336058 | −2.05689 | −1.56481 | −0.91197 | −3.12641 | 6.962401 | NA |
| 0.164651 | NA | −0.00724 | 0.637621 | 0.007241 | −2.47709 | −1.34165 | −2.24758 | 7.329701 | NA |
| −0.20034 | NA | 1.125167 | 0.819514 | −2.40595 | −2.90297 | −1.89799 | −1.25743 | 6.736247 | NA |
| −1.40046 | NA | 0.763362 | 1.271867 | −4.47416 | −1.65114 | −0.89428 | −2.62857 | 6.648338 | NA |
| −2.53599 | NA | −0.81062 | 0.91406 | −2.81991 | −2.62507 | −1.54481 | 0.900773 | 6.414261 | NA |
| −1.35231 | NA | 0.090527 | 1.200677 | −1.67801 | −1.38096 | −0.64645 | −2.71865 | 7.082237 | NA |
| −1.90098 | NA | −0.28788 | 0.840465 | −1.96935 | −2.04655 | −1.15214 | 0.511963 | 6.538155 | NA |
| −1.57452 | −0.57574 | 0.689351 | 0.710027 | −1.07305 | −1.99647 | −0.94431 | −1.54479 | 6.720134 | NA |
| −0.69594 | NA | 0.755238 | 0.64828 | −0.02143 | −2.12443 | −1.28477 | −2.8047 | 7.325313 | NA |
| −0.88009 | NA | 1.103914 | 0.776715 | −2.19875 | −2.21079 | −1.68949 | 0.995513 | 6.628749 | NA |
| 0.661452 | NA | 0.988615 | 0.893938 | −2.81384 | −2.25619 | −1.1363 | −2.05076 | 6.727182 | NA |
| 0.016779 | NA | 0.103263 | 0.662166 | −2.37051 | −1.5824 | −1.00262 | 3.195912 | 7.018467 | NA |
| −1.05728 | NA | −0.57059 | 0.419019 | −1.4958 | −2.20916 | −1.14905 | 0.414829 | 6.803663 | NA |
| −2.33089 | NA | 0.040102 | 0.482945 | −3.04638 | −2.1876 | −1.17318 | 0.999481 | 6.33666 | NA |
| −1.27125 | NA | −0.39061 | 1.293028 | −1.24938 | −1.99974 | −1.1825 | 0.27613 | 6.511751 | NA |
| −2.14843 | NA | −0.40859 | 0.702792 | −3.17694 | −1.69584 | −0.91658 | 2.511646 | 6.423472 | NA |
| −0.15068 | NA | 0.011984 | 0.738562 | −0.55122 | −2.64912 | −1.51154 | 1.622992 | 7.196182 | NA |
| 0.559645 | NA | 1.362285 | 1.403536 | 0.307846 | −2.0683 | −1.65372 | −1.91124 | 6.931544 | NA |
| NA | NA | 0.195809 | 0.345565 | −2.95283 | NA | NA | −0.08288 | 6.208383 | NA |
| −1.29719 | NA | −0.06789 | 0.601965 | −4.29226 | −1.773 | −1.11675 | −0.17645 | 6.754228 | NA |
| −1.42271 | NA | −0.47991 | 0.420944 | −2.90267 | −2.70964 | −1.98576 | −0.02453 | 6.816789 | NA |
| 0.669609 | NA | 0.007728 | 0.576942 | −0.61605 | −1.23436 | −0.64493 | 0.927408 | 7.260572 | NA |
| −2.561 | NA | −1.26639 | 0.672257 | −3.08235 | −2.78494 | −2.38169 | 0.322419 | 6.065557 | NA |
| −1.23761 | NA | −0.38022 | 1.194254 | −3.46179 | −1.63502 | −0.61753 | −0.77648 | 6.774171 | NA |
| −2.88621 | NA | −0.72861 | 1.062683 | −1.88041 | −4.77862 | −2.73963 | 2.682654 | 6.398934 | NA |
| 1.015342 | NA | 1.173611 | 1.272039 | −1.83711 | −1.27164 | −0.87373 | −0.66252 | 7.033141 | NA |
| −2.08841 | NA | −0.1795 | 0.551187 | −2.01363 | −2.05757 | −1.18327 | −0.90087 | 6.622616 | NA |
| −1.95684 | NA | 0.106214 | 0.451812 | −1.30394 | −5.45765 | −4.81062 | −1.89161 | 5.990007 | NA |
| −2.58348 | NA | 0.328308 | 1.217481 | −2.34868 | −4.02836 | −2.4272 | −0.92853 | 6.232787 | NA |
| 1.151418 | NA | −0.51087 | 0.235905 | −0.23734 | −2.92312 | −1.8496 | −2.97417 | 6.453317 | NA |
| 1.158488 | NA | −0.56424 | 0.563848 | 0.577634 | −2.41641 | −1.23825 | −2.90541 | 6.662399 | NA |
| 0.919692 | NA | −0.81865 | 0.596534 | 0.762178 | −1.81576 | −1.69326 | −2.76181 | 6.906076 | NA |
| 0.720525 | 0.791723 | 0.122806 | 1.833708 | 2.050883 | 1.68737 | −0.30741 | −0.61179 | 5.305723 | 0.196641 |
| 0.329528 | 1.795218 | −0.37159 | 1.377149 | 1.785734 | 1.13146 | −0.83527 | −0.1761 | 4.55217 | 2.310519 |
| 0.511754 | 1.546962 | 0.000626 | 1.83961 | 2.092297 | 1.241306 | −0.69695 | −1.00214 | 4.712324 | 2.31166 |

| cholester | citric acid | creatinin [symbol] | cycloleuci [symbol] | daidzein | dehydrat [symbol] | dehydroa [symbol] | D-erythrit [symbol] | D-gluconi [symbol] |
|---|---|---|---|---|---|---|---|---|
| 4.558409 | 4.34108 | 4.27279 | 0.031189 | −5.92254 | −1.95201 | −1.32248 | −1.10241 | 0.773953 |
| 4.637647 | 4.272324 | 2.222934 | −0.14216 | −4.63541 | −2.09817 | −1.0558 | −1.10594 | 0.598481 |
| 4.616975 | 4.192398 | 2.296336 | −0.07132 | −3.73247 | −1.74479 | −1.53903 | −1.04148 | 0.868415 |
| 4.723548 | 4.118895 | 1.080102 | −0.33169 | −3.68434 | −2.40412 | −1.95313 | −1.49911 | 0.699786 |
| 4.767857 | 5.099322 | 1.6758 | 0.470251 | NA | −2.19336 | −2.03374 | −0.46255 | 1.534871 |
| 4.26501 | 3.896121 | 2.083411 | 0 | −4.3899 | −1.30335 | −2.34405 | −0.72192 | 0.743331 |
| 4.733435 | 4.327355 | 3.241245 | −0.22897 | −4.97747 | −1.83335 | −1.05274 | −0.95021 | 1.076246 |
| 4.6496 | 4.30497 | 2.846684 | −0.12549 | NA | −1.43548 | −1.07439 | −1.1065 | 0.970593 |
| 5.050852 | 4.334803 | 3.420408 | 0.314223 | −4.29876 | −1.8634 | −1.14567 | −0.82356 | 0.831302 |
| 4.573762 | 3.959606 | 3.16671 | 0.049557 | −4.36781 | −1.56145 | −0.60404 | −0.90509 | 1.216536 |
| 4.502471 | 4.44624 | 1.390198 | 0.135202 | −4.18119 | −1.78155 | −1.43668 | −1.09117 | 0.938578 |
| 4.741622 | 4.414945 | 2.019469 | 0.528018 | −5.0128 | −1.22797 | −1.11683 | −1.0632 | 1.138863 |
| 4.546175 | 4.160189 | 1.633543 | −0.28916 | −5.73228 | −1.3823 | −1.54322 | −0.83813 | 0.793418 |
| 4.720198 | 4.127386 | 1.543041 | 0.166894 | −4.75904 | −1.84987 | −2.27737 | −0.74091 | 1.123446 |
| 4.893786 | 4.273333 | 7 | 0.637837 | −4.86145 | −1.443 | −2.38366 | −0.87107 | 0.636759 |
| 4.927926 | 4.030323 | 1.948779 | −0.31931 | −5.5514 | −1.18153 | −1.31093 | −1.02142 | 1.44269 |
| 4.583169 | 3.899475 | 2.872153 | −0.02913 | −6.23545 | −2.17101 | −1.34206 | −0.90296 | 0.826263 |
| 4.591991 | 4.057978 | 3.279815 | 0.274983 | −4.93529 | −1.74048 | −0.56017 | −0.7914 | 1.073878 |
| 4.598744 | 4.273016 | 1.962084 | 0.064814 | −5.58774 | −2.41537 | −1.07485 | −1.16164 | 0.998633 |
| 4.774296 | 4.632533 | 1.563818 | 0.185125 | NA | −1.71271 | −2.10301 | −0.84886 | 1.454248 |
| 5.084001 | 4.027891 | 1.016758 | −0.27696 | −4.78901 | −2.04834 | −1.64727 | −0.93829 | 0.978327 |
| 4.28121 | 0.965053 | −1.06852 | −1.4168 | NA | −4.13771 | −2.65484 | −1.69089 | −3.85736 |
| 4.784404 | 4.096562 | 2.888728 | 0.555721 | −3.82481 | −0.9409 | −0.82988 | −1.01537 | 1.251437 |
| 4.740354 | 4.438509 | 2.188294 | −0.58242 | −3.88657 | −0.64556 | −0.56457 | −1.24163 | 1.076094 |
| 5.091717 | 4.266359 | 1.687131 | 0.129117 | NA | −1.98159 | −1.99367 | −0.87685 | 0.961122 |
| 4.391826 | 1.977888 | 2.365461 | −0.27964 | NA | −2.70554 | −1.98377 | −1.32524 | 0.156895 |
| 4.804024 | 4.264756 | 2.262456 | 0.046308 | −4.54366 | −2.24848 | −0.93958 | −1.28773 | 0.996345 |
| 4.119952 | 2.374781 | −0.02249 | −0.179 | NA | −1.66999 | −2.80225 | −1.00043 | 0.479873 |
| 4.907587 | 4.467533 | 0.786748 | 0.078703 | −5.54951 | −2.19794 | −0.85707 | −0.46679 | 0.7764 |
| 4.568971 | 3.723224 | 2.068403 | −0.02867 | −3.97026 | −1.18523 | −1.62338 | −1.275 | 0.613681 |
| 4.015849 | 2.285036 | 0.322658 | −0.22248 | −5.28184 | −2.439 | −1.76525 | −G.97329 | 0.473657 |
| 4.497768 | 3.062718 | 0.442673 | −0.55592 | −6.13353 | −2.36494 | −1.42668 | −1.07486 | −0.04636 |
| 4.835267 | 3.955085 | 0.829251 | −1.3111 | NA | −2.18495 | −1.30988 | −1.25299 | 0.906437 |
| 4.987855 | 4.101732 | 1.168129 | −0.9113 | −6.69189 | −1.71545 | −1.80213 | −1.1045 | 1.076069 |
| 4.886299 | 4.153973 | 1.117255 | −1.39365 | NA | −2.09094 | −2.22573 | −1.21092 | 0.816515 |
| 2.545737 | 4.162874 | 0.965646 | 0.246739 | −2.30536 | −1.03858 | −4.74151 | 0.484046 | 1.916104 |
| 1.952916 | 3.834195 | 1.512761 | 1.022564 | −1.53556 | −0.58697 | −3.16738 | 1.059461 | 2.649705 |
| 2.158801 | 4.1243 | 1.583246 | 1.350362 | −1.5976 | −0.23965 | −2.95826 | 1.372298 | 2.730241 |

| D-glucose | D-glucose | D-malic a [symbol] | D-maltito [symbol] | D-mannit [symbol] | D-manno [symbol] | docosahe [symbol] | D-pinnito [symbol] | D-ribose | D-ribose- [symbol] |
|---|---|---|---|---|---|---|---|---|---|
| 8.945905 | −1.58684 | 2.7455 | NA | −0.42863 | 3.573185 | 1.770023 | −0.41893 | 2.054855 | 2.195729 |
| 9.02157 | −0.38278 | 1.782206 | NA | 0.804808 | 3.24793 | 2.08635 | −0.53337 | 3.4955 | 1.661839 |
| 9.036484 | −0.6848 | 1.904876 | NA | 1.082332 | 2.32.9466 | 2.108985 | −0.17637 | 3.82088 | 1.518734 |
| 8.961742 | −1.45995 | 1.471922 | NA | 0.762394 | 2.535827 | 1.064378 | 0.298036 | 2.813689 | 0.000271 |
| 9.555118 | −0.85436 | 2.673265 | NA | 1.403393 | 4.553491 | 2.095822 | −0.19272 | 3.115007 | −0.30933 |
| 8.761556 | −1.01182 | 1.919009 | NA | 0.616227 | 1.98623 | 2.2.51741 | 0.215695 | 3.732185 | 0.968484 |
| 9.023548 | −0.28584 | 2.081582 | NA | 0.915187 | 3.121545 | 2.644639 | −2.02756 | 3.836103 | 2.398554 |
| 9.010901 | −0.71929 | 2.266531 | NA | 0.616732 | 3.12056 | 1.915656 | −0.68299 | 2.538152 | 2.2372 |
| 9.204446 | −0.73638 | 1.929748 | NA | 1.226823 | 3.234573 | 2.419379 | −0.43523 | 3.411769 | 2.120299 |
| 9.03904 | −0.1653 | 1.768861 | NA | 0.88017 | 2.897235 | 1.752061 | 0.067183 | 3.860473 | 1.678384 |
| 9.050701 | −1.19761 | 1.818062 | NA | 0.766016 | 2.637047 | 1.453429 | −0.16717 | 2.762737 | 0.790077 |
| 9.156066 | −1.15343 | 1.952894 | NA | 1.346768 | 2.574634 | 2.161658 | −0.0461 | 2.985666 | −1.09282 |
| 8.858769 | −0.84641 | 2.063895 | NA | 0.577527 | 2.670019 | 2.226511 | −0.15314 | 3.172247 | 1.259405 |
| 8.953222 | −1.03275 | 1.613655 | NA | 0.58558 | 3.13486 | 2.551661 | −0.51656 | 3.737845 | 1.506491 |
| 9.169048 | −1.12706 | 1.195546 | NA | 1.436148 | 3.138668 | 2.074887 | −0.48134 | 2.855739 | −0.67527 |
| 9.160457 | −1.1959 | 1.784756 | NA | −0.27728 | 2.629842 | 1.787298 | −0.07691 | 1.000621 | −1.13906 |
| 8.893648 | −0.61987 | 1.735174 | NA | 1.027579 | 3.378701 | 1.645382 | −0.76156 | 3.096087 | 2.100765 |
| 8.992061 | −0.73285 | 1.943831 | NA | 1.283645 | 3.198336 | 2.442952 | −0.83427 | 3.764106 | 1.969241 |
| 8.847394 | −1.20929 | 1.790758 | NA | 0.456864 | 2.925799 | 1.681722 | −0.14764 | 3.248702 | 1.559321 |
| 8.62461 | −1.32738 | 2.030179 | NA | 1.417694 | 3.746915 | 2.185761 | −0.00497 | 2.446574 | −1.67374 |
| 9.201372 | −1.74032 | 1.750722 | NA | 0.425807 | 2.72049 | 2.289442 | 0.153933 | 2.450184 | −2.94537 |
| 9.249952 | −3.09243 | 1.048486 | NA | −0.68701 | 2.680902 | 1.115507 | −0.66626 | 2.936681 | −5.55787 |
| 9.178186 | −1.29679 | 1.847347 | NA | 1.283502 | 2.592444 | 1.513718 | −0.26861 | 2.755436 | 1.261801 |
| 8.68956 | −1.63878 | 2.771003 | NA | −0.37101 | 2.981225 | 1.147659 | 0.308705 | 1.403378 | −0.85531 |
| 8.978645 | −1.03324 | 1.430677 | NA | 1.775304 | 3.283716 | 1.893592 | −0.30714 | 1.768535 | −1.77213 |
| 9.107771 | −4.63835 | 1.445144 | NA | 0.960316 | 2.512828 | 1.536131 | −0.18942 | 2.606882 | −0.51757 |
| 9.231782 | −1.72888 | 1.888112 | NA | 0.640877 | 3.187492 | 1.975038 | −0.20762 | 2.520562 | 0.884972 |
| 9.632136 | −2.02255 | 0.933141 | NA | 1.338506 | 3.386981 | 1.740156 | −0.15138 | 2.530989 | −2.04392 |
| 8.573509 | −0.67979 | 1.764202 | NA | 0.584345 | 3.273694 | 2.714181 | −1.07016 | 3.241073 | 0.376221 |
| 8.795 | −1.87598 | 1.428354 | NA | 1.138677 | 2.534172 | 1.282109 | −0.03402 | 2.084547 | 0.145019 |
| 9.11S8S3 | −1.67346 | 1.221902 | NA | −0.22171 | 2.302669 | 1.422706 | −0.22911 | 4.188947 | 0.641251 |
| 9.059038 | −0.97346 | 1.484347 | NA | 0.820165 | 2.629451 | 2.153009 | −0.28725 | 3.851804 | 1.481899 |
| 9.179364 | −2.45879 | 2.608964 | NA | 1.074626 | 2.355211 | 0.487862 | 1.70897 | −0.58163 | −2.37154 |
| 9.252943 | −1.27135 | 2.420631 | NA | 1.100997 | 2.598204 | 0.739315 | 1.926169 | −0.33343 | −1.7511 |
| 9.14501 | −1.67606 | 2.146604 | NA | 1.068759 | 2.816302 | 1.213558 | 1.72242 | −0.3014 | −3.56156 |
| 3.984106 | NA | 2.799536 | 0.044497 | 0.874795 | 0.159627 | 3.051603 | 5.564241 | −2.29744 | NA |
| 4.644457 | −4.76954 | 3.326773 | 1.014622 | 1.520057 | 0.73735 | 2.469714 | 6.200545 | −1.79614 | −4.86284 |
| 4.734362 | −3.82057 | 3.385449 | 0.92.128 | 1.518227 | 0.779687 | 2.781786 | 6.287916 | −1.64023 | −5.12217 |

| D-ribulos [symbol] | D-threitol | eicosaper | ethanola [symbol] | ferulic aci [symbol] | fructose | fumaric a [symbol] | galactinol | galactitol |
|---|---|---|---|---|---|---|---|---|
| 2.243636 | NA | −0.60938 | −1.34667 | NA | 1.145991 | 1.057223 | NA | 0.770605 |
| 1.860888 | NA | −0.38741 | −0.69431 | NA | 1.402298 | 0.573637 | NA | 0.83358 |
| 1.596822 | NA | −0.40372 | −0.84039 | NA | 0.122176 | 0.426702 | NA | 0.547008 |
| 0.863998 | NA | −0.981 | −0.6025 | NA | 1.115998 | 0.305205 | NA | 0.587571 |
| 0.554843 | NA | −0.55472 | −0.59851 | NA | 3.20519 | 1.051422 | NA | 1.388048 |
| 1.708798 | NA | −0.38325 | −0.81549 | NA | 1.905558 | 0.489099 | NA | 1.23383 |
| 2.783212 | NA | −0.2094 | −1.92439 | NA | 1.000846 | 0.633639 | NA | 0.919731 |
| 2.367074 | NA | −0.54415 | −0.94396 | NA | 2.099375 | 0.469548 | NA | 0.964297 |
| 2.277504 | NA | −0.18579 | −0.62003 | NA | 1.880516 | 0.479736 | NA | 1.016031 |
| 1.928924 | NA | −1.0063 | −0.96211 | NA | 1.243887 | −0.06642 | NA | 0.744088 |
| 0.724675 | NA | −0.73099 | −1.13782 | NA | 1.88826 | 0.384126 | NA | 0.880911 |
| 0.447442 | NA | −0.45514 | −0.51372 | NA | 1.469994 | 0.373778 | NA | 1.256639 |
| 1.767982 | NA | −0.48467 | −1.09223 | NA | 1.822876 | 0.496074 | NA | 1.44661 |
| 1.23175 | NA | −0.31267 | −0.68284 | NA | 2.460561 | 0.245799 | NA | 1.267802 |
| 0.451499 | NA | −0.49523 | −0.76188 | NA | 1.389446 | −0.36901 | NA | 1.041653 |
| 0.186957 | NA | −0.74852 | −0.62564 | NA | 1.933733 | 0.255294 | NA | 1.044137 |
| 2.200961 | NA | −0.70976 | −1.1297 | NA | 1.259841 | 0.168929 | NA | 0.850932 |
| 2.048639 | NA | −0.29279 | −1.06585 | NA | 2.222562 | 0.630111 | NA | 0.904586 |
| 1.603236 | NA | −0.66776 | −1.5584 | NA | 0.746875 | 0.174666 | NA | 0.774813 |
| −0.05707 | NA | −0.41672 | −0.70036 | NA | 2.281782 | 0.392232 | NA | 1.344331 |
| 1.074392 | NA | −0.12024 | −0.04427 | NA | 1.861207 | 0.794533 | NA | 1.288454 |
| 0.113245 | −1.85037 | −0.86632 | 1.115681 | NA | 0.929096 | −0.00615 | NA | −0.60688 |
| 1.30215 | NA | −1.04075 | −0.84015 | NA | 2.751811 | 0.260879 | NA | 1.090594 |
| −0.09108 | NA | −1.68597 | −0.43919 | NA | 2.030911 | 1.237056 | NA | 1.106118 |
| −0.138 | NA | −0.6564 | −0.56599 | NA | 2.579796 | 0.147513 | NA | 1.510528 |
| −0.41182 | NA | −1.00948 | 0.072603 | NA | 0.697743 | 0.451947 | NA | 0.168903 |
| 0.915597 | NA | −0.42517 | −0.37483 | NA | 1.286363 | 0.627704 | NA | 0.677021 |
| −0.32996 | NA | −0.95736 | 0.519442 | NA | 1.47605 | 0.547199 | NA | 0.573243 |
| 0.966974 | NA | −0.41928 | −0.56551 | NA | 2.343868 | 0.761608 | NA | 1.519316 |
| 0.594468 | NA | −1.50562 | −1.15359 | NA | 1.359828 | 0.15177 | NA | 0.674002 |
| 1.085467 | NA | −0.76418 | 0.10415 | NA | 1.706675 | 0.401604 | NA | 0.506397 |
| 1.846327 | NA | −0.23849 | 0.255352 | NA | 1.31606 | 0.386457 | NA | −0.07533 |
| 0.541185 | NA | −2.2153 | −0.91278 | NA | 1.93618 | 1.329075 | 0.310836 | 1.298792 |
| 0.732054 | NA | −1.83898 | −0.99702 | NA | 2.405845 | 1.156167 | 0.444669 | 1.554051 |
| 0.203432 | NA | −1.70402 | −0.72929 | NA | 2.003189 | 1.028303 | 0.904962 | 1.476737 |
| NA | −0.74496 | 2.916986 | 0.33305 | −1.60558 | 5.486612 | −1.33509 | 5.591386 | 3.705748 |
| −4.90633 | 0.002133 | 2.421194 | 0.036587 | −0.86819 | 6.039919 | −0.61203 | 6.368546 | 4.40263 |
| −4.45886 | −0.00063 | 2.737268 | 0.125842 | −0.71743 | 6.149099 | −0.70285 | 6.367806 | 4.472912 |

| galactoni [symbol] | gamma-li [symbol] | genistein | glucosyl g [symbol] | glyceric a [symbol] | glycerol | glycerol 3 [symbol] | glycerol [symbol] | glycine | glycolic a [symbol] |
|---|---|---|---|---|---|---|---|---|---|
| 0.080932 | −1.49688 | −6.00896 | NA | −1.51769 | 4.766028 | 4.125072 | 0.035127 | 5.17342 | −3.34101 |
| −0.93696 | −1.04064 | −3.69077 | NA | −1.38616 | 5.428496 | 3.262042 | 0.283942 | 4.619065 | −3.52413 |
| −0.66019 | −1.04286 | −2.72368 | NA | −1.20476 | 5.134796 | 3.350334 | −0.45752 | 4.845828 | −3.50838 |
| −0.74124 | −1.69415 | −3.28645 | NA | −1.82744 | 4.267902 | 2.61871 | 0.354104 | 4.345682 | −3.15366 |
| 0.357234 | −1.16829 | NA | NA | −1.3656 | 5.189138 | 3.062845 | −0.25622 | 5.048589 | −2.91378 |
| −0.66596 | −0.91901 | −4.33906 | NA | −1.82789 | 5.31366 | 3.428423 | 0.177563 | 4.986261 | −3.17268 |
| −0.54465 | −0.37987 | −3.69615 | NA | −1.04799 | 5.483016 | 3.79905 | 0.427416 | 4.908472 | −3.42886 |
| −0.83478 | −1.30393 | NA | NA | −1.23223 | 5.414652 | 3.570305 | −0.50035 | 4.888168 | −2.92943 |
| −0.25868 | −0.73162 | −3.45979 | NA | −0.9901 | 5.221906 | 3.517947 | 0.26717 | 4.898316 | −3.30409 |
| −0.75995 | −1.40926 | −3.30432 | NA | −0.89338 | 4.811712 | 3.560333 | −0.21282 | 5.135555 | −3.29046 |
| −0.34866 | −1.43717 | MA | NA | −1.45223 | 4.402818 | 3.096624 | 0.51377 | 4.489271 | −2.93379 |
| 0.130254 | −1.30922 | NA | NA | −1.71064 | 5.225811 | 2.715913 | 0.179179 | 5.111109 | −2.79633 |
| −0.53692 | −0.92578 | NA | NA | −1.94224 | 5.458507 | 3.434119 | −0.52379 | 4.786963 | −3.32389 |
| −0.62558 | −0.77169 | −3.98471 | NA | −1.84538 | 5.570122 | 3.094622 | 0.562682 | 4.662805 | −3.56975 |
| −0.77485 | −1.0031 | −3.98672 | NA | −1.71004 | 5.4808217 | 2.608383 | −0.14001 | 4.918411 | −3.25034 |
| −0.42935 | −1.56302 | −6.19143 | NA | −1.82857 | 5.324254 | 2.814975 | −0.08286 | 4.99615 | −3.02055 |
| −0.97339 | −1.34438 | −7.37544 | NA | −1.168 | 4.865155 | 3.36474 | −0.12633 | 4.888623 | −2.93182 |
| −0.90458 | −0.69857 | −3.70004 | NA | −1.04261 | 5.307549 | 3.768653 | 0.387059 | 4.909573 | −3.21358 |
| −0.73651 | −1.11346 | −4.02301 | NA | −1.27407 | 4.894447 | 2.954889 | −0.34679 | 4.525806 | −2.84709 |
| 0.180313 | −1.3557 | NA | NA | −1.75973 | 5.462692 | 2.791872 | −0.7243 | 5.084268 | −2.7908 |
| −0.75236 | −0.67502 | −5.41051 | NA | −2.11665 | 5.546599 | 3.307295 | 0.358777 | 4.87639 | −3.38105 |
| −3.49197 | −1.22653 | NA | NA | −2.4647 | 5.879951 | 2.735943 | 0.325371 | 4.405572 | −4.51391 |
| −0.60559 | −1.46385 | −3.17489 | NA | −1.20996 | 4.45871 | 3.136506 | 0 | 5.089178 | −3.32061 |
| −0.53897 | −1.9004 | −4.17254 | NA | −1.53561 | 4.8875 | 3.038031 | 0.187797 | 5.397354 | −2.92314 |
| −0.59801 | −1.66331 | NA | NA | −1.85296 | 5.445093 | 2.577883 | 0.596224 | 4.943218 | −2.61627 |
| −0.75729 | −1.65001 | −4.75203 | NA | −1.31841 | 5.56871 | 3.10709 | 0.202004 | 4.93381 | −3.41592 |
| −1.05866 | −1.1568 | −3.77634 | NA | −1.46854 | 5.529027 | 3.252329 | 0.41685 | 4.895456 | −2.67198 |
| −0.89713 | −0.70207 | NA | NA | −2.12276 | 5.39629 | 3.064053 | 0.625827 | 4.976918 | −2.75868 |
| −0.12039 | −0.53329 | −4.00359 | NA | −2.02681 | 5.73355 | 3.45757 | 0.591548 | 4.837646 | −3.5342 |
| −0.52587 | −1.93085 | −3.67693 | NA | −1.56455 | 3.931034 | 2.988031 | 0.075743 | 4.72748 | −3.3575 |
| −1.4972 | −1.19389 | NA | NA | −1.53896 | 5.161047 | 2.906039 | −0.01067 | 4.74544 | −3.57612 |
| −1.11242 | −0.44368 | NA | NA | −1.78527 | 5.527037 | 3.323121 | 0.132775 | 4.489447 | −3.71661 |
| −0.15186 | −2.56595 | −6.64439 | NA | −2.52406 | 4.827183 | 2.530078 | 0.694008 | 4.630709 | −3.69893 |
| −0.32242 | −1.97164 | −5.4765 | NA | −2.51587 | 4.780553 | 3.003958 | −0.35774 | 4.90932 | −3.72216 |
| −0.60394 | −1.4787 | NA | NA | −2.56287 | 5.034335 | 2.871674 | 0.854271 | 4.881451 | −3.51868 |
| 0.037979 | −0.34719 | −1.38823 | 1.201449 | 0.687876 | 6.631398 | 3.177566 | 0.238555 | 1.715257 | −4.00775 |
| 0.142062 | −0.80141 | −0.54768 | 1.964637 | 1.283862 | 6.598455 | 3.698428 | −0.24618 | 2.01755 | −3.28164 |
| 0.57281 | −0.922 | −1.02461 | 2.002748 | 1.215868 | 6.662966 | 3.872238 | −0.02176 | 2.16063 | −3.17651 |

| guanine* | heptadec [symbol] | hypotauri [symbol] | hypoxant [symbol] | inosine | inosine-5- [symbol] | inositol p [symbol] | L-(−)-fuco [symbol] | L-2-amino [symbol] |
|---|---|---|---|---|---|---|---|---|
| NA | 1.827528 | 2.102.509 | −1.00907 | 3.535409 | 2.613371 | −1.15417 | 0.113261 | 0.195174 |
| NA | 1.670586 | 1.491707 | −1.35135 | 2.766222 | NA | −1.78592 | 0.2.62851 | 0.142779 |
| NA | 1.776335 | 3.048733 | −1.56231 | 2.914596 | NA | −1.63371 | 0.226166 | 0.4643 |
| NA | 1.462064 | 2.301535 | −1.4316 | 3.352256 | NA | −1.69658 | −0.10793 | 0.853062 |
| NA | 1.645656 | −0.01124 | −1.03994 | 4.065915 | NA | −1.5485 | 0.569324 | 0.566655 |
| NA | 2.098623 | 1.096694 | −0.23741 | 3.93244 | NA | −1.63237 | 0.102171 | 0.231889 |
| NA | 2.267113 | 1.307115 | −0.45558 | 4.154453 | NA | −1.67748 | 0.774156 | 0.771373 |
| NA | 1.952539 | −4.24711 | −1.34454 | 3.289251 | −0.8962 | −1.67854 | 0.212264 | 0.753413 |
| NA | 2.137249 | 1.632351 | −1.4415 | 2.740926 | NA | −1.59422 | 0.580791 | 0.212219 |
| NA | 1.670621 | 1.149408 | −1.45697 | 3.2515 | NA | −2.66101 | 0.306044 | 0.432172 |
| NA | 1.345457 | 3.129911 | −2.60405 | 2.291482 | NA | −1.95213 | 0.264499 | 0.579209 |
| NA | 1.62453 | 1.449677 | −1.39758 | 3.630279 | NA | −1.45803 | 0.276733 | 0.578687 |
| NA | 2.197661 | 2.098312 | −0.10251 | 3.724924 | NA | −1.94834 | 0.016533 | 0.132745 |
| NA | 1.857738 | 1.429974 | −0.34872 | 3.920633 | NA | −1.67335 | 0.437703 | 0.243991 |
| NA | 1.471594 | 1.790896 | −1.90909 | 3.343456 | NA | −2.3346 | 0.60287 | 0.330908 |
| NA | 0.656152 | 2.02109 | −3.31886 | 1.517722 | NA | −1.71608 | 0.042227 | 0.07772 |
| NA | 1.947057 | 0.484532 | −1.14637 | 3.434105 | −1.82379 | −2.02978 | 0.098051 | 0.449764 |
| NA | 1.788026 | 1.649225 | −1.18587 | 3.162677 | −2.33499 | −1.677 | 0.468174 | 0.492322 |
| NA | 1.468195 | 2.035235 | −1.73161 | 2.549018 | NA | −1.77462 | 0.096786 | 0.889065 |
| NA | 0.932975 | 1.346499 | −1.59416 | 3.460665 | NA | −2.286 | 0.215428 | 0.453151 |
| NA | 0.603615 | 1.031144 | −2.84461 | 0.310879 | NA | −2.71379 | −2.08053 | −0.36726 |
| NA | 0.576079 | −6.39486 | −0.13261 | 2.556098 | NA | −1.32703 | −0.22377 | −0.13258 |
| NA | 1.373341 | 2.008132 | −1.6517 | 3.04513 | NA | −2.64949 | 0.298687 | 0.381787 |
| NA | 0.877049 | 2.486777 | −3.23647 | 1.548057 | NA | −1.96126 | 0.149611 | 0.500924 |
| NA | 0.297231 | 0.8617 | −2.96884 | 2.073736 | NA | −2.92572 | 0.305926 | 0.191842 |
| NA | −2.18734 | 0.617565 | −2.9976 | 1.185715 | NA | −1.43632 | −0.1008 | −0.42149 |
| NA | 1.273577 | 1.106293 | −2.06174 | 2.598669 | −2.0494 | −2.23335 | 0.049561 | 0.748566 |
| NA | 0.383784 | 0.741135 | −3.14062 | 2.608719 | NA | −2.36916 | −1.95205 | −0.07116 |
| NA | 1.847567 | 1.946503 | −1.13545 | 2.495179 | NA | −2.77981 | 0.602798 | 0.395691 |
| NA | 1.13568 | 1.974679 | −2.44525 | 2.036861 | NA | −2.91937 | −0.24802 | 0.283087 |
| NA | 1.532889 | 0.510595 | −0.3667 | 4.125023 | NA | −1.29138 | 0.014499 | 0.107425 |
| NA | 1.484795 | 0.501543 | −1.13122 | 4.207536 | NA | −1.47835 | −0.05826 | −0.78627 |
| NA | 0.765171 | 0.790369 | −1.52995 | 0.300732 | NA | −2.28597 | −2.51121 | 0.688304 |
| NA | 0.44512 | 1.721529 | −1.17408 | 0.372288 | NA | −1.87123 | −0.69231 | 0.555747 |
| NA | 0.028476 | 0.950562 | −1.47562 | −0.07377 | NA | −2.52009 | −2.0852 | 0.49528 |
| 3.583824 | 2.607673 | −1.28999 | −2.45044 | 2.141321 | NA | −1.81486 | −2.44098 | −0.54184 |
| 3.9763231 | −2.72052 | −1.59722 | −2.01251 | 2.860295 | NA | −2.64542 | −1.88211 | 0.000142 |
| 4.192804 | 3.186571 | −1.53263 | −1.41562 | 2.899392 | NA | −2.50235 | −1.77864 | 0.123626 |

| lactic acid | L-alanine | L-asparag [symbol] | L-citrullin [symbol] | L-cystein [symbol] | L-cystine | L-glutami [symbol] | L-glutami [symbol] | lignoceric [symbol] | linoleic a [symbol] |
|---|---|---|---|---|---|---|---|---|---|
| 7.777312 | 2.026031 | NA | NA | 2.141862 | 0.955975 | 3.894867 | 5.37418 | −1.62198 | 3.59885 |
| 7.828593 | 1.777723 | NA | NA | 1.661573 | −0.09664 | 3.146965 | 5.453991 | −1.86951 | 4.38521 |
| 7.816272 | 1.778192 | NA | NA | 1.458007 | 0.154869 | 3.136976 | 5.383406 | −1.73039 | 3.921481 |
| 7.939356 | 1.165773 | NA | NA | 2.185164 | 0.54854 | 2.299852 | 4.852643 | −1.34363 | 2.920583 |
| 8.398938 | 1.850014 | NA | NA | 2.759215 | 1.265649 | 2.679531 | 5.834823 | NA | 3.845204 |
| 8.532079 | 2.116407 | NA | NA | 1.12625 | −1.68174 | 2.80835 | 5.570777 | −1.16917 | 4.233698 |
| 7.75167 | 1.683362 | 2.049932 | NA | 1.911907 | 0.715356 | 3.61272 | 5.29605 | −2.86014 | 4.554307 |
| 7.95145 | 2.176475 | 1.702573 | NA | 2.249877 | 0.650139 | 3.563426 | 5.399797 | −1.43857 | 4.265495 |
| 7.870321 | 2.360146 | NA | NA | 1.836158 | 0.816225 | 3.214079 | 5.745324 | −1.84142 | 4.225123 |
| 7.736762 | 2.494203 | 2.440612 | NA | 1.79909 | −0.04137 | 3.392432 | 5.768042 | −3.18931 | 3.974561 |
| 7.601362 | 1.622656 | NA | NA | 1.891487 | 1.028443 | 2.678456 | 5.359054 | −3.25181 | 3.615563 |
| 8.227597 | 2.198871 | NA | NA | 1.839135 | 0.7874 | 2.266079 | 5.922819 | −1.27557 | 3.737033 |
| 8.44147 | 1.883869 | NA | NA | 1.793449 | −1.06555 | 2.868308 | 5.443985 | −1.67457 | 4.138354 |
| 8.367569 | 1.975906 | NA | NA | 1.550386 | 0.200077 | 2.722128 | 5.575349 | −1.41701 | 4.586071 |
| 8.197507 | 2.241864 | NA | NA | 1.720392 | 0.724063 | 2.29423 | 5.715071 | −1.29613 | 4.332812 |
| 8.527765 | 2.09997 | NA | NA | 2.008808 | 0.531184 | 2.680071 | 5.842308 | NA | 3.853415 |
| 7.782714 | 1.957829 | 1.7851 | NA | 1.876928 | 0.285527 | 3.126742 | 5.375247 | −1.79573 | 3.99155 |
| 7.609349 | 2.146792 | NA | NA | 1.59068 | −0.10862 | 3.496407 | 5.57035 | −2.22988 | 4.598659 |
| 7.614472 | 1.429552 | NA | NA | 2.071733 | 0.794612 | 2.99885 | 5.323719 | −1.4251 | 4.001918 |
| 8.319967 | 2.21534 | NA | NA | 2.077233 | 0.670406 | 2.625719 | 5.801959 | −2.31618 | 4.40839 |
| 8.607241 | 1.932608 | NA | NA | 1.385436 | −0.87204 | 2.451422 | 5.490784 | −1.49851 | 4.43604 |
| 8.454913 | 2.129183 | NA | NA | −1.12963 | −1.89101 | 2.876456 | 4.560877 | NA | 3.403215 |
| 8.002244 | 2.428357 | NA | NA | 2.043528 | 1.240053 | 2.926722 | 5.784194 | −1.95102 | 3.346494 |
| 8.338924 | 2.643362 | NA | NA | 1.723338 | −0.08222 | 3.48869 | 6.016096 | −1.68144 | 2.487947 |
| 8.490696 | 2.265024 | NA | NA | 2.042674 | 0.936553 | 2.306682 | 5.89298 | −1.62328 | 4.005958 |
| 8.300112 | 2.189683 | NA | NA | 0.295592 | −1.21725 | 3.335971 | 5.436126 | NA | 3.865907 |
| 7.792466 | 1.71251 | NA | NA | 2.05739 | 1.034013 | 3.24453 | 5.613567 | −1.49762 | 4.563301 |
| 8.343722 | 2.146864 | NA | 3.166433 | −0.53065 | −0.01209 | 2.330817 | 5.300835 | NA | 3.899829 |
| 8.348698 | 1.47559 | NA | NA | 1.953859 | 0.480782 | 2.851086 | 5.579968 | −1.67969 | 4.499907 |
| 7.651243 | 1.79653 | NA | NA | 1.634458 | 0.720214 | 2.619283 | 5.436465 | −1.92933 | 2.923038 |
| 8.552524 | 1.988569 | NA | NA | −0.2079 | −2.31427 | 2.800656 | 5.191972 | −2.75439 | 3.337299 |
| 8.147507 | 1.828207 | NA | NA | −0.18046 | −1.80956 | 2.864622 | 4.901659 | −2.04189 | 4.32173 |
| 8.173286 | 1.752871 | NA | NA | 1.934544 | 0.195726 | 2.399391 | 4.918372 | NA | 2.773076 |
| 8.280455 | 1.90893 | NA | NA | 2.36701 | 1.044276 | 2.39031 | 5.24402 | −1.22565 | 2.909519 |
| 8.139347 | 1.615908 | NA | NA | 1.999529 | 0.934702 | 2.302428 | 5.196702 | NA | 3.547318 |
| 5.430968 | 1.264226 | 2.287285 | 0.615886 | −1.91796 | NA | 1.926096 | −3.39623 | 2.004125 | 7.038192 |
| 5.77842 | 1.10479 | NA | 1.099506 | −1.03434 | −7.41767 | 2.733088 | −3.87793 | 1.257711 | 6.398715 |
| 5.825695 | 1.483264 | 2.838057 | 1.207328 | −1.14211 | −6.69565 | 2.641487 | −3.72587 | NA | 6.831817 |

| L-isoieuci [symbol] | L-leucine | L-methio [symbol] | L-methio [symbol] | L-ornithin [symbol] | L-phenyla [symbol] | L-proline | L-pyroglu [symbol] | L-serine |
|---|---|---|---|---|---|---|---|---|
| 3.333478 | 3.959588 | 3.610313 | NA | 3.991747 | 1.296019 | 3.656872 | 0.071001 | 4.419246 |
| 3.935337 | 4.620085 | 3.318206 | NA | 4.133465 | 1.398822 | 3.564127 | −0.45548 | 4.267986 |
| 3.409686 | 3.963022 | 3.712208 | NA | 4.194751 | 1.239692 | 3.794357 | −0.36165 | 4.557749 |
| 3.683332 | 4.345187 | 3.028096 | NA | 4.036653 | 1.384198 | 3.088693 | −0.8961 | 3.943858 |
| 3.776936 | 4.287746 | 3.267919 | NA | 4.235914 | 1.671799 | 3.300375 | −0.05716 | 4.401179 |
| 3.94246 | 4.649291 | 3.862863 | NA | 4.56795 | 1.733953 | 4.429699 | −0.18991 | 4.80513 |
| 3.23296 | 3.462049 | 3.50764 | NA | 4.129431 | 1.220391 | 3.020778 | −0.02818 | 4.388482 |
| 3.256645 | 3.955313 | 3.349593 | NA | 3.716589 | 1.269138 | 3.353742 | −0.04015 | 4.455433 |
| 3.728469 | 4.531004 | 3.605567 | NA | 4.225337 | 1.438904 | 3.666257 | −0.14808 | 4.573815 |
| 3.71902 | 4.529371 | 4.007 | NA | 5.477844 | 1.345498 | 4.203623 | 0.1467 | 4.947761 |
| 3.918759 | 4.415166 | 3.410396 | NA | 4.541807 | 1.20383 | 3.494013 | −0.46271 | 4.228809 |
| 3.57946 | 4.256967 | 3.933255 | NA | 4.558848 | 1.736231 | 4.034773 | −0.23618 | 4.861768 |
| 3.776991 | 4.372679 | 3.285019 | NA | 4.097656 | 1.426526 | 3.634691 | −0.20349 | 4.390364 |
| 3.538748 | 4.323633 | 3.447096 | NA | 4.068958 | 1.297033 | 3.72942 | −0.2547 | 4.278085 |
| 3.319257 | 4.210042 | 3.819977 | NA | 4.326306 | 1.452222 | 3.909696 | −0.46606 | 4.596709 |
| 3.70327 | 4.522041 | 3.709601 | NA | 4.206848 | 1.60269 | 3.629341 | −0.14057 | 4.59954 |
| 3.144503 | 3.820327 | 3.452644 | NA | 3.94484 | 0.975965 | 3.59453 | −0.31297 | 4.532468 |
| 3.615949 | 4.325388 | 3.677558 | NA | 4.457762 | 1.174599 | 3.556893 | 0.02335 | 4.568043 |
| 3.825085 | 4.235264 | 3.238386 | NA | 4.18606 | 1.266605 | 3.017342 | −0.40514 | 4.0707 |
| 3.567107 | 4.319182 | 3.492307 | NA | 4.339163 | 1.611346 | 3.840502 | −0.26304 | 4.598766 |
| 3.756789 | 4.627884 | 3.727655 | NA | 4.381691 | 1.877586 | 3.797113 | −0.19521 | 4.470914 |
| 3.217892 | 3.83127 | 2.06965 | NA | 3.516613 | 0.515343 | 0.762644 | −0.61079 | 3.711307 |
| 4.125532 | 4.895468 | 4.434456 | NA | 4.705246 | 1.732979 | 4.666669 | −0.24863 | 5.050534 |
| 3.926485 | 4.590979 | 3.944928 | NA | 4.747191 | 2.450438 | 4.500268 | 0.148852 | 5.090806 |
| 3.811516 | 4.653402 | 3.818491 | NA | 4.155692 | 1.676782 | 3.719728 | −0.38783 | 4.685442 |
| 3.236243 | 3.96609 | 3.699018 | NA | 3.687282 | 1.143471 | 3.080619 | 0.089538 | 4.42261 |
| 3.996865 | 4.682858 | 3.212602 | NA | 4.296807 | 1.568214 | 3.178621 | −0.02917 | 4.378084 |
| 3.907278 | 4.658187 | 3.412893 | NA | 3.869527 | 1.696823 | 3.365037 | −0.17569 | 4.262993 |
| 3.662462 | 4.460882 | 3.314083 | NA | 4.015873 | 1.488973 | 3.358914 | −0.31224 | 4.355191 |
| 3.795398 | 4.548407 | 4.064234 | NA | 4.2671 | 1.52318 | 4.035976 | −0.56444 | 4.638481 |
| 3.8528 | 4.614219 | 3.648917 | NA | 4.187772 | 1.399734 | 3.776014 | −0.50929 | 4.381537 |
| 3.754653 | 4.412508 | 3.422103 | NA | 3.661569 | 1.447416 | 3.093087 | −0.67183 | 4.046037 |
| 3.77333 | 4.448803 | 2.87288 | NA | 3.632565 | 1.535332 | 2.761624 | −0.47057 | 3.896856 |
| 4.130616 | 4.702628 | 3.257042 | NA | 3.985298 | 1.624676 | 3.19762 | −0.15849 | 4.204716 |
| 4.026729 | 4.641653 | 3.092924 | NA | 3.774729 | 1.625542 | 2.975556 | −0.51045 | 4.103639 |
| 1.93145 | 2.742751 | 7.159139 | 2.202229 | −1.51848 | −0.07058 | 3.286603 | 0.573149 | 1.750904 |
| 2.367556 | 3.173637 | 6.358093 | 2.034577 | −0.12311 | 0.514448 | 3.855105 | −1.31825 | 2.184341 |
| 2.725565 | 3.614186 | 6.688649 | 2.381603 | 0.013934 | 0.692644 | 4.217879 | −1.18118 | 2.406743 |

| L-threoni [symbol] | L-threoni [symbol] | L-tryptop [symbol] | L-tyrosine | L-valine | malonic a [symbol] | maltose | melibiose | methylph [symbol] | mucic aci [symbol] |
|---|---|---|---|---|---|---|---|---|---|
| 2.124232 | 3.472275 | 4.909446 | 1.161682 | 2.87955 | NA | −2.16044 | NA | 1.167804 | −1.03696 |
| 2.092455 | 3.544304 | 4.594976 | 0.704689 | 3.564401 | NA | −0.35166 | NA | 0.932491 | 0.935781 |
| 1.587554 | 3.740683 | 5.067747 | NA | 2.914896 | −3.37591 | −0.36774 | NA | 0.931855 | 1.120363 |
| 1.26653 | 3.045878 | 5.077085 | 0.318569 | 3.368926 | NA | −0.19164 | NA | 0.774031 | 0.918008 |
| 2.188471 | 3.650224 | 5.140717 | NA | 3.637191 | NA | −0.60046 | NA | 1.024432 | −0.47091 |
| 1.008468 | 3.940023 | 4.877069 | 1.456967 | 3.757148 | NA | −0.35127 | NA | 0.461846 | −1.35077 |
| 2.532554 | 3.594683 | 4.435139 | NA | 2.835574 | NA | −0.27481 | NA | 1.468769 | −1.07637 |
| 2.564507 | 3.486015 | 4.465469 | 0.893124 | 3.104355 | NA | −1.06677 | NA | 0.928757 | −1.29742 |
| 2.281917 | 3.64808 | 4.360542 | NA | 3.56611 | NA | −0.48512 | NA | 1.367028 | −0.65299 |
| 2.281595 | 3.996164 | 4.833037 | 0.732295 | 3.582991 | −2.38571 | −0.74007 | NA | 1.342198 | −0.70619 |
| 1.964875 | 3.344246 | 4.937687 | NA | 3.644655 | NA | −0.35447 | NA | 0.560493 | −1.11865 |
| 1.558712 | 3.957575 | 5.414535 | 1.303412 | 3.513285 | NA | −0.29273 | NA | 0.830547 | −1.24049 |
| 1.402728 | 3.355654 | 3.771506 | 0.660511 | 3.264608 | NA | −0.92707 | NA | 0.373287 | −1.29012 |
| 1.534641 | 3.631176 | 4.489128 | 0.383549 | 3.26573 | NA | −0.63985 | NA | 0.956842 | −1.14845 |
| 1.638331 | 3.576604 | 4.492077 | NA | 3.143633 | NA | −0.51049 | NA | 0.974531 | 0.927883 |
| 2.413066 | 3.496846 | 3.781761 | NA | 2.6543 | NA | −0.3583 | NA | 1.088642 | −1.03458 |
| 2.145285 | 3.640608 | 4.340018 | 0.240301 | 3.434999 | NA | −0.3224 | NA | 0.912451 | −0.91305 |
| 2.257661 | 3.218691 | 4.483374 | 0.315086 | 3.297446 | NA | −0.5443 | NA | 1.184716 | −0.65728 |
| 1.34448 | 3.71096 | 4.755107 | 0.896475 | 3.445329 | NA | −1.14108 | NA | 0.800576 | −1.04495 |
| 1.011541 | 3.49222 | 3.59897 | 0.600321 | 3.380083 | NA | 0.049117 | NA | 0.936363 | −1.05633 |
| 1.107493 | 2.738907 | −1.31326 | −0.17858 | 2.608395 | NA | 0.474473 | NA | 0.14855 | 0.640555 |
| 1.847538 | 4.092537 | 5.28127 | 1.59106 | 3.776292 | NA | −0.45319 | NA | 0.988583 | 1.191684 |
| 1.364659 | 3.669191 | 5.404229 | 1.862949 | 3.442451 | −3.12354 | −0.0883 | NA | 1.007394 | −0.92849 |
| 1.129002 | 3.793156 | 4.664399 | NA | 3.58642 | NA | −0.15218 | NA | 0.979227 | −0.68004 |
| 2.012058 | 3.425607 | 4.198367 | 0.029564 | 2.840449 | NA | 0.204441 | NA | 0.226114 | 0.364774 |
| 2.265388 | 3.546895 | 5.004744 | 0.442832 | 3.481109 | NA | 0.311938 | NA | 1.126355 | −1.08131 |
| 1.786907 | 3.509413 | 0.356887 | 0.565885 | 3.522425 | NA | 0.012091 | NA | 0.560843 | 1.859124 |
| 1.68401 | 3.205123 | 3.743547 | 0.315921 | 3.395033 | NA | −0.27665 | NA | 0.640377 | −0.84057 |
| 1.708839 | 3.765413 | 4.917744 | 0.531359 | 3.352254 | NA | −0.34368 | NA | 0.84304 | 0.791357 |
| 1.051659 | 3.64026 | −3.41203 | 0.863046 | 3.482551 | NA | −0.00194 | NA | 0.194835 | −2.81366 |
| 1.260902 | 3.40816 | 0.242806 | 0.845802 | 3.334561 | NA | 0.309988 | NA | 0.48309 | 0.116281 |
| 0.572536 | 3.181059 | 4.961274 | 0.78654 | 3.497597 | NA | −0.04531 | NA | 0.208094 | −0.79488 |
| 1.140483 | 3.457973 | 5.101195 | 0.885882 | 3.806471 | −1.8267 | −0.35926 | NA | 0.618369 | −0.13486 |
| 0.95043 | 3.406165 | 5.349554 | 1.077612 | 3.666231 | NA | 0.157426 | NA | 0.582631 | −0.91699 |
| −0.49935 | 0.313844 | 2.710799 | −0.76102 | 1.04371 | −0.99132 | −1.05892 | −0.38277 | −0.65841 | −1.20277 |
| 0.324653 | 0.888255 | 3.625139 | 0.03452 | 1.643329 | −0.19396 | −1.67969 | 0.53058 | −0.43528 | 0.023892 |
| G.275166 | 1.065261 | 3.679318 | 0.142048 | 1.849005 | −0.37516 | −1.58839 | 0.30964 | −0.07077 | −0.22198 |

| myo-inos [symbol] | myristic a [symbol] | nicotinam [symbol] | nicotinic [symbol] | N-methyl [symbol] | nonade ca [symbol] | oleamide | oleanitril [symbol] | oleic acid |
|---|---|---|---|---|---|---|---|---|
| 5.769294 | 1.795884 | 0.187093 | −2.91339 | 0.447657 | NA | 3.529198 | −1.78368 | 3.530502 |
| 4.82798 | 2.317944 | NA | −2.62478 | 0.109151 | NA | 3.591029 | −1.32649 | 4.094037 |
| 5.00726 | 1.358375 | NA | −5.64737 | 0.646901 | NA | 3.80996 | −0.7672 | 3.657924 |
| 4.62501 | 0.785532 | −2.08674 | −3.25156 | 0.148827 | NA | 4.062792 | −0.59152 | 3.088523 |
| 5.20069 | 1.767842 | −1.88548 | −6.15318 | −1.08478 | NA | 4.483666 | −1.18874 | 3.959446 |
| 5.097038 | 2.300422 | −1.78583 | −2.90089 | 0.411437 | NA | 4.33388 | −0.83146 | 3.958057 |
| 5.209986 | 2.429848 | NA | 1.208301 | 0.476409 | NA | 3.975053 | −1.0533 | 4.360246 |
| 5.396856 | 2.353553 | NA | −3.49836 | −1.29715 | NA | 4.038771 | −1.95483 | 4.036611 |
| 5.083779 | 2.062974 | −0.42892 | −0.97756 | −0.12903 | NA | 4.12102 | −1.35734 | 4.095408 |
| 5.209901 | 1.517197 | NA | −2.79026 | 0.753673 | NA | 3.853656 | −1.10408 | 3.719545 |
| 4.768612 | 1.475732 | −2.15078 | −5.94942 | 0.741604 | NA | 3.779164 | −0.21367 | 3.402267 |
| 4.949185 | 1.780609 | −2.4789 | 0.543701 | 1.780134 | NA | 3.991297 | −1.66772 | 3.483704 |
| 5.036078 | 2.45707 | NA | −4.24674 | 0.296383 | NA | 4.708372 | −1.09154 | 3.969319 |
| 5.133884 | 2.587024 | −2.1605 | −3.31954 | −0.58281 | NA | 4.032086 | −1.1138 | 4.201011 |
| 5.109776 | 2.283939 | NA | −2.42984 | 0.268461 | NA | 3.755412 | −2.05659 | 3.659459 |
| 5.0068 | 1.803472 | −0.7916 | 0.980223 | 0.334105 | NA | 3.866556 | −1.73672 | 3.710368 |
| 5.13865 | 2.257547 | NA | −2.6372 | 0.729621 | NA | 3.73859 | −0.40381 | 4.261701 |
| 4.818926 | 2.113098 | NA | −8.0259 | 0.324464 | NA | 3.630357 | −1.26435 | 3.716528 |
| 5.196279 | 2.26917 | −2.51733 | 0.689063 | 0.949412 | −1.85747 | 4.61776 | −1.40701 | 4.150404 |
| 4.177057 | 2.860584 | NA | −2.60516 | 0.795963 | NA | 4.469122 | 0.991712 | 4.313697 |
| 3.413579 | 2.296141 | 0.39865 | 0.479966 | 1.024913 | NA | 3.317701 | −1.131 | 4.071726 |
| 5.021255 | 1.090042 | −1.46075 | −6.06853 | 0.224834 | NA | 3.955047 | −1.52841 | 3.45667 |
| 5.168869 | 0.796566 | −2.10885 | −10.527 | 0.697188 | NA | 3.575837 | −1.82286 | 2.362671 |
| 5.067346 | 2.275386 | NA | −2.17979 | 0.796335 | NA | 3.971958 | −1.91961 | 3.876112 |
| 5.142523 | 2.061947 | −0.54649 | 0.157822 | 0.63223 | −2.33913 | 3.180891 | −2.2256 | 3.801487 |
| 5.119606 | 2.515039 | −0.45426 | −2.68387 | 0.684366 | NA | 3.797758 | −1.02447 | 4.353818 |
| 4.606167 | 1.403208 | NA | 0.656852 | 0.941991 | −1.92081 | 2.960363 | 0.104248 | 3.577447 |
| 5.140631 | 2.067607 | −0.78085 | −4.20599 | −0.4826 | NA | 4.22641 | 0.128957 | 4.212061 |
| 4.807251 | 0.690652 | −1.32492 | −4.56122 | 0.444679 | NA | 3.721286 | −1.00737 | 2.749108 |
| 4.361274 | 1.418127 | −1.46204 | 0.001112 | 0.475433 | −2.70018 | 3.259606 | −1.51392 | 4.145267 |
| 4.196434 | 2.188876 | −0.95248 | −3.2185 | 0.776281 | NA | 3.393406 | 0.340585 | 4.269911 |
| 3.854614 | 0.802758 | NA | 0.586157 | −0.07477 | NA | 3.906551 | −0.38162 | 2.519258 |
| 3.997277 | 0.992353 | NA | 0.762184 | −0.45373 | NA | 4.140334 | −0.52134 | 2.819268 |
| 3.76468 | 1.469395 | −1.73391 | −2.77394 | 0.30607 | NA | 3.606967 | −0.60358 | 3.223004 |
| 3.794465 | 5.001031 | −1.13713 | −1.59065 | 1.314817 | −0.21317 | 2.114876 | −3.85049 | 6.005117 |
| 4.389091 | 4.458519 | −0.82019 | −0.86685 | 2.762539 | 0.006722 | 1.536585 | −4.28255 | 5.450671 |
| 4.511204 | 4.803293 | −0.83733 | −0.76359 | −0.1903 | −0.15912 | 1.789497 | −4.2521 | 5.754004 |

| O-phosph [symbol] | oxalic aci [symbol] | palmitic a [symbol] | palmitole | phosphat [symbol] | phosphen [symbol] | phosphoe [symbol] | phytosph [symbol] | porphine | propylen [symbol] |
|---|---|---|---|---|---|---|---|---|---|
| 1.889286 | 0.210533 | 5.513913 | 2.303678 | 1.768966 | 2.001724 | NA | NA | 0.539647 | 1.08708 |
| 1.334004 | 0.481502 | 6.120077 | 3.186783 | 7.406145 | 2.569944 | 1.58478 | NA | 0.775834 | −2.27492 |
| 1.408759 | −0.19627 | 5.662661 | 2.230593 | 7.424582 | 2.233574 | NA | NA | 0.651002 | 0.568715 |
| 0.986305 | 0.256776 | 5.015982 | 1.694367 | 7.068946 | 2.672506 | NA | NA | 0.657731 | 0.043991 |
| 0.522641 | 0.890176 | 5.69646 | 2.574979 | 7.38096 | 2.80744 | NA | NA | 0.345102 | −1.85674 |
| 0.903459 | 0.01546 | 5.98175 | 3.007281 | 6.620046 | 2.267721 | NA | NA | 0.438763 | −0.49975 |
| 1.752497 | 1.148665 | 6.274786 | 3.759548 | 7.621726 | 2.36674 | 1.679001 | NA | 0.549786 | 1.381302 |
| 1.343515 | 0.019981 | 5.848639 | 3.191124 | 7.468892 | 1.894421 | 0.485535 | NA | 0.107141 | −1.8572 |
| 1.091503 | 0.421861 | 6.11832 | 2.907673 | 7.64039 | 2.813445 | 1.698497 | NA | 0.544266 | −1.13062 |
| 0.977832 | 0.631342 | 5.743142 | 2.044932 | 7.478202 | 2.250231 | 1.501047 | NA | 0.313437 | −1.62507 |
| 1.153799 | 0.254499 | 5.532198 | 2.501908 | 7.345209 | 2.457165 | NA | NA | 0.702597 | −1.73196 |
| 0.687663 | 0.200052 | 5.449464 | 2.138779 | 6.984811 | 2.882101 | NA | NA | 0.676722 | −2.00398 |
| 0.371334 | −0.4516 | 5.998258 | 3.484234 | 6.697406 | 1.931442 | NA | NA | 0.211719 | −2.38549 |
| 0.868499 | 0.402089 | 6.369487 | 3.669639 | 6.901344 | 2.380495 | NA | NA | 0.536416 | −2.10966 |
| 0.61083 | 0.165263 | 6.027841 | 3.669475 | 7.255306 | 2.798175 | NA | NA | 0.301107 | −1.24981 |
| 0.723683 | 0.136579 | 5.451244 | 2.642513 | 7.218664 | 2.305734 | NA | NA | 0.488499 | −2.30422 |
| 1.229369 | 0.565122 | 5.665055 | 2.617706 | 7.391247 | 2.081957 | 1.210385 | NA | 0.311084 | 1.213618 |
| 1.77477 | 0.987586 | 6.217747 | 3.008549 | 7.768664 | 2.538779 | NA | NA | 0.796705 | −1.75304 |
| 1.016619 | 0.066243 | 5.709585 | 2.953638 | 7.213676 | 2.017566 | 1.93828 | NA | 0.541648 | 1.152285 |
| 0.096541 | 0.028312 | 5.887488 | 2.860243 | 7.187246 | 2.361571 | NA | NA | 0 | −1.5475 |
| 1.026467 | 0.869014 | 6.495145 | 3.566875 | 7.343019 | 2.753071 | NA | NA | 1.251137 | −0.80487 |
| −0.7827 | 1.574048 | 6.220397 | 2.360445 | 6.959508 | 2.005774 | NA | NA | 2.063106 | −0.70568 |
| 1.201771 | −0.15395 | 5.259196 | 1.950676 | 7.171455 | 2.504732 | −1.27686 | NA | 0.540531 | −2.39147 |
| 1.49585 | 0 | 4.604459 | 1.319746 | 7.454073 | 2.555292 | NA | NA | 0.87166 | 0.438745 |
| 0.405381 | 0.454167 | 5.756728 | 2.992196 | 7.422707 | 2.63908 | NA | NA | 0.540307 | −0.15259 |
| 1.29623 | 0.511949 | 5.619798 | 2.150444 | 6.639918 | 1.897536 | NA | NA | 1.483391 | 0.586599 |
| 1.525237 | 1.34826 | 6.206732 | 3.515383 | 7.578858 | 2.479777 | NA | NA | 1.101285 | 0.97097 |
| 0.736416 | 1.249119 | 5.642638 | 1.855343 | 7.100853 | 2.162838 | NA | NA | 1.852362 | −1.49783 |
| 1.591289 | 0.830923 | 6.306628 | 3.203957 | 7.362649 | 2.654326 | NA | NA | 0.951026 | 0.59306 |
| 1.214027 | 0.095402 | 4.870976 | 1.144717 | 7.12526 | 2.23773 | NA | NA | 0.662171 | 0.629275 |
| −0.08179 | 0.278525 | 5.7427 | 1.628771 | 6.459924 | 1.761587 | NA | NA | 1.373671 | −1.52639 |
| 0.605149 | 0.614819 | 6.305379 | 2.694028 | 6.860546 | 1.940824 | NA | NA | 1.623461 | −1.78098 |
| 0.814075 | 0.208458 | 4.647258 | 1.899136 | 6.995292 | 2.343157 | NA | NA | 0.567572 | −1.43988 |
| 1.073141 | 0.378919 | 4.818161 | 1.903878 | 7.210999 | 2.383032 | NA | NA | 0.653185 | −1.43771 |
| 1.196098 | 0.608542 | 5.172655 | 2.417732 | 7.381095 | 2.420727 | NA | NA | 0.94893 | −2.42011 |
| −0.95069 | 0.272829 | 7.507285 | 5.213699 | 6.336884 | 1.182523 | NA | 1.616972 | 2.682072 | −2.00573 |
| −1.12387 | −0.54886 | 6.959088 | 4.671141 | 5.814534 | 0.523922 | NA | 0.987151 | 1.308598 | −2.61763 |
| −1.22441 | 0.046474 | 7.290106 | 5.050698 | 6.259332 | 0.759272 | NA | 1.405155 | 2.642553 | −2.82974 |

| pyruvic a [symbol] | raffinose | ribitol | ribonic ac [symbol] | salicylic a [symbol] | scyllo-ino [symbol] | sedohept [symbol] | sedohept [symbol] | serotonin |
|---|---|---|---|---|---|---|---|---|
| 3.672882 | NA | 1.242759 | 2.065638 | −2.97971 | −0.33664 | 6.371183 | 1.945979 | 2.234991 |
| 3.190498 | NA | 1.273667 | 2.502672 | −3.25448 | NA | 4.657906 | 1.867661 | 2.037985 |
| 3.431553 | NA | 0.88099 | −0.60206 | −2.22767 | NA | 5.172011 | 1.468096 | 1.937588 |
| 2.933488 | NA | 0.044632 | 2.079421 | −2.55909 | NA | 5.624749 | 0.417201 | 2.536089 |
| 3.602652 | NA | 1.915925 | 1.256424 | −2.66876 | NA | 6.181612 | −0.88208 | 2.795983 |
| 2.759685 | NA | 0.145974 | 2.955389 | −2.58183 | NA | 5.344388 | 0.645232 | 1.949448 |
| 3.083252 | NA | 0.049724 | 3.027801 | −3.27292 | NA | 4.615279 | 2.229406 | 2.261524 |
| 3.238899 | NA | 0.112459 | 0.831082 | −3.36535 | −0.52379 | 5.497546 | 2.223046 | 1.180959 |
| 3.760989 | NA | 1.780791 | 2.748918 | −0.03647 | NA | 4.9883 | 1.876446 | 2.030375 |
| 3.264339 | NA | 0.116385 | 3.294289 | −3.09301 | NA | 4.635563 | 2.038332 | 2.434515 |
| 2.780165 | NA | 0.163553 | −0.21406 | −0.21482 | NA | 5.698703 | 0.210162 | 2.670783 |
| 3.730774 | NA | 1.629578 | 3.364739 | −2.50504 | NA | 5.650936 | −0.83161 | 2.665641 |
| 2.696471 | NA | 0.113234 | 1.116769 | −2.94566 | NA | 5.033488 | 0.613512 | 1.278435 |
| 2.418238 | NA | −0.11764 | 1.480864 | −3.10069 | NA | 4.630733 | −0.25969 | 2.369906 |
| 3.401948 | NA | 1.191827 | 2.93432 | −0.28247 | NA | 4.705923 | 0.176389 | 3.655988 |
| 3.333959 | NA | 0.395453 | −4.56222 | −2.72122 | −1.05435 | 5.390456 | −1.49445 | 3.062192 |
| 3.243542 | NA | 0.837245 | 0.79627 | −2.82058 | NA | 5.124871 | 2.345032 | 1.373 |
| 3.106425 | NA | −0.41467 | 3.244436 | −3.89279 | NA | 4.619792 | 2.003349 | 2.192282 |
| 3.177455 | NA | −0.02248 | 2.547088 | −2.60803 | NA | 5.844069 | 1.426233 | 1.053119 |
| 3.328924 | NA | 1.627312 | 1.024644 | −2.8533 | NA | 7.298092 | −0.40758 | 2.783071 |
| 3.391539 | NA | 1.506655 | 3.071517 | −0.04479 | NA | 5.49014 | 0.476964 | 1.97389 |
| 0.709213 | NA | 0.638933 | 2.036587 | −2.60726 | NA | 4.823694 | −1.24079 | NA |
| 3.817507 | NA | 1.587524 | 3.340884 | −0.02159 | NA | 4.598974 | 0.882803 | 2.791716 |
| 3.754614 | NA | 1.979781 | −4.07942 | −2.26206 | NA | 5.600841 | 0.098281 | 3.073255 |
| 3.373987 | NA | 1.607085 | 0.461496 | −0.42463 | NA | 4.979544 | −0.50221 | 3.395846 |
| 2.88244 | NA | 1.003922 | 3.09601 | −0.7762 | NA | 4.679001 | 0.417085 | −0.56249 |
| 3.196606 | NA | 0.063659 | 5.170211 | −2.0522 | NA | 5.800695 | 0.697059 | 2.519501 |
| 2.873695 | NA | 0.865573 | 2.730919 | −3.62279 | NA | 5.246407 | −0.05507 | 2.040416 |
| 3.030379 | NA | 0.512193 | 5.069396 | −1.0354 | NA | 4.898547 | −0.73294 | 2.654789 |
| 3.259815 | NA | 1.439423 | 3.051821 | −1.06995 | NA | 5.474617 | 1.00286 | 2.359432 |
| 2.830014 | NA | 0.236902 | 2.741079 | −0.97718 | NA | 4.950436 | 0.345438 | 1.355234 |
| 1.95985 | NA | −0.3019 | 2.547504 | −1.24544 | NA | 5.337444 | 1.167738 | 1.358868 |
| 3.436853 | NA | 1.703423 | 1.979816 | −0.23932 | NA | 5.207392 | −2.80024 | 3.219833 |
| 3.436193 | NA | 1.758081 | 2.475192 | −0.19745 | NA | 5.334059 | 0.179181 | 3.065647 |
| 3.361407 | NA | 1.674773 | 2.244295 | −3.40793 | NA | 5.322864 | −2.52809 | 1.784304 |
| 0.617357 | 1.768223 | 1.364433 | 1.590359 | 0.937834 | −2.4763 | 2.35435 | −0.57793 | −3.28432 |
| 1.486064 | 2.859158 | 1.938384 | 2.002927 | 1.275587 | −1.3057 | 2.852002 | −0.4957 | −5.13017 |
| 1.395661 | 2.938787 | 2.066099 | 1.840962 | 1.934069 | −1.31056 | 2.860451 | −0.44799 | −4.04519 |

| shikimic a [symbol] | spermidi [symbol] | stearic aci [symbol] | succinic a [symbol] | sucrose | taurine* | urea | uridine | uridine 5 [symbol] - | xylitol |
|---|---|---|---|---|---|---|---|---|---|
| NA | −0.55803 | 3.87785 | 0.691988 | −0.19126 | 5.768576 | 7.458941 | −1.02296 | 0.935818 | −2.21333 |
| NA | −1.38775 | 3.997184 | 0.190787 | −0.5244 | 4.806461 | 7.485508 | −0.99508 | −0.10482 | −2.40082 |
| NA | −1.63999 | 3.889292 | 0.372656 | 0.589849 | 4.547756 | 7.525273 | −1.09583 | 0 | −2.02505 |
| NA | −2.32063 | 3.444422 | 0.338403 | −0.11278 | 4.363151 | 7.266104 | −1.62338 | −0.24515 | −2.19093 |
| NA | −2.74279 | 3.822495 | 1.446966 | −0.33359 | 5.43392 | 7.421207 | −0.90037 | −1.02558 | −2.13991 |
| NA | −2.69672 | 4.099609 | 0.990787 | 0.134414 | 5.080028 | 7.338654 | −1.12866 | −4.15292 | −2.34675 |
| NA | −1.03246 | 3.864244 | 0.137551 | −0.41194 | 5.505323 | 7.649363 | −0.68917 | −0.05402 | −2.27471 |
| NA | −0.62855 | 3.655829 | 0.520274 | −0.57652 | 5.747295 | 7.377375 | −1.08784 | 0.518856 | −2.87415 |
| NA | −1.75666 | 4.198131 | −0.02695 | 0.127427 | 5.580809 | 6.616057 | −1.03052 | −0.71871 | −2.59771 |
| NA | −1.25687 | 3.90199 | −0.11627 | −0.0775 | 5.434291 | 6.834089 | −1.01368 | −0.19798 | −2.73572 |
| NA | −4.80114 | 3.781718 | −0.00852 | 0.644999 | 3.519471 | 6.596696 | −2.58332 | −0.26132 | −1.53168 |
| NA | −2.46842 | 3.725077 | 0.516204 | 0.357627 | 5.20989 | 6.305985 | −1.55162 | −1.19266 | −2.39878 |
| NA | −1.87995 | 3.857451 | 1.345712 | 0.089362 | 4.854233 | 6.665299 | −1.13075 | −2.36921 | −2.25689 |
| NA | −2.11626 | 4.158905 | 1.117239 | 0.276237 | 4.749798 | 7.151537 | −0.83707 | −2.91867 | −2.76845 |
| NA | −2.19895 | 3.77178 | 0.32368 | −0.13119 | 5.104587 | 7.342906 | −1.85702 | 0.195903 | −1.69988 |
| NA | −1.45306 | 3.602685 | 1.741793 | 0.174277 | 5.136807 | 6.903471 | −1.15785 | −0.44543 | −2.42296 |
| NA | −1.10691 | 3.714448 | −0.23648 | −0.44926 | 5.328509 | 7.348025 | −1.40844 | 0.010251 | −2.79782 |
| NA | −1.45095 | 4.048295 | 0.328368 | 0.348381 | 4.743878 | 7.444664 | −0.6552 | −0.61798 | −1.44481 |
| NA | −1.34666 | 3.707587 | 0.05684 | 0.103143 | 4.799348 | 5.527741 | −1.53654 | −1.06121 | −2.37936 |
| NA | −2.57508 | 3.767573 | 1.174639 | −0.31258 | 5.184092 | 7.362693 | −1.53089 | −1.46985 | −2.68455 |
| NA | −2.14977 | 4.545058 | 1.026089 | −0.01935 | 4.41753 | 7.147879 | −1.34391 | −2.06271 | −1.2374 |
| NA | −3.53982 | 5.211902 | 0.159603 | −0.0569 | 5.084063 | 7.63245 | −2.74442 | −3.35027 | −2.07982 |
| NA | −1.91345 | 3.645425 | −0.36113 | 0.821109 | 5.044405 | 7.115872 | −1.72118 | 0.095925 | −2.84035 |
| NA | −1.37558 | 3.352657 | 2.199478 | 0.392171 | 4.944683 | 6.444604 | −0.55532 | −0.23777 | −1.72515 |
| NA | NA | 3.756785 | 1.295801 | 0.027693 | 5.209372 | 7.628166 | −1.34873 | −1.14739 | −2.59289 |
| NA | −1.49158 | 4.02531 | 0.902677 | −0.15876 | 5.422583 | 7.721027 | −1.00202 | −1.30533 | −2.89334 |
| NA | −1.81041 | 3.915862 | 0.060706 | 0.263228 | 5.235798 | 6.896056 | −1.81568 | −1.20753 | −2.0326 |
| NA | −2.1946 | 4.432092 | 0.510139 | −0.20743 | 3.976258 | 7.935438 | −1.29658 | −2.91761 | −0.79502 |
| NA | −1.67431 | 4.066284 | 1.251782 | −0.0386 | 4.395476 | 6.759083 | −0.36136 | −0.87426 | −1.58016 |
| NA | −1.93228 | 3.379803 | −0.68785 | 0.376017 | 4.323286 | 7.199871 | −1.79503 | −0.53386 | −2.02686 |
| NA | −2.41213 | 4.555552 | 0.806203 | 0.234745 | 5.114885 | 7.490157 | −1.85278 | −2.46886 | −2.24879 |
| NA | −2.22384 | 4.812772 | 0.260694 | −0.11291 | 3.920298 | 7.530501 | −1.46243 | −3.85241 | −0.84338 |
| NA | −2.24589 | 3.195296 | 4.433379 | 0.160197 | 4.413916 | 6.133594 | 0.795383 | −0.04866 | −1.84837 |
| NA | −1.07279 | 3.367553 | 4.385638 | 0.208948 | 4.648573 | 7.34081 | 0.7273 | −0.29788 | −1.7706 |
| NA | −4.63526 | 3.452339 | 4.386568 | 0.317712 | 4.26633 | 7.617622 | 1.042357 | 0.513927 | −1.72772 |
| −0.11681 | −4.08634 | 4.832158 | 0.768777 | 8.880539 | −0.22317 | 1.804376 | −0.81889 | −3.03766 | 0.709948 |
| 0.408537 | −1.97883 | 4.310546 | 1.383728 | 8.795754 | 1.796702 | 1.844584 | 0.120892 | −1.21629 | 1.28866 |
| 0.538945 | −1.83164 | 4.663138 | 1.367527 | 9.015677 | −0.01293 | 1.820907 | 0.087692 | −2.13154 | 1.397888 |

| Unknown | Unknown | Unknown | Unknown | Unknown | Unknown | Unknown | Unknown | Unknown |
|---|---|---|---|---|---|---|---|---|
| −0.59213 | 0.666865 | 1.300526 | −2.23833 | 1.92169 | −3.65232 | −3.1666 | −0.07503 | −2.04162 |
| 0.102619 | 0.243192 | 1.091282 | −1.01355 | 2.51503 | −1.90214 | −2.4821 | −0.6927 | −2.45866 |
| 0.070576 | 1.2842 | 1.277265 | −2.12479 | 2.106634 | 6.539202 | −5.30362 | −0.71912 | −2.34026 |
| 0.415357 | 0.741705 | 1.097985 | −0.87208 | 2.592532 | −0.40473 | −4.95402 | −0.18675 | −2.45065 |
| −0.41114 | −0.20526 | 2.364342 | −0.11444 | 2.950076 | −0.71417 | −2.14752 | −1.97165 | −2.22704 |
| −0.36385 | 0.021787 | 1.254412 | −0.75794 | 2.372124 | −1.54509 | −2.41558 | −1.28753 | −2.27207 |
| 0.13562 | −0.01953 | 1.498312 | −1.90842 | 2.27309 | 6.656923 | −5.22428 | −0.77421 | −2.41747 |
| −1.01874 | −0.03634 | 2.273637 | −1.18395 | −2.63685 | −1.59235 | −1.64355 | −1.5355 | −2.13768 |
| −0.00691 | 0.570058 | 1.86846 | −0.96069 | −2.37281 | −1.73792 | −2.63159 | −1.00815 | −2.0477 |
| −0.5523 | −0.09845 | 2.009564 | −0.97291 | −2.84979 | −1.40796 | −2.71209 | −1.32651 | −1.85461 |
| 0.802109 | 1.572939 | 0.818361 | −1.08515 | 2.309442 | −0.8778 | −5.11895 | 0.033521 | −2.58069 |
| 0.099288 | 0.432817 | 1.592199 | −0.71868 | 2.981225 | 7.282299 | −4.573 | −0.94174 | −1.98188 |
| −0.6701 | 0.866029 | 2.286768 | −1.94705 | 2.276222 | −2.65808 | −2.67639 | −1.82099 | −2.60158 |
| −0.11108 | 0.308142 | 1.028978 | −0.39942 | 2.367792 | −1.68252 | −2.31945 | −1.15578 | −2.52394 |
| 0.212589 | 0.695005 | 1.802308 | −0.20945 | −2.52363 | −1.62525 | −2.89769 | −0.97478 | −2.42157 |
| −0.51429 | 0.879619 | 1.912794 | −1.75496 | 2.437542 | 2.975181 | −4.9915 | −1.32785 | −1.85604 |
| −0.77373 | −0.15832 | 1.155342 | −2.2299 | 1.955271 | 6.382851 | −2.43935 | −1.15517 | −2.22109 |
| 0.378138 | 0.090693 | 1.129601 | −2.08121 | −2.78837 | −1.44049 | −5.33992 | −0.27641 | −2.26219 |
| −0.31 | 0.655895 | 1.131413 | −4.64907 | −2.53452 | 6.458847 | −5.53885 | −1.14449 | −2.55778 |
| 0.38467 | 0.446902 | 2.544513 | −0.56002 | 2.821918 | −1.0168 | −4.66011 | −1.56354 | −2.25645 |
| 0.869631 | −0.24703 | 1.419727 | −0.44221 | −2.07463 | −0.16662 | −5.04091 | −1.89531 | −1.99654 |
| 0.054319 | 0.645818 | −4.49801 | 1.264855 | 0.06627 | 2.500139 | NA | −0.12593 | −0.90535 |
| −0.11863 | 0.752061 | 1.244632 | −1.41786 | 2.346909 | −1.41994 | −1.22006 | −1.06706 | −2.14769 |
| 0.12018 | 0.877587 | 1.449228 | −4.89191 | 2.453567 | 7.884623 | −4.95724 | −0.78501 | −1.77701 |
| −0.26456 | 0.37341 | 2.037255 | −0.53526 | 2.856456 | −1.43927 | −2.53473 | −1.43479 | −2.28776 |
| −0.37691 | −0.8729 | 1.59322 | −2.598 | −0.93612 | 0.431704 | NA | −0.12614 | −1.62229 |
| 0.176905 | −0.17921 | 1.78881 | −4.14521 | −2.02505 | 0.284402 | −5.07717 | 0.028473 | −2.10144 |
| 1.004824 | −0.19382 | 1.363076 | 1.749785 | −1.29414 | 3.517152 | NA | 1.434732 | −0.81946 |
| 0.511644 | 0.626485 | 1.007285 | −1.47863 | −1.6602 | −0.39894 | −4.33761 | −0.38708 | −2.3983 |
| 0.176151 | 0.443725 | 1.13157 | −5.91134 | 2.325077 | −0.92975 | −5.19213 | −0.63796 | −2.43499 |
| −0.29157 | −0.34013 | 1.419031 | −0.6458 | −0.94455 | 0.280483 | NA | 0.374333 | −1.69273 |
| 0.807837 | −0.58854 | −5.46129 | 0.904085 | −1.72995 | 0.472604 | NA | 0.347634 | −1.72236 |
| −0.15246 | 0.00014 | 0.886685 | −1.11314 | −2.23648 | −1.07152 | −2.46884 | −0.20602 | −2.62429 |
| 0 | 0.32087 | 1.260647 | −1.25052 | −2.42599 | −1.61015 | −2.20475 | −0.98572 | −2.62632 |
| 0.460633 | −0.33419 | 1.190858 | −1.56422 | 2.500727 | −1.16803 | −4.80694 | NA | −2.66228 |
| −0.0272 | −2.37842 | −1.96275 | −0.8709 | −1.68204 | −0.18622 | NA | 0.092843 | −2.90646 |
| −1.27101 | −1.26031 | −0.11706 | −5.50443 | −1.73041 | −0.40309 | −5.85852 | −0.31198 | NA |
| −0.60836 | −1.21933 | −2.76249 | −3.59229 | −1.59849 | −0.32188 | −5.2177 | −0.01712 | NA |

| Unknown | Unknown | Unknown | Unknown | Unknown | Unknown | Unknown | Unknown | Unknown | Unknown |
|---|---|---|---|---|---|---|---|---|---|
| −0.78129 | 2.31556 | 0.468245 | −1.19204 | 8.518736 | 2.448115 | 1.870572 | 6.007062 | −3.54086 | 0.259422 |
| −2.62069 | 1.056904 | −0.85222 | −1.68525 | 8.567079 | 1.542608 | 1.153766 | −1.09914 | −3.82233 | 0.228042 |
| −4.04933 | 1.345573 | 0.566053 | −2.04105 | 8.592446 | 1.500274 | 3.215436 | 5.656804 | −2.88278 | −0.65142 |
| −5.20447 | −0.76142 | −1.98798 | 0.653231 | 8.628716 | 3.089426 | 2.812829 | 5.29129 | −3.6706 | −0.41289 |
| −4.1948 | 1.296221 | 0.422838 | −1.91354 | 8.744902 | 2.264836 | 1.411026 | 5.593012 | −2.15819 | 0.900469 |
| −3.65575 | −0.0409 | −0.94382 | −3.9804 | 8.374814 | 2.794642 | 2.390547 | −0.93123 | −3.84935 | −0.31675 |
| −1.96069 | −0.90607 | 0.476905 | −1.35423 | 8.137434 | −1.0652 | 2.610375 | 5.853244 | −3.99738 | −0.10757 |
| NA | 1.948553 | 0.099814 | −1.31275 | 8.261479 | 1.090626 | 1.57405 | 5.680998 | −2.99298 | −0.3928 |
| −3.19077 | 1.306911 | −0.84178 | −0.43667 | 7.940211 | 0.575889 | 1.412252 | 0.253851 | −3.50316 | 0.429064 |
| −6.15456 | 1.173101 | −0.28738 | −3.5904 | 8.176791 | 1.433403 | 2.040095 | −1.1946 | −4.08907 | −0.45689 |
| −4.13795 | −1.6452 | −1.34346 | −2.88161 | 7.009537 | 1.545949 | 1.808679 | 5.575029 | −3.30224 | 0.009462 |
| −3.48891 | −1.38996 | −1.18959 | −1.65276 | 5.10602 | 1.258573 | −0.33618 | 5.234395 | −3.65674 | −0.16825 |
| −4.83703 | 0.728772 | −0.44478 | −1.07902 | 7.119145 | 2.114025 | 2.261877 | 4.897435 | −3.33607 | −0.28999 |
| −4.82276 | 0.017809 | −1.27072 | −0.80777 | 8.04155 | 2.559932 | 1.486063 | −0.83193 | −3.69791 | 0.090803 |
| −4.20683 | 0.540657 | −1.59279 | −1.75604 | 7.371375 | 2.199206 | 2.411149 | −1.01913 | −3.8363 | −0.54112 |
| −0.99492 | −1.44541 | −1.04494 | −1.97414 | 8.703297 | 0.394502 | 1.489538 | 0.201394 | −3.26785 | 0.185509 |
| −1.5918 | 1.469945 | 0.564313 | −1.78358 | 8.29523 | 0.977357 | 1.492439 | 0.164733 | −1.63386 | 0.429822 |
| −3.84817 | 0.980448 | −0.11872 | −3.2151 | 7.584008 | 1.811854 | 2.845807 | −5.73445 | −3.73193 | −0.19773 |
| −3.10028 | −1.45124 | 0.216714 | −1.3527 | 8.149597 | 2.33017 | 1.680077 | 5.438226 | −3.84903 | −0.1896 |
| −1.15604 | 0.409009 | −0.686 | −1.94013 | 8.343697 | 2.286754 | 2.743751 | −0.24687 | −2.35659 | −0.03194 |
| NA | 0.2.78544 | 0.018452 | −1.2681 | 7.165525 | 1.571188 | 1.705413 | 0.524713 | −3.19281 | −0.43119 |
| −3.71069 | 1.651534 | −1.52272 | −0.3246 | 7.120082 | 2.784391 | −0.00821 | 0.450343 | −3.02321 | −0.73794 |
| −2.467 | 1.662908 | −0.64179 | −1.80481 | 8.465726 | 2.218681 | 2.720279 | −0.86822 | −1.46721 | −0.80018 |
| −0.66788 | 0.758568 | 0.360032 | −0.21011 | 8.2424 | 1.542742 | 1.453112 | −0.50232 | −3.46772 | −0.48399 |
| −2.88295 | 0.364284 | −0.14697 | −0.73759 | 8.460991 | 2.578925 | 1.148529 | 0.009116 | −3.55066 | 1.104633 |
| 0.053541 | 1.800137 | 0.301711 | −0.82044 | 7.159001 | 1.522072 | −1.35217 | −1.37558 | −3.49882 | −0.95584 |
| −1.46162 | 0.775236 | 0.686825 | −1.70905 | 8.400153 | 2.089146 | 1.531471 | 5.806254 | −3.66341 | −0.04564 |
| −3.96236 | 0.940773 | −1.26738 | 0.508484 | 7.366429 | 2.196549 | −0.59805 | 0.610252 | −2.5456 | −0.34968 |
| −4.70701 | −1.66048 | −2.19633 | −1.24014 | 7.571903 | −0.54755 | 1.155956 | 0.070018 | −3.63331 | 1.078589 |
| −2.24126 | 0.993712 | 0.559599 | −3.49155 | 8.301659 | 2.033792 | 2.038575 | −1.59219 | −3.72778 | −0.98466 |
| NA | −0.10563 | −1.10626 | −1.44483 | 7.546181 | 1.707374 | 2.014191 | 4.655229 | −3.48514 | 0.341283 |
| −3.64938 | −0.00101 | −1.0556 | −0.47578 | 2.555856 | 1.69244 | −1.02482 | −0.19896 | −2.98525 | 0.00101 |
| −5.6213 | −0.18057 | −0.04949 | −1.5678 | 8.141178 | 1.378819 | 2.187684 | −1.07446 | −3.92823 | −0.61088 |
| −4.60732 | −2.20696 | −0.30916 | −1.75182 | 8.392895 | 1.76109 | 1.732985 | −0.7402 | −3.97236 | −1.27273 |
| NA | 0.985217 | 0.497405 | −2.05494 | 8.303249 | 2.106205 | 1.681826 | −0.5326 | −3.78844 | −0.95947 |
| NA | 2.082681 | −3.03104 | −3.16799 | 0.90049 | 1.831227 | −0.82944 | −0.62198 | −0.64622 | 1.892187 |
| −6.86253 | 2.675719 | −3.23295 | −3.65537 | −0.13122 | 0.633315 | −1.42674 | −0.95499 | −0.47369 | 2.376403 |
| NA | 1.138352 | −2.43263 | −3.62248 | 0.311832 | 1.621778 | −1.00292 | −0.0986 | −0.37042 | 2.364378 |

| Unknown | Unknown | Unknown | Unknown | Unknown | Unknown | Unknown | Unknown | Unknown |
|---|---|---|---|---|---|---|---|---|
| −0.31781 | 0.249508 | −0.62621 | −1.27741 | NA | −3.10359 | −0.82614 | 1.075282 | 1.531785 |
| −0.66932 | −0.01902 | −0.68741 | −2.2954 | NA | −2.98261 | −1.5579 | 1.494211 | 0.509777 |
| −0.53453 | 0.98575 | −0.28421 | −2.03371 | NA | −2.0709 | −1.21709 | −0.15718 | −0.38133 |
| −2.21745 | 0.176313 | −0.55636 | −1.6583 | NA | −2.45889 | −1.70807 | 3.107029 | −2.89458 |
| −0.44945 | NA | 0.048477 | −0.13114 | NA | −2.61919 | −0.85255 | −1.47434 | −2.92466 |
| −0.33188 | −0.70006 | −0.21576 | −0.92186 | NA | −3.04517 | −1.65545 | 0.666595 | 0.80898 |
| 0.032449 | 0.263261 | −1.27587 | −1.76152 | NA | −2.80597 | −2.7798 | 0.461984 | 0.807347 |
| −0.82669 | 0.398589 | −1.8637 | 0.160409 | NA | −3.73051 | −2.69578 | 0.937208 | 1.317457 |
| −0.01366 | 0.170954 | −1.20361 | −0.19569 | NA | −3.57187 | −1.19587 | 1.285953 | 0.976233 |
| −0.89447 | −0.05986 | −1.30247 | 0.290363 | NA | −2.5667 | −0.63861 | 0.587611 | 0.517294 |
| −0.3186 | −7.5441 | −0.11549 | −1.13679 | NA | −2.09584 | −1.65272 | 2.955986 | −3.44347 |
| −0.43526 | 0.008216 | 0.540253 | −0.09416 | NA | −3.01832 | −1.42468 | 0.928571 | 0.755327 |
| −0.40264 | 0.059299 | 0.447246 | 0.27457 | NA | −2.21902 | −1.81748 | 1.091316 | 0.988868 |
| −0.31188 | −0.36604 | −0.45104 | −0.8261 | NA | −2.43424 | −2.01555 | 2.915063 | 2.824873 |
| −0.60859 | −0.48604 | 0.818312 | 0.3096 | NA | −3.99847 | −1.47367 | −1.89757 | −0.77903 |
| −0.70425 | −0.31151 | −1.42184 | −0.45779 | NA | −2.1256 | −2.44686 | 0.459413 | 0.366809 |
| −0.73668 | 0.236838 | −3.14615 | −1.20277 | NA | −3.19444 | −1.58817 | 1.590747 | 0.703503 |
| −0.30279 | 0.009934 | −0.38837 | −1.38633 | NA | −2.58762 | −1.29462 | 0.56761 | −0.35692 |
| −0.45189 | −0.32796 | 0.105534 | −0.46404 | NA | −2.26476 | −0.93031 | −1.54597 | −0.60871 |
| −1.01793 | −0.36443 | 0.875537 | 0.277108 | NA | −3.0177 | −2.31715 | 1.767094 | 1.729946 |
| −0.42501 | −0.95071 | 0.256962 | −1.00525 | NA | −2.43617 | −1.05064 | −0.18686 | −2.37929 |
| −0.34359 | −4.62007 | −0.39494 | −1.65528 | NA | −1.92589 | 0.793117 | 0.060233 | −0.1327 |
| −0.34049 | −0.03403 | −0.93434 | −0.34271 | NA | −2.88653 | −0.99768 | 0.139893 | 0.193556 |
| −0.57257 | −0.44817 | −1.78002 | 0.623408 | NA | −2.01676 | −0.94193 | −0.20662 | 0.288877 |
| 0.7003 | 0.357297 | −0.5133 | −0.79447 | NA | −3.05721 | −1.4019 | 1.360095 | 1.114375 |
| −1.00488 | −1.88453 | −0.45295 | −0.42522 | NA | −2.06247 | −2.71655 | 0.543582 | 0.737562 |
| −0.42344 | −0.02847 | −2.55014 | −0.99785 | NA | −2.42385 | −1.07046 | 0.173983 | −1.02652 |
| 0.051698 | NA | 0.281221 | −1.15308 | −0.40104 | −1.79854 | −0.59362 | −0.98429 | 2.665986 |
| −0.07456 | 0.386007 | 0.295267 | −1.27475 | NA | −3.27623 | −1.90402 | 0 | −0.77957 |
| −1.03614 | −0.69021 | −0.67329 | −1.98516 | NA | −2.58069 | −0.46652 | −0.01605 | −0.74622 |
| −0.39655 | −2.84535 | −2.69403 | −1.84506 | NA | −3.63772 | −0.61779 | −0.70355 | −1.26746 |
| −0.51601 | NA | −2.75143 | −1.12855 | −1.10176 | −1.74493 | −0.20288 | 2.557627 | −0.21786 |
| −0.84277 | 0.512585 | 0.096509 | −1.34204 | NA | −2.80382 | −1.058 | 2.841722 | 2.739889 |
| −0.69675 | 0.963407 | 0.295949 | −0.88453 | NA | −2.67346 | −1.02243 | 2.919628 | −1.70548 |
| −0.62674 | −0.02848 | 0.07092 | −1.34359 | NA | −2.21964 | −0.90105 | 0.797658 | 0.274232 |
| −0.83192 | −0.2332 | −3.26182 | −2.63947 | −2.18131 | −3.64641 | −0.92115 | −0.2252 | 1.782565 |
| −1.07333 | 0.293473 | −1.14271 | −0.80769 | −2.13357 | −3.18495 | −1.14206 | 0.254743 | 1.331233 |
| −1.06988 | 0.500312 | −1.01405 | −0.68133 | −0.9117 | −3.22496 | −0.56016 | −0.33595 | −1.1854 |

| Unknown | Unknown | Unknown | Unknown | Unknown | Unknown | Unknown | Unknown | Unknown | Unknown |
|---|---|---|---|---|---|---|---|---|---|
| −0.63489 | −1.23201 | −1.15303 | 0.951555 | 2.279898 | −1.07031 | −0.58587 | −1.29011 | −2.72345 | −1.90292 |
| −3.26187 | −0.14312 | −0.54631 | 1.020944 | 1.818291 | −1.42557 | −0.60538 | −1.23825 | −3.8579 | −7.99422 |
| −2.27464 | −0.23185 | −0.86813 | 1.078586 | 1.75085 | −1.79195 | −0.42015 | −1.11551 | −3.1511 | −1.36721 |
| −2.4814 | −0.76112 | −0.44848 | 0.707389 | 2.153068 | −1.37229 | −0.74256 | −1.18068 | −4.34501 | NA |
| −2.97619 | −2.75116 | −1.55679 | 0.447907 | 2.621312 | −0.20078 | −0.21061 | −0.18686 | −3.09309 | −3.26195 |
| −0.81118 | 2.132708 | −1.90602 | 0.508468 | 2.084344 | −1.30079 | −0.72767 | −0.82203 | −3.41553 | −2.6654 |
| −0.23736 | −2.30898 | −0.4479 | 1.20718 | 2.493894 | −0.76688 | −0.62392 | −0.82544 | −2.34102 | −3.19049 |
| −0.74408 | 1.760259 | −1.9998 | 0.041752 | 2.297045 | −1.01038 | −0.51352 | 1.058641 | NA | −3.0947 |
| −2.37988 | −0.21706 | −1.68307 | 0.476783 | 2.356046 | −1.0758 | −0.69238 | −0.09231 | −3.79398 | −3.50691 |
| −2.46786 | 2.536771 | −1.79561 | 0.162473 | 1.589305 | −1.35295 | −0.46203 | 0.380182 | −4.02789 | −2.29007 |
| −2.38543 | −0.61637 | −0.52682 | 1.236455 | 1.544028 | −1.85415 | −0.49733 | −1.77466 | −2.78973 | −2.38845 |
| −2.31875 | 2.746634 | −2.20937 | 0.515423 | 2.175457 | −0.73597 | −0.67627 | −1.09269 | −2.82794 | −3.02604 |
| −1.77217 | −0.90259 | −1.73333 | −0.02596 | 2.249773 | −1.50206 | −0.63606 | 0.131613 | −6.1153 | −3.58722 |
| −2.54886 | −2.26161 | −1.79442 | 1.018784 | 2.126984 | −1.70007 | −0.83103 | −1.19563 | −3.68474 | −1.83981 |
| −2.43308 | −0.68704 | −3.02545 | 0.480687 | 1.927097 | −0.92216 | −0.60445 | −0.39873 | −3.6876 | −3.7132 |
| −2.6492 | −0.49895 | −2.34516 | 0.080824 | 2.21549 | −1.37535 | −0.60219 | 0.298134 | NA | −1.40268 |
| −3.30431 | −0.81903 | −2.23753 | 0.35432 | 2.069465 | −1.37815 | −0.83569 | −0.61552 | −2.85846 | −1.81929 |
| −3.22938 | 2.341795 | −0.18698 | 1.516172 | 1.8012 | −1.30706 | −0.54203 | −1.43077 | −3.35112 | −2.30021 |
| −2.63178 | −0.95391 | −1.73112 | 0.740138 | 2.46355 | −1.6533 | −0.75334 | −0.71535 | NA. | NA |
| −2.26869 | 2.706563 | −1.60125 | 0.051755 | 1.78461 | −0.91944 | −0.53575 | −0.33931 | −3.61499 | −5.42477 |
| −0.6649 | 2.299713 | −0.07277 | 1.212431 | 1.501778 | −1.81111 | −0.59192 | −1.94802 | −3.93596 | −1.33836 |
| −0.84006 | −1.73793 | −0.54132 | 0.597796 | 1.72812 | −1.94591 | −0.65628 | −1.23796 | NA | 0.864114 |
| −1.45465 | 2.625962 | −1.9339 | 0.969446 | 1.945189 | −1.28096 | −0.691 | −0.87591 | −3.1756 | −1.86536 |
| −2.6231 | 2.732088 | −0.99955 | 0.690308 | 0.849549 | −0.55683 | −0.16789 | −0.67752 | −2.28815 | −1.94675 |
| −2.38928 | −0.61003 | −2.53817 | 0.11069 | 1.756263 | −1.32388 | −0.56913 | 0 | −5.78764 | −1.59662 |
| −2.83686 | 2.862388 | −0.97369 | 0.513085 | 1.108692 | −1.59364 | −0.99759 | −2.12863 | NA | 0.861679 |
| −2.85978 | 2.058159 | −0.2488 | 0.990722 | 1.588373 | −1.38226 | −0.72785 | −1.41982 | −3.77835 | −0.54868 |
| −0.48337 | 2.431574 | −1.17758 | 1.02581 | 1.184027 | −2.35585 | −0.81843 | −1.14105 | −3.92217 | −1.54654 |
| 0.102408 | 2.52009 | −0.1703 | 1.527655 | 1.827495 | −1.77266 | −0.89389 | −1.53544 | −4.22869 | −2.99024 |
| −1.32209 | −1.22155 | −1.42935 | 1.018696 | 0.891542 | −1.67397 | −0.84275 | −1.74167 | −2.4705 | −2.68606 |
| −1.66335 | 2.380158 | −1.51796 | 0.727103 | 2.470979 | −2.82575 | −0.43118 | −2.85682 | NA | 0 |
| −2.02686 | 1.517194 | −1.06556 | 0.873232 | 2.644569 | −2.19092 | −0.65212 | −1.41957 | NA. | 0.079594 |
| −2.46847 | 2.05076 | −0.43344 | 0.495576 | 1.484813 | −3.17512 | −0.66808 | −1.90318 | −4.25321 | −3.39175 |
| −2.35291 | 2.502406 | −1.27776 | 0.504033 | 1.983708 | −3.10883 | −0.83686 | −0.74271 | −4.66775 | −2.51085 |
| −2.592 | 2.643771 | −0.45204 | 0.822612 | 1.456357 | −1.56625 | −0.66924 | −2.0029 | −4.03855 | −2.00521 |
| −0.71775 | 4.214543 | −1.72305 | 0.796192 | 0.459436 | −4.66443 | −0.31815 | −4.03294 | NA | −1.02822 |
| −0.00014 | −0.86012 | −2.42448 | 1.331269 | 0.890389 | −3.38869 | −0.22817 | −6.32933 | NA | −4.93527 |
| −0.06199 | −0.54138 | −2.07318 | 1.284477 | 1.053314 | −2.96778 | −0.5485 | −3.73082 | NA | −3.09597 |

| Unknown | Unknown | Unknown | Unknown | Unknown | Unknown | Unknown | Unknown | Unknown |
|---|---|---|---|---|---|---|---|---|
| −0.82873 | −2.06772 | −1.76532 | 0.385574 | 0.549377 | 2.429592 | −0.18029 | −1.21578 | −1.50937 |
| −2.65805 | −1.72581 | −2.80465 | 1.026168 | 0.118919 | 0.678423 | −1.95169 | 1.097932 | −2.86028 |
| 0.126001 | −1.47942 | −3.04529 | 0.663347 | 0.422175 | −0.88434 | −0.16742 | −1.00633 | −3.08344 |
| −0.08598 | −2.00576 | −2.30993 | 0.399618 | 0.100644 | −0.10137 | −0.79841 | −0.32316 | 0.880089 |
| −3.55861 | −1.80498 | −0.83716 | −0.87443 | −4.38024 | 0.475109 | −0.25932 | 0.622724 | 1.082861 |
| 0.536617 | −1.81474 | −2.40941 | 0.77899 | −0.14221 | −0.41464 | −0.63106 | 5.118941 | 1.746676 |
| −3.88721 | −2.06369 | −2.74808 | 0.108985 | −0.08848 | −0.61577 | −0.42488 | −0.46903 | −1.60337 |
| −0.26147 | −2.17897 | −3.4711 | −0.13321 | −2.63385 | −3.25589 | −0.40861 | 5.824687 | 0.559957 |
| −0.29873 | −1.99419 | −2.87711 | 1.066084 | 0.168821 | 0.576171 | 0.443871 | 5.489295 | −3.06973 |
| −0.63301 | −2.00021 | −3.36162 | 0.060248 | −1.44481 | 0.817725 | 0.496649 | 5.399131 | 1.83251 |
| −0.15007 | −1.63812 | −2.38002 | 0.748817 | 0.665955 | 0.368395 | 0.521914 | −1.67689 | −1.83513 |
| 0.987788 | −1.93181 | −2.07261 | 1.014839 | 0.295171 | −0.14405 | −0.54359 | −1.91805 | 1.029346 |
| −0.12884 | −2.13611 | −2.84503 | 0.433084 | −5.38407 | −1.22433 | −0.73264 | 4.979242 | 1.153194 |
| −0.51202 | −2.21316 | −2.27232 | 0.622161 | −0.53543 | −0.43118 | −0.45143 | 4.765737 | 1.708796 |
| −0.19543 | −2.14511 | −2.55638 | 0.51062 | −9.32393 | −0.76677 | −1.70169 | 5.063928 | 0.84604 |
| 0.624386 | −1.69489 | −2.46869 | 0.359793 | −1.5399 | −0.92516 | −0.69338 | 5.216113 | −0.86599 |
| 0.323346 | −2.03768 | −2.85092 | −0.11219 | −0.93327 | −0.13345 | 0.003509 | 5.299157 | 1.13319 |
| −0.01257 | −2.20029 | −2.66122 | 1.027335 | 1.081397 | 0.735258 | 0.505741 | 0.92134 | −2.662 |
| 0.28048 | −1.29452 | −3.01186 | 0.942608 | 0.045678 | −0.2107 | 0.08245 | −0.14091 | 1.296831 |
| −0.40095 | −1.55758 | −0.98109 | −0.00339 | −5.63161 | 0.621229 | −0.51158 | −1.9983 | 0.398053 |
| 2.192638 | −1.45382 | −1.96812 | 0.943353 | 1.627376 | −0.2125 | −0.50851 | −0.00019 | 0.416019 |
| −0.02511 | 0.254232 | −1.08081 | 0.776172 | 1.459233 | 0.185947 | −1.45187 | 0.126302 | 0.990853 |
| 0.206732 | −1.3179 | −2.11201 | 0.470923 | −0.82005 | 0.711145 | −0.24928 | −1.26531 | 0.709978 |
| 0.822976 | −1.46512 | −1.87918 | 0.647202 | 0.582327 | 0.224717 | −0.32956 | −1.99042 | −0.65522 |
| −0.49519 | −2.05541 | −2.41553 | 0.568937 | −1.51187 | −0.13411 | −0.46729 | 0.537041 | −0.25539 |
| 0.183476 | −1.23248 | −1.44975 | 0.60559 | 0.822277 | −0.23857 | 0.39802 | −1.1796 | −3.30139 |
| 1.560512 | −1.47458 | −2.28542 | 0.920718 | 0.973329 | −0.39417 | −2.35883 | 5.087193 | −2.59841 |
| −0.50761 | −1.27066 | −1.30009 | 1.6232.69 | 2.007442 | −0.18579 | −1.67756 | 0.231563 | −2.62495 |
| −2.05413 | −2.41215 | −2.55846 | 0.645645 | 1.219043 | −0.00959 | −0.3683 | 0.092396 | −2.83413 |
| 0.191084 | −1.41236 | −1.39108 | 0.8192.77 | 0.712101 | −0.44642 | −0.70979 | −0.59084 | 0.053457 |
| 0.868249 | −1.31414 | −2.15749 | 0.868016 | 0.883712 | −0.48027 | 0.505713 | 0.614961 | 2.151271 |
| −0.58623 | −1.15894 | −1.86259 | 1.21127 | 1.46443 | 0.31906 | 0.390971 | 0.592082 | −2.91161 |
| 0.594745 | −1.30444 | −2.57646 | −0.44318 | 0.45149 | 0.327088 | −0.45409 | −2.45892 | −3.84724 |
| 0.516208 | −1.16888 | −2.47038 | 0.319744 | 0.206945 | 0.031373 | −0.0381 | −0.30805 | −3.65192 |
| 0.549513 | −1.76605 | −2.0107 | 0.768691 | 0.955225 | −0.06439 | −0.31505 | −2.20592 | −3.16251 |
| 0.769144 | −0.46942 | −2.49548 | 0.969878 | −0.64774 | 1.238053 | 0.457008 | 2.820874 | −4.6327 |
| 1.195355 | 0.347068 | −3.34965 | 0.436105 | −1.39737 | 1.288459 | 1.285855 | 3.639786 | −4.19979 |
| 1.39142 | 0.378082 | −3.45975 | 1.116205 | −0.81028 | 1.460623 | 1.466022 | 3.486148 | −5.05411 |

| Unknown | Unknown | Unknown | Unknown | Unknown | Unknown | Unknown | Unknown | Unknown | Unknown |
|---|---|---|---|---|---|---|---|---|---|
| −0.56645 | −0.97346 | −0.43652 | −2.00481 | 0.451522 | −1.63383 | −0.86883 | 1.441699 | −1.12717 | −1.56611 |
| −0.33146 | −1.21833 | −0.88315 | −1.35676 | 0.210253 | −0.62565 | −0.73892 | 1.504496 | −1.21577 | −1.02641 |
| −0.59768 | −1.49054 | −0.72828 | −0.54232 | 2.041381 | −0.75791 | −0.91462 | 1.480842 | −1.33807 | −0.22499 |
| −0.38556 | −1.36349 | −1.35911 | −1.11542 | 1.866274 | −0.82 | −1.05778 | 1.649925 | −1.3939 | −2.84876 |
| −0.22867 | −1.33116 | −0.60117 | 0.204554 | 0.542758 | −0.64333 | −2.53023 | 2.192444 | −0.81078 | 1.085837 |
| −0.6079 | −1.20592 | −0.62152 | −0.9428 | 1.400236 | −1.01578 | −1.23985 | 0.929733 | −1.1834 | −0.7137 |
| −0.43423 | −1.25343 | −0.66115 | −1.68666 | 0.274757 | −1.23865 | −1.57546 | 1.568955 | −0.52299 | 0.400452 |
| −0.42251 | −0.62894 | −0.7617 | −0.81831 | −0.0134 | −2.24866 | −0.69542 | 1.519769 | −1.28032 | 0.539423 |
| −0.44811 | −1.068 | −0.54475 | −1.15501 | 0.313041 | −0.90668 | −0.58377 | 1.660574 | −1.09762 | 0.008595 |
| −0.64022 | −1.29849 | −0.42087 | −1.20418 | 0.112314 | −0.88062 | −1.01781 | 1.350355 | −1.54652 | 0.092089 |
| ~0.15846 | −1.33278 | −0.89616 | 0.032756 | 2.614445 | 0.053811 | −0.83824 | 1.725426 | −0.79801 | 0.229061 |
| −0.06037 | −0.76057 | −0.24239 | −0.69547 | −0.00822 | −0.80134 | −1.32375 | 1.518582 | −1.24104 | −0.41089 |
| −0.80824 | −1.38819 | −0.77535 | 0.186817 | −0.29658 | −1.08812 | −1.0675 | 1.190895 | −1.09627 | −0.51513 |
| 3.766838 | −1.02031 | −0.65405 | −0.94927 | −2.03735 | −0.54257 | −1.67937 | 1.374286 | −0.85433 | −0.42551 |
| −0.00201 | −0.80011 | −0.61138 | −0.18614 | 2.276838 | −0.30968 | −0.89532 | 1.732531 | −1.0304 | 0.116782 |
| 0.850917 | −1.1562 | −0.35602 | −0.28355 | −0.09493 | −0.70469 | −0.72409 | 1.646649 | −1.38638 | 0.638987 |
| −0.56141 | −0.97489 | −0.67183 | −2.61755 | 0.127511 | −1.50933 | −2.40796 | 1.508726 | −0.9309 | 0.330675 |
| 0.40851 | −1.65916 | −0.33407 | −1.44727 | 0.281033 | 0.0915 | −1.46464 | 1.267758 | −0.93083 | −1.57111 |
| 3.147543 | −2.21182 | −0.80746 | −1.2219 | 1.763662 | −0.9934 | −0.92639 | 1.484205 | −1.00992 | −0.95743 |
| −0.71524 | −1.28482 | −0.94583 | 1.019531 | 0.604661 | −1.34112 | −1.77169 | 1.711637 | −1.83224 | 0.729982 |
| −0.0017 | −1.14838 | −0.554 | −2.05461 | −3.77324 | 0.479658 | −1.42083 | 1.45952 | −1.65781 | −1.00519 |
| 3.052576 | −1.2329 | −1.05329 | −1.58604 | 1.139388 | −0.95855 | −1.56002 | 0.508507 | NA | 0.24511 |
| 0.012.444 | −1.21636 | −0.38501 | −0.55818 | 0.36989 | −0.78752 | −0.55868 | 1.61423 | −1.16709 | 1.706956 |
| 0.499659 | −0.43402 | −0.16933 | −1.05238 | −0.02785 | −0.75375 | −0.7258 | 1.392599 | −1.23344 | 1.134872 |
| −0.02085 | −0.9753 | −0.37519 | −0.28909 | 0.41713 | −0.32647 | −1.11524 | 1.728093 | −2.56121 | 0.05749 |
| −0.35157 | −1.24543 | −0.33976 | −1.52518 | 1.345737 | −1.68511 | −0.74362 | 0.867192 | −2.14305 | −2.38293 |
| 2.555494 | −1.13743 | −0.33893 | −1.79523 | −0.1109 | −0.48935 | −1.92733 | 1.548394 | −1.51985 | −2.07392 |
| −2.39614 | −1.58163 | −0.49821 | −2.18401 | −1.88869 | 1.195435 | −0.97237 | 1.829137 | −0.90951 | −0.16964 |
| −0.41722 | −1.1277 | −0.59276 | −0.88316 | 0.238449 | −0.39594 | −2.21863 | 1.57945 | −1.34548 | 1.173364 |
| −0.56602 | −1.30863 | −0.58258 | −1.23054 | 0.273756 | −1.02474 | −0.94754 | 1.270125 | −1.66373 | 1.476064 |
| 4.226441 | −0.322 | −0.6741 | −0.94314 | 1.833423 | −0.56555 | −1.05155 | 0.781342 | −1.48569 | 0.174041 |
| 0.584613 | −2.378 | −0.71727 | −1.85401 | −0.514 | 0.260075 | −1.13653 | 0.879551 | −0.98188 | −0.02327 |
| 1.232826 | −2.23484 | −1.14992 | −1.59169 | 0.750961 | −0.24718 | −1.20049 | 1.737111 | −2.57165 | −3.55057 |
| 1.454576 | −1.63251 | −0.89962 | −0.92454 | 2.292055 | 0.16467 | −0.97256 | 1.862259 | −1.91316 | −0.36834 |
| −0.8383 | −1.91906 | −1.00166 | −1.45913 | 2.264168 | −0.18581 | −0.89196 | 1.494218 | −2.32896 | −3.58458 |
| 0.279773 | −3.87247 | −2.98571 | −3.0822 | 2.886382 | −1.50103 | −4.77027 | −2.13914 | NA | 1.793734 |
| 0.933294 | −3.73834 | −2.93252 | −2.07027 | 3.442486 | −0.83014 | −3.59508 | −1.61129 | NA | 2.037907 |
| −1.0428 | −3.91824 | −2.58843 | −2.42903 | 3.552116 | −0.6259 | −3.46682 | −1.23529 | NA | 2.321348 |

| Unknown | Unknown | Unknown | Unknown | Unknown | Unknown | Unknown | Unknown | Unknown |
|---|---|---|---|---|---|---|---|---|
| −0.92192 | −3.21832 | 1.345695 | −3.23639 | NA | NA | 2.220671 | 2.12307 | 5.101982 |
| −2.3054 | −0.81816 | 0.698575 | −3.75653 | NA | NA | 2.328357 | 2.320929 | 5.416561 |
| −0.8492 | −0.49901 | 0.606157 | −3.57008 | NA | NA | 2.244538 | 2.262344 | 5.592718 |
| −1.36707 | −0.402 | 1.914959 | −3.48877 | NA | NA | 2.127309 | 1.678001 | 5.190565 |
| −1.17429 | −0.28649 | 1.397912 | −2.60006 | NA | 0.521257 | 3.537403 | 2.567024 | 5.387744 |
| −1.30336 | −0.49029 | 1.565752 | −2.69648 | NA | NA | 1.904675 | 1.465731 | 5.49488 |
| −0.96035 | −1.00882 | −0.22375 | −2.81385 | NA | NA | 2.320464 | 2.094269 | 5.140769 |
| −1.34593 | 0.446084 | 1.182231 | −3.80859 | NA | NA | 2.795155 | 1.742429 | 5.11157 |
| −1.23747 | −0.81062 | 1.21198 | −3.3623 | NA | NA | 2.683212 | 2.045833 | 5.39715 |
| −1.99923 | 0.760762 | 0.537376 | −4.56495 | NA | NA | 2.296054 | 2.091846 | 5.426372 |
| −1.49034 | −0.56803 | 1.502106 | −3.32733 | NA | NA | 2.62616 | 2.649292 | 5.39023 |
| −1.46821 | −0.23575 | 1.264807 | −4.40888 | NA | NA | 2.312697 | 2.14461 | 5.639031 |
| −2.20691 | 1.596295 | 0.511993 | −4.26039 | NA | 1.331022 | 2.802486 | 1.118246 | 5.153324 |
| −1.17979 | −3.73692 | −0.82374 | −3.26921 | NA | 1.996904 | 2.444348 | 2.301531 | 5.403479 |
| −1.12297 | −0.65075 | 1.455568 | −3.34944 | NA | NA | 2.252044 | 1.145813 | 5.496902 |
| −1.62027 | 1.439934 | 1.37669 | −3.7497 | NA | NA | 2.994007 | 1.45167 | 5.456341 |
| −1.27577 | −0.81558 | 0.117853 | −4.21734 | NA | NA | 2.661312 | 2.067799 | 5.173665 |
| −0.50591 | 1.464077 | −0.22112 | −3.24272 | NA | 1.731944 | 2.367474 | 2.301433 | 5.522873 |
| −3.25493 | 1.256648 | 1.085243 | −3.02998 | NA | NA | 2.333571 | 2.121985 | 5.102927 |
| −0.95586 | −2.71485 | 3.204206 | −2.93437 | NA | NA | 3.061059 | 1.775292 | 4.722666 |
| −0.94144 | −0.54114 | 0.620746 | −4.40176 | NA | NA | 2.374395 | 1.853936 | 5.570475 |
| −1.01137 | −0.47992 | 0.508193 | NA | NA | NA | 1.925642 | 1.625262 | 3.806398 |
| −0.88618 | −0.16121 | 1.176454 | −3.5868 | NA | NA | 2.518185 | 2.52715 | 5.233985 |
| −2.07756 | −0.48749 | 1.773604 | −3.68859 | NA | NA | 1.537871 | 2.396483 | 4.267311 |
| −1.28337 | −3.33387 | 0.882369 | −3.84584 | NA | NA | 3.352662 | 1.985971 | 4.419677 |
| −2.00898 | 1.251523 | 1.814464 | NA | −2.34912 | NA | 2.645524 | 1.202659 | 4.105484 |
| −1.27802 | −0.76502 | −1.23008 | −3.68766 | NA | NA | 2.370026 | 1.971702 | 5.650492 |
| −1.80122 | −0.44109 | 1.467653 | −3.6551 | NA | NA | 2.33941 | 1.531744 | 3.807894 |
| −1.362 | −0.69858 | 1.701551 | −3.32919 | NA | NA | 2.455691 | 1.419616 | 4.010563 |
| −1.09428 | −0.69137 | 1.277035 | −4.18514 | NA | 1.471876 | 1.819639 | 1.755194 | 5.174346 |
| −2.02654 | −0.83258 | −1.61927 | −4.97645 | NA | NA | 1.500801 | 2.332762 | 3.89786 |
| −1.24761 | −0.73512 | 1.086058 | −4.77641 | NA | NA | 2.644686 | 2.06727 | 4.043202 |
| −1.12303 | −0.60022 | 1.800013 | −3.82724 | NA | NA | 2.396112 | 1.572483 | 5.03732 |
| 0.06637 | −0.73857 | 1.623383 | −3.53263 | 1.100306 | 0.056586 | 2.29042 | 0.547865 | 5.215389 |
| −0.28938 | −0.36034 | 1.954762 | −4.24234 | NA | NA | 0.471761 | 1.87468 | 3.315288 |
| 0.574985 | 4.056646 | −1.8674 | NA | 2.580167 | 6.146721 | 1.656412 | 1.740976 | −0.22812 |
| 0.058744 | −0.62965 | −1.71966 | −5.33525 | 3.276876 | 6.665999 | 2.205184 | 2.428767 | 0.287651 |
| 0.571227 | 3.864171 | −1.58792 | −4.90701 | 3.345499 | 6.800064 | 2.325239 | 2.354653 | 0.453857 |

| Unknown | Unknown | Unknown | Unknown | Unknown | Unknown | Unknown | Unknown | Unknown | Unknown |
|---|---|---|---|---|---|---|---|---|---|
| −1.10248 | 3.798621 | −0.44943 | 6.721052 | NA | 0.951564 | 1.434907 | 3.469701 | −2.43274 | −0.02141 |
| −0.71453 | 3.743174 | −0.33567 | 4.246808 | NA | 1.421547 | 1.578569 | 3.276358 | −2.92895 | 0.17516 |
| −2.16921 | 3.823347 | 0.627514 | 4.193625 | NA | 1.012402 | 1.830845 | 3.586423 | −2.18763 | 0.039829 |
| 0.331151 | 3.586057 | 0.454206 | 4.104662 | NA | 1.195473 | 0.883349 | 3.217935 | −2.89852 | −0.00027 |
| −0.65089 | 4.386762 | 1.140861 | 6.50935 | NA | 1.106517 | 0.974206 | 3.598715 | −1.486 | 0.234321 |
| −0.09558 | 3.126231 | 1.001556 | 2.914662 | NA | 1.431163 | 1.858595 | 2.486797 | −1.55759 | 0.325852 |
| −0.68223 | 3.931657 | −1.6467 | 3.890375 | NA | 1.513634 | 1.10606 | 3.366271 | −2.19947 | 0.202522 |
| −0.96805 | 3.715242 | 0.264818 | 4.725957 | NA | 0.886248 | 1.317293 | 2.977319 | −2.0431 | −0.47235 |
| −0.23407 | 4.068121 | −0.10774 | 4.575796 | NA | 1.240451 | 1.461543 | 3.322592 | −1.98062 | 0.53617 |
| −0.42569 | 3.770135 | 0.526363 | 4.231643 | NA | 1.29083 | 1.470571 | 3.100072 | −2.0608 | 0.22572 |
| −1.87468 | 3.861286 | −0.02815 | 4.188856 | NA | 1.671633 | 0.979008 | 3.558278 | −3.50325 | 0.569208 |
| −1.32197 | 3.862337 | 0.431492 | 6.059924 | NA | 1.252887 | 1.033787 | 3.268206 | −2.87337 | 0.820043 |
| 0 | 3.544768 | 0.506042 | 4.140185 | NA | 1.289733 | 1.892468 | 2.793506 | −1.70989 | 0.017951 |
| −1.91504 | 3.661629 | −0.05697 | 3.570614 | NA | 1.918008 | 1.726729 | 2.86293 | −1.77289 | 0.343136 |
| −1.31949 | 3.951435 | −0.45217 | 4.437105 | NA | 0.936192 | 0.660681 | 3.1968 | −2.17109 | 0.51095 |
| −0.60877 | 3.923751 | 0.264201 | 4.292291 | NA | 1.680699 | 0.378385 | 3.280942 | −3.18965 | −0.04223 |
| −2.03559 | 3.755259 | −0.4643 | 5.608825 | NA | 1.524625 | 1.377428 | 3.186158 | −3.60314 | 0.088993 |
| −1.97085 | 3.642157 | 0 | 3.493982 | NA | 2.01269 | 1.460299 | 3.32746 | −1.95963 | −0.04615 |
| −0.59076 | 3.641119 | 0.17185 | 4.935059 | NA | 1.343324 | 1.100196 | 3.102864 | −3.23982 | −0.02287 |
| −0.67692 | 3.749309 | 0.365067 | 7.582993 | NA | 0.141206 | 0.838847 | 3.033193 | −2.10157 | 0.650014 |
| −1.86839 | 3.439608 | 0.587334 | 4.175027 | NA | 1.880906 | 0.8191 | 2.997609 | −1.47312 | −0.9281 |
| −2.09094 | 2.562118 | −0.86089 | 1.648657 | NA | 2.17722 | 0.974768 | 2.333154 | −0.27863 | −2.72778 |
| −1.12379 | 4.089285 | −0.27995 | 4.809603 | NA | 1.307194 | 0.995102 | 3.650444 | −3.24779 | 0.801603 |
| −1.16467 | 4.007909 | 0.67253 | 5.938329 | NA | 1.482861 | 0.405243 | 3.677645 | −2.67335 | 0.190779 |
| −0.97727 | 4.176731 | 0.514005 | 4.155414 | NA | 1.831741 | 0.047037 | 3.365934 | −2.62232 | 0.331742 |
| −1.66317 | 3.073537 | 0.275983 | 5.785713 | NA | 2.37967 | 1.370181 | 2.800767 | −1.69777 | −1.57759 |
| −1.34969 | 3.711143 | −0.14962 | 4.177208 | NA | 2.028879 | 0.635603 | 3.360017 | −3.02451 | −0.33732 |
| −2.01312 | 3.513925 | 0.277805 | 5.837735 | NA | 1.90163 | 0.526906 | 3.568396 | −1.15453 | −2.15411 |
| −1.80713 | 3.90472 | −0.47851 | 3.594646 | NA | 1.655954 | 1.549163 | 3.423647 | −1.55516 | 0.432571 |
| −1.62848 | 3.464525 | 0.020491 | 5.819442 | NA | 1.385919 | 0.455565 | 3.24607 | −2.78443 | 0.240605 |
| −2.25168 | 3.050681 | 0.099902 | 3.247658 | NA | 1.507794 | 1.712113 | 2.807857 | 0.085948 | −1.53162 |
| −1.60597 | 2.900844 | 0.113681 | 2.610771 | NA | 2.414895 | 1.585694 | 2.784181 | −0.78214 | −2.00286 |
| −1.81847 | 4.014917 | 0.228271 | 4.188044 | NA | 1.424646 | 0.7288 | 3.639171 | −1.99494 | −1.00345 |
| −0.53586 | 4.20385 | 0.324614 | 4.204928 | NA | 0.549226 | 0.976264 | 3.681233 | −2.95461 | 0.008778 |
| −1.81756 | 3.942241 | −0.04488 | 4.565442 | NA | 1.680945 | 0.726971 | 3.660401 | −2.84165 | −0.07842 |
| −2.33646 | 1.070434 | 2.357243 | −1.34362 | 1.866873 | −3.81345 | −2.18757 | −2.23506 | NA | −4.91552 |
| −1.77679 | 1.776275 | 3.029347 | −2.32392 | 1.581942 | −4.29586 | −1.73921 | −2.93003 | NA | −4.27679 |
| −1.66527 | 1.805478 | 3.086295 | −3.86219 | 1.948596 | −3.91841 | −1.4531 | −1.1733 | NA | −4.48212 |

| Unknown | Unknown | Unknown | Unknown | Unknown | Unknown | Unknown | Unknown | Unknown |
|---|---|---|---|---|---|---|---|---|
| 1.221567 | −1.68945 | −1.96737 | −2.36029 | 2.742316 | −0.56371 | −0.09115 | −1.54759 | −1.3829 |
| 0.6868 | 0.360688 | −2.09551 | −1.65593 | 1.78985 | −0.92551 | 0.507281 | −0.99643 | −0.48396 |
| −0.82094 | −0.65623 | −1.98211 | −2.22289 | 1.958919 | −0.94557 | 0.85167 | −1.31909 | −0.50287 |
| −1.44266 | −1.30286 | −3.44292 | −2.69291 | 1.552046 | −1.11569 | 1.495047 | −2.25468 | −2.0076 |
| 0.761475 | 0.230535 | −0.75987 | −2.11556 | 2.13257 | −0.48352 | 0.333412 | −1.8329 | −2.52185 |
| 0.102347 | −0.3837 | −1.70892 | −1.85182 | 2.044947 | −1.44848 | 1.519935 | −1.75863 | −3.65533 |
| 0.43245 | 0.239901 | −1.71643 | −1.83949 | 2.173036 | −0.76272 | 1.004076 | −0.00635 | 0.006346 |
| 0.499252 | −2.01293 | −2.17428 | −2.14726 | 2.364688 | −0.93942 | −0.73654 | −0.88498 | −2.49271 |
| −0.41139 | −1.61542 | −1.62909 | −2.15844 | 2.040648 | −0.35148 | 0.859423 | −0.83505 | 0.489689 |
| −1.67816 | −0.89684 | −3.01999 | −1.4695 | 2.159974 | −0.36421 | 0.319381 | −0.66476 | 0.329862 |
| −0.74102 | −0.67224 | −2.39466 | −3.06106 | −0.19886 | −0.78519 | 0.814273 | −1.48108 | −0.89558 |
| 0.500212 | −0.1188 | −1.53448 | −2.16961 | 1.890505 | −0.81423 | 0.860055 | −2.56086 | −3.15774 |
| −0.27006 | −1.09115 | −0.76241 | −1.71279 | 2.011055 | −1.20824 | 1.603574 | −1.35986 | −1.52661 |
| 0.68891 | 0.255754 | −1.40302 | −1.4418 | 1.90324 | −1.30663 | 1.839623 | −1.82666 | −2.88525 |
| 0.208486 | −0.26175 | −1.28765 | −2.01756 | 2.099883 | −0.91764 | 0.92943 | −1.21918 | 0.276765 |
| 0.7732 | −2.15078 | −2.24765 | −2.2314 | 2.052519 | −0.81637 | 0.106684 | −3.91305 | −1.79135 |
| −0.30654 | −1.44899 | −1.94481 | −2.16947 | 1.959032 | −0.76731 | 0.676857 | −1.00685 | −1.6858 |
| −0.31614 | 0.527919 | −2.15589 | −1.34538 | 2.063194 | −0.97622 | 0.799297 | −0.66489 | −0.55354 |
| −0.15809 | 0.213406 | −2.17398 | −2.00924 | 1.768282 | −1.03928 | 0.427469 | −1.52049 | −2.23192 |
| 1.218668 | 0.563374 | −1.47247 | −2.73101 | 2.164827 | −0.72619 | −1.25275 | −2.57155 | −1.61341 |
| 0.027285 | 0.478329 | −1.67327 | −2.09612 | −1.86844 | −1.89685 | 2.366907 | −3.24536 | −0.98519 |
| −2.34001 | −1.68685 | −0.95398 | 0.614696 | 0.272037 | NA | −0.4729 | −2.11571 | −0.70518 |
| 0.35831 | −0.41568 | −1.7321 | −0.01835 | 1.956999 | −0.64227 | 0.71015 | −1.41964 | −2.19768 |
| −0.11898 | −0.2235 | −2.94174 | −2.38783 | 2.125806 | −0.63128 | 0.410043 | −3.13335 | −2.36355 |
| −0.6667 | −1.25225 | −1.73571 | 0.308592 | 2.018427 | −0.86049 | 0.670418 | −2.83457 | −2.35513 |
| 0.434834 | 0 | −1.90648 | 2.174115 | 2.059526 | −2.06196 | 0.86355 | −1.13299 | −0.90972 |
| 0.535601 | 0.128583 | −2.03499 | 0.152933 | 2.047391 | −1.06342 | 0.775448 | −2.88924 | −0.96975 |
| −0.57983 | −3.73295 | −3.84836 | 1.325353 | −1.51082 | −1.37385 | −0.32169 | −2.4743 | 0.94108 |
| −0.00337 | 0.519491 | −0.58802 | −1.68716 | 2.100591 | −1.14994 | 1.955703 | −1.7406 | −0.65173 |
| 0.729785 | 0.172206 | −2.13642 | −0.28514 | 1.767555 | −1.04301 | 0.855891 | −2.23064 | 0.01364 |
| −0.90148 | −0.77026 | −2.89168 | −2.35254 | 1.320453 | −1.83187 | −0.12711 | −0.10685 | −2.5147 |
| −0.72656 | −0.04166 | −0.34478 | 0.96374 | −2.25099 | −2.52778 | 0.606788 | −0.29955 | −0.51223 |
| −2.52263 | −2.66251 | −5.84213 | 0.883262 | −1.6267 | −1.09018 | 1.500177 | −1.96846 | −4.68508 |
| −0.28113 | −0.57601 | −4.09155 | −0.8056 | 0.931676 | 0.85869 | 1.706513 | −1.23539 | −0.6975 |
| −0.3825 | −2.50829 | −4.81071 | −2.95738 | −1.13837 | −1.24143 | 0.881646 | −1.49469 | −3.59025 |
| 0.052502 | −1.88765 | −0.30349 | −0.89943 | 0.839005 | −5.35006 | −3.61422 | −1.5057 | −1.44437 |
| −0.26426 | −1.27508 | −0.66938 | −2.50739 | 1.416456 | −3.3189 | −2.46173 | −1.29057 | −0.26105 |
| −0.7325 | −0.95572 | −0.30616 | −2.14765 | 1.528653 | −3.8031 | −1.98278 | −1.10999 | −0.48839 |

| Unknown | Unknown | Unknown | Unknown | Unknown | Unknown | Unknown | Unknown | Unknown | Unknown |
|---|---|---|---|---|---|---|---|---|---|
| −0.02746 | −2.41201 | −0.17879 | −1.31451 | −1.20516 | −1.53567 | −1.89243 | −3.00455 | −4.00963 | −3.27004 |
| −0.30971 | −2.74023 | −0.13657 | −1.76059 | −2.1769 | −1.69754 | −2.63878 | −3.00647 | −2.44376 | −1.88402 |
| −0.27833 | −2.4209 | 0.038606 | −1.06283 | −1.82357 | −2.03235 | −2.60063 | −2.37466 | −2.98629 | −2.31875 |
| 0.029326 | −2.31613 | −0.04487 | −1.81199 | −1.1788 | −1.26133 | −0.42128 | NA | −2.85935 | −2.48158 |
| 0.339186 | −3.23065 | 0.677937 | −1.34881 | −0.97838 | −1.37691 | −2.55689 | NA | −2.66996 | −2.1968 |
| 0.215779 | −2.22499 | 0.072509 | −0.43147 | −1.67966 | −2.99463 | 0.428731 | −1.63286 | −2.06974 | −2.46894 |
| 0.391622 | −2.5241 | 0.122983 | −1.05003 | −1.5665 | −1.85149 | 0.05443 | −3.14193 | −3.00129 | −2.81685 |
| 0.321724 | −3.43767 | 0.232539 | −1.10658 | −0.98069 | −2.40357 | −3.9041 | NA | −4.1476 | −2.96237 |
| −0.17523 | −2.53289 | −0.29347 | −1.12608 | −1.28611 | −0.6739 | −1.34272 | −1.94992 | −3.24448 | −2.09178 |
| −0.89648 | −2.62782 | −0.53015 | −1.2622 | −3.39913 | −2.09861 | −3.44469 | −5.0428 | −3.27806 | −2.45863 |
| −0.43702 | −2.33303 | 0.070899 | 2.743799 | −1.96784 | −1.70705 | −0.26692 | −7.5921 | −1.85989 | −1.67755 |
| −0.15972 | −2.10588 | 0.406701 | −1.4613 | −2.12798 | −2.03915 | −2.57363 | −4.76042 | −2.60573 | −2.05659 |
| 0.013735 | −2.80964 | −0.11296 | −0.64117 | −1.68882 | −2.44751 | 0.095705 | −4.02312 | −1.91949 | −2.68879 |
| 0.523588 | −2.47349 | 0.133373 | −0.72922 | −1.75145 | −2.40899 | 0.175779 | −3.17995 | −2.85978 | −2.4377 |
| −0.00604 | −2.28502 | 0.30228 | −1.52128 | −2.49742 | −2.61377 | −0.49545 | −2.98772 | −2.42683 | −2.45281 |
| 0.088696 | −2.40788 | −0.36885 | −1.84989 | −1.27264 | −1.29494 | −2.49385 | NA | −2.72848 | −2.05314 |
| 0 | −2.55801 | 0.002529 | −1.6301 | −1.75075 | −2.41473 | −0.09016 | −4.09312 | −3.52879 | −2.41742 |
| −0.23845 | −2.50705 | −0.09948 | −1.19981 | −1.74791 | −1.02206 | −2.89685 | −2.75034 | −2.87634 | −2.29277 |
| 0.184114 | −2.66659 | 0.29772 | −1.80169 | −0.9403 | −1.9895 | −0.43848 | −4.59286 | −2.39498 | −2.48433 |
| −1.04522 | −4.67865 | −0.23139 | −1.30115 | 1.757194 | −3.34331 | −0.09204 | −5.53964 | −2.89767 | −4.3693 |
| −0.90933 | −2.18027 | −0.9719 | −0.39862 | −2.90005 | −0.55838 | −2.68366 | −3.29672 | −3.08287 | −2.24771 |
| 1.444387 | −2.16983 | 1.094879 | 0.58805 | 0.378671 | −0.55633 | −1.83996 | NA | NA | −2.57878 |
| −0.96265 | −2.30061 | −0.82071 | −1.9099 | −2.9535 | −2.17376 | −0.08282 | −2.39849 | −2.41876 | −2.04337 |
| −1.01721 | −1.83756 | −0.8819 | −2.13077 | 1.322408 | −2.87086 | −0.14742 | −3.06637 | −3.1482 | −1.90082 |
| −0.63131 | −2.56905 | −0.52794 | −1.7939 | 1.751243 | −1.10299 | −2.89324 | −7.94311 | −1.78845 | −1.76637 |
| −0.57516 | −3.36203 | −1.27085 | −0.8185 | 1.985767 | −1.82987 | −2.26778 | −4.98003 | −2.04882 | −2.12106 |
| −0.6248 | −2.28128 | −0.38715 | −1.53958 | −0.62486 | −2.30523 | −0.97093 | −3.20168 | −3.64042 | −2.26962 |
| −0.68104 | −1.93424 | −0.68741 | −0.7425 | 1.447845 | −2.19826 | −2.51552 | NA | −1.84237 | −1.93245 |
| −0.21986 | −2.59151 | −0.35718 | −0.71442 | −3.18448 | −1.2578 | −0.0201 | −1.91648 | −2.69737 | −2.42503 |
| −1.26021 | −2.23783 | −1.10157 | −2.57872 | 1.335309 | NA | −0.46536 | −2.96759 | −2.33199 | −2.51868 |
| 0.515821 | −2.26111 | 0.560262 | 0.833984 | −0.59601 | 0.176946 | −0.20089 | −4.10026 | −2.61189 | −2.15678 |
| 0.624138 | −2.42688 | 0.311549 | 0.572527 | −0.77527 | −0.55632 | 0.454812 | −6.33195 | −1.81041 | −1.86085 |
| −1.10319 | −2.23706 | −0.31536 | −2.28646 | 1.215609 | −0.42914 | −0.90654 | −4.35146 | −2.34738 | −1.48803 |
| −1.20274 | −2.38484 | −0.2833 | −1.83063 | −2.60032 | −0.14471 | −0.29587 | −3.95574 | −2.80487 | −1.98561 |
| −1.01092 | −2.00686 | −0.51387 | −1.9481 | 1.421399 | −2.01677 | −0.45709 | −4.8815 | −1.38471 | −1.22467 |
| −0.76561 | NA | −4.78145 | 4.0249 | 2.773221 | −1.65343 | −0.52943 | −4.40213 | NA | NA |
| −1.05956 | NA | −3.15226 | 4.778855 | 2.258412 | −2.43206 | −0.19803 | −2.83226 | −2.99882 | NA |
| −0.18455 | NA | −2.80224 | 3.67739 | 2.602906 | −2.52386 | −0.17343 | −2.74728 | −4.68272 | NA |

| Unknown | Unknown | Unknown | Unknown | Unknown | Unknown | Unknown | Unknown | Unknown |
|---|---|---|---|---|---|---|---|---|
| −2.34849 | −2.00684 | −4.18491 | −1.44321 | 3.638691 | 3.343032 | −0.29766 | NA | −1.2809 |
| −2.78655 | −1.88116 | −2.31942 | −0.59315 | 4.128089 | 1.379746 | −0.8133 | NA | −3.17778 |
| −2.88492 | −2.87159 | −4.30089 | −1.41878 | 4.255568 | 2.001362 | −0.9235 | NA | −3.04257 |
| −2.61258 | NA | −2.61258 | −4.33149 | 4.849689 | 2.631851 | −0.82752 | NA | −1.11209 |
| −1.76975 | −1.37809 | −2.99976 | −1.39643 | 3.595663 | 3.258272 | −0.69697 | NA | −0.9493 |
| −3.64909 | −2.15824 | −3.09548 | −0.409 | 5.075293 | 2.855691 | −1.20543 | NA | −1.02474 |
| −2.78517 | −2.31691 | −4.25572 | −0.85931 | 4.846906 | 2.806658 | −0.78049 | NA | −1.32972 |
| −2.22949 | −2.48483 | −4.04302 | −2.51444 | 3.321289 | 2.849759 | −0.58942 | NA | −0.5511 |
| −2.72629 | −2.51769 | −4.66721 | −0.78736 | 4.453459 | 2.101494 | −0.66357 | NA | −2.71435 |
| −2.29257 | −2.96536 | −1.7511 | −1.08724 | 4.069592 | 1.466229 | −0.93811 | NA | −3.06719 |
| −2.54181 | −2.63668 | −0.96267 | −1.31823 | 3.804558 | 0.926701 | −2.24466 | NA | −2.98727 |
| −2.7527 | −2.92723 | −3.26102 | −0.67928 | 4.173016 | 2.006109 | −1.14085 | NA | −2.87515 |
| −3.39894 | −1.73417 | −2.14459 | −0.62808 | 5.011047 | 2.80671 | −1.02307 | NA | −2.09464 |
| −4.03733 | −2.51135 | −3.62859 | −0.58074 | 5.187546 | 2.93068 | −1.07384 | NA | −2.55507 |
| −3.15574 | −3.02647 | −4.44402 | −1.297 | 4.322217 | 2.216816 | −0.93644 | NA | −2.72745 |
| −2.38518 | −1.41469 | −1.6899 | −1.72942 | 3.43083 | 3.178833 | −0.99987 | NA | −2.96351 |
| −2.6713 | −2.6119 | −1.75091 | −1.10453 | 4.443629 | 2.271781 | −0.84646 | NA | −1.45368 |
| −2.38741 | −2.75203 | −1.98684 | −0.80834 | 4.394718 | 2.207412 | −0.74676 | NA | −1.52169 |
| −3.98709 | −2.14597 | −4.15012 | −1.80429 | 4.044117 | 1.88904 | −0.92043 | NA | −1.45219 |
| −2.12468 | −2.46694 | −3.4982 | −2.02854 | 2.074309 | 2.429284 | −0.94809 | NA | −2.08455 |
| −2.34311 | −2.75964 | −2.49827 | 0.041527 | 5.622237 | 3.367981 | −1.23639 | NA | −2.22892 |
| NA | NA | −5.29725 | −4.95787 | 2.537431 | 1.960789 | −2.20247 | NA | −3.85026 |
| −1.70395 | −1.60021 | −1.59565 | −1.15255 | 4.014324 | 1.364408 | −0.82172 | NA | −2.72998 |
| −2.37572 | −2.39975 | −1.12165 | −1.27385 | 3.52708 | 1.025956 | −1.18582 | NA | −3.18784 |
| −2.62888 | −4.43065 | −1.12833 | −0.68702 | 4.033092 | 1.454901 | −0.63385 | NA | −2.8522 |
| −1.57736 | NA | −1.19374 | −3.35296 | 2.295876 | 1.894016 | −1.61732 | NA | −3.53875 |
| −2.65788 | −2.37427 | −4.14292 | −0.9024 | 4.358283 | 2.249152 | −0.55267 | NA | −1.56967 |
| −2.60083 | NA | −4.03806 | −2.67131 | 2.619631 | 0.199118 | −1.66483 | NA | −2.20555 |
| −3.86241 | −1.89902 | −3.03579 | −0.04748 | 5.195297 | 2.995731 | −0.59711 | NA | −2.51056 |
| −2.79361 | −2.45492 | −2.29819 | −1.07746 | 4.019243 | 1.437945 | −1.24271 | NA | −2.93611 |
| −2.80388 | NA | −3.09087 | −2.85325 | 3.088756 | 0.533757 | −1.78942 | NA | −1.94016 |
| −1.68103 | NA | −1.7638 | −1.6619 | 3.975652 | 1.937219 | −2.03856 | NA | −2.23809 |
| −2.94258 | −0.94931 | −1.71542 | −3.48355 | 4.795924 | 2.428844 | −0.93189 | NA | −1.41927 |
| −2.86315 | −0.56103 | −4.09743 | −0.5035 | 4.892044 | 2.65293 | −0.46963 | NA | −0.94778 |
| −1.93653 | −1.42326 | −0.8763 | −0.79483 | 4.097336 | 1.242978 | −0.88269 | NA | −1.69145 |
| NA | NA | NA | NA | 0.443625 | 0.397675 | 0.252133 | 0.769634 | −3.25785 |
| NA | NA | NA | −4.46484 | 1.172388 | 1.076274 | 0.002135 | 1.289032 | −1.74186 |
| −6.17889 | NA | NA | −4.2.6222 | 0.693402 | 1.206799 | 0.048286 | 1.47538 | −1.96536 |

| Unknown | Unknown | Unknown | Unknown | Unknown | Unknown | Unknown | Unknown | Unknown | Unknown |
|---|---|---|---|---|---|---|---|---|---|
| −1.74014 | −1.97539 | NA | −1.51182 | NA | 0.15307 | 0.08167 | −0.52164 | −0.43577 | −3.56082 |
| −0.80542 | −1.76996 | NA | NA | NA | 0.241456 | 0.019022 | −0.94473 | −0.20028 | −3.43604 |
| −0.30578 | −2.18183 | MA | NA | NA | 0.060516 | −0.19816 | −1.3304 | −0.06855 | −3.98641 |
| −0.17954 | −1.19437 | NA | NA | NA | −1.79336 | 0.035525 | −1.43565 | −0.96391 | −2.93087 |
| 0.363131 | −0.28146 | NA | NA | NA | 0.235032 | −0.26959 | −0.53617 | −0.35599 | −5.24949 |
| 0.251661 | −1.17395 | NA | −0.41334 | NA | −2.20683 | −0.76374 | −1.70716 | −0.02638 | −1.84348 |
| −0.12793 | −0.95466 | NA | −1.73513 | NA | 0.758639 | −0.27022 | −1.74292 | 0.270962 | −2.63134 |
| −0.27019 | −0.94599 | NA | NA | NA | −0.40305 | −0.0981 | −0.98671 | −0.38102 | −2.84185 |
| −0.29944 | −0.88515 | NA | NA | NA | 0.176219 | 0.295324 | −0.92314 | 0.120162 | −3.45413 |
| −0.60351 | −1.0644 | NA | NA | NA | 0.053999 | −0.0351 | −0.68056 | −0.61506 | −2.80782 |
| −0.38472 | −2.39544 | −3.23804 | −1.26591 | NA | 0.129099 | 0.222822 | −0.59063 | −0.65763 | NA |
| −0.01004 | −1.75123 | NA | NA | NA | −0.31574 | 0.23669 | −0.61894 | −0.14149 | −3.12724 |
| 0.610443 | −1.43566 | NA | NA | NA | −1.4929 | −0.97745 | −1.59364 | −0.0501 | −1.5438 |
| −0.03748 | −0.82422 | NA | −0.01781 | NA | −2.48635 | −0.20556 | −1.56674 | 0.333455 | −1.45083 |
| −0.06859 | −0.72807 | NA | NA | NA | −1.63952 | 0.002013 | −1.9224 | −0.13555 | NA |
| −0.16342 | −1.02297 | NA | −0.12595 | NA | 0.079216 | 0.413669 | −0.45649 | −0.55634 | NA |
| −0.23614 | −1.52964 | NA | NA | NA | 0.147106 | −0.48065 | −0.89274 | −0.56422 | −2.56116 |
| −0.40059 | −1.43882 | NA | −1.54863 | NA | −1.10181 | −1.33045 | −0.75864 | 0.077567 | −3.02288 |
| −0.76295 | −1.8414 | NA | NA | NA | −0.03955 | 0.018988 | −1.63919 | −0.50498 | −3.90307 |
| 0.350582 | −0.40354 | NA | NA | NA | −0.7381 | 0.136121 | −1.90834 | −0.21296 | NA |
| 0.794417 | −1.61133 | NA | NA | NA | −1.49286 | 0.24217 | −1.74439 | 0.095809 | −1.35814 |
| 0.232943 | NA | NA | NA | NA | −2.38639 | −0.91621 | −1.78066 | −1.9905 | NA |
| −0.42132 | −1.40725 | NA | NA | NA | 0.099148 | −0.22091 | −0.79284 | −0.73988 | −2.33383 |
| −0.75324 | −1.51907 | NA | NA | NA | 0.535541 | −0.34647 | −0.15073 | −1.75301 | NA |
| −0.10039 | −0.4039 | NA | NA | NA | 0.298669 | −1.43425 | −0.33285 | −0.35688 | NA |
| −1.45729 | −2.06473 | NA | NA | NA | 0.382658 | −1.17023 | 0.259993 | −0.77261 | NA |
| 0.174268 | −1.90159 | NA | NA | NA | −0.54 | −1.68647 | −1.02095 | −0.26081 | −3.41672 |
| −0.9985 | −3.5915 | NA | NA | NA | 0.140809 | −0.77276 | −0.46942 | −0.85782 | NA |
| 0.152761 | −0.93773 | NA | NA | NA | −2.86213 | −0.18854 | −1.89679 | 0.273845 | −1.65114 |
| −0.29705 | −2.51472 | NA | NA | NA | −0.55783 | 0.125995 | −0.98232 | −1.07049 | −2.55426 |
| −0.32337 | NA | NA | NA | NA | −2.07198 | 0.144854 | −1.29063 | −0.9071 | NA |
| 0.141488 | −2.87005 | NA | −0.87815 | NA | −0.07257 | 0.057855 | −0.78463 | −0.16231 | −3.4977 |
| −0.6256 | 1.252214 | NA | NA | NA | 0 | 0.168104 | −0.42227 | −2.05245 | −2.57844 |
| −0.35563 | 1.526059 | NA | NA | NA | −1.59848 | 0.173667 | −1.09389 | −1.63696 | −2.13593 |
| −0.77904 | 0.955959 | NA | NA | NA | 0.292045 | 0.777958 | −0.22727 | −1.30983 | −2.5693 |
| −0.70173 | −6.39534 | −0.78489 | 0.377135 | −0.09006 | 0.231518 | 1.460592 | −1.7806 | 1.699449 | −4.01824 |
| −1.87609 | −0.70828 | 0.139159 | NA | 0.094001 | −0.34639 | 2.158374 | −1.2181 | 1.048203 | −2.31029 |
| −1.52156 | −0.71491 | 0.342834 | NA | 0.232006 | −2.40266 | 2.227017 | −1.1584 | 1.365606 | −3.75654 |

| Unknown | Unknown | Unknown | Unknown | Unknown | Unknown | Unknown | Unknown | Unknown |
|---|---|---|---|---|---|---|---|---|
| −0.34637 | −2.59439 | NA | NA | −2.97318 | −1.05411 | −2.8021 | 1.013161 | −0.50952 |
| −0.65686 | −3.70247 | NA | NA | −1.64085 | −1.66194 | −2.44141 | −0.1186 | −2.66993 |
| 0.122695 | −2.73176 | NA | NA | −1.92251 | −3.04385 | −4.24158 | 0.557407 | −2.73122 |
| −0.35534 | −1.86783 | NA | NA | −3.37657 | −1.25738 | −3.09028 | 0.234959 | −3.09451 |
| −0.5756 | −0.61583 | NA | NA | −3.87929 | −1.22237 | −4.21896 | −0.23442 | −0.95874 |
| −0.71382 | −0.39292 | NA | NA | −2.12754 | −1.50264 | −2.31244 | 2.258303 | −1.18851 |
| −1.10902 | −0.78442 | NA | NA | NA | −0.81822 | −2.31613 | 0.737128 | −2.86804 |
| −2.19357 | −2.17315 | NA | NA | −2.93498 | −2.34712 | −2.97868 | 0.403599 | −1.27519 |
| −0.17263 | −2.8098 | NA | NA | −1.66886 | −1.08307 | −2.48433 | −1.25346 | −0.8153 |
| 0.176575 | 0.049421 | NA | NA | −3.51601 | −2.66666 | −2.45131 | −1.80414 | −1.75776 |
| 0.674785 | −1.95678 | NA | NA | −2.51641 | −1.16741 | −2.83453 | −0.64099 | −0.68576 |
| −0.21723 | −1.79367 | NA | NA | −2.54206 | −1.31484 | −2.36977 | −0.11887 | −1.29625 |
| −0.64532 | −0.73215 | NA | NA | −2.54265 | −2.16057 | −2.92926 | 0.243595 | −0.93687 |
| −3.71683 | −0.3828S | NA | NA | −1.96449 | −1.74212 | −2.17535 | 2.286012 | −4.19296 |
| −0.55954 | −1.93848 | NA | NA | −4.63387 | −2.11106 | −2.22686 | 0.688049 | −2.53603 |
| 0.534443 | −2.85619 | NA | NA | −2.5145 | −2.22383 | −5.11152 | 2.64382 | −0.8011 |
| −0.51771 | −1.01224 | NA | NA | −1.92545 | −2.59147 | −2.59486 | 0.483441 | −1.41663 |
| 0.138309 | −2.86154 | NA | NA | −1.8404 | −1.68327 | −2.40296 | −0.39087 | −2.0292 |
| −0.32751 | −2.72417 | NA | NA | −0.672 | −1.75341 | −4.65863 | 0.119111 | −2.38419 |
| −1.9554 | −4.71704 | NA | NA | −1.83675 | −1.99099 | −5.33873 | −2.96735 | −1.85271 |
| 0.357127 | 0 | NA | NA | −1.47162 | −1.72513 | −2.51374 | −1.02225 | −3.02694 |
| −0.20738 | NA | NA | NA | −3.53588 | −1.24481 | NA | −0.73118 | −0.41087 |
| 0.503939 | 0.184208 | NA | NA | −2.54531 | −1.62549 | −2.65489 | −1.42568 | −1.9156 |
| 0.544368 | 0.219669 | NA | NA | −1.71135 | −2.03202 | NA | −1.50154 | −1.19054 |
| −2.26047 | −3.74066 | NA | NA | −0.8059 | −1.29844 | −4.70548 | −1.97016 | −0.9239 |
| −2.50035 | −4.2959 | NA | NA | −4.59842 | −2.12069 | −4.63611 | −2.11574 | −2.49982 |
| 0.395217 | 0.078713 | NA | NA | −2.13239 | −1.50368 | −3.54368 | −1.91968 | −3.03995 |
| −0.38529 | −0.76676 | NA | NA | −1.68714 | −1.13573 | NA | NA | −0.83603 |
| −0.21412 | −4.66833 | NA | NA | 0.141164 | −1.2907 | −1.83659 | −0.99488 | −2.24408 |
| 0.263312 | −0.0562 | NA | NA | −2.95924 | −1.41724 | −2.96285 | −1.92293 | −2.71219 |
| −0.66594 | −1.04667 | NA | NA | −2.80333 | −0.90513 | −5.27623 | −2.14951 | −2.052 |
| −1.69712 | −2.17838 | NA | NA | −1.62023 | −1.43246 | −3.21608 | −1.66348 | −2.31548 |
| 0.227905 | 0.17673 | NA | NA | −2.70976 | −1.32857 | NA | −2.48798 | −1.39643 |
| 0.041454 | −0.00375 | NA | NA | NA | −2.64947 | −3.74347 | −0.95378 | −0.91641 |
| 0.645511 | 0.445329 | NA | NA | −2.71864 | −1.25731 | −4.48693 | −1.92977 | −0.8138 |
| 8.307928 | −5.64557 | 0.073118 | 0.136384 | 0.577748 | −0.08959 | −2.72552 | 0 | −1.32921 |
| 8.257331 | NA | −0.20067 | 0.674402 | 1.21001 | −0.58476 | −2.27216 | 0.636934 | 5.713376 |
| 8.48129 | NA | 0.107712 | 0.832721 | 1.251773 | −3.12661 | −2.2483 | −2.74357 | −2.99686 |

| Unknown | Unknown | Unknown | Unknown | Unknown | Unknown | Unknown | Unknown |
|---|---|---|---|---|---|---|---|
| 0.328618 | −1.81853 | NA | 1.699016 | −0.02264 | −4.24934 | −1.87299 | NA |
| 0.453956 | −1.38578 | NA | 0.88372 | NA | −2.54429 | −1.1405 | NA |
| 0.407776 | −2.43644 | NA | 1.022952 | −0.97262 | −2.50538 | −1.88726 | NA |
| 0.270701 | −1.16133 | NA | 0.459983 | NA | −2.44139 | −2.01621 | NA |
| −2.82836 | −1.38036 | NA | −0.16694 | NA | −3.52758 | −2.54329 | −4.34741 |
| 0.019734 | −2.48969 | NA | 0.804197 | NA | −3.36081 | −1.69207 | NA |
| −0.24268 | −2.487 | NA | 1.232287 | −0.63924 | −2.48241 | −1.96664 | NA |
| −0.55215 | −2.58165 | NA | 0.596108 | NA | −4.98219 | −3.10397 | NA |
| 0.006911 | −2.17016 | NA | 1.29659 | −0.66262 | −2.16184 | −1.60821 | −4.25929 |
| 0.319861 | −2.98194 | NA | 1.088387 | −0.66856 | −3.05834 | −2.91505 | NA |
| 0.183754 | −1.18113 | NA | −1.27479 | NA | −2.06133 | −3.27211 | NA |
| 0.554914 | −1.75381 | NA | 0.385224 | NA | −1.93372 | −0.82223 | −5.5818 |
| −1.82006 | −1.60732 | NA | 0.205902 | NA | −2.57987 | −2.10456 | NA |
| −0.84665 | −1.74738 | NA | 0.801468 | NA | −2.64764 | −0.52501 | NA |
| −0.21355 | −2.54756 | NA | 0.636044 | NA | −2.99038 | −2.2182 | NA |
| −0.20236 | −0.9392 | NA | 0.696276 | NA | −2.45767 | −1.89167 | NA |
| −0.08386 | −1.03069 | NA | 0.843158 | −1.3353 | −3.07703 | −1.28392 | −5.28433 |
| 0.343058 | −2.11043 | NA | 1.721163 | −0.05568 | −2.77269 | −2.20769 | NA |
| 0 | −2.28766 | NA | 0.763288 | NA | −2.59196 | −2.06763 | NA |
| −2.90504 | −2.39861 | NA | NA | NA | −2.44663 | −2.97985 | NA |
| −1.25029 | −2.04293 | NA | −2.20097 | NA | −1.48982 | −1.22332 | NA |
| −3.35027 | NA | NA | −2.08642 | NA | −4.73934 | −1.08193 | NA |
| 0.071318 | −2.5714 | NA | 0.914157 | NA | −3.08345 | −2.80482 | NA |
| −0.03563 | −2.55681 | NA | 0.598375 | NA | −1.92484 | −2.0556 | NA |
| −1.49391 | −0.8579 | NA | −1.50093 | NA | −2.18652 | −0.78074 | −4.99584 |
| −0.93294 | −4.6767 | NA | 0.707487 | NA | NA | −1.41121 | NA |
| −0.10176 | −1.43224 | NA | 0.791259 | −1.07456 | −2.05164 | −1.66912 | NA |
| −2.1916 | NA | NA | NA | NA | −3.88877 | −1.45078 | NA |
| −0.23878 | −0.51463 | NA | 0.640737 | NA | −2.24665 | −1.81821 | NA |
| 0.17549 | −2.91 | NA | 0.576859 | NA | −2.08324 | −2.44296 | NA |
| −1.79083 | −2.77169 | NA | −3.17399 | NA | −4.16516 | −1.41876 | NA |
| 0.071194 | −2.6359 | NA | −3.20673 | NA | −4.23725 | −1.84446 | NA |
| 0.482494 | −4.08936 | NA | −1.91659 | NA | −0.96287 | −1.84518 | NA |
| −0.07624 | −4.54739 | NA | −2.3873 | NA | −0.8174 | −3.12201 | NA |
| 0.84204 | −2.22971 | NA | 0.140939 | NA | −0.61796 | −0.68612 | −2.18605 |
| −3.03766 | −2.07485 | −2.65339 | −2.58835 | NA | −1.39902 | −1.80827 | 4.02778 |
| −0.98031 | −2.65068 | −1.40297 | −0.55047 | NA | −0.52191 | −1.10971 | 4.837727 |
| −2.13154 | −1.44472 | −1.502 | −0.4513 | NA | −0.58923 | −1.55111 | 4.767473 |

| Unknown | Unknown | Unknown | Unknown | Unknown | Unknown | Unknown |
|---|---|---|---|---|---|---|
| 2.625259 | −1.43135 | NA | 1.895428 | −0.06488 | NA | −2.58923 |
| 2.821389 | −0.54387 | NA | 1.969576 | 0.439955 | NA | −2.54778 |
| 2.910098 | −0.69287 | NA | 2.182837 | 0.460341 | NA | NA |
| 2.892513 | −0.66476 | NA | 1.753378 | 0.009645 | NA | NA |
| 2.884689 | −0.99296 | NA | 1.934787 | −0.20542 | NA | NA |
| 3.049774 | −2.48482 | NA | 2.530715 | 0.470413 | NA | −0.95995 |
| 2.936685 | −0.70817 | NA | 2.049365 | −0.10162 | NA | −2.45498 |
| 2.668742 | −1.54315 | NA | 1.567455 | 0.697392 | NA | −4.68943 |
| 3.301302 | −0.35881 | NA | 2.404924 | 0.761856 | NA | −2.16779 |
| 2.8504 | −1.2428 | NA | 2.163525 | 0.415716 | NA | −0.71591 |
| 2.595459 | −1.03424 | NA | 1.670837 | 0.022425 | NA | −2.90669 |
| 3.207976 | −0.55418 | NA | 2.329188 | 0.588406 | NA | −0.75073 |
| 3.0734 | −1.6798 | NA | 2.482405 | −0.17411 | NA | −3.10431 |
| 3.08511 | −1.93406 | NA | 2.266488 | 0.442746 | NA | NA |
| 3.15026 | −0.73349 | NA | 2.022205 | 0.320293 | NA | 0.456935 |
| 2.991093 | −0.9558 | NA | 1.766201 | 0.292922 | NA | NA |
| 2.82282 | −0.91785 | NA | 1.765305 | 0.32578 | NA | −2.53135 |
| 2.836803 | −0.5558 | NA | 2.012704 | 0.245638 | NA | −2.71573 |
| 2.715771 | −1.05179 | NA | 1.956069 | −0.17824 | NA | −0.69877 |
| 2.665413 | −1.24461 | NA | 1.650338 | −0.40651 | NA | NA |
| 3.378926 | −1.43699 | NA | 2.544595 | 0.733495 | NA | −4.63493 |
| 0.991934 | NA | NA | 0.157759 | NA | NA | NA |
| 3.022258 | −0.35457 | NA | 1.899708 | 0.633595 | NA | −2.81163 |
| 3.119645 | −1.62975 | NA | 2.276905 | 0.583927 | NA | −0.2483 |
| 3.20671 | −0.66592 | NA | 2.076403 | 0.852408 | NA | NA |
| 1.885118 | −1.82746 | NA | 0.992551 | −1.59875 | NA | NA |
| 2.905072 | −0.80939 | NA | 2.222196 | −0.53641 | NA | −0.70046 |
| 1.092598 | −2.35918 | NA | NA | NA | NA | NA |
| 3.328686 | −1.20091 | NA | 2.377937 | 0.826229 | NA | NA |
| 2.740923 | −0.59833 | NA | 1.739125 | 0.816294 | NA | −0.88032 |
| 2.166027 | NA | 0.893372 | 0.893372 | −1.49555 | NA | NA |
| 1.79522 | −4.71695 | 0.451584 | 0.451584 | −4.75332 | NA | NA |
| 2.713723 | −1.33548 | NA | 1.291927 | 0.451438 | NA | −2.34964 |
| 2.919165 | −1.12623 | 1.635434 | 1.635434 | 0.615465 | NA | −3.13734 |
| 2.902128 | −1.73947 | 1.52328 | 1.52328 | 0.652593 | NA | −3.2794 |
| NA | −3.25132 | −0.27754 | −0.27754 | NA | −1.57385 | −3.03868 |
| NA | −3.37556 | 1.271828 | 1.271828 | NA | −2.11595 | −3.25813 |
| NA | −3.37923 | 1.552851 | 1.552851 | NA | −1.81417 | −1.67409 |

[symbol]

indicates data missing or illegible when filed

1. A composition or product combination comprising:
a) a bacteria selected from the group consisting of: Bacteriodetes, Anaerofilum, Anaerotruncus, Christensenella, Pseudoramibacter eubacterium, Holdemania, Clostridiales, and a mixture of two or more of the listed bacteria; and
b) a taurine precursor and/or a 5-Aminovaleric acid (5AV) precursor, wherein components (a) and (b) are provided in the same formulation or are provided in separate formulations in a product combination.

2. The composition or product combination of claim 1, wherein the bacteria is selected from the group consisting of Bacteroides, Butyricimonas, Paraprevotallacae, and a mixture of two or more of the listed bacteria.

3. The composition or product combination of claim 1, wherein the bacteria is selected from the group consisting of Bacteriodetes, Anaerofilum, Anaerotruncus, Christensenella, Pseudoramibacter eubacterium, Holdemania, and a mixture of two or more of the listed bacteria.

4. The composition or product combination of claim 1, wherein the bacteria is selected from the group consisting of Bacteroidetes, Holdemania, and a mixture of of the listed bacteria.

5. The composition or product combination of claim 1, wherein the bacteria comprises a Bacteroidetes selected from the group consisting of Bacteroidaceae, Paraprevotellacae, Banesiellaceae, Rikenellaceae, Odoribacteraceae, Bacteroides, Butyricimonas, and a mixture of two or more of the listed bacteria.

6. The composition or product combination of claim 1, wherein the bacteria comprises a Bacteroidetes selected from the group consisting of Bacteroides ovatus, Parabacteroides merdae, and Bacteroides thetaiotaomicron, and a mixture of two or more of the listed bacteria.

7. The composition or product combination of claim 1, wherein the bacteria comprises a Clostridiales selected from the group consisting of Lachnospiraceae and Clostridium.

8. The composition or product combination of claim 1, wherein the composition or product combination does not comprise any bacteria of Eggerthella, Alstipes, Burkolderiales, Enterococcaceae, Clostridium, and Ruminococcus.

9. The composition or product combination of claim 1, wherein the composition or product combination does not comprise any bacteria of Ruminococcaceace, Oscillospira, and Suterella.

10. The composition or product combination of claim 1, wherein the composition or product combination does not comprise Eisenbergiela tayi.

11. The composition or product combination of claim 1, comprising the taurine precursor and the 5 AV precursor.

12. The composition or product combination of claim 1, wherein the taurine precursor is selected from the group consisting of: cysteine, cysteine sulfinic acid, homocysteine, cystathionine, hypotaurine, and a mixture of two or more of the listed items.

13. The composition or product combination of claim 1, wherein the 5AV precursor is selected from the group consisting of: lysine, cadaverine, 1-piperideme, and a mixture of two or more of the listed items.

14. The composition or product combination of claim 1, wherein the composition or product combination comprises Bacteroides and Parabacteroides.

15. The composition or product combination of claim 1, wherein the composition or product combination comprises Bacteroides ovatus and Parabacteroides merdae.

16. The composition or product combination of claim 1, wherein the composition or product combination comprises Bacteroides ovatus and Bacteroides thetaiotaomicron.

17. The composition or product combination of claim 1, wherein the composition or product combination comprises Parabacteroides merdae and Bacteroides thetaiotaomicron.

18. The composition or product combination of claim 1, wherein the composition or product combination comprises Bacteroides ovatus, Parabacteroides merdae, and Bacteroides thetaiotaomicron.

19. The composition or product combination of claim 1, further comprising Lactobacillus reuteri.

20. The composition or product combination of claim 1, consisting essentially of:
the bacteria; and
the taurine precursor and/or the 5AV precursor.

21-118. (canceled)


SEQUENCE LISTING (42 sequences)

SEQ ID NO: 1 - DNA, 19 bp, Artificial
  Forward oligo
gtgccagcmgccgcggtaa

SEQ ID NO: 2 - DNA, 20 bp, Artificial
  Reverse oligo
ggactachvgggtwtctaat

SEQ ID NO: 3 - DNA, 21 bp, Artificial
  Forward oligo
aataacttggatgttcccacg

SEQ ID NO: 4 - DNA, 19 bp, Artificial
  Reverse oligo
ggccctggtatagtcatgg

SEQ ID NO: 5 - DNA, 19 bp, Artificial
  Forward oligo
cagcagccataacgaagag

SEQ ID NO: 6 - DNA, 19 bp, Artificial
  Reverse oligo
tacttcctcactagcagcc

SEQ ID NO: 7 - DNA, 22 bp, Artificial
  Forward oligo
acaaactacattgttcggtgac

SEQ ID NO: 8 - DNA, 21 bp, Artificial
  Reverse oligo
agcatggtgtaatcttcttgc

SEQ ID NO: 9 - DNA, 19 bp, Artificial
  Forward oligo
taatgcctggcatgatgtg

SEQ ID NO: 10 - DNA, 21 bp, Artificial
  Reverse oligo
actgatattgtcaccatagcg

SEQ ID NO: 11 - DNA, 19 bp, Artificial
  Forward oligo
tacttccgtttctgtgagc

SEQ ID NO: 12 - DNA, 25 bp, Artificial
  Reverse oligo
gttactaacagtagtggatgtagag

SEQ ID NO: 13 - DNA, 22 bp, Artificial
  Forward oligo
aatcagtgatgtcattgaggac

SEQ ID NO: 14 - DNA, 20 bp, Artificial
  Reverse oligo
accaaagtattaccagcgga

SEQ ID NO: 15 - DNA, 19 bp, Artificial
  Forward oligo
taaacagcgaaggcaagga

SEQ ID NO: 16 - DNA, 19 bp, Artificial
  Reverse oligo
tgtgatctcgtcagtgctg

SEQ ID NO: 17 - DNA, 20 bp, Artificial
  Forward oligo
aagttctgcagtgaagagga

SEQ ID NO: 18 - DNA, 19 bp, Artificial
  Reverse oligo
gcagaagaactcattgccc

SEQ ID NO: 19 - DNA, 19 bp, Artificial
  Forward oligo
gtgcacaagatttgcactg

SEQ ID NO: 20 - DNA, 20 bp, Artificial
  Reverse oligo
tcaccaatctgctgtttctc

SEQ ID NO: 21 - DNA, 19 bp, Artificial
  Forward oligo
ctcctgctatgtcggagac

SEQ ID NO: 22 - DNA, 19 bp, Artificial
  Reverse oligo
actctagcaagctttggca

SEQ ID NO: 23 - DNA, 150 bp, Unknown
  Bacteria of family Lachnospiraceae
tacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctag

SEQ ID NO: 24 - DNA, 150 bp, Bacteroides ovatus
tacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtgg

SEQ ID NO: 25 - DNA, 150 bp, Unknown
  Bacteria of family Ruminococcaceae
tacgtagggagcgagcgttgtccggaattactgggtgtaaagggagcgtaggcggggtctcaagtcgaatgttaaatctaccggctcaactggtagctgcgttcgaaactggggctcttgagtgaagtagaggcaggcggaattcctagt

SEQ ID NO: 26 - DNA, 150 bp, Unknown
  Bacteria of family Ruminococcaceae
tacgtagggagcgagcgttgtccggaattactgggtgtaaagggagcgtaggcgggaaagcaagttggaagtgaaatgcatgggcttaacccatgagctgctttcaaaactgtttttcttgagtgaagtagaggcaggcggaattcctag

SEQ ID NO: 27 - DNA, 150 bp, Unknown
  Bacteria of genus Holdemania
tacgtaggtggcgagcgttatccggaattattgggcgtaaagggtgcgcaggcggtttgttaagtttaaggtgaaagcgtggggcttaaccccatatagccttagaaactgacagactagagtacaggagagggcaatggaattccatgt

SEQ ID NO: 28 - DNA, 150 bp, Unknown
  Bacteria of family Lachnospiraceae
tacgtagggggcaagcgttatccggatttactgggtgtaaagggagcgtagacggcatggcaagccagatgtgaaaacccagggctcaaccttgggattgcatttggaactgtcaggctggagtgcaggagaggtaagcggaattcctag

SEQ ID NO: 29 - DNA, 150 bp, Unknown
  Bacteria of genus Parabacteroides
tacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtgg

SEQ ID NO: 30 - DNA, 150 bp, Unknown
  Bacteria of family Lachnospiraceae
tacgtagggggcaagcgttatccggatttactgggtgtaaagggagcgtagacggcatgacaagccagatgtgaaaacccagggctcaaccctgggactgcatttggaactgccaggctggagtgcaggagaggtaagcggaattcctag

SEQ ID NO: 31 - DNA, 150 bp, Unknown
  Bacteria of family Lachnospiraceae
tacgtagggggcaagcgttatccggatttactgggtgtaaagggagcgtagacggcacagcaagtctgaagtgaaatccccgggctcaacccgggaactgctttggaaactgttgggctggagtgctggagaggcaagcggaattcctag

SEQ ID NO: 32 - DNA, 150 bp, Unknown
  Bacteria of genus Bacteroides
tacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtgg

SEQ ID NO: 33 - DNA, 150 bp, Unknown
  Bacteria of family Rikenellaceae
tacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatgg

SEQ ID NO: 34 - DNA, 150 bp, Unknown
  Bacteria of family Ruminococcaceae
tacgtaggtggcaagcgttgtccggatttactgggtgtaaagggcgtgtaggcgggtagacaagtcagatgtgaaataccggggctcaactccggggctgcatttgaaactgtatatcttgagtgtcggagaggaaagcggaattcctag

SEQ ID NO: 35 - DNA, 150 bp, Unknown
  Bacteria of genus Paraprevotella
tacggaagatgcgagcgttatccggatttattgggtttaaagggagcgtaggcgggcttttaagtcagcggtcaaatgtcacggctcaaccgtggccagccgttgaaactgcaagccttgagtctgcacagggcacatggaattcgtggt

SEQ ID NO: 36 - DNA, 150 bp, Unknown
  Bacteria of genus Coprococcus
tacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctag

SEQ ID NO: 37 - DNA, 150 bp, Unknown
  Bacteria of genus Paraprevotella
tacggaagatgcgagcgttatccggatttattgggtttaaagggagcgtaggcgggcttttaagtcagcggtcaaatgtcgtggctcaaccatgtcaagccgttgaaactgtaagccttgagtctgcacagggcacatggaattcgtggt

SEQ ID NO: 38 - DNA, 150 bp, Unknown
  Bacteria of family Rikenellaceae
tacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatgg

SEQ ID NO: 39 - DNA, 120 bp, Unknown
  Bacteria of genus Bacteroides
atccggatttattgggtttaaagggagcgtaggtggacagttaagtcagttgtgaaagtttgcggctcaaccgtaaaattgcagttgatactggctgtcttgagtacagtagaggtgggc

SEQ ID NO: 40 - DNA, 120 bp, Eisenbergiella tayi
atccggatttattgggtttaaagggtgcgtaggtggtgatttaagtcagcggtgaaagtttgtggctcaaccataaaattgccgttgaaactgggttacttgagtgtgtttgaggtaggc

SEQ ID NO: 41 - DNA, 120 bp, Eisenbergiella tayi
atccggatttactgggtgtaaagggagcgtagacggcatggcaagccagatgtgaaaacccagggctcaaccttgggattgcatttggaactgccaggctggagtgcaggagaggtaagc

SEQ ID NO: 42 - DNA, 120 bp, Parabacteroides merdae
atccggatttactgggtgtaaagggagcgtagacggcatggcaagccagatgtgaaaacccagggctcaaccttgggattgcatttggaactgtcaggctggagtgcaggagaggtaagc